SEQUENCE LISTING (208 sequences)

SEQ ID NO: 1 - DNA, 20 bp, Homo sapiens
  source 1..20: mol_type = genomic DNA
atgatctctgctgccagtag

SEQ ID NO: 2 - DNA, 20 bp, Homo sapiens
  source 1..20: mol_type = genomic DNA
atcgcgcctggatgtcaacg

SEQ ID NO: 3 - DNA, 20 bp, Homo sapiens
  source 1..20: mol_type = genomic DNA
attgtgcagtgcatcggatt

SEQ ID NO: 4 - DNA, 20 bp, Homo sapiens
  source 1..20: mol_type = genomic DNA
gtcaaacagtactgctacgg

SEQ ID NO: 5 - protein, 209 aa, synthetic construct
LEPGEKPYKCPECGKSFSRSDKLTEHQRTHTGEKPYKCPECGKSFSTKNSLTEHQRTHTGEKPYKCPECGKSFSQSGDLRRHQRTHTGEKPYKCPECGKSFSTTGALTEHQRTHTGEKPYKCPECGKSFSDSGNLRVHQRTHTGEKPYKCPECGKSFSQRAHLERHQRTHTGEKPYKCPECGKSFSTSGELVRHQRTHTGEKPTGKKTS

SEQ ID NO: 6 - protein, 209 aa, synthetic construct
LEPGEKPYKCPECGKSFSSPADLTRHQRTHTGEKPYKCPECGKSFSDPGHLVRHQRTHTGEKPYKCPECGKSFSRSDNLVRHQRTHTGEKPYKCPECGKSFSQSSSLVRHQRTHTGEKPYKCPECGKSFSTSHSLTEHQRTHTGEKPYKCPECGKSFSRNDALTEHQRTHTGEKPYKCPECGKSFSRNDALTEHQRTHTGEKPTGKKTS

SEQ ID NO: 7 - protein, 209 aa, synthetic construct
LEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTS

SEQ ID NO: 8 - protein, 209 aa, synthetic construct
LEPGEKPYKCPECGKSFSHTGHLLEHQRTHTGEKPYKCPECGKSFSTTGNLTVHQRTHTGEKPYKCPECGKSFSDKKDLTRHQRTHTGEKPYKCPECGKSFSQSGDLRRHQRTHTGEKPYKCPECGKSFSQRAHLERHQRTHTGEKPYKCPECGKSFSRSDKLTEHQRTHTGEKPYKCPECGKSFSRTDTLRDHQRTHTGEKPTGKKTS

SEQ ID NO: 9 - protein, 209 aa, synthetic construct
LEPGEKPYKCPECGKSFSHTGHLLEHQRTHTGEKPYKCPECGKSFSTSGNLTEHQRTHTGEKPYKCPECGKSFSTSGNLVRHQRTHTGEKPYKCPECGKSFSQLAHLRAHQRTHTGEKPYKCPECGKSFSERSHLREHQRTHTGEKPYKCPECGKSFSDPGHLVRHQRTHTGEKPYKCPECGKSFSTHLDLIRHQRTHTGEKPTGKKTS

SEQ ID NO: 10 - protein, 209 aa, synthetic construct
LEPGEKPYKCPECGKSFSDPGALVRHQRTHTGEKPYKCPECGKSFSERSHLREHQRTHTGEKPYKCPECGKSFSRSDHLTNHQRTHTGEKPYKCPECGKSFSERSHLREHQRTHTGEKPYKCPECGKSFSRSDHLTNHQRTHTGEKPYKCPECGKSFSDPGHLVRHQRTHTGEKPYKCPECGKSFSRSDKLVRHQRTHTGEKPTGKKTS

SEQ ID NO: 11 - protein, 209 aa, synthetic construct
LEPGEKPYKCPECGKSFSRNDALTEHQRTHTGEKPYKCPECGKSFSDCRDLARHQRTHTGEKPYKCPECGKSFSDPGHLVRHQRTHTGEKPYKCPECGKSFSQSGHLTEHQRTHTGEKPYKCPECGKSFSREDNLHTHQRTHTGEKPYKCPECGKSFSTKNSLTEHQRTHTGEKPYKCPECGKSFSRADNLTEHQRTHTGEKPTGKKTS

SEQ ID NO: 12 - protein, 209 aa, synthetic construct
LEPGEKPYKCPECGKSFSRSDKLTEHQRTHTGEKPYKCPECGKSFSRRDELNVHQRTHTGEKPYKCPECGKSFSRSDHLTNHQRTHTGEKPYKCPECGKSFSSPADLTRHQRTHTGEKPYKCPECGKSFSRSDHLTNHQRTHTGEKPYKCPECGKSFSSKKALTEHQRTHTGEKPYKCPECGKSFSTHLDLIRHQRTHTGEKPTGKKTS

SEQ ID NO: 13 - protein, 209 aa, synthetic construct
LEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTS

SEQ ID NO: 14 - protein, 209 aa, synthetic construct
LEPGEKPYKCPECGKSFSQSGDLRRHQRTHTGEKPYKCPECGKSFSQSGHLTEHQRTHTGEKPYKCPECGKSFSERSHLREHQRTHTGEKPYKCPECGKSFSDPGHLVRHQRTHTGEKPYKCPECGKSFSRNDTLTEHQRTHTGEKPYKCPECGKSFSRADNLTEHQRTHTGEKPYKCPECGKSFSTHLDLIRHQRTHTGEKPTGKKTS

SEQ ID NO: 15 - protein, 209 aa, synthetic construct
LEPGEKPYKCPECGKSFSHTGHLLEHQRTHTGEKPYKCPECGKSFSSKKALTEHQRTHTGEKPYKCPECGKSFSDCRDLARHQRTHTGEKPYKCPECGKSFSHTGHLLEHQRTHTGEKPYKCPECGKSFSRNDALTEHQRTHTGEKPYKCPECGKSFSQSGDLRRHQRTHTGEKPYKCPECGKSFSDSGNLRVHQRTHTGEKPTGKKTS

SEQ ID NO: 16 - protein, 209 aa, synthetic construct
LEPGEKPYKCPECGKSFSQSSSLVRHQRTHTGEKPYKCPECGKSFSRSDHLTNHQRTHTGEKPYKCPECGKSFSQLAHLRAHQRTHTGEKPYKCPECGKSFSQSSNLVRHQRTHTGEKPYKCPECGKSFSRSDNLVRHQRTHTGEKPYKCPECGKSFSRSDELVRHQRTHTGEKPYKCPECGKSFSQLAHLRAHQRTHTGEKPTGKKTS

SEQ ID NO: 17 - skipped

SEQ ID NO: 18 - protein, 96 aa, synthetic construct
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 19 - protein, 385 aa, synthetic construct
SKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 20-21 - skipped (2 entries)

SEQ ID NO: 22 - protein, 367 aa, synthetic construct
MAPKKKRKVGIHGVPAAGSSGSLEPGEKPYKCPECGKSFSRSDKLTEHQRTHTGEKPYKCPECGKSFSTKNSLTEHQRTHTGEKPYKCPECGKSFSQSGDLRRHQRTHTGEKPYKCPECGKSFSTTGALTEHQRTHTGEKPYKCPECGKSFSDSGNLRVHQRTHTGEKPYKCPECGKSFSQRAHLERHQRTHTGEKPYKCPECGKSFSTSGELVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 23 - protein, 367 aa, synthetic construct
MAPKKKRKVGIHGVPAAGSSGSLEPGEKPYKCPECGKSFSSPADLTRHQRTHTGEKPYKCPECGKSFSDPGHLVRHQRTHTGEKPYKCPECGKSFSRSDNLVRHQRTHTGEKPYKCPECGKSFSQSSSLVRHQRTHTGEKPYKCPECGKSFSTSHSLTEHQRTHTGEKPYKCPECGKSFSRNDALTEHQRTHTGEKPYKCPECGKSFSRNDALTEHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 24 - protein, 367 aa, synthetic construct
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 25 - protein, 367 aa, synthetic construct
MAPKKKRKVGIHGVPAAGSSGSLEPGEKPYKCPECGKSFSHTGHLLEHQRTHTGEKPYKCPECGKSFSTTGNLTVHQRTHTGEKPYKCPECGKSFSDKKDLTRHQRTHTGEKPYKCPECGKSFSQSGDLRRHQRTHTGEKPYKCPECGKSFSQRAHLERHQRTHTGEKPYKCPECGKSFSRSDKLTEHQRTHTGEKPYKCPECGKSFSRTDTLRDHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 26 - protein, 367 aa, synthetic construct
MAPKKKRKVGIHGVPAAGSSGSLEPGEKPYKCPECGKSFSHTGHLLEHQRTHTGEKPYKCPECGKSFSTSGNLTEHQRTHTGEKPYKCPECGKSFSTSGNLVRHQRTHTGEKPYKCPECGKSFSQLAHLRAHQRTHTGEKPYKCPECGKSFSERSHLREHQRTHTGEKPYKCPECGKSFSDPGHLVRHQRTHTGEKPYKCPECGKSFSTHLDLIRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 27 - protein, 367 aa, synthetic construct
MAPKKKRKVGIHGVPAAGSSGSLEPGEKPYKCPECGKSFSDPGALVRHQRTHTGEKPYKCPECGKSFSERSHLREHQRTHTGEKPYKCPECGKSFSRSDHLTNHQRTHTGEKPYKCPECGKSFSERSHLREHQRTHTGEKPYKCPECGKSFSRSDHLTNHQRTHTGEKPYKCPECGKSFSDPGHLVRHQRTHTGEKPYKCPECGKSFSRSDKLVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 28 - protein, 661 aa, synthetic construct
MAPKKKRKVGIHGVPAAGSSGSLEPGEKPYKCPECGKSFSRNDALTEHQRTHTGEKPYKCPECGKSFSDCRDLARHQRTHTGEKPYKCPECGKSFSDPGHLVRHQRTHTGEKPYKCPECGKSFSQSGHLTEHQRTHTGEKPYKCPECGKSFSREDNLHTHQRTHTGEKPYKCPECGKSFSTKNSLTEHQRTHTGEKPYKCPECGKSFSRADNLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 29 - protein, 661 aa, synthetic construct
MAPKKKRKVGIHGVPAAGSSGSLEPGEKPYKCPECGKSFSRSDKLTEHQRTHTGEKPYKCPECGKSFSRRDELNVHQRTHTGEKPYKCPECGKSFSRSDHLTNHQRTHTGEKPYKCPECGKSFSSPADLTRHQRTHTGEKPYKCPECGKSFSRSDHLTNHQRTHTGEKPYKCPECGKSFSSKKALTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 30 - protein, 661 aa, synthetic construct
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 31 - protein, 661 aa, synthetic construct
MAPKKKRKVGIHGVPAAGSSGSLEPGEKPYKCPECGKSFSQSGDLRRHQRTHTGEKPYKCPECGKSFSQSGHLTEHQRTHTGEKPYKCPECGKSFSERSHLREHQRTHTGEKPYKCPECGKSFSDPGHLVRHQRTHTGEKPYKCPECGKSFSRNDTLTEHQRTHTGEKPYKCPECGKSFSRADNLTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 32 - protein, 661 aa, synthetic construct
MAPKKKRKVGIHGVPAAGSSGSLEPGEKPYKCPECGKSFSHTGHLLEHQRTHTGEKPYKCPECGKSFSSKKALTEHQRTHTGEKPYKCPECGKSFSDCRDLARHQRTHTGEKPYKCPECGKSFSHTGHLLEHQRTHTGEKPYKCPECGKSFSRNDALTEHQRTHTGEKPYKCPECGKSFSQSGDLRRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 33 - protein, 661 aa, synthetic construct
MAPKKKRKVGIHGVPAAGSSGSLEPGEKPYKCPECGKSFSQSSSLVRHQRTHTGEKPYKCPECGKSFSRSDHLTNHQRTHTGEKPYKCPECGKSFSQLAHLRAHQRTHTGEKPYKCPECGKSFSQSSNLVRHQRTHTGEKPYKCPECGKSFSRSDNLVRHQRTHTGEKPYKCPECGKSFSRSDELVRHQRTHTGEKPYKCPECGKSFSQLAHLRAHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYA

SEQ ID NO: 34-37 - skipped (4 entries)

SEQ ID NO: 38 - DNA, 627 bp, synthetic construct
  source 1..627: mol_type = other DNA
ctggagcccggcgagaaaccctacaagtgccccgagtgcggcaaatccttctctagaagcgacaaactgaccgaacatcagaggacccacaccggcgagaagccttataagtgtcccgaatgcggcaaatccttcagcaccaagaactctctgacagaacaccagagaacacataccggagagaaaccttataaatgccccgagtgcggcaagtccttctcccagtccggcgatctgaggagacaccaaagaacacataccggcgaaaagccttacaagtgccccgagtgtggaaagagcttctccaccaccggcgctctgaccgagcaccagagaacacacaccggcgagaaaccctataaatgtcccgagtgtggcaaatccttcagcgacagcggcaatctgagagtgcaccaaagaacccataccggcgaaaaaccctacaaatgccccgagtgcggcaaatccttcagccagagggcccatctggagaggcaccaaaggacacacaccggagaaaagccctacaagtgtcccgagtgtggaaaaagctttagcacaagcggcgagctggtgaggcatcaaaggacccacaccggcgaaaagcccaccggcaaaaagaccagc

SEQ ID NO: 39 - DNA, 627 bp, synthetic construct
  source 1..627: mol_type = other DNA
ctggagcccggcgagaagccctacaagtgccccgagtgcggaaagtccttcagctcccccgccgatctgacaagacatcagagaacccataccggcgagaaaccttacaaatgccccgaatgtggcaagtcctttagcgatcccggacatctggtgaggcaccagaggacacacaccggcgaaaagccctataaatgtcccgagtgtggaaagagcttttctagaagcgacaatctcgtgagacaccagagaacccacaccggagagaagccttacaagtgccccgagtgcggcaaatccttcagccagagctcctctctggtgaggcaccaaaggacccacaccggcgagaaaccttataagtgtcccgagtgtggcaaaagcttcagcacctcccactctctgaccgagcatcaaagaacccacaccggcgaaaaaccttataaatgccccgagtgtggcaaatccttcagcagaaatgacgctctgacagagcaccaaagaacacataccggagaaaagccctacaaatgccccgagtgtggaaaatccttttctagaaacgatgctctgaccgaacaccaaagaacacacaccggcgaaaagcctaccggaaaaaagaccagc

SEQ ID NO: 40 - DNA, 627 bp, synthetic construct
  source 1..627: mol_type = other DNA
ctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagc

SEQ ID NO: 41 - DNA, 627 bp, synthetic construct
  source 1..627: mol_type = other DNA
ctggagcccggcgaaaagccttataaatgtcccgaatgcggcaagagctttagccacaccggccatctgctggaacaccaaaggacccataccggcgaaaagccctataagtgccccgagtgtggcaagagcttcagcaccaccggcaatctgacagtccatcagaggacccacaccggagagaaaccctataaatgccccgagtgtggaaagtccttctccgacaagaaggatctgacaagacaccagaggacccataccggcgagaaaccctacaaatgccccgagtgcggcaaatccttctcccagagcggcgatctgaggagacatcaaagaacacataccggcgaaaaaccctataagtgccccgaatgcggcaagtccttcagccagagggcccatctggaaaggcatcagaggacacacaccggcgagaagccttacaaatgtcccgagtgcggaaagagcttctctagaagcgacaagctgaccgagcatcagaggacccacaccggagaaaaaccttacaagtgccccgagtgcggcaaaagcttcagcagaaccgacacactgagagatcaccaaaggacacacaccggcgagaaacccaccggcaaaaagaccagc

SEQ ID NO: 42 - DNA, 627 bp, synthetic construct
  source 1..627: mol_type = other DNA
ctggagcccggcgagaagccttataagtgccccgagtgtggcaagagctttagccacaccggccatctgctggagcatcaaaggacacacaccggagaaaagccctataagtgccccgagtgtggcaaatccttcagcacctccggcaatctcaccgaacaccagagaacacacaccggagaaaaaccttacaaatgtcccgagtgtggaaagagcttttccaccagcggcaatctggtgagacatcaaagaacacataccggcgaaaaaccctataaatgccccgagtgtggaaaatccttctcccaactggcccatctgagggcccaccagaggacacataccggagaaaaaccctacaaatgccccgaatgcggaaaaagcttctccgagagaagccatctgagagagcaccaaaggacccataccggagaaaagccttacaagtgtcccgagtgcggaaaaagctttagcgatcccggacatctggtgagacatcagagaacccacaccggcgaaaagccttataaatgtcccgaatgtggcaagtcctttagcacccatctggatctgattagacaccaaagaacccacaccggcgagaaacccaccggaaaaaagaccagc

SEQ ID NO: 43 - DNA, 627 bp, synthetic construct
  source 1..627: mol_type = other DNA
ctggagcccggcgaaaagccttacaaatgtcccgagtgcggaaagtccttcagcgaccccggcgctctggtgagacatcaaagaacacataccggcgagaaaccttataaatgccccgaatgtggaaaatccttcagcgaaagaagccatctgagggaacaccagaggacccacaccggcgaaaaaccttataagtgccccgaatgcggaaaaagcttttctagaagcgatcatctgaccaaccatcagagaacacacaccggcgaaaagccctataaatgtcccgagtgtggcaaatcctttagcgagaggtcccatctgagagagcaccagaggacacataccggagagaagccctacaagtgccccgagtgtggcaagagctttagcagaagcgaccatctgaccaatcatcaaaggacccacaccggagagaagccttacaagtgtcccgagtgcggaaagtccttttccgatcccggccacctcgtgaggcaccaaagaacccataccggcgagaaaccctacaaatgccccgagtgtggaaagagcttctccagaagcgacaagctggtgaggcatcagaggacacacaccggcgaaaaacccaccggcaagaaaaccagc

SEQ ID NO: 44 - DNA, 627 bp, synthetic construct
  source 1..627: mol_type = other DNA
ctggagcccggagagaagccctacaaatgccccgagtgtggaaagagcttctctagaaatgacgctctgacagaacaccagaggacccataccggcgagaaaccttacaaatgccccgagtgcggaaaaagctttagcgattgcagagatctggctagacatcagagaacacacaccggcgagaagccctataagtgccccgaatgcggcaagagctttagcgaccccggccatctggtgagacatcaaaggacacataccggagaaaaaccttacaagtgccccgagtgcggaaagtccttctcccagagcggccatctcaccgagcatcaaaggacccacaccggcgaaaagccttataaatgtcccgaatgtggcaagtccttctctagagaggataatctgcacacccatcagaggacccacaccggcgaaaagccttataaatgccccgaatgtggaaagtccttttccaccaagaactctctgaccgagcatcagaggacacacaccggagagaaaccctataaatgtcccgagtgtggcaagagcttcagcagagccgacaatctgacagagcaccaaagaacacataccggcgaaaagcccaccggcaaaaagaccagc

SEQ ID NO: 45 - DNA, 627 bp, synthetic construct
  source 1..627: mol_type = other DNA
ctggagcccggcgagaaaccctacaagtgccccgagtgtggcaaatccttctctagatccgacaaactgaccgaacatcagaggacccataccggcgaaaaaccttataaatgtcccgagtgcggaaagtccttctctagaagggacgagctgaacgtgcatcagagaacacataccggcgagaagccctataaatgccccgaatgcggcaaaagcttctctagaagcgatcatctgaccaaccaccagagaacccataccggagaaaagccttacaagtgtcccgaatgtggaaaatccttcagctcccccgccgatctgaccagacaccaaaggacccacaccggcgagaagccctataaatgccccgagtgcggcaagagcttttccagatccgaccatctgaccaatcatcaaagaacccacaccggcgaaaagccttataaatgtcccgagtgcggcaaatccttttccagcaagaaggctctgaccgagcatcaaaggacccataccggcgagaagccttacaaatgccccgagtgtggaaagtcctttagcacccatctggatctgattagacaccagaggacacacaccggagagaaacccaccggcaaaaagaccagc

SEQ ID NO: 46 - DNA, 627 bp, synthetic construct
  source 1..627: mol_type = other DNA
ctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagc

SEQ ID NO: 47 - DNA, 627 bp, synthetic construct
  source 1..627: mol_type = other DNA
ctggagcccggcgagaaaccctacaagtgccccgagtgtggaaaaagctttagccaaagcggcgatctgaggagacaccaaagaacacacaccggcgagaagccctacaaatgtcccgagtgcggaaagagcttcagccagagcggccatctgaccgagcatcagagaacccataccggcgaaaaaccttataagtgccccgagtgtggaaagtccttctccgagagatcccatctgagagaacaccagaggacacacaccggcgaaaaaccttataagtgtcccgagtgcggaaagtccttcagcgatcccggccatctggtgagacatcaaaggacacataccggcgaaaaaccttataagtgtcccgaatgcggcaagagctttagcagaaacgacacactcaccgaacaccagaggacccacaccggcgagaaaccctacaaatgccccgagtgcggcaaatccttttctagagccgacaatctgaccgaacaccagaggacccataccggagaaaagccttacaaatgtcccgagtgtggcaaatccttctccacccatctggatctgattagacaccaaagaacacataccggagaaaagcccaccggaaaaaagaccagc

SEQ ID NO: 48 - DNA, 627 bp, synthetic construct
  source 1..627: mol_type = other DNA
ctggagcccggcgaaaaaccctataagtgccccgaatgtggaaagagcttcagccataccggccatctgctggaacaccaaaggacacacaccggcgagaaaccttacaagtgtcccgagtgcggaaaaagcttctcctccaaaaaggctctcaccgagcaccagagaacacataccggcgaaaagccttataagtgccccgagtgtggcaaatccttttccgactgtagagatctggccagacatcaaagaacccacaccggagagaaaccttataaatgccccgagtgcggcaagtcctttagccataccggccatctgctggagcaccagaggacccataccggcgagaagccttacaaatgccccgagtgcggcaaaagcttcagcagaaatgacgctctgaccgagcatcaaaggacccataccggcgaaaagccctacaagtgtcccgagtgtggaaagtccttctcccagagcggcgatctgaggagacaccagagaacacacaccggcgagaaaccctataaatgtcccgagtgcggaaagagctttagcgacagcggcaatctgagggtgcatcaaagaacacacaccggcgaaaaacccaccggaaaaaagacaagc

SEQ ID NO: 49 - DNA, 538 bp, synthetic construct
  source 1..538: mol_type = other DNA
caccggcgaaaagccttataagtgccccgagtgcggcaagtccttctctagaagcgatcacctcaccaatcatcagaggacacataccggagagaagccctataagtgccccgagtgcggcaagagctttagccagctggctcatctgagagctcaccaaagaacccataccggcgagaagccttacaaatgccccgagtgtggaaaatccttttcccagtccagcaacctcgtcagacatcaaaggacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagc

SEQ ID NO: 50 - skipped

SEQ ID NO: 51 - DNA, 288 bp, synthetic construct
  source 1..288: mol_type = other DNA
gacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtg

SEQ ID NO: 52 - DNA, 1155 bp, synthetic construct
  source 1..1155: mol_type = other DNA
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 53-54 - skipped (2 entries)

SEQ ID NO: 55 - DNA, 1345 bp, synthetic construct
  source 1..1345: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaaaccctacaagtgccccgagtgcggcaaatccttctctagaagcgacaaactgaccgaacatcagaggacccacaccggcgagaagccttataagtgtcccgaatgcggcaaatccttcagcaccaagaactctctgacagaacaccagagaacacataccggagagaaaccttataaatgccccgagtgcggcaagtccttctcccagtccggcgatctgaggagacaccaaagaacacataccggcgaaaagccttacaagtgccccgagtgtggaaagagcttctccaccaccggcgctctgaccgagcaccagagaacacacaccggcgagaaaccctataaatgtcccgagtgtggcaaatccttcagcgacagcggcaatctgagagtgcaccaaagaacccataccggcgaaaaaccctacaaatgccccgagtgcggcaaatccttcagccagagggcccatctggagaggcaccaaaggacacacaccggagaaaagccctacaagtgtcccgagtgtggaaaaagctttagcacaagcggcgagctggtgaggcatcaaaggacccacaccggcgaaaagcccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 56 - DNA, 1345 bp, synthetic construct
  source 1..1345: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaagccctacaagtgccccgagtgcggaaagtccttcagctcccccgccgatctgacaagacatcagagaacccataccggcgagaaaccttacaaatgccccgaatgtggcaagtcctttagcgatcccggacatctggtgaggcaccagaggacacacaccggcgaaaagccctataaatgtcccgagtgtggaaagagcttttctagaagcgacaatctcgtgagacaccagagaacccacaccggagagaagccttacaagtgccccgagtgcggcaaatccttcagccagagctcctctctggtgaggcaccaaaggacccacaccggcgagaaaccttataagtgtcccgagtgtggcaaaagcttcagcacctcccactctctgaccgagcatcaaagaacccacaccggcgaaaaaccttataaatgccccgagtgtggcaaatccttcagcagaaatgacgctctgacagagcaccaaagaacacataccggagaaaagccctacaaatgccccgagtgtggaaaatccttttctagaaacgatgctctgaccgaacaccaaagaacacacaccggcgaaaagcctaccggaaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - DNA, 1345 bp, synthetic construct
  source 1..1345: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 58 - DNA, 1345 bp, synthetic construct
  source 1..1345: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttataaatgtcccgaatgcggcaagagctttagccacaccggccatctgctggaacaccaaaggacccataccggcgaaaagccctataagtgccccgagtgtggcaagagcttcagcaccaccggcaatctgacagtccatcagaggacccacaccggagagaaaccctataaatgccccgagtgtggaaagtccttctccgacaagaaggatctgacaagacaccagaggacccataccggcgagaaaccctacaaatgccccgagtgcggcaaatccttctcccagagcggcgatctgaggagacatcaaagaacacataccggcgaaaaaccctataagtgccccgaatgcggcaagtccttcagccagagggcccatctggaaaggcatcagaggacacacaccggcgagaagccttacaaatgtcccgagtgcggaaagagcttctctagaagcgacaagctgaccgagcatcagaggacccacaccggagaaaaaccttacaagtgccccgagtgcggcaaaagcttcagcagaaccgacacactgagagatcaccaaaggacacacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - DNA, 1345 bp, synthetic construct
  source 1..1345: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaagccttataagtgccccgagtgtggcaagagctttagccacaccggccatctgctggagcatcaaaggacacacaccggagaaaagccctataagtgccccgagtgtggcaaatccttcagcacctccggcaatctcaccgaacaccagagaacacacaccggagaaaaaccttacaaatgtcccgagtgtggaaagagcttttccaccagcggcaatctggtgagacatcaaagaacacataccggcgaaaaaccctataaatgccccgagtgtggaaaatccttctcccaactggcccatctgagggcccaccagaggacacataccggagaaaaaccctacaaatgccccgaatgcggaaaaagcttctccgagagaagccatctgagagagcaccaaaggacccataccggagaaaagccttacaagtgtcccgagtgcggaaaaagctttagcgatcccggacatctggtgagacatcagagaacccacaccggcgaaaagccttataaatgtcccgaatgtggcaagtcctttagcacccatctggatctgattagacaccaaagaacccacaccggcgagaaacccaccggaaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - DNA, 1345 bp, synthetic construct
  source 1..1345: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaagccttacaaatgtcccgagtgcggaaagtccttcagcgaccccggcgctctggtgagacatcaaagaacacataccggcgagaaaccttataaatgccccgaatgtggaaaatccttcagcgaaagaagccatctgagggaacaccagaggacccacaccggcgaaaaaccttataagtgccccgaatgcggaaaaagcttttctagaagcgatcatctgaccaaccatcagagaacacacaccggcgaaaagccctataaatgtcccgagtgtggcaaatcctttagcgagaggtcccatctgagagagcaccagaggacacataccggagagaagccctacaagtgccccgagtgtggcaagagctttagcagaagcgaccatctgaccaatcatcaaaggacccacaccggagagaagccttacaagtgtcccgagtgcggaaagtccttttccgatcccggccacctcgtgaggcaccaaagaacccataccggcgagaaaccctacaaatgccccgagtgtggaaagagcttctccagaagcgacaagctggtgaggcatcagaggacacacaccggcgaaaaacccaccggcaagaaaaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 61 - DNA, 2227 bp, synthetic construct
  source 1..2227: mol_type = other DNA
aggaaataagagagaaaagaagagtaagaagaaatataagagccaccatggcccccaagaagaagcggaaggtgggcatccacggcgtgcccgccgccggcagcagcggatccctggagcccggagagaagccctacaaatgccccgagtgtggaaagagcttctctagaaatgacgctctgacagaacaccagaggacccataccggcgagaaaccttacaaatgccccgagtgcggaaaaagctttagcgattgcagagatctggctagacatcagagaacacacaccggcgagaagccctataagtgccccgaatgcggcaagagctttagcgaccccggccatctggtgagacatcaaaggacacataccggagaaaaaccttacaagtgccccgagtgcggaaagtccttctcccagagcggccatctcaccgagcatcaaaggacccacaccggcgaaaagccttataaatgtcccgaatgtggcaagtccttctctagagaggataatctgcacacccatcagaggacccacaccggcgaaaagccttataaatgccccgaatgtggaaagtccttttccaccaagaactctctgaccgagcatcagaggacacacaccggagagaaaccctataaatgtcccgagtgtggcaagagcttcagcagagccgacaatctgacagagcaccaaagaacacataccggcgaaaagcccaccggcaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - DNA, 2227 bp, synthetic construct
  source 1..2227: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaaaccctacaagtgccccgagtgtggcaaatccttctctagatccgacaaactgaccgaacatcagaggacccataccggcgaaaaaccttataaatgtcccgagtgcggaaagtccttctctagaagggacgagctgaacgtgcatcagagaacacataccggcgagaagccctataaatgccccgaatgcggcaaaagcttctctagaagcgatcatctgaccaaccaccagagaacccataccggagaaaagccttacaagtgtcccgaatgtggaaaatccttcagctcccccgccgatctgaccagacaccaaaggacccacaccggcgagaagccctataaatgccccgagtgcggcaagagcttttccagatccgaccatctgaccaatcatcaaagaacccacaccggcgaaaagccttataaatgtcccgagtgcggcaaatccttttccagcaagaaggctctgaccgagcatcaaaggacccataccggcgagaagccttacaaatgccccgagtgtggaaagtcctttagcacccatctggatctgattagacaccagaggacacacaccggagagaaacccaccggcaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - DNA, 2227 bp, synthetic construct
  source 1..2227: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 64 - DNA, 2080 bp, synthetic construct
  source 1..2080: mol_type = other DNA
agtgtggaaaaagctttagccaaagcggcgatctgaggagacaccaaagaacacacaccggcgagaagccctacaaatgtcccgagtgcggaaagagcttcagccagagcggccatctgaccgagcatcagagaacccataccggcgaaaaaccttataagtgccccgagtgtggaaagtccttctccgagagatcccatctgagagaacaccagaggacacacaccggcgaaaaaccttataagtgtcccgagtgcggaaagtccttcagcgatcccggccatctggtgagacatcaaaggacacataccggcgaaaaaccttataagtgtcccgaatgcggcaagagctttagcagaaacgacacactcaccgaacaccagaggacccacaccggcgagaaaccctacaaatgccccgagtgcggcaaatccttttctagagccgacaatctgaccgaacaccagaggacccataccggagaaaagccttacaaatgtcccgagtgtggcaaatccttctccacccatctggatctgattagacaccaaagaacacataccggagaaaagcccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - DNA, 1929 bp, synthetic construct
  source 1..1929: mol_type = other DNA
gccttataagtgccccgagtgtggcaaatccttttccgactgtagagatctggccagacatcaaagaacccacaccggagagaaaccttataaatgccccgagtgcggcaagtcctttagccataccggccatctgctggagcaccagaggacccataccggcgagaagccttacaaatgccccgagtgcggcaaaagcttcagcagaaatgacgctctgaccgagcatcaaaggacccataccggcgaaaagccctacaagtgtcccgagtgtggaaagtccttctcccagagcggcgatctgaggagacaccagagaacacacaccggcgagaaaccctataaatgtcccgagtgcggaaagagctttagcgacagcggcaatctgagggtgcatcaaagaacacacaccggcgaaaaacccaccggaaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - DNA, 1779 bp, synthetic construct
  source 1..1779: mol_type = other DNA
gacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67-70 - skipped (4 entries)

SEQ ID NO: 71 - DNA, 20 bp, synthetic construct
  source 1..20: mol_type = other DNA
  misc_difference 1: note = a, c, t, g, unknown or other
  misc_difference 3: note = a, c, t, g, unknown or other
nbndccdshagrkgghrshv

SEQ ID NO: 72 - DNA, 20 bp, synthetic construct
  source 1..20: mol_type = other DNA
  misc_difference 18: note = a, c, t, g, unknown or other
  misc_difference 20: note = a, c, t, g, unknown or other
vhsrhggkrgahsdccdnbn

SEQ ID NO: 73-74 - skipped (2 entries)

SEQ ID NO: 75 - DNA, 21 bp, Homo sapiens
  source 1..21: mol_type = genomic DNA
gctggaaaccttgcacctcgg

SEQ ID NO: 76 - DNA, 21 bp, Homo sapiens
  source 1..21: mol_type = genomic DNA
ctgctgccagtagagggcaca

SEQ ID NO: 77 - DNA, 21 bp, Homo sapiens
  source 1..21: mol_type = genomic DNA
gcccagagagggggcggaggg

SEQ ID NO: 78 - DNA, 21 bp, Homo sapiens
  source 1..21: mol_type = genomic DNA
acgcggggagcaaccaatcgc

SEQ ID NO: 79 - DNA, 21 bp, Homo sapiens
  source 1..21: mol_type = genomic DNA
actggcagcagagatcatcgc

SEQ ID NO: 80 - DNA, 21 bp, Homo sapiens
  source 1..21: mol_type = genomic DNA
gggggcaggagcaggagcgtc

SEQ ID NO: 81 - DNA, 21 bp, Homo sapiens
  source 1..21: mol_type = genomic DNA
cagccttagcgaggcgccctg

SEQ ID NO: 82 - DNA, 21 bp, Homo sapiens
  source 1..21: mol_type = genomic DNA
actcacaggacaaggatgcgg

SEQ ID NO: 83 - DNA, 21 bp, Homo sapiens
  source 1..21: mol_type = genomic DNA
agcaaaagaaaatggtaggcg

SEQ ID NO: 84 - DNA, 21 bp, Homo sapiens
  source 1..21: mol_type = genomic DNA
actcagccgggcagccgagca

SEQ ID NO: 85 - DNA, 21 bp, Homo sapiens
  source 1..21: mol_type = genomic DNA
cgtaccaggctgcagggcgcc

SEQ ID NO: 86 - DNA, 21 bp, Homo sapiens
  source 1..21: mol_type = genomic DNA
agagtggaggaaagaagggta

SEQ ID NO: 87 - protein, 386 aa, Homo sapiens
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 88 - protein, 22 aa, synthetic construct
MAPKKKRKVGIHGVPAAGSSGS

SEQ ID NO: 89 - protein, 29 aa, synthetic construct
SGGKRPAATKKAGQAKKKGSYPYDVPDYA

SEQ ID NO: 90 - protein, 9 aa, synthetic construct
YPYDVPDYA

SEQ ID NO: 91 - protein, 1123 aa, synthetic construct
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYASSGGKRPAATKKAGQAKKKKGSYPYDVPDYA

SEQ ID NO: 92 - protein, 1123 aa, synthetic construct
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYASSGGKRPAATKKAGQAKKKKGSYPYDVPDYA

SEQ ID NO: 93 - DNA, 3332 bp, synthetic construct
  source 1..3332: mol_type = other DNA
gcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 94 - DNA, 3012 bp, synthetic construct
  source 1..3012: mol_type = other DNA
tataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 95 - protein, 118 aa, synthetic construct
PSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGS

SEQ ID NO: 96 - DNA, 20 bp, Homo sapiens
  source 1..20: mol_type = genomic DNA
tgccacttccccactaaccc

SEQ ID NO: 97 - DNA, 20 bp, Homo sapiens
  source 1..20: mol_type = genomic DNA
ggccacacaaggaagctgca

SEQ ID NO: 98 - DNA, 20 bp, Homo sapiens
  source 1..20: mol_type = genomic DNA
ccacacaaggaagctgcagg

SEQ ID NO: 99 - DNA, 20 bp, Homo sapiens
  source 1..20: mol_type = genomic DNA
tgattggaatgcaacccgaa

SEQ ID NO: 100 - DNA, 20 bp, Homo sapiens
  source 1..20: mol_type = genomic DNA
ttttgcccttgctaccccaa

SEQ ID NO: 101 - DNA, 20 bp, Homo sapiens
  source 1..20: mol_type = genomic DNA
agctgatggtatccactagg

SEQ ID NO: 102 - DNA, 20 bp, Homo sapiens
  source 1..20: mol_type = genomic DNA
cacatccaagaatgtagtgg

SEQ ID NO: 103 - DNA, 20 bp, Homo sapiens
  source 1..20: mol_type = genomic DNA
gatacagccacaaagctcac

SEQ ID NO: 104 - DNA, 20 bp, Homo sapiens
  source 1..20: mol_type = genomic DNA
attacataacagaatccagg

SEQ ID NO: 105 - DNA, 20 bp, Homo sapiens
  source 1..20: mol_type = genomic DNA
cccttgactgtgctgccacc

SEQ ID NO: 106 - DNA, 20 bp, Homo sapiens
  source 1..20: mol_type = genomic DNA
cagacgaggaacctgaaccc

SEQ ID NO: 107 - DNA, 20 bp, Homo sapiens
  source 1..20: mol_type = genomic DNA
agaatcccttggggtagcaa

SEQ ID NO: 108 - DNA, 20 bp, Homo sapiens
  source 1..20: mol_type = genomic DNA
cagcactctcgctgaccgca

SEQ ID NO: 109 - DNA, 20 bp, Homo sapiens
  source 1..20: mol_type = genomic DNA
gttgagtcatgtgtactctg

SEQ ID NO: 110 - DNA, 20 bp, Homo sapiens
  source 1..20: mol_type = genomic DNA
aggaacaggatgttacaact

SEQ ID NO: 111 - DNA, 20 bp, Homo sapiens
  source 1..20: mol_type = genomic DNA
ggggccactagggacaggat

SEQ ID NO: 112 - DNA, 3650 bp, synthetic construct
  source 1..3650: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 113 - DNA, 3650 bp, synthetic construct
  source 1..3650: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaaaccttacaagtgccccgagtgcggaaagagcttcagcagaagcgacaaactggtgaggcatcaaaggacacataccggagagaagccctataagtgccccgaatgtggcaaatccttttcccagagggctcatctggaaagacaccagaggacccataccggcgaaaaaccctacaaatgtcccgagtgtggaaagagcttttccgatcccggccatctggtcagacatcagaggacacataccggcgaaaagccttacaagtgtcccgaatgcggaaaatccttctccagaagcgacaagctggtgaggcaccaaagaacccacaccggcgaaaaaccctataaatgccccgagtgcggcaagtcctttagccagctggcccatctgagagcccaccagagaacacacaccggagagaagccttataagtgtcccgagtgcggaaagtccttctctagagccgacaatctgaccgaacatcaaaggacacacaccggcgagaaaccttataaatgccccgagtgcggaaaaagcttttccgactgcagagatctggctagacaccagagaacccacaccggcgagaaacccaccggcaaaaagaccagcgctagcggcagcggcggcggcagcggcggcgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggagacccaccccgacagcgagaccgccttcgagatcaagagcagcgtgagcagcggcggcaagcggcccgccgccaccaagaaggccggccaggccaagaagaagaagggcagctacccctacgacgtgcccgactacgccgggtcctacccgtacgacgtgcccgattacgccgccaccaacttctcgctgctgaagcaggccggagatgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgcctgaagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 114 - skipped

SEQ ID NO: 115 - DNA, 627 bp, synthetic construct
  source 1..627: mol_type = other DNA
ctggagcccggcgagaaaccctataaatgccccgaatgcggaaaaagcttcagccagtccagctctctggtgagacatcagaggacacacaccggcgaaaagccttataagtgccccgagtgcggcaagtccttctctagaagcgatcacctcaccaatcatcagaggacacataccggagagaagccctataagtgccccgagtgcggcaagagctttagccagctggctcatctgagagctcaccaaagaacccataccggcgagaagccttacaaatgccccgagtgtggaaaatccttttcccagtccagcaacctcgtcagacatcaaaggacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagc

SEQ ID NO: 116 - DNA, 2227 bp, synthetic construct
  source 1..2227: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaaaccctacaagtgccccgagtgtggaaaaagctttagccaaagcggcgatctgaggagacaccaaagaacacacaccggcgagaagccctacaaatgtcccgagtgcggaaagagcttcagccagagcggccatctgaccgagcatcagagaacccataccggcgaaaaaccttataagtgccccgagtgtggaaagtccttctccgagagatcccatctgagagaacaccagaggacacacaccggcgaaaaaccttataagtgtcccgagtgcggaaagtccttcagcgatcccggccatctggtgagacatcaaaggacacataccggcgaaaaaccttataagtgtcccgaatgcggcaagagctttagcagaaacgacacactcaccgaacaccagaggacccacaccggcgagaaaccctacaaatgccccgagtgcggcaaatccttttctagagccgacaatctgaccgaacaccagaggacccataccggagaaaagccttacaaatgtcccgagtgtggcaaatccttctccacccatctggatctgattagacaccaaagaacacataccggagaaaagcccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - DNA, 2227 bp, synthetic construct
  source 1..2227: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgaaaaaccctataagtgccccgaatgtggaaagagcttcagccataccggccatctgctggaacaccaaaggacacacaccggcgagaaaccttacaagtgtcccgagtgcggaaaaagcttctcctccaaaaaggctctcaccgagcaccagagaacacataccggcgaaaagccttataagtgccccgagtgtggcaaatccttttccgactgtagagatctggccagacatcaaagaacccacaccggagagaaaccttataaatgccccgagtgcggcaagtcctttagccataccggccatctgctggagcaccagaggacccataccggcgagaagccttacaaatgccccgagtgcggcaaaagcttcagcagaaatgacgctctgaccgagcatcaaaggacccataccggcgaaaagccctacaagtgtcccgagtgtggaaagtccttctcccagagcggcgatctgaggagacaccagagaacacacaccggcgagaaaccctataaatgtcccgagtgcggaaagagctttagcgacagcggcaatctgagggtgcatcaaagaacacacaccggcgaaaaacccaccggaaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 118 - DNA, 2227 bp, synthetic construct
  source 1..2227: mol_type = other DNA
aggaaataagagagaaaagaagagtaagaagaaatataagagccaccatggcccccaagaagaagcggaaggtgggcatccacggcgtgcccgccgccggcagcagcggatccctggagcccggcgagaaaccctataaatgccccgaatgcggaaaaagcttcagccagtccagctctctggtgagacatcagaggacacacaccggcgaaaagccttataagtgccccgagtgcggcaagtccttctctagaagcgatcacctcaccaatcatcagaggacacataccggagagaagccctataagtgccccgagtgcggcaagagctttagccagctggctcatctgagagctcaccaaagaacccataccggcgagaagccttacaaatgccccgagtgtggaaaatccttttcccagtccagcaacctcgtcagacatcaaaggacccataccggcgaaaagccttacaagtgtcccgagtgcggaaagtccttctctagatccgacaacctcgtgaggcaccagagaacccacaccggcgagaaaccttacaaatgtcccgagtgtggcaaaagcttttctagaagcgacgagctggtgagacatcaaagaacccataccggcgaaaaaccttataagtgtcccgagtgcggcaaatcctttagccagctggcccatctgagggcccaccagagaacacataccggcgaaaaacccaccggcaaaaagacaagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119 - skipped

SEQ ID NO: 120 - protein, 18 aa, synthetic construct
EGRGSLLTCGDVEENPGP

SEQ ID NO: 121 - protein, 1092 aa, synthetic construct
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGSYPYDVPDYA

SEQ ID NO: 122 - protein, 1092 aa, synthetic construct
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSSGGKRPAATKKAGQAKKKKGSYPYDVPDYAGSYPYDVPDYAATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYA

SEQ ID NO: 123 - DNA, 355 bp, Homo sapiens
  source 1..355: mol_type = genomic DNA
cagagaaggaggaagttaattcacattcttaattttttctaagggcaaaaaaaaaaaaaaaatgcaccagctcattttccatctctgcttgggtcatcagtgtgcattgtgagcctgtacaaaggccttagacggggaatgctgccgagagcatcaccttttatgtcttcttttatatgaaatgtgccacttccccactaaccctggctctgggctctgcctctgctctcctgatggtgtgtttatggtggattcagcattctgggccacacaaggaagctgcagggggtgtccaagttcacatgtccccgcattccaggcgaatgtttctgacattgagcaatgatatggctct

SEQ ID NO: 124 - protein, 46 aa, synthetic construct
ATNFSLLKQAGDVEENPGPTSAGKLGSGEGRGSLLTCGDVEENPGP

SEQ ID NO: 125 - protein, 19 aa, synthetic construct
ATNFSLLKQAGDVEENPGP

SEQ ID NO: 126 - protein, 18 aa, synthetic construct
ATNFSLLKQAGDVEENPG

SEQ ID NO: 127 - protein, 27 aa, synthetic construct
PTSAGKLGSGEGRGSLLTCGDVEENPG

SEQ ID NO: 128 - skipped

SEQ ID NO: 129 - protein, 652 aa, synthetic construct
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 130 - DNA, 2234 bp, synthetic construct
  source 1..2234: mol_type = other DNA
gggaaataagagagaaaagaagagtaagaagaaatataagagccaccatggcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcagcggcggcaagcggcccgccgccaccaagaaggccggccaggccaagaagaagaagggcagctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 131 - RNA, 627 nt, synthetic construct
  source 1..627: mol_type = other RNA
ctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagc

SEQ ID NO: 132 - RNA, 1155 nt, synthetic construct
  source 1..1155: mol_type = other RNA
agcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggc

SEQ ID NO: 133 - protein, 690 aa, synthetic construct
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGSYPYDVPDYAGSYPYDVPDYAATNFSLLKQAGDVEENPG

SEQ ID NO: 134 - protein, 366 aa, synthetic construct
PLEGSSGSGSPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 135 - protein, 6 aa, Simian virus 40
PKKKRK

SEQ ID NO: 136 - protein, 15 aa, synthetic construct
KRPAATKKAGQAKKK

SEQ ID NO: 137 - protein, 13 aa, synthetic construct
ASGSGGGSGGARD

SEQ ID NO: 138 - protein, 10 aa, synthetic construct
ASGSGGGSGG

SEQ ID NO: 139 - protein, 358 aa, synthetic construct
MAPKKKRKVGIHGVPAAGSSGSLEPGEKPYKCPECGKSFSRSDKLTEHQRTHTGEKPYKCPECGKSFSTKNSLTEHQRTHTGEKPYKCPECGKSFSQSGDLRRHQRTHTGEKPYKCPECGKSFSTTGALTEHQRTHTGEKPYKCPECGKSFSDSGNLRVHQRTHTGEKPYKCPECGKSFSQRAHLERHQRTHTGEKPYKCPECGKSFSTSGELVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 140 - protein, 358 aa, synthetic construct
MAPKKKRKVGIHGVPAAGSSGSLEPGEKPYKCPECGKSFSSPADLTRHQRTHTGEKPYKCPECGKSFSDPGHLVRHQRTHTGEKPYKCPECGKSFSRSDNLVRHQRTHTGEKPYKCPECGKSFSQSSSLVRHQRTHTGEKPYKCPECGKSFSTSHSLTEHQRTHTGEKPYKCPECGKSFSRNDALTEHQRTHTGEKPYKCPECGKSFSRNDALTEHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 141 - protein, 358 aa, synthetic construct
MAPKKKRKVGIHGVPAAGSSGSLEPGEKPYKCPECGKSFSRSDKLVRHQRTHTGEKPYKCPECGKSFSQRAHLERHQRTHTGEKPYKCPECGKSFSDPGHLVRHQRTHTGEKPYKCPECGKSFSRSDKLVRHQRTHTGEKPYKCPECGKSFSQLAHLRAHQRTHTGEKPYKCPECGKSFSRADNLTEHQRTHTGEKPYKCPECGKSFSDCRDLARHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 142 - protein, 358 aa, synthetic construct
MAPKKKRKVGIHGVPAAGSSGSLEPGEKPYKCPECGKSFSHTGHLLEHQRTHTGEKPYKCPECGKSFSTTGNLTVHQRTHTGEKPYKCPECGKSFSDKKDLTRHQRTHTGEKPYKCPECGKSFSQSGDLRRHQRTHTGEKPYKCPECGKSFSQRAHLERHQRTHTGEKPYKCPECGKSFSRSDKLTEHQRTHTGEKPYKCPECGKSFSRTDTLRDHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 143 - protein, 358 aa, synthetic construct
MAPKKKRKVGIHGVPAAGSSGSLEPGEKPYKCPECGKSFSHTGHLLEHQRTHTGEKPYKCPECGKSFSTSGNLTEHQRTHTGEKPYKCPECGKSFSTSGNLVRHQRTHTGEKPYKCPECGKSFSQLAHLRAHQRTHTGEKPYKCPECGKSFSERSHLREHQRTHTGEKPYKCPECGKSFSDPGHLVRHQRTHTGEKPYKCPECGKSFSTHLDLIRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 144 - protein, 358 aa, synthetic construct
MAPKKKRKVGIHGVPAAGSSGSLEPGEKPYKCPECGKSFSDPGALVRHQRTHTGEKPYKCPECGKSFSERSHLREHQRTHTGEKPYKCPECGKSFSRSDHLTNHQRTHTGEKPYKCPECGKSFSERSHLREHQRTHTGEKPYKCPECGKSFSRSDHLTNHQRTHTGEKPYKCPECGKSFSDPGHLVRHQRTHTGEKPYKCPECGKSFSRSDKLVRHQRTHTGEKPTGKKTSASGSGGGSGGDAKSLTAWSRTLVTFKDVFVDFTREEWKLLDTAQQILYRNVMLENYKNLVSLGYQLTKPDVILRLEKGEEPWLVEREIHQETHPDSETAFEIKSSVSGGKRPAATKKAGQAKKKKGS

SEQ ID NO: 145 - protein, 652 aa, synthetic construct
MAPKKKRKVGIHGVPAAGSSGSLEPGEKPYKCPECGKSFSRNDALTEHQRTHTGEKPYKCPECGKSFSDCRDLARHQRTHTGEKPYKCPECGKSFSDPGHLVRHQRTHTGEKPYKCPECGKSFSQSGHLTEHQRTHTGEKPYKCPECGKSFSREDNLHTHQRTHTGEKPYKCPECGKSFSTKNSLTEHQRTHTGEKPYKCPECGKSFSRADNLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 146 - protein, 652 aa, synthetic construct
MAPKKKRKVGIHGVPAAGSSGSLEPGEKPYKCPECGKSFSRSDKLTEHQRTHTGEKPYKCPECGKSFSRRDELNVHQRTHTGEKPYKCPECGKSFSRSDHLTNHQRTHTGEKPYKCPECGKSFSSPADLTRHQRTHTGEKPYKCPECGKSFSRSDHLTNHQRTHTGEKPYKCPECGKSFSSKKALTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 147 - protein, 652 aa, synthetic construct
MAPKKKRKVGIHGVPAAGSSGSLEPGEKPYKCPECGKSFSQSGDLRRHQRTHTGEKPYKCPECGKSFSQSGHLTEHQRTHTGEKPYKCPECGKSFSERSHLREHQRTHTGEKPYKCPECGKSFSDPGHLVRHQRTHTGEKPYKCPECGKSFSRNDTLTEHQRTHTGEKPYKCPECGKSFSRADNLTEHQRTHTGEKPYKCPECGKSFSTHLDLIRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 148 - protein, 652 aa, synthetic construct
MAPKKKRKVGIHGVPAAGSSGSLEPGEKPYKCPECGKSFSHTGHLLEHQRTHTGEKPYKCPECGKSFSSKKALTEHQRTHTGEKPYKCPECGKSFSDCRDLARHQRTHTGEKPYKCPECGKSFSHTGHLLEHQRTHTGEKPYKCPECGKSFSRNDALTEHQRTHTGEKPYKCPECGKSFSQSGDLRRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 149 - protein, 652 aa, synthetic construct
MAPKKKRKVGIHGVPAAGSSGSLEPGEKPYKCPECGKSFSQSSSLVRHQRTHTGEKPYKCPECGKSFSRSDHLTNHQRTHTGEKPYKCPECGKSFSQLAHLRAHQRTHTGEKPYKCPECGKSFSQSSNLVRHQRTHTGEKPYKCPECGKSFSRSDNLVRHQRTHTGEKPYKCPECGKSFSRSDELVRHQRTHTGEKPYKCPECGKSFSQLAHLRAHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSSGGKRPAATKKAGQAKKKKGS

SEQ ID NO: 150-153 - skipped (4 entries)

SEQ ID NO: 154 - protein, 209 aa, synthetic construct
LEPGEKPYKCPECGKSFSTSGELVRHQRTHTGEKPYKCPECGKSFSERSHLREHQRTHTGEKPYKCPECGKSFSRNDTLTEHQRTHTGEKPYKCPECGKSFSRADNLTEHQRTHTGEKPYKCPECGKSFSTSGSLVRHQRTHTGEKPYKCPECGKSFSSPADLTRHQRTHTGEKPYKCPECGKSFSDSGNLRVHQRTHTGEKPTGKKTS

SEQ ID NO: 155 - protein, 209 aa, synthetic construct
LEPGEKPYKCPECGKSFSRSDKLVRHQRTHTGEKPYKCPECGKSFSQRANLRAHQRTHTGEKPYKCPECGKSFSRADNLTEHQRTHTGEKPYKCPECGKSFSQSSSLVRHQRTHTGEKPYKCPECGKSFSDKKDLTRHQRTHTGEKPYKCPECGKSFSSPADLTRHQRTHTGEKPYKCPECGKSFSQSGHLTEHQRTHTGEKPTGKKTS

SEQ ID NO: 156 - protein, 209 aa, synthetic construct
LEPGEKPYKCPECGKSFSQSGDLRRHQRTHTGEKPYKCPECGKSFSQSGDLRRHQRTHTGEKPYKCPECGKSFSERSHLREHQRTHTGEKPYKCPECGKSFSTTGNLTVHQRTHTGEKPYKCPECGKSFSHRTTLTNHQRTHTGEKPYKCPECGKSFSDSGNLRVHQRTHTGEKPYKCPECGKSFSQSSSLVRHQRTHTGEKPTGKKTS

SEQ ID NO: 157 - DNA, 627 bp, synthetic construct
  source 1..627: mol_type = other DNA
cttgagcccggagaaaagccatacaaatgtcctgaatgcggaaagtcattttctacgagcggcgaactcgtgcggcaccaaaggactcataccggcgaaaagccttacaaatgcccggagtgcggaaaaagcttctccgagcgctcgcacttgcgggaacaccagcgaacccacacaggggagaaaccgtataagtgcccagagtgcggcaaatcgttctcccggaacgacaccctgaccgaacaccaacgcactcatactggcgaaaaaccttacaagtgccctgagtgtggaaagagcttctcccgcgccgataacctgaccgagcaccagcggacccataccggggaaaagccgtacaagtgtccggaatgcggcaaaagcttcagcacctcgggttccctggtccggcatcagagaactcacaccggagagaaaccctataagtgtcctgagtgcgggaagtccttttcatcgcccgcggacctgactagacaccagaggacccacaccggggagaagccctacaagtgccccgaatgtggaaagtccttctccgactccggcaacctccgggtgcaccagcgcacccacactggagagaagccgaccggaaagaaaacttcc

SEQ ID NO: 158 - DNA, 627 bp, synthetic construct
  source 1..627: mol_type = other DNA
ctggaacccggagaaaaaccttataagtgccctgaatgcggaaagtcattctcgaggtcggacaagctcgtgcggcaccagaggacacacaccggggaaaagccatacaagtgtcccgaatgtggaaagtccttcagccaacgcgccaacctgagagctcatcagcggactcacactggcgaaaaaccgtacaaatgccccgaatgcggcaaaagcttctcccgcgccgacaacttgaccgagcaccagcggacccataccggcgaaaagccgtacaagtgcccggagtgtgggaagtcgttcagccagtcctcttccctcgtgcgccaccaacgcacccatactggggagaagccctataagtgtcctgagtgtggcaaatcattcagcgataagaaggatcttacccggcaccaacggactcataccggagagaagccttacaagtgccccgagtgcggaaagagcttctcgtccccggcggacctgactagacaccagcgcacccacaccggagaaaagccctacaagtgcccagagtgcgggaagtccttttcccaatccggtcacctgactgagcaccagagaacccacacgggagagaaaccgaccggaaagaaaacctcc

SEQ ID NO: 159 - DNA, 627 bp, synthetic construct
  source 1..627: mol_type = other DNA
ttggaacccggagaaaagccatacaaatgccccgaatgcggaaagtcgttcagccagtccggcgacctcagacggcaccaacggactcacaccggcgaaaaaccgtacaagtgcccagagtgcggcaaaagctttagccagtcgggcgatctgcggagacatcagcgcactcacactggtgaaaagccctacaagtgtcctgagtgcgggaagtccttcagcgagcgctcccatcttcgcgagcaccagagaacccacactggagaaaaaccttataagtgccctgagtgtggcaaatccttctcaaccaccggcaacctgactgtgcaccagcggacccacacaggggagaagccttacaagtgcccggagtgtgggaagtcattctcccatcggacgaccctgaccaaccaccagaggacccatactggcgaaaagccgtataagtgtccggagtgcggaaagagcttctccgactccggaaacctcagggtgcaccaacgcacccacaccggagagaagccgtacaaatgtcccgaatgtggaaagtccttctcccaatcctcttcgctggtccggcaccagcgaactcataccggggaaaagcccaccggaaagaaaacctcg

SEQ ID NO: 160 - protein, 659 aa, synthetic construct
MAPKKKRKVGIHGVPAAGSSGSLEPGEKPYKCPECGKSFSTSGELVRHQRTHTGEKPYKCPECGKSFSERSHLREHQRTHTGEKPYKCPECGKSFSRNDTLTEHQRTHTGEKPYKCPECGKSFSRADNLTEHQRTHTGEKPYKCPECGKSFSTSGSLVRHQRTHTGEKPYKCPECGKSFSSPADLTRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGSYPYDVPDYA

SEQ ID NO: 161 - protein, 659 aa, synthetic construct
MAPKKKRKVGIHGVPAAGSSGSLEPGEKPYKCPECGKSFSRSDKLVRHQRTHTGEKPYKCPECGKSFSQRANLRAHQRTHTGEKPYKCPECGKSFSRADNLTEHQRTHTGEKPYKCPECGKSFSQSSSLVRHQRTHTGEKPYKCPECGKSFSDKKDLTRHQRTHTGEKPYKCPECGKSFSSPADLTRHQRTHTGEKPYKCPECGKSFSQSGHLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGSYPYDVPDYA

SEQ ID NO: 162 - protein, 659 aa, synthetic construct
MAPKKKRKVGIHGVPAAGSSGSLEPGEKPYKCPECGKSFSQSGDLRRHQRTHTGEKPYKCPECGKSFSQSGDLRRHQRTHTGEKPYKCPECGKSFSERSHLREHQRTHTGEKPYKCPECGKSFSTTGNLTVHQRTHTGEKPYKCPECGKSFSHRTTLTNHQRTHTGEKPYKCPECGKSFSDSGNLRVHQRTHTGEKPYKCPECGKSFSQSSSLVRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGSYPYDVPDYA

SEQ ID NO: 163 - protein, 650 aa, synthetic construct
MAPKKKRKVGIHGVPAAGSSGSLEPGEKPYKCPECGKSFSTSGELVRHQRTHTGEKPYKCPECGKSFSERSHLREHQRTHTGEKPYKCPECGKSFSRNDTLTEHQRTHTGEKPYKCPECGKSFSRADNLTEHQRTHTGEKPYKCPECGKSFSTSGSLVRHQRTHTGEKPYKCPECGKSFSSPADLTRHQRTHTGEKPYKCPECGKSFSDSGNLRVHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGS

SEQ ID NO: 164 - protein, 650 aa, synthetic construct
MAPKKKRKVGIHGVPAAGSSGSLEPGEKPYKCPECGKSFSRSDKLVRHQRTHTGEKPYKCPECGKSFSQRANLRAHQRTHTGEKPYKCPECGKSFSRADNLTEHQRTHTGEKPYKCPECGKSFSQSSSLVRHQRTHTGEKPYKCPECGKSFSDKKDLTRHQRTHTGEKPYKCPECGKSFSSPADLTRHQRTHTGEKPYKCPECGKSFSQSGHLTEHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGS

SEQ ID NO: 165 - protein, 650 aa, synthetic construct
MAPKKKRKVGIHGVPAAGSSGSLEPGEKPYKCPECGKSFSQSGDLRRHQRTHTGEKPYKCPECGKSFSQSGDLRRHQRTHTGEKPYKCPECGKSFSERSHLREHQRTHTGEKPYKCPECGKSFSTTGNLTVHQRTHTGEKPYKCPECGKSFSHRTTLTNHQRTHTGEKPYKCPECGKSFSDSGNLRVHQRTHTGEKPYKCPECGKSFSQSSSLVRHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGSGGKRPAATKKAGQAKKKKGS

SEQ ID NO: 166 - DNA, 2255 bp, synthetic construct
  source 1..2255: mol_type = other DNA
aggaaataagagagaaaagaagagtaagaagaaatataagagccaccatggcccccaagaagaagcggaaggtgggcatccacggcgtgcccgccgccggcagcagcggatcccttgagcccggagaaaagccatacaaatgtcctgaatgcggaaagtcattttctacgagcggcgaactcgtgcggcaccaaaggactcataccggcgaaaagccttacaaatgcccggagtgcggaaaaagcttctccgagcgctcgcacttgcgggaacaccagcgaacccacacaggggagaaaccgtataagtgcccagagtgcggcaaatcgttctcccggaacgacaccctgaccgaacaccaacgcactcatactggcgaaaaaccttacaagtgccctgagtgtggaaagagcttctcccgcgccgataacctgaccgagcaccagcggacccataccggggaaaagccgtacaagtgtccggaatgcggcaaaagcttcagcacctcgggttccctggtccggcatcagagaactcacaccggagagaaaccctataagtgtcctgagtgcgggaagtccttttcatcgcccgcggacctgactagacaccagaggacccacaccggggagaagccctacaagtgccccgaatgtggaaagtccttctccgactccggcaacctccgggtgcaccagcgcacccacactggagagaagccgaccggaaagaaaacttccgcctccggttcgggaggaggctcaggaggagcgagagattccaaggtcgagaacaagaccaagaagctgcgggtgttcgaggcctttgctggcatcggagcccagaggaaggccctcgagaaggtccgcaaggatgagtacgagatcgtgggactcgcggagtggtacgtgcccgccattgtgatgtaccaggccatccataacaacttccacactaagctggagtacaagtccgtgtcccgggaggaaatgattgactacctggagaataagaccctgtcatggaactctaagaaccccgtgtcgaacggttactggaagagaaagaaggatgacgaactgaagattatctacaacgcgatcaagctgagcgagaaggagggcaacatcttcgacatccgggacctctacaagcgcaccttgaagaacatcgatctgctgacctactccttcccgtgccaagacctgagccagcagggcatccagaaggggatgaaacggggctccggtactcgcagcggcttgctgtgggaaattgagcgggccctggatagcaccgagaagaacgacttgccgaagtatcttctcatggaaaacgtcggggctctccttcacaagaagaacgaggaagaactgaaccagtggaagcaaaagctggaatccctcggataccagaactccattgaggtcctgaacgccgccgacttcggatcgtcgcaagccagacggagggtgttcatgattagcactctgaacgaattcgtggaactgccgaagggcgacaagaagcctaagtccatcaagaaggtgctgaacaagatcgtgtccgagaaggacattctcaacaatctgctgaagtacaacctgacagagttcaagaaaaccaagtccaacatcaacaaggcctccttgattggttactcaaagttcaacagcgagggatacgtgtacgaccccgaattcactggacccactctgaccgcctccggagcaaactctaggattaagatcaaggacggctccaacatccggaagatgaactccgacgaaacctttctgtacatcggcttcgactcgcaagacggaaagcgcgtgaacgagatcgaatttcttaccgaaaaccagaagatcttcgtgtgcggcaattcaatctccgtggaagtcctggaagcgattatcgacaagatcggaggcagtggtggaaagcgcccagcagccactaagaaggccggacaggccaagaagaagaagggatcctacccttacgatgtgccggattacgcttgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 167 - DNA, 2255 bp, synthetic construct
  source 1..2255: mol_type = other DNA
aggaaataagagagaaaagaagagtaagaagaaatataagagccaccatggcccccaagaagaagcggaaggtgggcatccacggcgtgcccgccgccggcagcagcggatccctggaacccggagaaaaaccttataagtgccctgaatgcggaaagtcattctcgaggtcggacaagctcgtgcggcaccagaggacacacaccggggaaaagccatacaagtgtcccgaatgtggaaagtccttcagccaacgcgccaacctgagagctcatcagcggactcacactggcgaaaaaccgtacaaatgccccgaatgcggcaaaagcttctcccgcgccgacaacttgaccgagcaccagcggacccataccggcgaaaagccgtacaagtgcccggagtgtgggaagtcgttcagccagtcctcttccctcgtgcgccaccaacgcacccatactggggagaagccctataagtgtcctgagtgtggcaaatcattcagcgataagaaggatcttacccggcaccaacggactcataccggagagaagccttacaagtgccccgagtgcggaaagagcttctcgtccccggcggacctgactagacaccagcgcacccacaccggagaaaagccctacaagtgcccagagtgcgggaagtccttttcccaatccggtcacctgactgagcaccagagaacccacacgggagagaaaccgaccggaaagaaaacctccgcctccggttcgggaggaggctcaggaggagcgagagattccaaggtcgagaacaagaccaagaagctgcgggtgttcgaggcctttgctggcatcggagcccagaggaaggccctcgagaaggtccgcaaggatgagtacgagatcgtgggactcgcggagtggtacgtgcccgccattgtgatgtaccaggccatccataacaacttccacactaagctggagtacaagtccgtgtcccgggaggaaatgattgactacctggagaataagaccctgtcatggaactctaagaaccccgtgtcgaacggttactggaagagaaagaaggatgacgaactgaagattatctacaacgcgatcaagctgagcgagaaggagggcaacatcttcgacatccgggacctctacaagcgcaccttgaagaacatcgatctgctgacctactccttcccgtgccaagacctgagccagcagggcatccagaaggggatgaaacggggctccggtactcgcagcggcttgctgtgggaaattgagcgggccctggatagcaccgagaagaacgacttgccgaagtatcttctcatggaaaacgtcggggctctccttcacaagaagaacgaggaagaactgaaccagtggaagcaaaagctggaatccctcggataccagaactccattgaggtcctgaacgccgccgacttcggatcgtcgcaagccagacggagggtgttcatgattagcactctgaacgaattcgtggaactgccgaagggcgacaagaagcctaagtccatcaagaaggtgctgaacaagatcgtgtccgagaaggacattctcaacaatctgctgaagtacaacctgacagagttcaagaaaaccaagtccaacatcaacaaggcctccttgattggttactcaaagttcaacagcgagggatacgtgtacgaccccgaattcactggacccactctgaccgcctccggagcaaactctaggattaagatcaaggacggctccaacatccggaagatgaactccgacgaaacctttctgtacatcggcttcgactcgcaagacggaaagcgcgtgaacgagatcgaatttcttaccgaaaaccagaagatcttcgtgtgcggcaattcaatctccgtggaagtcctggaagcgattatcgacaagatcggaggcagtggtggaaagcgcccagcagccactaagaaggccggacaggccaagaagaagaagggatcctacccttacgatgtgccggattacgcttgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 168 - DNA, 2255 bp, synthetic construct
  source 1..2255: mol_type = other DNA
aggaaataagagagaaaagaagagtaagaagaaatataagagccaccatggcccccaagaagaagcggaaggtgggcatccacggcgtgcccgccgccggcagcagcggatccttggaacccggagaaaagccatacaaatgccccgaatgcggaaagtcgttcagccagtccggcgacctcagacggcaccaacggactcacaccggcgaaaaaccgtacaagtgcccagagtgcggcaaaagctttagccagtcgggcgatctgcggagacatcagcgcactcacactggtgaaaagccctacaagtgtcctgagtgcgggaagtccttcagcgagcgctcccatcttcgcgagcaccagagaacccacactggagaaaaaccttataagtgccctgagtgtggcaaatccttctcaaccaccggcaacctgactgtgcaccagcggacccacacaggggagaagccttacaagtgcccggagtgtgggaagtcattctcccatcggacgaccctgaccaaccaccagaggacccatactggcgaaaagccgtataagtgtccggagtgcggaaagagcttctccgactccggaaacctcagggtgcaccaacgcacccacaccggagagaagccgtacaaatgtcccgaatgtggaaagtccttctcccaatcctcttcgctggtccggcaccagcgaactcataccggggaaaagcccaccggaaagaaaacctcggcctccggttcgggaggaggctcaggaggagcgagagattccaaggtcgagaacaagaccaagaagctgcgggtgttcgaggcctttgctggcatcggagcccagaggaaggccctcgagaaggtccgcaaggatgagtacgagatcgtgggactcgcggagtggtacgtgcccgccattgtgatgtaccaggccatccataacaacttccacactaagctggagtacaagtccgtgtcccgggaggaaatgattgactacctggagaataagaccctgtcatggaactctaagaaccccgtgtcgaacggttactggaagagaaagaaggatgacgaactgaagattatctacaacgcgatcaagctgagcgagaaggagggcaacatcttcgacatccgggacctctacaagcgcaccttgaagaacatcgatctgctgacctactccttcccgtgccaagacctgagccagcagggcatccagaaggggatgaaacggggctccggtactcgcagcggcttgctgtgggaaattgagcgggccctggatagcaccgagaagaacgacttgccgaagtatcttctcatggaaaacgtcggggctctccttcacaagaagaacgaggaagaactgaaccagtggaagcaaaagctggaatccctcggataccagaactccattgaggtcctgaacgccgccgacttcggatcgtcgcaagccagacggagggtgttcatgattagcactctgaacgaattcgtggaactgccgaagggcgacaagaagcctaagtccatcaagaaggtgctgaacaagatcgtgtccgagaaggacattctcaacaatctgctgaagtacaacctgacagagttcaagaaaaccaagtccaacatcaacaaggcctccttgattggttactcaaagttcaacagcgagggatacgtgtacgaccccgaattcactggacccactctgaccgcctccggagcaaactctaggattaagatcaaggacggctccaacatccggaagatgaactccgacgaaacctttctgtacatcggcttcgactcgcaagacggaaagcgcgtgaacgagatcgaatttcttaccgaaaaccagaagatcttcgtgtgcggcaattcaatctccgtggaagtcctggaagcgattatcgacaagatcggaggcagtggtggaaagcgcccagcagccactaagaaggccggacaggccaagaagaagaagggatcctacccttacgatgtgccggattacgcttgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 169 - protein, 204 aa, synthetic construct
LEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTS

SEQ ID NO: 170 - protein, 204 aa, synthetic construct
LEPGEKPYKCPECGKSFSQKSSLIAHQRTHTGEKPYKCPECGKSFSHKNALQNHQRTHTGEKPYKCPECGKSFSQSSNLVRHQRTHTGEKPYKCPECGKSFSRNDALTEHQRTHTGEKPYKCPECGKSFSDKKDLTRHQRTHTGEKPYKCPECGKSFSQAGHLASHQRTHTGEKPYKCPECGKSFSDKKDLTRHQRTHTGKKTS

SEQ ID NO: 171 - protein, 204 aa, synthetic construct
LEPGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGEKPYKCPECGKSFSREDNLHTHQRTHTGKKTS

SEQ ID NO: 172 - protein, 204 aa, synthetic construct
LEPGEKPYKCPECGKSFSRSDHLTTHQRTHTGEKPYKCPECGKSFSQKSSLIAHQRTHTGEKPYKCPECGKSFSRRDELNVHQRTHTGEKPYKCPECGKSFSQSGDLRRHQRTHTGEKPYKCPECGKSFSQAGHLASHQRTHTGEKPYKCPECGKSFSTSGNLTEHQRTHTGEKPYKCPECGKSFSQAGHLASHQRTHTGKKTS

SEQ ID NO: 173 - DNA, 612 bp, synthetic construct
  source 1..612: mol_type = other DNA
ctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctct

SEQ ID NO: 174 - DNA, 612 bp, synthetic construct
  source 1..612: mol_type = other DNA
cttgaacccggggagaagccctacaagtgcccggaatgcggaaagagcttcagccagaagtcctcgctgatcgcgcaccagaggactcacaccggcgaaaagccatacaagtgtcctgagtgtggcaaatccttctcgcacaagaacgcactgcagaaccaccagagaacccacaccggggaaaagccgtataagtgccccgaatgtggaaagtcgttcagccagtcatccaacctcgtgcgccatcaaaggactcataccggagagaaaccttacaaatgccctgaatgcggcaaatctttctcccggaatgatgccctgaccgagcaccagcgcactcacacgggagagaagccgtacaaatgtccggagtgcggaaagtccttctccgacaagaaggacttgaccagacaccagcggacccatactggcgaaaaaccctataagtgtccagagtgcgggaagtcctttagccaagccggtcacctcgcttcccaccaacggacccacacaggagaaaagccttataaatgccccgagtgcggcaaaagcttctccgataagaaggacctgactcggcatcagcgcacccataccggaaagaaaacctca

SEQ ID NO: 175 - DNA, 612 bp, synthetic construct
  source 1..612: mol_type = other DNA
ctggagcctggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccaccagagaacccatactggcgagaagccatacaaatgcccggaatgtggaaagagtttctcgcgcgaggacaacctccacacccatcagcgcacccataccggcaaaaagacctct

SEQ ID NO: 176 - DNA, 612 bp, synthetic construct
  source 1..612: mol_type = other DNA
ctggaacccggagagaaaccctacaaatgcccagagtgcggcaaatcgttctcacggtccgatcacctcaccacccaccagaggacccataccggggagaagccttacaagtgtcctgagtgtggaaagtccttcagccaaaagtcctcgctgatcgcacaccagcgcacgcacactggggaaaagccatataaatgcccggagtgtggcaaatccttctcccgccgcgacgaactgaacgtgcaccagagaacccacactggagagaagccgtataagtgtccggagtgcggaaagagcttctcgcaatccggggaccttcggagacatcagaggacacacactggcgaaaagccctataagtgccctgagtgcgggaagtcctttagccaagccggtcacctggcctcccaccaacggactcacaccggcgaaaaaccgtacaagtgccccgaatgcggaaagtcgttctctacctccggaaacttgaccgaacaccagcggacccacaccggagaaaagccgtacaaatgtcctgaatgcggcaaaagcttcagccaggccggtcatctcgcgagccatcagcggactcatactggcaaaaagacctca

SEQ ID NO: 177 - protein, 367 aa, synthetic construct
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 178 - protein, 367 aa, synthetic construct
MAPKKKRKVGIHGVPAAGSSGSLEPGEKPYKCPECGKSFSQKSSLIAHQRTHTGEKPYKCPECGKSFSHKNALQNHQRTHTGEKPYKCPECGKSFSQSSNLVRHQRTHTGEKPYKCPECGKSFSRNDALTEHQRTHTGEKPYKCPECGKSFSDKKDLTRHQRTHTGEKPYKCPECGKSFSQAGHLASHQRTHTGEKPYKCPECGKSFSDKKDLTRHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 179 - protein, 367 aa, synthetic construct
MAPKKKRKVGIHGVPAAGSSGSLEPGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGEKPYKCPECGKSFSREDNLHTHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 180 - protein, 367 aa, synthetic construct
MAPKKKRKVGIHGVPAAGSSGSLEPGEKPYKCPECGKSFSRSDHLTTHQRTHTGEKPYKCPECGKSFSQKSSLIAHQRTHTGEKPYKCPECGKSFSRRDELNVHQRTHTGEKPYKCPECGKSFSQSGDLRRHQRTHTGEKPYKCPECGKSFSQAGHLASHQRTHTGEKPYKCPECGKSFSTSGNLTEHQRTHTGEKPYKCPECGKSFSQAGHLASHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 181 - protein, 1082 aa, synthetic construct
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYA

SEQ ID NO: 182 - protein, 1082 aa, synthetic construct
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYA

SEQ ID NO: 183 - protein, 358 aa, synthetic construct
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 184 - protein, 358 aa, synthetic construct
MAPKKKRKVGIHGVPAAGSSGSLEPGEKPYKCPECGKSFSQKSSLIAHQRTHTGEKPYKCPECGKSFSHKNALQNHQRTHTGEKPYKCPECGKSFSQSSNLVRHQRTHTGEKPYKCPECGKSFSRNDALTEHQRTHTGEKPYKCPECGKSFSDKKDLTRHQRTHTGEKPYKCPECGKSFSQAGHLASHQRTHTGEKPYKCPECGKSFSDKKDLTRHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 185 - protein, 358 aa, synthetic construct
MAPKKKRKVGIHGVPAAGSSGSLEPGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGEKPYKCPECGKSFSREDNLHTHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 186 - protein, 358 aa, synthetic construct
MAPKKKRKVGIHGVPAAGSSGSLEPGEKPYKCPECGKSFSRSDHLTTHQRTHTGEKPYKCPECGKSFSQKSSLIAHQRTHTGEKPYKCPECGKSFSRRDELNVHQRTHTGEKPYKCPECGKSFSQSGDLRRHQRTHTGEKPYKCPECGKSFSQAGHLASHQRTHTGEKPYKCPECGKSFSTSGNLTEHQRTHTGEKPYKCPECGKSFSQAGHLASHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 187 - protein, 1073 aa, synthetic construct
MAPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGS

SEQ ID NO: 188 - protein, 1073 aa, synthetic construct
MAPKKKRKVGIHGVPAAGSSGSLEPGEKPYKCPECGKSFSERSHLREHQRTHTGEKPYKCPECGKSFSRSDNLVRHQRTHTGEKPYKCPECGKSFSDCRDLARHQRTHTGEKPYKCPECGKSFSTSGELVRHQRTHTGEKPYKCPECGKSFSTTGNLTVHQRTHTGEKPYKCPECGKSFSRSDKLVRHQRTHTGEKPYKCPECGKSFSRTDTLRDHQRTHTGKKTSASGSGGGSGGARDDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPSSGGKRPAATKKAGQAKKKKGSYPYDVPDYAGSATNFSLLKQAGDVEENPGPTSAGKLGSGEGRGSLLTCGDVEENPGPLEGSSGSGSPKKKRKVGIHGVPAAGSSGSLEPGEKPYKCPECGKSFSRSDDLVRHQRTHTGEKPYKCPECGKSFSREDNLHTHQRTHTGEKPYKCPECGKSFSRSDHLTTHQRTHTGEKPYKCPECGKSFSQRANLRAHQRTHTGEKPYKCPECGKSFSQLAHLRAHQRTHTGEKPYKCPECGKSFSQRANLRAHQRTHTGEKPYKCPECGKSFSERSHLREHQRTHTGEKPTGKKTSASGSGGGSGGARDSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGGPSGGKRPAATKKAGQAKKKKGS

SEQ ID NO: 189 - DNA, 1376 bp, synthetic construct
  source 1..1376: mol_type = other DNA
aggaaataagagagaaaagaagagtaagaagaaatataagagccaccatggcccccaagaagaagcggaaggtgggcatccacggcgtgcccgccgccggcagcagcggatccctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 190 - DNA, 21 bp, Mus sp.
  source 1..21: mol_type = genomic DNA
aacacagttcagccgagcgct

SEQ ID NO: 191 - DNA, 21 bp, Mus sp.
  source 1..21: mol_type = genomic DNA
cgaacaaccgtacagaaaggg

SEQ ID NO: 192 - DNA, 21 bp, Mus sp.
  source 1..21: mol_type = genomic DNA
gtaaacagtaatagcgcagca

SEQ ID NO: 193 - DNA, 1376 bp, synthetic construct
  source 1..1376: mol_type = other DNA
aggaaataagagagaaaagaagagtaagaagaaatataagagccaccatggcccccaagaagaagcggaaggtgggcatccacggcgtgcccgccgccggcagcagcggatcccttgaacccggggagaagccctacaagtgcccggaatgcggaaagagcttcagccagaagtcctcgctgatcgcgcaccagaggactcacaccggcgaaaagccatacaagtgtcctgagtgtggcaaatccttctcgcacaagaacgcactgcagaaccaccagagaacccacaccggggaaaagccgtataagtgccccgaatgtggaaagtcgttcagccagtcatccaacctcgtgcgccatcaaaggactcataccggagagaaaccttacaaatgccctgaatgcggcaaatctttctcccggaatgatgccctgaccgagcaccagcgcactcacacgggagagaagccgtacaaatgtccggagtgcggaaagtccttctccgacaagaaggacttgaccagacaccagcggacccatactggcgaaaaaccctataagtgtccagagtgcgggaagtcctttagccaagccggtcacctcgcttcccaccaacggacccacacaggagaaaagccttataaatgccccgagtgcggcaaaagcttctccgataagaaggacctgactcggcatcagcgcacccataccggaaagaaaacctcagctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 194 - DNA, 1376 bp, synthetic construct
  source 1..1376: mol_type = other DNA
aggaaataagagagaaaagaagagtaagaagaaatataagagccaccatggcccccaagaagaagcggaaggtgggcatccacggcgtgcccgccgccggcagcagcggatccctggagcctggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccaccagagaacccatactggcgagaagccatacaaatgcccggaatgtggaaagagtttctcgcgcgaggacaacctccacacccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 195 - DNA, 1376 bp, synthetic construct
  source 1..1376: mol_type = other DNA
aggaaataagagagaaaagaagagtaagaagaaatataagagccaccatggcccccaagaagaagcggaaggtgggcatccacggcgtgcccgccgccggcagcagcggatccctggaacccggagagaaaccctacaaatgcccagagtgcggcaaatcgttctcacggtccgatcacctcaccacccaccagaggacccataccggggagaagccttacaagtgtcctgagtgtggaaagtccttcagccaaaagtcctcgctgatcgcacaccagcgcacgcacactggggaaaagccatataaatgcccggagtgtggcaaatccttctcccgccgcgacgaactgaacgtgcaccagagaacccacactggagagaagccgtataagtgtccggagtgcggaaagagcttctcgcaatccggggaccttcggagacatcagaggacacacactggcgaaaagccctataagtgccctgagtgcgggaagtcctttagccaagccggtcacctggcctcccaccaacggactcacaccggcgaaaaaccgtacaagtgccccgaatgcggaaagtcgttctctacctccggaaacttgaccgaacaccagcggacccacaccggagaaaagccgtacaaatgtcctgaatgcggcaaaagcttcagccaggccggtcatctcgcgagccatcagcggactcatactggcaaaaagacctcagctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 196 - DNA, 3521 bp, synthetic construct
  source 1..3521: mol_type = other DNA
gggaaataagagagaaaagaagagtaagaagaaatataagagccaccatggccccaaagaagaagagaaaggtcggaattcatggcgtgcccgcagccggcagcagcggttccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgccgggtccgccaccaacttctcgctgctgaagcaggccggagacgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 197 - DNA, 3521 bp, synthetic construct
  source 1..3521: mol_type = other DNA
gggaaataagagagaaaagaagagtaagaagaaatataagagccaccatggccccaaagaagaagagaaaggtcggaattcatggcgtgcccgcagccggcagcagcggttccctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctacccctacgacgtgcccgactacgccgggtccgccaccaacttctcgctgctgaagcaggccggagacgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagctacccctacgacgtgcccgactacgcctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 198 - skipped

SEQ ID NO: 199 - DNA, 21 bp, Homo sapiens
  source 1..21: mol_type = genomic DNA
acggggaatgctgccgagagc

SEQ ID NO: 200 - DNA, 21 bp, Homo sapiens
  source 1..21: mol_type = genomic DNA
acctgaaccctggaaattata

SEQ ID NO: 201 - DNA, 21 bp, Homo sapiens
  source 1..21: mol_type = genomic DNA
tagacggggaatgctgccgag

SEQ ID NO: 202 - DNA, 21 bp, Homo sapiens
  source 1..21: mol_type = genomic DNA
tgacattgagcaatgatatgg

SEQ ID NO: 203 - DNA, 21 bp, Homo sapiens
  source 1..21: mol_type = genomic DNA
agcaaaagaaaatggtaggcg

SEQ ID NO: 204 - DNA, 21 bp, Homo sapiens
  source 1..21: mol_type = genomic DNA
acggggaatgctgccgagagc

SEQ ID NO: 205 - DNA, 21 bp, Homo sapiens
  source 1..21: mol_type = genomic DNA
acggggaatgctgccgagagc

SEQ ID NO: 206 - DNA, 21 bp, Homo sapiens
  source 1..21: mol_type = genomic DNA
agcaaaagaaaatggtaggcg

SEQ ID NO: 207 - protein, 5 aa, synthetic construct
GGGGS

SEQ ID NO: 208 - DNA, 3467 bp, synthetic construct
  source 1..3467: mol_type = other DNA
gggaaataagagagaaaagaagagtaagaagaaatataagagccaccatggccccaaagaagaagagaaaggtcggaattcatggcgtgcccgcagccggcagcagcggttccctggagcccggcgagaaaccttacaaatgccccgagtgcggcaagagcttcagcagaagcgacgatctggtgaggcaccaaagaacccacaccggcgaaaaaccttacaagtgtcccgaatgcggaaagtccttcagcagagaggacaatctgcacacccaccagagaacacacaccggagaaaagccttacaagtgccccgaatgcggcaaatccttttctagaagcgatcatctgaccacccaccaaagaacacataccggcgagaagccttacaaatgtcccgagtgcggaaagtccttctcccagagagccaatctgagggctcatcaaaggacccataccggcgaaaagccctacaaatgccccgagtgcggaaaatccttcagccagctggcccatctgagagcccaccaaaggacacacaccggagagaaaccctataagtgccccgagtgtggaaaaagcttttcccagagggccaatctgagggcccatcagaggacccataccggagagaagccttataaatgtcccgagtgcggaaaaagcttcagcgagaggagccatctgagggaacatcaaagaacccacaccggcgaaaaacccaccggaaaaaagaccagcgctagcggcagcggcggcggcagcggcggcgcccgggacagcaaggtggagaacaagaccaagaagctgcgggtgttcgaggccttcgccggcatcggcgcccagcggaaggccctggagaaggtgcggaaggacgagtacgagatcgtgggcctggccgagtggtacgtgcccgccatcgtgatgtaccaggccatccacaacaacttccacaccaagctggagtacaagagcgtgagccgggaggagatgatcgactacctggagaacaagaccctgagctggaacagcaagaaccccgtgagcaacggctactggaagcggaagaaggacgacgagctgaagatcatctacaacgccatcaagctgagcgagaaggagggcaacatcttcgacatccgggacctgtacaagcggaccctgaagaacatcgacctgctgacctacagcttcccctgccaggacctgagccagcagggcatccagaagggcatgaagcggggcagcggcacccggagcggcctgctgtgggagatcgagcgggccctggacagcaccgagaagaacgacctgcccaagtacctgctgatggagaacgtgggcgccctgctgcacaagaagaacgaggaggagctgaaccagtggaagcagaagctggagagcctgggctaccagaacagcatcgaggtgctgaacgccgccgacttcggcagcagccaggcccggcggcgggtgttcatgatcagcaccctgaacgagttcgtggagctgcccaagggcgacaagaagcccaagagcatcaagaaggtgctgaacaagatcgtgagcgagaaggacatcctgaacaacctgctgaagtacaacctgaccgagttcaagaaaaccaagagcaacatcaacaaggccagcctgatcggctacagcaagttcaacagcgagggctacgtgtacgaccccgagttcaccggccccaccctgaccgccagcggcgccaacagccggatcaagatcaaggacggcagcaacatccggaagatgaacagcgacgagaccttcctgtacatcggcttcgacagccaggacggcaagcgggtgaacgagatcgagttcctgaccgagaaccagaagatcttcgtgtgcggcaacagcatcagcgtggaggtgctggaggccatcatcgacaagatcggcggccccagcggcggcaagcggcccgccgccaccaagaaggccggccaggccaagaagaagaagggcagcgggtccgccaccaacttctcgctgctgaagcaggccggagacgtggaagaaaaccctggacctaccagtgccggaaagctcggtagcggagagggtcggggaagcctgcttacttgcggcgacgtggaagagaaccccggtccgctggagggttcgtccggctccggatcccccaagaagaagcggaaggtgggcatccacggcgtgcccgccgccggcagcagcggatccctggagcctggagagaaaccctacaaatgcccggaatgcgggaagtccttttccgaacgctcgcacctgagggaacaccagagaactcacaccggcgaaaaaccctataagtgcccagaatgcggaaagagcttttcacggtcggacaacctcgtgcggcaccaacgcactcataccggagagaagccgtacaagtgtcctgagtgcggaaagtcattctccgactgccgggatttggcccgccaccaaagaacacacactggcgaaaagccctacaagtgcccggagtgtggaaagtccttcagcacttccggagagctggtccggcaccagaggacccacaccggggagaagccttacaaatgtccagagtgcggtaaaagcttctccaccaccggcaacctcaccgtgcaccagcggacccacactggagaaaagccgtataaatgccccgaatgcggcaagagcttctcgcgatccgataagcttgtgcggcatcagagaacgcacactggggaaaagccttataagtgtccggagtgcggcaaatccttctcccgcactgacaccctgcgggaccatcagcgcacccataccggcaaaaagacctctgctagcggcagcggcggcggcagcggcggcgcccgggacgacgccaagagcctgaccgcctggagccggaccctggtgaccttcaaggacgtgttcgtggacttcacccgggaggagtggaagctgctggacaccgcccagcagatcctgtaccggaacgtgatgctggagaactacaagaacctggtgagcctgggctaccagctgaccaagcccgacgtgatcctgcggctggagaagggcgaggagccctggctggtggagcgggagatccaccaggaaacccaccccgacagcgaaaccgccttcgagatcaagagcagcgtgcccagcagcggcggcaagcggcccgccgccaccaagaaggccggccaggccaagaagaagaagggcagctgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0256782 A1
(43) Pub. Date: Sep. 3, 2026

(54) COMBINATION THERAPIES COMPRISING MYC MODULATION

(71) Applicant: FLAGSHIP PIONEERING INNOVATIONS V, INC., Cambridge, MA (US)

(72) Inventors: Abigail Elizabeth Witt, Charlestown, MA (US); Jeremiah Dale Farelli, Marblehead, MA (US); Adam Walter Scheidegger, Somerville, MA (US); William Thomas Senapedis, Millis, MA (US); Jodi Michelle Kennedy, Dedham, MA (US); Houda Belaghzal, Cambridge, MA (US); Defne Yarar, Brookline, MA (US); Eugine Lee, Cambridge, MA (US); Kayleigh Gallagher, Worcester, MA (US)

(21) Appl. No.: 18/877,552

(22) PCT Filed: Jun. 22, 2023
(86) PCT No.: PCT/US2023/068894

§ 371 Date: Dec. 20, 2024

Related U.S. Application Data
(60) Provisional application No. 63/354,646, filed on Jun. 22, 2022.
(60) Provisional application No. 63/489,629, filed on Mar. 10, 2023.

(51) Int. Cl. A61K 31/506; A61K 9/00; A61K 31/47; A61K 48/00; A61P 35/00

(52) U.S. Cl. CPC A61K 31/506; A61K 9/0019; A61K 31/47; A61K 48/0058; A61P 35/00

(57) ABSTRACT

The present disclosure relates to combination therapies. The combination therapy may comprise administration of an expression repressor and a kinase inhibitor. In some embodiments, the expression repressor comprises a targeting moiety that binds a MYC promoter, anchor sequence, or super-enhancer. In some embodiments, the expression repressor comprises an effector moiety that represses transcription or methylates DNA. The compositions and methods can be used, for example, to treat cancers such as HCC.

### CROSS-REFERENCE TO RELATED APPLICATIONS

This application claims priority to U.S. Provisional Application 63/354,646 filed on Jun. 22, 2022, and U.S. Provisional Application 63/489,629 filed on Mar. 10, 2023, the entire contents of which are hereby incorporated by reference.

### SEQUENCE LISTING

The instant application contains a Sequence Listing which has been submitted electronically in XML format and is hereby incorporated by reference in its entirety. Said XML copy, created on Jun. 19, 2023, is named O2057-7034WO_SL.xml and is 282,044 bytes in size.

### BACKGROUND

Mis-regulation of gene expression is the underlying cause of many diseases (e.g., in mammals, e.g., humans) e.g., neoplasia, neurological disorders, metabolic disorders and obesity. The mis-regulation of the transcription factor MYC plays a central role in a variety of human tumors and chronic liver diseases. MYC protein is considered “undruggable” due to various factors, e.g., lack of a defined ligand binding site, physiological function essential to the maintenance of normal tissues. Techniques geared towards modulating the MYC gene expression provides a viable alternative approach in treating these diseases. There is a need for novel tools, systems, and methods to stably alter, e.g., decrease, expression of disease associated genes such as MYC.

### SUMMARY

The disclosure provides, for instance, combination therapies comprising an expression repressor and a kinase inhibitor.

The disclosure provides, among other things, expression repressors and expression repressor systems that may be used to modulate, e.g., decrease, expression of a target gene, e.g., MYC.

In some aspects, the disclosure provides a system comprising a first expression repressor comprising a first targeting moiety and optionally a first effector moiety, wherein the first expression repressor binds to a transcription regulatory element (e.g., a promoter or transcription start site (TSS)) operably linked to a target gene, e.g., MYC or to a sequence proximal to the transcription regulatory element, and a second expression repressor comprising a second targeting moiety and optionally a second effector moiety, wherein the second expression repressor binds to an anchor sequence of an anchor sequence mediated conjunction (ASMC) comprising a target gene, e.g., MYC or to a sequence proximal to the anchor sequence.

In some aspects, the disclosure is directed to a nucleic acid encoding the first expression repressor, second expression repressor, both, or a component thereof (e.g., a gRNA, a mRNA). In some embodiments, the nucleic acid encoding the expression repressor system is a multi-cistronic sequence. In some embodiments, the multi-cistronic sequence is a bi-cistronic sequence.

In some aspects, the disclosure is directed to a method of treating a condition associated with over-expression of a target gene e.g., MYC in a subject, comprising administering an expression repressor, or a system, nucleic acid, or vector described herein to the subject, thereby treating the condition.

Additional features of any of the aforesaid methods or compositions include one or more of the following enumerated embodiments.
Those skilled in the art will recognize or be able to ascertain using no more than routine experimentation, many equivalents to the specific embodiments of the disclosure described herein. Such equivalents are intended to be encompassed by the following enumerated embodiments.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Dec. 15, 2020. When one gene or protein references a plurality of sequence accession numbers, all of the sequence variants are encompassed.

### ENUMERATED EMBODIMENTS

1. An expression repressor comprising:
   a targeting moiety that binds to a MYC promoter, and
   optionally, an effector moiety,
   wherein the expression repressor is capable of decreasing expression of MYC.
2. The expression repressor of embodiment 1, wherein the targeting moiety binds a genomic locus that is within 1400, 1200, 1000, 800, 600, 400, or 200 nt upstream or downstream of SEQ ID NO: 4, 199, or 201.
3. The expression repressor of embodiment 1, wherein the targeting moiety binds a genomic locus comprising at least 14, 15, 16, 17, 18, 19, or 20 nucleotides of the sequence of SEQ ID NO: 4, 77, 82, 83, 85, 199, or 201.
4. An expression repressor comprising:
   a targeting moiety that binds a genomic locus comprising at least 14, 15, 16, 17, 18, 19, or 20 nucleotides of the sequence of SEQ ID NO: 3, 97, 98, 99, 100, 101, 102, 103, 104, 105, 106, 107, 109, 110, 75, 76, 78, 79, 80, 81, 84, 85, 86, 190, 191, 192, 200, or 202 and
   optionally, an effector moiety,
   wherein the expression repressor is capable of decreasing expression of MYC.
5. An expression repressor comprising:
   a targeting moiety that binds a genomic locus comprising at least 16, 17, 18, 19, or 20 nucleotides of the sequence of SEQ ID NO: 2, 77, 82, 83, 199, or 201 and
   optionally, an effector moiety,
   wherein the expression repressor is capable of decreasing expression of MYC.
6. An expression repressor comprising:
   a targeting moiety that binds a MYC locus, and
   an effector moiety comprising MQ1 or a fragment or variant thereof,
   wherein the expression repressor is capable of decreasing expression of MYC.
7. An expression repressor comprising:
   a targeting moiety that binds a locus in MYC super enhancer region,
   optionally an effector moiety, e.g., an effector moiety comprising a DNA methyltransferase, wherein optionally the effector moiety comprises MQ1 or a fragment or variant thereof,
   wherein the expression repressor is capable of decreasing expression of MYC.
8. An expression repressor comprising:
   a targeting moiety that binds a locus in MYC super enhancer region,
   an effector moiety comprising a transcription repressor, wherein optionally the effector moiety comprises KRAB or a fragment or variant thereof,
   wherein the expression repressor is capable of decreasing expression of MYC.
9. The expression repressor of embodiment 7 or 8, wherein the targeting moiety binds a genomic locus comprising at least 14, 15, 16, 17, 18, 19, or 20 nucleotides of the sequence of any of SEQ ID NO: 96-110, 83, 199, 201.
10. The expression repressor of any of embodiments 7-9, wherein the targeting moiety binds a genomic locus comprising at least 14, 15, 16, 17, 18, 19, or 20 nucleotides of the sequence of GRCh37: chr8:129162465-129212140, using the hg19 reference genome.
11. The expression repressor of any of embodiments 7-10, wherein the targeting moiety binds a genomic locus comprising at least 14, 15, 16, 17, 18, 19, or 20 nucleotides of the sequence of SEQ ID NO: 96 or 108.
12. The expression repressor of any of embodiments 7-11, wherein the targeting moiety comprises a zinc finger domain or a TAL effector domain.
13. An expression repressor comprising:
   a targeting moiety that binds a locus, e.g., a MYC locus,
   a first effector moiety comprising EZH2 or a fragment or variant thereof, and
   a second effector moiety comprising KRAB or a fragment or variant thereof,
   wherein the expression repressor is capable of decreasing expression at the locus, e.g., decreasing expression of MYC.
14. The expression repressor of embodiment 13, wherein the targeting moiety binds the MYC promoter, super enhancer region, or anchor sequence.
15. The expression repressor of embodiment 13 or 14, wherein the targeting moiety comprises a TAL effector domain, a CRISPR/Cas domain, or a zinc finger domain.
16. The expression repressor of any of embodiments 13-15, wherein the first effector moiety is N-terminal of the second effector, or wherein the first effector is C-terminal of the second effector moiety.
17. An expression repressor comprising:
   a targeting moiety that binds a MYC locus, wherein the targeting moiety comprises a zinc finger domain, and
   optionally, an effector moiety,
   wherein the expression repressor is capable of decreasing expression of MYC.
18. An expression repressor comprising:
   a targeting moiety comprising a CRISPR/Cas domain, e.g., comprising a catalytically inactive CRISPR/Cas protein, that binds to a transcription regulatory element (e.g., a promoter, an enhancer, a super enhancer, or transcription start site (TSS)) operably linked to a MYC gene or a sequence proximal to said transcription regulatory element; and
   an effector moiety comprising MQ1 or a functional variant or fragment thereof.
19. An expression repressor comprising:
   a targeting moiety comprising a CRISPR/Cas domain, e.g., comprising a catalytically inactive CRISPR/Cas protein, that binds to a transcription regulatory element (e.g., a promoter, an enhancer, or transcription start site (TSS)) operably linked to a MYC gene or a sequence proximal to said transcription regulatory element; and
   an effector moiety comprising MQ1 or a functional variant or fragment thereof.
20. An expression repressor comprising:
   a targeting moiety comprising a CRISPR/Cas domain, e.g., comprising a catalytically inactive CRISPR/Cas protein, that binds to a transcription regulatory element (e.g., a promoter, an enhancer, or transcription start site (TSS)) operably linked to a MYC gene or a sequence proximal to said transcription regulatory element; and
   an effector moiety comprising KRAB or a functional variant or fragment thereof.
21. An expression repressor comprising:
   a targeting moiety comprising a CRISPR/Cas domain, e.g., comprising a catalytically inactive CRISPR/Cas protein, that binds to an anchor sequence of an anchor sequence mediated conjunction (ASMC) comprising a MYC gene or to a sequence proximal to the anchor sequence; and
   an effector moiety comprising KRAB or a functional variant or fragment thereof.
22. An expression repressor comprising:
   a targeting moiety comprising a zinc finger domain that binds to a transcription regulatory element (e.g., a promoter, an enhancer, or transcription start site (TSS)) operably linked to a MYC gene or a sequence proximal to said transcription regulatory element; and
   an effector moiety comprising MQ1 or a functional variant or fragment thereof.
23. An expression repressor comprising:
   a targeting moiety comprising a zinc finger domain that binds to a transcription regulatory element (e.g., a promoter, an enhancer, or transcription start site (TSS)) operably linked to a MYC gene or a sequence proximal to said transcription regulatory element; and
   an effector moiety comprising KRAB or a functional variant or fragment thereof.
24. An expression repressor comprising:
   a targeting moiety that binds a mouse genomic locus comprising at least 14, 15, 16, 17, 18, 19, or 20 nucleotides of the sequence of any of SEQ ID NOs: 190-192 and
   optionally, an effector moiety,
wherein the expression repressor is capable of decreasing expression of MYC.
25. The expression repressor of claim 24, wherein the effector moiety comprises a DNA methyltransferase, e.g., MQ1 or a fragment or variant thereof.
26. The expression repressor of embodiments 24 or 25, wherein the targeting moiety comprises a TAL effector domain, a CRISPR/Cas domain, a zinc finger domain, a tetR domain, a meganuclease domain, or an oligonucleotide.
27. The expression repressor of any of embodiments 24-26, wherein the targeting moiety comprises a zinc finger domain or a TAL effector domain.
28. The expression repressor of any of embodiments 24-27, wherein the expression repressor comprises an amino acid sequence chosen from any of SEQ ID NOs: 160-165, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
29. The expression repressor of any of embodiments 24-28, wherein the expression repressor is encoded by a nucleotide sequence chosen from any of SEQ ID NOs: 166-168, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
30. The expression repressor of any of embodiments 24-29, wherein the targeting moiety comprises an amino acid sequence according to any of SEQ ID NOs: 154-156, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
31. The expression repressor of any of embodiments 24-30, wherein the targeting moiety comprises a nucleic acid sequence according to any of SEQ ID NOs: 157-159, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
32. The expression repressor of any of embodiments 24-31, wherein the effector moiety is a durable effector moiety.
33. The expression repressor of any of embodiments 24-32, wherein the effector moiety is a transient effector moiety.
34. The expression repressor of any of embodiments 24-33, wherein the expression repressor is a fusion molecule.
35. The expression repressor of any of embodiments 24-34, wherein the targeting moiety comprises a zinc finger domain, and the effector moiety comprises an epigenetic modifying moiety, e.g., a DNA methyltransferase, e.g., MQ1 or a fragment or variant thereof.
36. The expression repressor of any of embodiments 18-20, 22, or 23, wherein the regulatory element is part of a cluster of regulatory elements.
37. The expression repressor of any embodiments 18-20, 22, or 23, wherein the regulatory element is located in a non-coding region.
38. The expression repressor of any embodiments 18-20, 22, or 23, wherein the regulatory element is a distal enhancer e.g., located at least 1,000 nt away from a target gene promoter, e.g., MYC.
39. The expression repressor of any embodiments 18-20, 22, 23 or 36-38, wherein the regulatory element increases the expression of a target gene, e.g., MYC.
40. The expression repressor of any embodiments 18-20, 22, 23, or 36-39, wherein the regulatory element contains one or more mutations.
41. The expression repressor of any embodiments 18-20, 22, 23, or 36-40, wherein the regulatory element contains at least one disease-associated single nucleotide polymorphism (SNP).
42. The expression repressor of any of embodiments 18-20, 22, 23, or 36-41, wherein the transcription regulatory element interacts with the promoter of target gene, e.g., MYC through an enhancer docking site.
43. The expression repressor of embodiment 42, wherein the enhancer docking site comprises a nucleotide sequence of according to any of SEQ ID NOs: 71 or 72 herein, or SEQ ID NOs: 73 or 74 as described in International Application WO/2022/132195, which is herein incorporated by reference in its entirety.
44. An expression repressor comprising:
   a targeting moiety comprising a zinc finger domain that binds to an anchor sequence of an anchor sequence mediated conjunction (ASMC) comprising a MYC gene or to a sequence proximal to the anchor sequence; and
   an effector moiety comprising KRAB or a functional variant or fragment thereof.
45. The expression repressor of any of embodiments 1-23 or 36-43, wherein the expression repressor comprises an amino acid sequence chosen from any of SEQ ID NOs: 22-37, 129, 133, 134, 139-149, or 177-186 as described in International Application WO/2022/132195, which is herein incorporated by reference in its entirety, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
46. The expression repressor of any of embodiments 1-23 or 36-45, wherein the expression repressor is encoded by a nucleotide sequence chosen from any of SEQ ID NOs: 55-66, 130, 189, or 193-197 herein, or SEQ ID NOs: 67-70 as described in International Application WO/2022/132195, which is herein incorporated by reference in its entirety, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
47. The expression repressor of any of embodiments 1-23 or 36-46, wherein the targeting moiety comprises an amino acid sequence according to any of SEQ ID NOs: 5-16, or 169-172, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
48. The expression repressor of any of the preceding embodiments, wherein the effector moiety comprises an amino acid sequence according to SEQ ID NOs: 18 19, or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto
49. The expression repressor of any of embodiments 1-12, 17-19, 22, 36-42, or 44-47, wherein the effector moiety is a durable effector moiety.
50. The expression repressor of any of embodiments 1-23, or 36-48 wherein the effector moiety is a transient effector moiety.
51. The expression repressor of any of embodiments 1-12, 17-19, 22, 36-42, or 44-48, wherein the effector moiety comprises a DNA methyltransferase, e.g., MQ1 or a fragment or variant thereof.
52. The expression repressor of any of embodiments 1-23, 36-47 or 49, wherein the effector moiety comprises a transcription repressor, e.g., comprises KRAB or a fragment or variant thereof.
53. The expression repressor of any of the preceding embodiments, wherein the targeting moiety comprises a TAL effector domain, a CRISPR/Cas domain, a zinc finger domain, a tetR domain, a meganuclease domain, or an oligonucleotide.
54. The expression repressor of embodiment 53, wherein the CRISPR/Cas domain binds a gRNA, e.g., a gRNA that binds a genomic locus comprising at least 14, 15, 16, 17, 18, 19, or 20 nucleotides of the sequence of any of SEQ ID NOs: 1-4, e.g., wherein the gRNA comprises a sequence that comprises at least 14, 15, 16, 17, 18, 19, or 20 nucleotides of the sequence of any of SEQ ID NOs: 1-4.
55. The expression repressor of embodiment 53, wherein the CRISPR/Cas domain binds a gRNA, e.g., a gRNA that binds a genomic locus comprising at least 14, 15, 16, 17, 18, 19, or 20 nucleotides of the sequence of any of SEQ ID NOs: 96-110, e.g., wherein the gRNA comprises a sequence that comprises at least 14, 15, 16, 17, 18, 19, or 20 nucleotides of the sequence of any of SEQ ID NOs: 96-110.
56. The expression repressor of any of embodiments 53-55, wherein the CRISPR/Cas domain comprises a Cas protein or Cpf1 protein chosen from Table 1 as described in International Application WO/2022/132195 or a variant (e.g., mutant) of any thereof.
57. The expression repressor of any of embodiments 53-56, wherein the CRISPR/Cas domain comprises a catalytically inactive CRISPR/Cas protein, e.g., dCas9.
58. The expression repressor of embodiment 53, wherein the zinc finger domain binds a genomic locus comprising at least 14, 15, 16, 17, 18, 19, or 20 nucleotides of the sequence of any of SEQ ID NOs: 96-110, e.g., wherein the gRNA comprises a sequence that comprises at least 14, 15, 16, 17, 18, 19, or 20 nucleotides of the sequence of any of SEQ ID NOs: 96-110.
59. The expression repressor of any of embodiments 17, 22, 26-53, or 57, wherein the zinc finger domain comprises 1, 2, 3, 4, 5, 6, 7, 8, 9, or 10 zinc fingers (and optionally no more than 11, 10, 9, 8, 7, 6, or 5 zinc fingers).
60. The expression repressor of any of embodiments 17, 22, 26-53, 57, or 58, wherein the zinc finger domain comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6- 7, 7-10, 7-9, 7-8, 8-10, 8-9, or 9-10 zinc fingers.
61. The expression repressor of any of embodiments 17, 22, 26-53, or 57-59, wherein the zinc finger domain comprises 3 or 9 zinc fingers.
62. The expression repressor of any of the preceding embodiments, which is a fusion molecule.
63. The expression repressor of any of the preceding embodiments, which comprises a linker situated between the targeting domain and the effector domain, optionally wherein the linker comprises an amino sequence according to SEQ ID NO: 137 or SEQ ID NO: 138.
64. The expression repressor of any of embodiments 1-17, 20, 21, 23, 44-48, 50, or 52-57, wherein the targeting moiety comprises a catalytically inactive CRISPR/Cas domain (e.g., dCas9) and the effector moiety comprises a transcription repressor, e.g., KRAB or a fragment or variant thereof.
65. The expression repressor of any of embodiments 1-17, 20, 21, 23, 44-48, 50, 52, or 53-64, wherein the targeting moiety comprises a zinc finger domain, and the effector moiety comprises a transcription repressor, e.g., KRAB or a fragment or variant thereof.
66. The expression repressor of any of embodiments 17, 36-43, 45-47, 53, or 58-63, wherein the targeting moiety comprises a zinc finger domain, and the expression repressor does not comprise an effector moiety.
67. The expression repressor of any of embodiments 1-12, 18-19, 22, 36-43, 45-49, 51, or 53-57 wherein the targeting moiety comprises a catalytically inactive CRISPR/Cas domain (e.g., dCas9) and the effector moiety comprises an epigenetic modifying moiety, e.g., a DNA methyltransferase, e.g., MQ1 or a fragment or variant thereof.
68. The expression repressor of any of embodiments 1-12, 17-19, 22, 36-43, 45-49, 51, 53, or 58-63, wherein the targeting moiety comprises a zinc finger domain, and the effector moiety comprises an epigenetic modifying moiety, e.g., a DNA methyltransferase, e.g., MQ1 or a fragment or variant thereof.
69. The expression repressor of any of the preceding embodiments, which comprises an amino acid sequence of any of SEQ ID NOs: 22-34, 129, 133, 134, 139-149, or 177-186 herein, or any of SEQ ID NOs: 34-37 as described in International Application WO/2022/132195, which is herein incorporated by reference in its entirety, or a sequence having at least 80%, 85%, 90%, 95%, 96%, 97%, 98%, 99% identity thereto.
70. The expression repressor of any of the preceding embodiments, which: (i) comprises one or more nuclear localization signal sequences (NLS), or (ii) does not comprise an NLS.
71. The expression repressor of any of the preceding embodiments, comprising a first NLS at the N terminus, e.g., wherein the first NLS has a sequence of SEQ ID NO: 88.
72. The expression repressor of any of the preceding embodiments, comprising an NLS, e.g., a second NLS, at the C terminus, e.g., having a sequence of SEQ ID NO: 89.
73. The expression repressor of any of the preceding embodiments, wherein the first and the second NLS have the same sequence.
74. The expression repressor of any of embodiments 71-73, wherein the first and the second NLS have different sequences.
75. The expression repressor of any of the preceding embodiments, which comprises an epitope tag.
76. The expression repressor of embodiment 75, wherein the epitope tag is an HA tag.
77. The expression repressor of any of preceding embodiments, wherein the anchor sequence comprises the sequence of SEQ ID NO: 71 or 72, or a sequence with no more than 8, 7, 6, 5, 4, 3, 2, or 1 alterations relative thereto.
78. The expression repressor of any of embodiments 1-77, wherein the anchor sequence comprises a sequence according to SEQ ID NO: 73 or 74 as described in International Application WO/2022/132195, which is herein incorporated by reference in its entirety, or a sequence with no more than 8, 7, 6, 5, 4, 3, 2, or 1 alterations relative thereto.
79. The expression repressor of any of preceding embodiments, wherein the anchor sequence is on the same chromosome as the MYC gene.
80. The expression repressor of any of preceding embodiments, wherein the anchor sequence is upstream of the MYC gene (e.g., upstream of the TSS or upstream of the promoter).
81. The expression repressor of any of preceding embodiments, wherein the anchor sequence is at least 1, 5, 10, 50, 100, or 1000 kilobases away from the MYC gene (e.g., from the TSS or promoter of the MYC gene).
82. The expression repressor of any of preceding embodiments, wherein the anchor sequence is 0.1-0.5, 0.1-1, 0.1-5, 0.1-10, 0.1-50, 0.1-100, 0.1-500, 0.1-1000, 0.5-1, 0.5-5, 0.5-10, 0.5-50, 0.5-100, 0.5-500, 0.5-1000, 1-5, 1-10, 1-50, 1-100, 1-500, 1-1000, 5-10, 5-50, 5-100, 5-500, 5-1000, 10-50, 10-100, 10-500, 10-1000, 50-100, 50-500, 50-1000, 100-500, 100-1000, or 500-1000 kilobases away from the MYC gene (e.g., from the TSS or promoter of the MYC gene).
83. The expression repressor of any of embodiments 1-79 or 81-82, wherein the target sequence is downstream of the MYC gene (e.g., downstream of the TSS or downstream of the promoter).
84. The expression repressor of any of preceding embodiments, wherein the targeting moiety binds to a sequence at chromosome coordinates 128746342-128746364, 128746321-128746343, 128746525-128746547, 128748014-128748036, 129188878-129188900, 129188958-129188980, 129188960-129188982, 129189067-129189089, 129189457-129189479, 129189554-129189576, 129189679-129189701, 129209511-129209533, 129209643-129209665, 129209658-129209680, 129209856-129209878, 129189452-129189474, 129189190-129189212, 129189274-129189296, 129189421- 129189443, 128746405-128746425, 128748069-128748089, 129188825-129188845, or 129188822-129188842 or a sequence proximal thereto.
85. The expression repressor of any of the preceding embodiments, wherein binding of the expression repressor to the target gene locus, e.g., MYC, increases methylation at a site in the target gene locus, e.g., MYC, by 10, 20, 30, 40, 50, 60, 70, 80, 90, or 100% compared to methylation in the absence of the expression repressor, e.g., as measured by ELISA or as described in any of Examples 7 or 28 as described in International Application WO/2022/132195, which is herein incorporated by reference in its entirety, wherein optionally the site assayed for methylation is chr8:129188693-129189048 according to hg19 reference genome, e.g., comprises a sequence according to SEQ ID NO: 123.
86. The expression repressor of any of the preceding embodiments, wherein binding of the expression repressor to the target gene locus, e.g., MYC increases methylation at a site in the target gene locus, e.g., MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as described in Example 28 as described in International Application WO/2022/132195, which is herein incorporated by reference in its entirety.
87. The expression repressor of any of the preceding embodiments, wherein binding of the expression repressor to the MYC locus decreases expression of MYC in a cell by 10, 20, 30, 40, 50, 60, 70, 80, 90, or 100% compared to expression in the absence of the expression repressor, e.g., as measured by ELISA or as described in any of Examples 2-7 or 9 as described in International Application WO/2022/132195, which is herein incorporated by reference in its entirety.
88. The expression repressor of any of the preceding embodiments, wherein binding of the expression repressor to the MYC locus appreciably decreases expression of MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in any of Examples 2-7 or 9 as described in International Application WO/2022/132195, which is herein incorporated by reference in its entirety.
89. The expression repressor of any of the preceding embodiments, wherein binding of the expression repressor to the MYC locus appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
90. The expression repressor of any of embodiments 1-23 or 36-89, wherein the targeting moiety binds to a human genomic locus.
91. The expression repressor of any of embodiments 24-43 49, 51, 53, 56-57, 59-62, 66-68, 70-89, wherein the targeting moiety binds to a mouse genomic locus.
92. The expression repressor of any of the preceding embodiments, wherein binding of the expression repressor to the MYC locus decreases the viability of a cell comprising the MYC locus (e.g., cancer cells).
93. The expression repressor of any of the preceding embodiments, wherein contacting a plurality of cells with the expression repressor or a nucleic acid encoding the expression repressor decreases the viability of the plurality of cells.
94. The expression repressor of any of the preceding embodiments, wherein viability is decreased by 10, 20, 30, 40, 50, 60, 70, 80, 90, or 100% compared to viability in the absence of the first expression repressor, e.g., as measured by CellTiter Glo or as described in any of Examples 2-7 as described in International Application WO/2022/132195, which is herein incorporated by reference in its entirety.
95. The expression repressor of any of the preceding embodiments, wherein administration of the expression repressor results in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
96. The expression repressor of any preceding embodiments, wherein the plurality of cells comprises a plurality of cancer cells and a plurality of non-cancer cells and/or a plurality of infected cells and a plurality of uninfected cells.
97. The expression repressor of any of the preceding embodiments, wherein contacting the plurality of cells with the expression repressor or a nucleic acid encoding the expression repressor decreases the viability of the plurality of cancer cells more than it decreases the viability of the plurality of non-cancer cells.
98. The expression repressor of any of the preceding embodiments, wherein contacting the plurality of cells with the expression repressor or a nucleic acid encoding the expression repressor decreases the viability of the plurality of cancer cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of non-cancer cells.
99. The expression repressor of any of embodiments 92-97, wherein the cancer cells are lung cancer cells, gastric cancer cells, gastrointestinal cancer cells, colorectal cancer cells, pancreatic cancer cells, or hepatic cancer cells.
100. The expression repressor of any of embodiments 92-99, wherein the cancer is hepatocellular carcinoma (HCC), fibrolamellar hepatocellular carcinoma (FHCC), cholangiocarcinoma, angiosarcoma, secondary liver cancer, non-small cell lung cancer (NSCLC), adenocarcinoma, small cell lung cancer (SCLC), large cell (undifferentiated) carcinoma, triple negative breast cancer, gastric adenocarcinoma, endometrial carcinoma, or pancreatic carcinoma.
101. The expression repressor of any of the preceding embodiments, which, when contacted with a plurality of infected cells and a plurality of uninfected cells, decreases the viability of the plurality of infected cells more than it decreases the viability of the plurality of uninfected cells.
102. The expression repressor of any of preceding embodiments, wherein the infection is viral.
103. The expression repressor of embodiment 102, wherein the viral infection is hepatitis, e.g., hepatitis B.
104. The expression repressor of any of embodiments 92-103, wherein the infected cells are human hepatocytes.
105. The expression repressor of any of the preceding embodiments, which has an EC50 of 0.04-0.4, 0.04-0.1, 0.1-0.2, 0.2-0.3, or 0.3-0.4 μg/mL when tested in an assay for viability of cancer cells (e.g., HCC cells) using LNP delivery of mRNA encoding the expression repressor, e.g., in an assay according to Example 12 as described in International Application WO/2022/132195, which is herein incorporated by reference in its entirety.
106. The expression repressor of any of embodiments 1-104, which has an EC50 of 0.1-2.5, 0.5-2.2, 1.0-1.5, 1.2-2 μg/mL when tested in an assay for viability of cancer cells (e.g., lung cancer cells) using LNP delivery of mRNA encoding the expression repressor, e.g., in an assay according to Example 18 as described in International Application WO/2022/132195, which is herein incorporated by reference in its entirety.
107. The expression repressor of any of the preceding embodiments, which has an EC50 of 0.004-0.08, 0.004-0.01, 0.01-0.02, 0.02-0.04, or 0.04-0.08 μg/mL when tested in an assay for reducing MYC mRNA levels in cancer cells (e.g., HCC cells) using LNP delivery of mRNA encoding the expression repressor, e.g., in an assay according to Example 12 as described in International Application WO/2022/132195, which is herein incorporated by reference in its entirety.
108. The expression repressor of any of the preceding embodiments, which has an EC50 of 0.04-0.1, 0.04-0.09, 0.05-0.09, or 0.06-0.8 μg/mL when tested in an assay for reducing MYC mRNA levels in cancer cells (e.g., lung cancer cells) using LNP delivery of mRNA encoding the expression repressor, e.g., in an assay according to Example 18 as described in International Application WO/2022/132195, which is herein incorporated by reference in its entirety.
109. The expression repressor of any of the preceding embodiments, which reduces the level of a protein encoded by a target gene, e.g., MYC in a cell by at least 10%, at least 20%, at least 30%, at least 40%, at least 50%, at least 60%, at least 70%, at least 80%, or at least 90% compared to the protein level in an untreated cell.
110. The expression repressor of any of the preceding embodiments, which is capable of reducing tumor volume, e.g., in a human subject or in a mammalian model.
111. The expression repressor of any of preceding embodiments, wherein the expression repressor is capable of reducing tumor volume to a similar or greater degree compared to a chemotherapeutic agent, e.g., in a mammalian model, e.g., when measured at day 20 after initiation of treatment, e.g., wherein the expression repressor is administered every 5 days at a dose of 3 mg/kg.
112. The expression repressor of any of preceding embodiments, wherein the expression repressor is capable of reducing tumor volume compared to a PBS control, e.g., in a mammalian model, e.g., when measured at day 20 after initiation of treatment e.g., wherein the expression repressor is administered every 5 days for 4 doses followed by every 3 days for 3 doses at 1 mg/kg, 1.5 mg/kg, or 3 mg/kg.
113. The expression repressor of any of preceding embodiments, wherein the tumor volume is reduced by at least about 10%, 20%, 30%, or 40% compared to a control treated with PBS, e.g., at day 20 after start of treatment.
114. The expression repressor of any of embodiment 111-113, wherein the chemotherapeutic agent is sorafenib or cisplatin.
115. The expression repressor of any of preceding embodiments, wherein the system is capable of reducing tumor volume to a similar or greater degree compared to a small molecule MYC inhibitor.
116. The expression repressor of embodiment 115, wherein the small molecule MYC inhibitor is MYCi975 wherein optionally tumor volume is reduced by at least about 10%, 20%, 30%, or 40% compared to a control treated with the MYCi975, e.g., at day 20 after start of treatment.
117. The expression repressor of any of preceding embodiments, which does not cause a decrease in body weight compared to at the start of treatment, or which causes a decrease in body weight of less than 3%, 2%, or 1%.
118. A system comprising:
   a first expression repressor of any of the preceding embodiments, and
   a second expression repressor, e.g., a second expression repressor described herein, e.g., a second expression repressor of any of the preceding embodiments.
119. A system comprising:
   a first expression repressor comprising a first targeting moiety and optionally a first effector moiety, wherein the first expression repressor binds to a transcription regulatory element (e.g., a promoter, enhancer, or transcription start site (TSS)) operably linked to a MYC gene or to a sequence proximal to the transcription regulatory element, and
   a second expression repressor comprising a second targeting moiety and optionally a second effector moiety, wherein the second expression repressor binds to an anchor sequence of an anchor sequence mediated conjunction (ASMC) comprising a MYC gene or to a sequence proximal to the anchor sequence.
120. The system of embodiment 118 or 119,
   wherein the transcription regulatory element comprises a promoter, and
   wherein the anchor sequence comprises a CTCF binding motif.
121. The system of any of embodiments 118-120, wherein second expression repressor binds to a downstream region adjacent to the CTCF binding motif.
122. The system of any of embodiments 118-120, wherein second expression repressor binds to an upstream region adjacent to the CTCF binding motif.
123. The system of any of embodiments 118-122, wherein
   the first expression repressor comprises a targeting moiety that binds a genomic locus comprising at least 16, 17, 18, 19, or 20 nucleotides of the sequence of SEQ ID NOs: 2, 3, 4, 71, 72, 75-86, or 200-206 herein, or SEQ ID NOs: 73 or 74 as described in International Application WO/2022/132195, which is herein incorporated by reference in its entirety; and
   the second expression repressor comprises a targeting moiety that binds a genomic locus comprising at least 16, 17, 18, 19, or 20 nucleotides of the sequence of SEQ ID NO: 2, 3, 4, 71, 72, 75-86, or 200-206 herein, or SEQ ID NOs: 73 or 74 as described in International Application WO/2022/132195, which is herein incorporated by reference in its entirety.
124. The system of any of embodiments 118-123, wherein
   the first expression repressor comprises a targeting moiety that binds a genomic locus comprising at least 16, 17, 18, 19, or 20 nucleotides of the sequence of any of SEQ ID NO: 96-110.
125. The system of any of embodiments 118-124, wherein,
   the first expression repressor comprises a targeting moiety that binds a genomic locus comprising at least 16, 17, 18, 19, or 20 nucleotides of the sequence of SEQ ID NO: 71, SEQ ID NO: 72, or SEQ ID NO: 83; and
   the second expression repressor comprises a targeting moiety that binds a genomic locus comprising at least 16, 17, 18, 19, or 20 nucleotides of the sequence of SEQ ID NO: 77.
126. A system comprising:
   a first expression repressor comprising a first targeting moiety and optionally a first effector moiety, wherein the first expression repressor binds to a promoter operably linked to a MYC gene or to a sequence proximal to the promoter, and
   a second expression repressor comprising a second targeting moiety and optionally a second effector moiety, wherein the second expression repressor binds to an enhancer (e.g., a super-enhancer) of the MYC gene.
127. The system of embodiment 126, wherein,
   the first expression repressor comprises a targeting moiety that binds a genomic locus comprising at least 16, 17, 18, 19, or 20 nucleotides of the sequence of SEQ ID NO: 204, and
   the second expression repressor comprises a targeting moiety that binds a genomic locus comprising at least 16, 17, 18, 19, or 20 nucleotides of the sequence of any of SEQ ID NOs: 199 or 201.
128. A system for reducing MYC expression, the system comprising:
   a) a first expression repressor comprising:
      i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto and
      ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   b) a second expression repressor comprising:
      i) a second targeting moiety having an amino acid sequence according to SEQ ID NO: 7 169, or 171 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
      ii) a second effector moiety having an amino acid sequence according to SEQ ID NO: 18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
129. The system of embodiments 128, wherein the first expression repressor further comprises a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the first targeting moiety.
130. The system of embodiment 128 or 129, wherein the first expression repressor further comprises a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the first effector moiety.
131. The system of any of embodiments 128-130, wherein the second expression repressor further comprises a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the second targeting moiety.
132. The system of any of embodiments 128-131, wherein the second expression repressor further comprises a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the second effector moiety.
133. The system of any of embodiments 128-132, wherein the first expression repressor further comprises a first linker situated between the first targeting moiety and the first effector moiety, wherein optionally the first linker has an amino acid sequence according to SEQ ID NO: 137 or a sequence with at least 80, 85, 90, 95, 99, or 100% identity thereto.
134. The system of any of embodiments 128-133, wherein the second expression repressor further comprises a second linker situated between the second targeting moiety and the second effector moiety, wherein optionally the second linker has an amino acid sequence according to SEQ ID NO: 138 or a sequence with at least 80, 85, 90, 95, 99, or 100% identity thereto.
135. The system of any of embodiments 128-134, wherein the first expression repressor further comprises an amino acid sequence C-terminal of the first effector moiety, e.g., a sequence of up to 30, 25, 20, or 18 amino acids, e.g., a sequence according to SEQ ID NO: 126 or a sequence with at least 80, 85, 90, 95, 99, or 100% identity thereto.
136. The system of any of embodiments 128-132, wherein the second expression repressor further comprises an amino acid sequence N-terminal of the second targeting moiety, e.g., a sequence of up to 30, 25, 20, or 18 amino acids, or an amino acid P.
137. The system of any of embodiments 128-136, wherein the first expression repressor has an amino acid sequence according to SEQ ID NO: 30 or 129, or a sequence with at least 80, 85, 90, 95, 99, or 100% identity thereto.
138. The system of any of embodiments 128-137, wherein the second expression repressor has an amino acid sequence according to SEQ ID NO: 24, or a sequence with at least 80, 85, 90, 95, 99, or 100% identity thereto.
139. The system of any of embodiments 128-137, wherein the second targeting moiety comprises an amino acid sequence according to SEQ ID NO: 169, or a sequence with at least 80, 85, 90, 95, 99, or 100% identity thereto.
140. The system of any of embodiments 128-137, wherein the second targeting moiety comprises an amino acid sequence according to SEQ ID NO: 171, or a sequence with at least 80, 85, 90, 95, 99, or 100% identity thereto.
141. The system of any of embodiments 128-140, wherein the second expression repressor has an amino acid sequence according to SEQ ID NO: 177 or 183 or a sequence with at least 80, 85, 90, 95, 99, or 100% identity thereto.
142. The system of any of embodiments 128-140, wherein the second expression repressor has an amino acid sequence according to SEQ ID NO: 179, 185, or a sequence with at least 80, 85, 90, 95, 99, or 100% identity thereto.
143. A nucleic acid encoding the first expression repressor and second repressor of the system of any of embodiments 128-142.
144. A nucleic acid encoding a system for reducing MYC expression, the nucleic acid comprising:
   a) a first region encoding a first expression repressor, the first expression repressor comprising:
      i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
      ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   b) a second region encoding a second expression repressor, the second expression repressor comprising:
      i) a second targeting moiety having an amino acid sequence according to SEQ ID NO: 7 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
      ii) a second effector moiety having an amino acid sequence according to SEQ ID NO: 18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
145. The nucleic acid of embodiment 144, wherein the first region is 5′ of the second region.
146. The nucleic acid of embodiment 144, wherein the first region is 3′ of the second region.
147. The nucleic acid of embodiment 145 or 146, wherein the first region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the first targeting moiety.
148. The nucleic acid of any of embodiments 145-147, wherein the first region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the first effector moiety.
149. The nucleic acid of any of embodiments 145-148, wherein the second region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the second targeting moiety.
150. The nucleic acid of any of embodiments 145-149, wherein the second region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the second effector moiety.
151. The nucleic acid of any of embodiments 145-150, wherein the first region further comprises a nucleotide sequence encoding a first linker situated between the first targeting moiety and the first effector moiety, wherein optionally the first linker has an amino acid sequence according to SEQ ID NO: 137 or a sequence with at least 80, 85, 90, 95, 99, or 100% identity thereto.
152. The nucleic acid of any of embodiments 145-151, wherein the second region further comprises a nucleotide sequence encoding a second linker situated between the second targeting moiety and the second effector moiety, wherein optionally the second linker has an amino acid sequence according to SEQ ID NO: 138 or a sequence with at least 80, 85, 90, 95, 99, or 100% identity thereto.
153. The nucleic acid of any of embodiments 145-152, wherein the first region further comprises a nucleotide sequence encoding an amino acid sequence C-terminal of the first effector moiety, e.g., a sequence of up to 30, 25, 20, or 18 amino acids, e.g., a sequence according to SEQ ID NO: 126 or a sequence with at least 80, 85, 90, 95, 99, or 100% identity thereto.
154. The nucleic acid of any of embodiments 145-153, wherein the second region further comprises a nucleotide sequence encoding an amino acid sequence N-terminal of the second targeting moiety, e.g., a sequence of up to 30, 25, 20, or 18 amino acids, or an amino acid P.
155. The nucleic acid of any of embodiments 145-154, wherein the first expression repressor has an amino acid sequence according SEQ ID NO: 30 or 129, or a sequence with at least 80, 85, 90, 95, 99, or 100% identity thereto.
156. The nucleic acid of any of embodiments 145-155, wherein the second expression repressor has an amino acid sequence according to SEQ ID NO: 24, or a sequence with at least 80, 85, 90, 95, 99, or 100% identity thereto.
157. The nucleic acid of any of embodiments 145-156, wherein the first region comprises a nucleotide sequence encoding the first targeting moiety, wherein the nucleotide sequence encoding the first targeting moiety comprises a sequence according to SEQ ID NO: 46 or 131 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
158. The nucleic acid of any of embodiments 145-157, wherein the first region comprises a nucleotide sequence encoding the first effector moiety, wherein the nucleotide sequence encoding the first effector moiety comprises a sequence according to SEQ ID NO: 52 or 132, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
159. The nucleic acid of any of embodiments 145-158, wherein the second region comprises a nucleotide sequence encoding the second targeting moiety, wherein the nucleotide sequence encoding the second targeting moiety comprises a sequence according to SEQ ID NO: 40 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
160. The nucleic acid of any of embodiments 145-159, wherein the first region comprises a nucleotide sequence encoding the first effector moiety, wherein the nucleotide sequence encoding the first effector moiety comprises a sequence according to SEQ ID NO: 51, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
161. The nucleic acid of any of embodiments 145-160, wherein the first region comprises a nucleotide sequence according to SEQ ID NO: 63 or 130, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
162. The nucleic acid of any of embodiments 145-161, wherein the second region comprises a nucleotide sequence according to SEQ ID NO: 57, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
163. A nucleic acid encoding a system for reducing MYC expression, the nucleic acid comprising:
   a) a first region encoding a first expression repressor, the first expression repressor comprising:
      i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
      ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   b) a second region encoding a second expression repressor, the second expression repressor comprising:
      i) a second targeting moiety having an amino acid sequence according to SEQ ID NO:169 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
      ii) a second effector moiety having an amino acid sequence according to SEQ ID NO: 18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
164. A nucleic acid encoding a system for reducing MYC expression, the nucleic acid comprising:
   a) a first region encoding a first expression repressor, the first expression repressor comprising:
      i) a first targeting moiety having an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
      ii) a first effector moiety having an amino acid sequence according to SEQ ID NO: 19 or 87 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   b) a second region encoding a second expression repressor, the second expression repressor comprising:
      i) a second targeting moiety having an amino acid sequence according to SEQ ID NO:171 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
      ii) a second effector moiety having an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
165. The nucleic acid of embodiment 163 or 164, wherein the first region is 5′ of the second region.
166. The nucleic acid of embodiment 163 or 164, wherein the first region is 3′ of the second region.
167. The nucleic acid of any of embodiments 163-166, wherein the first region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the first targeting moiety.
168. The nucleic acid of any of embodiments 163-167, wherein the first region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the first effector moiety.
169. The nucleic acid of any of embodiments 163-168, wherein the second region further comprises a nucleotide sequence encoding a first nuclear localization signal, e.g., an SV40 NLS, e.g., a sequence according to SEQ ID NO: 135 or a sequence with at least 80, 85, 90, 95, 99, or 100% identity thereto, e.g., situated N-terminal of the second targeting moiety.
170. The nucleic acid of any of embodiments 163-169, wherein the second region further comprises a nucleotide sequence encoding a second nuclear localization signal, e.g., a nucleoplasmin NLS, e.g., a sequence according to SEQ ID NO: 136 or a sequence with at least 80, 85, 90, 95, 99, or 100% identity thereto, e.g., situated C-terminal of the second effector moiety.
171. The nucleic acid of any of embodiments 163-170, wherein the first region further comprises a nucleotide sequence encoding a first linker situated between the first targeting moiety and the first effector moiety, wherein optionally the first linker has an amino acid sequence according to SEQ ID NO: 137 or a sequence with at least 80, 85, 90, 95, 99, or 100% identity thereto.
172. The nucleic acid of any of embodiments 163-171, wherein the second region further comprises a nucleotide sequence encoding a second linker situated between the second targeting moiety and the second effector moiety, wherein optionally the second linker has an amino acid sequence according to SEQ ID NO: 138 or a sequence with at least 80, 85, 90, 95, 99, or 100% identity thereto.
173. The nucleic acid of any of embodiments 163-171, wherein the first region further comprises a nucleotide sequence encoding an amino acid sequence C-terminal of the first effector moiety, e.g., a sequence of up to 30, 25, 20, or 18 amino acids, e.g., a sequence according to SEQ ID NO: 126 or a sequence with at least 80, 85, 90, 95, 99, or 100% identity thereto.
174. The nucleic acid of any of embodiments 163-173, wherein the second region further comprises a nucleotide sequence encoding an amino acid sequence N-terminal of the second targeting moiety, e.g., a sequence of up to 30, 25, 20, or 18 amino acids, or an amino acid P.
175. The nucleic acid of any of embodiments 163-174, wherein the first expression repressor has an amino acid sequence according SEQ ID NO: 30 or 129, or a sequence with at least 80, 85, 90, 95, 99, or 100% identity thereto.
176. The nucleic acid of any of embodiments 144-175, wherein the second expression repressor has an amino acid sequence according to SEQ ID NO: 177, or 183 or a sequence with at least 80, 85, 90, 95, 99, or 100% identity thereto.
177. The nucleic acid of any of embodiments 144-176, wherein the second expression repressor has an amino acid sequence according to SEQ ID NO: 179, or 185, or a sequence with at least 80, 85, 90, 95, 99, or 100% identity thereto.
178. The nucleic acid of any of embodiments 144-177, wherein first expression repressor comprises an amino acid sequence according to SEQ ID NO: 30, or 129, or a sequence with at least 80, 85, 90, 95, 99, or 100% identity thereto and the second expression repressor has an amino acid sequence according to SEQ ID NO: 24, 141, 177, 179, 183, or 185, or a sequence with at least 80, 85, 90, 95, 99, or 100% identity thereto.
179. The nucleic acid of any of embodiments 144-178, wherein the first region comprises a nucleotide sequence encoding the first targeting moiety, wherein the nucleotide sequence encoding the first targeting moiety comprises a sequence according to SEQ ID NO: 46 or 131 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
180. The nucleic acid of any of embodiments 144-179, wherein the first region comprises a nucleotide sequence encoding the first effector moiety, wherein the nucleotide sequence encoding the first effector moiety comprises a sequence according to SEQ ID NO: 52 or 132, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
181. The nucleic acid of any of embodiments 144-180, wherein the second region comprises a nucleotide sequence according to SEQ ID NO: 173 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
182. The nucleic acid of any of embodiments 144-181, wherein the second region comprises a nucleotide sequence according to SEQ ID NO: 175 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
183. The nucleic acid of any of embodiments 144-182, wherein the second region comprises a nucleotide sequence encoding the second effector moiety, wherein the nucleotide sequence encoding the second effector moiety comprises a sequence according to SEQ ID NO: 51, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
184. The nucleic acid of any of embodiments 144-183, wherein the first region comprises a nucleotide sequence according to SEQ ID NO: 63 or 130, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
185. The nucleic acid of any of embodiments 144-184, wherein the second region comprises a nucleotide sequence according to SEQ ID NO: 189, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional.
186. The nucleic acid of any of embodiments 144-185, wherein the second region comprises a nucleotide sequence according to SEQ ID NO: 194, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein a poly-A sequence is optional
187. The nucleic acid of any of embodiments 144-186, wherein the first region comprises a nucleotide sequence encoding the first effector moiety, wherein the nucleotide sequence encoding the first effector moiety comprises a sequence according to SEQ ID NO: 52 or 132 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
188. The nucleic acid of any of embodiments 144-187, wherein the first region comprises a nucleotide sequence encoding the first targeting moiety, wherein the nucleotide sequence encoding the first targeting moiety comprises a sequence according to SEQ ID NO: 46 or 131, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
189. The nucleic acid of any of embodiments 144-188, wherein the second region comprises a nucleotide sequence encoding the second effector moiety, wherein the nucleotide sequence encoding the second effector moiety comprises a sequence according to SEQ ID NO: 51 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
190. The nucleic acid of any of embodiments 144-189, wherein the second region comprises a nucleotide sequence according to SEQ ID NO: 189, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
191. The nucleic acid of any of embodiments 144-190, wherein the second region comprises a nucleotide sequence according to SEQ ID NO: 194, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
192. The nucleic acid of any of embodiments 144-191, which has a nucleotide sequence according to SEQ ID NO: 93, 112, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
193. The nucleic acid of any of embodiments 144-192, which has a nucleotide sequence according to SEQ ID NO: 196 or 197, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
194. The system or nucleic acid of any of embodiments 118-193, wherein the first expression repressor comprises the first effector moiety.
195. The system or nucleic acid of any of embodiments 118-194, wherein the second expression repressor comprises the second effector moiety.
196. The system or nucleic acid of any of embodiments 118-195, wherein the first effector moiety has a different amino acid sequence from the second effector moiety.
197. The system or nucleic acid of any of embodiments 118-196, wherein the first effector moiety is a durable effector moiety.
198. The system or nucleic acid of any of embodiments 118-125 or 144-197, wherein the first effector moiety is a transient effector moiety.
199. The system or nucleic acid of any of embodiments 118-198, wherein the first effector moiety is an epigenetic modifying moiety.
200. The system or nucleic acid of any of embodiments 118-143, 163-197 or 199, wherein the first effector moiety comprises a histone methyltransferase.
201. The system or nucleic acid of embodiment 200, wherein the first effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof.
202. The system or nucleic acid of any of embodiments 118-143, 163-197, or 199, wherein the first effector moiety comprises a histone demethylase (e.g., a lysine demethylase).
203. The system or nucleic acid of embodiment 202, wherein the first effector moiety comprises a protein chosen from KDM1A (i.e., LSD1), KDM1B (i.e., LSD2), KDM2A, KDM2B, KDM5A, KDM5B, KDM5C, KDM5D, KDM4B, NO66 (or a functional variant or fragment of any thereof).
204. The system or nucleic acid of any of embodiments 118-143, 163-197, or 199, wherein the first effector moiety comprises a histone deacetylase.
205. The system or nucleic acid of embodiment 204, wherein the first effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.
206. The system or nucleic acid of any of embodiments 118-197 or 200, wherein the first effector moiety comprises a DNA methyltransferase.
207. The system or nucleic acid of embodiment 206, wherein the first effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.
208. The system or nucleic acid of any of embodiments 118-143, 160-196, or 198 or, wherein the first effector moiety is a transcription repressor moiety, e.g., comprising a transcription repressor.
209. The system or nucleic acid of embodiment 198 or 199, wherein the first effector moiety comprises a protein chosen from KRAB, MeCP2, HP1, RBBP4, REST, FOG1, SUZ12, or a functional variant or fragment of any thereof.
210. The system or nucleic acid of any of embodiments 118-209, wherein the first effector moiety promotes epigenetic modification of the transcription regulatory element or a sequence proximal thereto.
211. The system or nucleic acid of any of embodiments 118-210, wherein the first effector moiety catalyzes epigenetic modification of the transcription regulatory element or a sequence proximal thereto.
212. The system or nucleic acid of any of embodiments 118-125, 194, or 197-211, wherein the second expression repressor does not comprise an effector moiety.
213. The system or nucleic acid of any of embodiments 118-212, wherein the second effector moiety is a transient effector moiety.
214. The system or nucleic acid of any of embodiments 118-125 or 194-211, wherein the second effector moiety is a durable effector moiety.
215. The system or nucleic acid of any of embodiments 118-211 or 214, wherein the second effector moiety is an epigenetic modifying moiety.
216. The system or nucleic acid of any of embodiments 118-125, 194-211, or 214-215, wherein the second effector moiety comprises a histone methyltransferase.
217. The system or nucleic acid of embodiment 216, wherein the second effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof.
218. The system or nucleic acid of any of embodiments 118-125, 194-211, or 214-215, wherein the second effector moiety comprises a histone demethylase (e.g., a lysine demethylase).
219. The system or nucleic acid of embodiment 218, wherein the second effector moiety comprises a protein chosen from KDM1A (i.e., LSD1), KDM1B (i.e., LSD2), KDM2A, KDM2B, KDM5A, KDM5B, KDM5C, KDM5D, KDM4B, NO66 (or a functional variant or fragment of any thereof.
220. The system or nucleic acid of any of embodiments 118-125, 194-211, or 214-215, wherein the second effector moiety comprises a histone deacetylase.
221. The system or nucleic acid of embodiment 220, wherein the second effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.
222. The system or nucleic acid of any of embodiments 118-125, 194-211, or 214-215, wherein the second effector moiety comprises a DNA methyltransferase.
223. The system or nucleic acid of embodiment 222, wherein the second effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.
224. The system or nucleic acid of any of embodiments 118-211 or 213, wherein the second effector moiety is a transcription repressor moiety.
225. The system or nucleic acid of embodiment 224, wherein the second effector moiety promotes epigenetic modification of the anchor sequence or a sequence proximal thereto.
226. The system or nucleic acid of embodiment 223 or 224, wherein the second effector moiety binds to one or more endogenous epigenetic modifying proteins or one or more endogenous transcription modifying proteins.
227. The system or nucleic acid of any of embodiments 223-226, wherein the second effector moiety comprises KRAB, MeCP2, HP1, RBBP4, REST, FOG1, SUZ12, or a functional variant or fragment of any thereof.
228. The system or nucleic acid of any of embodiments 118-197, 199-207, 210-211, 213, or 224-227, wherein:
   the first effector moiety is a durable effector moiety, and
   the second effector moiety is a transient effector moiety.
229. The system or nucleic acid of embodiment 228, wherein the first effector moiety is an epigenetic modifying moiety.
230. The system or nucleic acid of embodiment 227 or 228, wherein the second effector moiety is a transcription repressor moiety.
231. The system or nucleic acid of any of embodiments 227-230, wherein:
   the first effector moiety comprises a histone methyltransferase, histone demethylase, histone deacetylase, DNA methyltransferase, a functional variant or fragment of any thereof, or a combination of any thereof, and
   the second effector moiety comprises a transcription repressor or a functional variant or fragment of any thereof.
232. The system or nucleic acid of any of embodiments 118-125, 194, 197, 199-207, 210-212, or 190, wherein:
   the first effector moiety comprises a histone methyltransferase, histone demethylase, histone deacetylase, DNA methyltransferase, a functional variant or fragment of any thereof, or a combination of any thereof, and
   the second expression repressor does not comprise a second effector moiety.
233. The system or nucleic acid of any of embodiments 118-125, 199-207, 210-211, 213 214, or 224-231 wherein:
   the first effector moiety comprises a SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, NO66, HDAC1, HDAC2. HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, a functional variant or fragment of any thereof, or a combination of any thereof, and
   the second effector moiety comprises KRAB, MeCP2, HP1, RBBP4, REST, FOG1, SUZ12, a functional variant or fragment of any thereof, or a combination of any thereof.
234. The system or nucleic acid of any of embodiments 118-197, 199, 206-207, 210-211, 213, 215, 224-231, or 233, wherein:
   the first effector moiety comprises a DNA methyltransferase, and
   the second effector moiety comprises a transcription repressor.
235. The system or nucleic acid of any of embodiments 118-125, 194, 197, 200, 206-207, 210-212, or 232 wherein:
   the first effector moiety comprises a DNA methyltransferase, and
   the second expression repressor does not comprise a second effector moiety.
236. The system or nucleic acid of any of embodiments 118-125, 200, 206-207, 210-235 wherein the first effector moiety comprises MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.
237. The system or nucleic acid of any of embodiments 118-211, 214, 224-234, or 236, wherein the second effector moiety comprises KRAB, MeCP2, HP1, RBBP4, REST, FOG1, SUZ12, or a functional variant or fragment of any thereof
238. The system or nucleic acid of any of embodiments 118-211, 199, 206-207, 210-211, 213, 224-234, or 236-237, wherein:
   the first effector moiety comprises MQ1 or a functional variant or fragment of any thereof, and
   the second effector comprises KRAB or a functional variant or fragment of any thereof.
239. The system or nucleic acid of any of embodiments 118-125, 194, 197, 199-207, or 210-212, 229, 232, 235, or 236, wherein:
   the first effector moiety comprises MQ1 or a functional variant or fragment of any thereof, and
   the second expression repressor does not comprise a second effector moiety.
240. The system or nucleic acid of any of embodiments 118-200 wherein the first expression repressor comprises an amino acid sequence chosen from any of SEQ ID NOs: 22-33, 129, 133, 134, 139-149, 177-180, or 183-186 herein, or SEQ ID NOs: 34-37 as described in International Application WO/2022/132195, which is herein incorporated by reference in its entirety, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
241. The system or nucleic acid of any of embodiments 118-198, 200, 206-211, 213-216, 222-223, 236-237, or 240, wherein the second expression repressor comprises an amino acid sequence chosen from any of SEQ ID NOs: 22-33, 129, 133, 134, 139-149, 177-180, or 183-186 herein, or SEQ ID NOs: 34-37 as described in International Application WO/2022/132195, which is herein incorporated by reference in its entirety, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
242. The system or nucleic acid of any of embodiments 118-198, 200, 206-211, 213-216, 222-223, 236-237, or 240-241, wherein the first expression repressor comprises an amino acid sequence of SEQ ID NO: 30, 129, 133, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second expression repressor comprises an amino acid sequence of SEQ ID NO: 24, 134, 141, 177, 179, 183, or 185, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
243. The system or nucleic acid of any of embodiments 118-198, 200, 206-211, 213-216, 222-223, 236-237, or 240-242, wherein the first expression repressor is encoded by a first nucleotide sequence of SEQ ID NO: 63 or 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second expression repressor are encoded by a second nucleotide sequence of SEQ ID NO: 57, 189, or 194, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
244. The system or nucleic acid of any of embodiments 118-198, 200, 206-211, 213-216, 222-223, 236-237, or 240-243, wherein the first and the second repressor are encoded by a nucleic acid sequence of SEQ ID NO: 93, 94, 112, 113, 196, or 197, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
245. The system or nucleic acid of embodiment 244 comprising an amino acid sequence of SEQ ID NO: 91, 92, 121, 122, 181, 182, 187, or 188, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
246. The system or nucleic acid of any of embodiments 118-197, 199, 206-207, 210-211, 213, 215, 224-231, 233-234, 236-237, or 240-244, wherein:
   the first expression repressor comprises from N-terminus to C-terminus:
   (i) a first nuclear localization signal, e.g., a SV40 NLS; e.g., a sequence according to SEQ ID NO: 135;
   (ii) a first targeting moiety, e.g., a zinc finger binding domain, e.g., ZF9; e.g., a sequence according to SEQ ID NO: 13;
   (iii) a first effector moiety, e.g., a DNA methyltransferase, e.g., MQ1; e.g., a sequence according to SEQ ID NO: 19 or 87;
   (iv) a second nuclear localization signal, e.g., a nucleoplasmin NLS; e.g., a sequence according to SEQ ID NO: 136;
   and the second expression repressor comprises, from N-terminus to C-terminus:
   (v) a third nuclear localization signal, e.g., a SV40NLS; e.g., a sequence according to SEQ ID NO: 135;
   (vi) a second targeting moiety, e.g., a zinc finger binding domain, e.g., ZF3; e.g., a sequence according to SEQ ID NO: 7;
   (vii) a second effector moiety, e.g., KRAB, e.g., a sequence according to SEQ ID NO: 18; and
   (viii) a fourth nuclear localization signal, e.g., a nucleoplasmin NLS, e.g., a sequence according to SEQ ID NO: 136.
247. The system or nucleic acid of any of embodiments 118-197, 199, 206-207, 210-211, 213, 215, 224-231, 233-234, 236-237, or 240-244, wherein:
   the first expression repressor comprises from N-terminus to C-terminus:
   (i) a first nuclear localization signal, e.g., a SV40 NLS; e.g., a sequence according to SEQ ID NO: 135;
   (ii) a first targeting moiety, e.g., a zinc finger binding domain, e.g., ZF9; e.g., a sequence according to SEQ ID NO: 13;
   (iii) a first effector moiety, e.g., a DNA methyltransferase, e.g., MQ1; e.g., a sequence according to SEQ ID NO: 19 or 87;
   (iv) a second nuclear localization signal, e.g., a nucleoplasmin NLS; e.g., a sequence according to SEQ ID NO: 136;
   and the second expression repressor comprises, from N-terminus to C-terminus:
   (v) a third nuclear localization signal, e.g., a SV40NLS; e.g., a sequence according to SEQ ID NO: 135;
   (vi) a second targeting moiety, e.g., a zinc finger binding domain, e.g., ZF54; e.g., a sequence according to SEQ ID NO: 169;
   (vii) a second effector moiety, e.g., KRAB, e.g., a sequence according to SEQ ID NO: 18; and
   (viii) a fourth nuclear localization signal, e.g., a nucleoplasmin NLS, e.g., a sequence according to SEQ ID NO: 136.
248. The system or nucleic acid of any of embodiments 118-197, 199, 206-207, 210-211, 213, 215, 224-231, 233-234, 236-237, or 240-244, wherein:
   the first expression repressor comprises from N-terminus to C-terminus:
   (i) a first nuclear localization signal, e.g., a SV40 NLS; e.g., a sequence according to SEQ ID NO: 135;
   (ii) a first targeting moiety, e.g., a zinc finger binding domain, e.g., ZF9; e.g., a sequence according to SEQ ID NO: 13;
   (iii) a first effector moiety, e.g., a DNA methyltransferase, e.g., MQ1; e.g., a sequence according to SEQ ID NO: 19 or 87;
   (iv) a second nuclear localization signal, e.g., a nucleoplasmin NLS; e.g., a sequence according to SEQ ID NO: 136;
   and the second expression repressor comprises, from N-terminus to C-terminus:
   (v) a third nuclear localization signal, e.g., a SV40NLS; e.g., a sequence according to SEQ ID NO: 135;
   (vi) a second targeting moiety, e.g., a zinc finger binding domain, e.g., ZF67; e.g., a sequence according to SEQ ID NO: 171;
   (vii) a second effector moiety, e.g., KRAB, e.g., a sequence according to SEQ ID NO:18; and
   (viii) a fourth nuclear localization signal, e.g., a nucleoplasmin NLS, e.g., a sequence according to SEQ ID NO: 136.
249. The system of any of embodiment 118-248, wherein the system is capable of the decreasing expression of MYC to a greater degree compared to the first expression repressor alone or the second expression repressor alone.
250. The system of any of embodiments 128-194, or 242-249, wherein the system is capable of decreasing expression of MYC to a greater degree compared to any of the expression repressors of SEQ ID NOs: 22, 23, 25-29, 31-33 herein, or SEQ ID NOs: 34-37 as described in International Application WO/2022/132195, which is herein incorporated by reference in its entirety, alone or in combination.
251. The system of any of embodiments 118-250, which is capable of reducing tumor volume, e.g., in a human subject or in a mammalian model.
252. The system of any of embodiments 128-193 or 242-209, wherein the system is capable of reducing tumor volume to a similar or greater degree compared to a chemotherapeutic agent, e.g., in a mammalian model, e.g., when measured at day 20 after initiation of treatment, e.g., wherein the expression repressor is administered every 5 days at a dose of 3 mg/kg, e.g., in a model system as described in Example 15 of International Application WO/2022/132195, which is herein incorporated by reference in its entirety.
253. The system of any of embodiments 128-193 or 242-252, wherein the system is capable of reducing tumor volume to a greater degree compared to a chemotherapeutic agent, e.g., in a mammalian model, e.g., when measured at day 15 after initiation of treatment, e.g., wherein the expression repressor is administered every 5 days at a dose of 6 mg/kg, e.g., in a model system as described in Example 14 of International Application WO/2022/132195, which is herein incorporated by reference in its entirety.
254. The system of any of embodiments 128-193 or 242-253, wherein the tumor volume is reduced by at least about 10%, 20%, 30%, 40%, 50%, or 60% compared to a control treated with PBS, e.g., at day 20 after start of treatment.
255. The system of embodiment 254 wherein the chemotherapeutic agent is sorafenib or cisplatin.
256. The system of any of embodiments 128-193 or 242-253, wherein the system is capable of reducing tumor volume to a similar or greater degree compared to a small molecule MYC inhibitor.
257. The system of embodiment 256 wherein the small molecule MYC inhibitor is MYCi975 wherein optionally tumor volume is reduced by at least about 10%, 20%, 30%, or 40% compared to a control treated with the MYCi975, e.g., at day 20 after start of treatment.
258. The system of any of embodiments 118-257, which does not cause a decrease in body weight compared to at the start of treatment, or which causes a decrease in body weight of less than 3%, 2%, or 1%.
259. The system or nucleic acid of any of embodiments 118-258, wherein the first targeting moiety is selected from a TAL effector domain, a CRISPR/Cas domain, a zinc finger domain, a tetR domain, a meganuclease, or an oligonucleotide.
260. The system or nucleic acid of any of embodiments 118-260, wherein the second targeting moiety is selected from a TAL effector domain, a CRISPR/Cas domain, a zinc finger domain, a tetR domain, a meganuclease, or an oligonucleotide.
261. The system or nucleic acid of any of embodiments 118-260, wherein the first targeting moiety comprises a CRISPR/Cas domain (e.g., a first CRISPR/Cas domain).
262. The system or nucleic acid of any of embodiments 118-261, wherein the second targeting moiety comprises a second CRISPR/Cas domain (e.g., a second CRISPR/Cas domain).
263. The system or nucleic acid of embodiment 262, wherein: i) the first CRISPR/Cas domain binds a first guide RNA, ii) the second CRISPR/Cas domain binds a second guide RNA, or iii) both (i) and (ii).
264. The system or nucleic acid of embodiment 262 or 263, wherein the first CRISPR/Cas domain does not bind the second guide RNA or binds with a K_{D} of at least 10, 20, 50, 100, 1000, or 10,000 nM, and the second CRISPR/Cas domain does not bind the first guide RNA, or binds with a K_{D} of at least 10, 20, 50, 100, 1000, or 10,000 nM.
265. The system or nucleic acid of any of embodiments 260-264, wherein the first CRISPR/Cas domain comprises a different amino acid sequence than the second CRISPR/Cas domain.
266. The system or nucleic acid of any of embodiments 260-265, wherein the first or second CRISPR/Cas domain comprises an amino acid sequence of a Cas protein or Cpf1 protein chosen from Table 1 as described in International Application WO/2022/132195 or a variant (e.g., mutant) of any thereof.
267. The system or nucleic acid of any of embodiments 260-266, wherein the first CRISPR/Cas domain comprises an amino acid sequence of a Cas protein or Cpf1 protein chosen from Table 1 as described in International Application WO/2022/132195 or a variant (e.g., mutant) of any thereof, and the second CRISPR/Cas domain comprises an amino acid sequence of a different Cas protein or Cpf1 protein chosen from Table 1 as described in International Application WO/2022/132195 or a variant (e.g., mutant) of any thereof.
268. The system or nucleic acid of any of embodiments 118-260, wherein the first targeting moiety comprises a zinc finger domain (e.g., a first zinc finger domain).
269. The system or nucleic acid of any of embodiments 118-260 or 268, wherein the second targeting moiety comprises a zinc finger domain (e.g., a second zinc finger domain).
270. The system or nucleic acid of any of embodiments 118-261 or 268-269, wherein the first targeting moiety comprises a first zinc finger domain and the second targeting moiety comprises a second zinc finger domain.
271. The system or nucleic acid of any of embodiments 268-270, wherein the first zinc finger domain and the second zinc finger domain bind the same genomic locus, e.g., have the same amino acid sequence.
272. The system or nucleic acid of any of embodiments 268-271, wherein the first zinc finger domain and the second zinc finger domain have different amino acid sequences or bind different genomic loci.
273. The system or nucleic acid of any of embodiments 118-261 or 267-272, wherein the first zinc finger molecule comprises at least 1, 2, 3, 4, 5, 7, 8, 9, or 10 zinc fingers (and optionally no more than 11, 10, 9, 8, 7, 6, or 5 zinc fingers).
274. The system or nucleic acid of any of embodiments 267-273, wherein the first zinc finger molecule comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6- 7, 7-10, 7-9, 7-8, 8-10, 8-9, or 9-10 zinc fingers.
275. The system or nucleic acid of any of embodiments 268-274, wherein the first zinc finger domain comprises 3 or 9 zinc fingers.
276. The system or nucleic acid of any of embodiments 268-275, wherein the second zinc finger domain comprises at least 1, 2, 3, 4, 5, 7, 8, 9, or 10 zinc fingers (and optionally no more than 11, 10, 9, 8, 7, 6, or 5 zinc fingers).
277. The system or nucleic acid of any of embodiments 268-276, wherein the second zinc finger domain comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6- 7, 7-10, 7-9, 7-8, 8-10, 8-9, or 9-10 zinc fingers.
278. The system or nucleic acid of any of embodiments 268-277, wherein the second zinc finger domain comprises 3 or 9 zinc fingers.
279. The system or nucleic acid of any of embodiments 118-278, wherein the first targeting moiety comprises a TAL effector domain (e.g., a first TAL effector domain).
280. The system or nucleic acid of any of embodiments 118-260 or 279 wherein the second targeting moiety comprises a TAL effector domain (e.g., a second TAL effector domain).
281. The system or nucleic acid of any of embodiments 279 or 280, wherein the first TAL effector domain comprises at least 2, 4, 6, 8, 10, 12, 14, 16, 18, 20, 22, 24, 26, 28, 30, 32, 34, 36, 38, or 40 central repeats (and optionally, no more than 45, 40, 35, 30, 25, 20, 15, or 10 central repeats).
282. The system or nucleic acid of any of embodiments 279-281, wherein the first TAL effector domain comprises 2-40, 5-40, 10-40, 15-40, 20-40, 25-40, 30-40, 35-40, 2-35, 5-35, 10-35, 15-35, 20-35, 25-35, 30-35, 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 central repeats.
283. The system or nucleic acid of any of embodiments 279-282, wherein the second TAL effector domain comprises at least 2, 4, 6, 8, 10, 12, 14, 16, 18, 20, 22, 24, 26, 28, 30, 32, 34, 36, 38, or 40 central repeats (and optionally, no more than 45, 40, 35, 30, 25, 20, 15, or 10 central repeats).
284. The system or nucleic acid of any of embodiments 279-283, wherein the second TAL effector domain comprises 2-40, 5-40, 10-40, 15-40, 20-40, 25-40, 30-40, 35-40, 2-35, 5-35, 10-35, 15-35, 20-35, 25-35, 30-35, 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 central repeats.
285. The system or nucleic acid of any of embodiments 118-284, wherein the first targeting moiety comprises a nucleic acid (e.g., a first nucleic acid).
286. The system of any of embodiments 129-285, wherein the second targeting moiety comprises a nucleic acid (e.g., a second nucleic acid).
287. The system or nucleic acid of any of embodiments 129-286, wherein the first targeting moiety comprises a polypeptide (e.g., a first polypeptide).
288. The system or nucleic acid of any of embodiments 129-287, wherein the second targeting moiety comprises a polypeptide (e.g., a second polypeptide).
289. The system of embodiment 287 or 288, wherein the nucleic acid is covalently attached to the polypeptide.
290. The system of embodiment 288 or 289, wherein the nucleic acid is non-covalently associated with the polypeptide.
291. The system or nucleic acid of any of embodiments 275-290, wherein the nucleic acid comprises a sequence that is complementary to the transcriptional regulatory element or a sequence proximal thereto, or comprises no more than 10, 9, 8, 7, 6, 5, 4, 3, 2, or 1 mismatches relative to the transcriptional regulatory element or a sequence proximal thereto.
292. The system or nucleic acid of any of embodiments 275-291, wherein the nucleic acid comprises a sequence that is complementary to the anchor sequence or a sequence proximal thereto, or comprises no more than 10, 9, 8, 7, 6, 5, 4, 3, 2, or 1 mismatches relative to the anchor sequence or a sequence proximal thereto.
293. The system of any of embodiments 275-292, wherein the nucleic acid comprises DNA, a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA, or other RNAi molecule.
294. The system of any of embodiments 275-293, wherein the nucleic acid comprises a gRNA.
295. The system of any of embodiments 275-294, wherein the nucleic acid comprises a sequence with at least 80, 85, 90, 95, 99, or 100% identity to any of SEQ ID NOs: 1-4, or has no more than 1, 2, 3, 4, 5, 6, 7, 8, 9, or 10 positions of difference thereto.
296. The system of any of embodiments 275-295, wherein the first nucleic acid comprises a sequence with at least 80, 85, 90, 95, 99, or 100% identity to any of SEQ ID NOs: 1-4 or has no more than 1, 2, 3, 4, 5, 6, 7, 8, 9, or 10 positions of difference thereto, and the second nucleic acid comprises a sequence with at least 80, 85, 90, 95, 99, or 100% identity to any of SEQ ID NOs: 1-4 or has no more than 1, 2, 3, 4, 5, 6, 7, 8, 9, or 10 positions of difference thereto.
297. The system of any of embodiments 275-295, wherein the first nucleic acid comprises a sequence with at least 80, 85, 90, 95, 99, or 100% identity to any of SEQ ID NOs: 96-110 or has no more than 1, 2, 3, 4, 5, 6, 7, 8, 9, or 10 positions of difference thereto, and the second nucleic acid comprises a sequence with at least 80, 85, 90, 95, 99, or 100% identity to any of SEQ ID NOs: 96-110 or has no more than 1, 2, 3, 4, 5, 6, 7, 8, 9, or 10 positions of difference thereto.
298. The system of any of embodiments 118-297, wherein the transcriptional regulatory element comprises a promoter.
299. The system of any of embodiments 118-298, wherein the transcriptional regulatory element comprises an enhancer; e.g., a super enhancer.
300. The system of any of embodiments 118-299, wherein the anchor sequence comprises a CTCF binding motif.
301. The system of any of embodiments 118-300, wherein the anchor sequence comprises a YY1 binding motif, e.g., a YY1 binding motif as described as described in International Application WO/2022/132195, which is herein incorporated by reference in its entirety.
302. The system of any of embodiments 118-301, wherein the anchor sequence comprises the sequence of SEQ ID NO: 71 or 72, or a sequence with no more than 8, 7, 6, 5, 4, 3, 2, or 1 alterations relative thereto.
303. The system of any of embodiments 118-302, wherein the anchor sequence comprises a sequence according to SEQ ID NO: 73 or 74 as described in International Application WO/2022/132195, which is herein incorporated by reference in its entirety, or a sequence with no more than 8, 7, 6, 5, 4, 3, 2, or 1 alterations relative thereto.
304. The system of any of embodiments 118-303, wherein the anchor sequence is on the same chromosome as the MYC gene.
305. The system of any of embodiments 118-304, wherein the anchor sequence is upstream of the MYC gene (e.g., upstream of the TSS or upstream of the promoter).
306. The system of any of embodiments 118-305, wherein the anchor sequence is at least 1, 5, 10, 50, 100, or 1000 kilobases away from the MYC gene (e.g., from the TSS or promoter of the MYC gene).
307. The system of any of embodiments 118-306, wherein the anchor sequence is 0.1-0.5, 0.1-1, 0.1-5, 0.1-10, 0.1-50, 0.1-100, 0.1-500, 0.1-1000, 0.5-1, 0.5-5, 0.5-10, 0.5-50, 0.5-100, 0.5-500, 0.5-1000, 1-5, 1-10, 1-50, 1-100, 1-500, 1-1000, 5-10, 5-50, 5-100, 5-500, 5-1000, 10-50, 10-100, 10-500, 10-1000, 50-100, 50-500, 50-1000, 100-500, 100-1000, or 500-1000 kilobases away from the MYC gene (e.g., from the TSS or promoter of the MYC gene).
308. The system of any of embodiments 118-303 or 305-307, wherein the anchor sequence is on a different chromosome than the MYC gene.
309. The system of any of embodiments 118-308, wherein the second targeting moiety binds to the anchor sequence or a sequence proximal to the anchor sequence with affinity sufficient to compete for binding with an endogenous polypeptide (e.g., CTCF or YY1).
310. The system of any of embodiments 118-309, wherein the first targeting moiety binds to a sequence at chromosome coordinates 128746342-128746364, 128746321-128746343, or 128746525-128746547, or a sequence proximal thereto.
311. The system of any of embodiments 118-309, wherein the first targeting moiety binds to a sequence at chromosome coordinates 128746405-128746425, 128748069-128748089, 129188825-129188845, or 129188822-129188842 or a sequence proximal thereto.
312. The system of any of embodiments 118-311, wherein the second targeting moiety binds to a sequence at chromosome coordinates 128748014-128748036, or a sequence proximal thereto.
313. The system of any of embodiments 118-311, wherein the second targeting moiety binds to a sequence at chromosome coordinates 128746405-128746425, 128748069-128748089, 129188825-129188845, or 129188822-129188842, or a sequence proximal thereto.
314. The system of any of embodiments 118-314, wherein the first expression repressor is a fusion molecule.
315. The system of any of embodiments 118-314, wherein the second expression repressor is a fusion molecule.
316. The system of any of embodiments 118-315, wherein the first expression repressor comprises a linker.
317. The system of any of embodiments 118-316, wherein the second expression repressor comprises a linker.
318. The system of any of embodiments 118-267 or 285-317, wherein:
   the first expression repressor comprises a targeting moiety comprising a first CRISPR/Cas molecule, e.g., comprising a first catalytically inactive CRISPR/Cas protein, and an effector moiety comprising an epigenetic modifying moiety; and
   the second expression repressor comprises a targeting moiety comprising a second CRISPR/Cas molecule, e.g., comprising a second catalytically inactive CRISPR/Cas protein, and an optionally an effector moiety comprising a transcription repressor.
319. The system of any of embodiments 118-260, 268-278, or 285-317 wherein:
   the first expression repressor comprises a targeting moiety comprising a first zinc finger domain, and an effector moiety comprising an epigenetic modifying moiety; and
   the second expression repressor comprises a targeting moiety comprising a second zinc finger domain, and optionally an effector moiety comprising a transcription repressor.
320. The system of any of embodiments 118-120, 262, 268, or 275-318, wherein:
   the first expression repressor comprises a targeting moiety comprising a CRISPR/Cas molecule, e.g., comprising a catalytically inactive CRISPR/Cas protein, and an effector moiety comprising an epigenetic modifying moiety; and
   the second expression repressor comprises a targeting moiety comprising a zinc finger domain, and optionally an effector moiety comprising a transcription repressor.
321. The system of any of embodiments 118-260, 268, or 275-318, wherein:
   the first expression repressor comprises a targeting moiety comprising a zinc finger domain, and an effector moiety comprising an epigenetic modifying moiety; and
   the second expression repressor comprises a targeting moiety comprising a CRISPR/Cas domain, e.g., comprising a catalytically inactive CRISPR/Cas protein, and optionally an effector moiety comprising a transcription repressor.
322. The system of any of embodiments 260, 268-278, or 275-318, wherein the zinc finger domain (e.g., the first or second zinc finger domain) comprises 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 zinc fingers, e.g., 3 or 9 zinc fingers.
323. The system of any of embodiments 322, wherein the epigenetic modifying moiety comprises a DNA methyltransferase.
324. The system of any of embodiments 118-323, wherein the epigenetic modifying moiety comprises MQ1 or a functional variant or fragment thereof.
325. The system of any of embodiments 118-324, wherein the second expression repressor comprises an effector moiety comprising a transcription repressor.
326. The system of any of embodiments 118-323, wherein the transcription repressor comprises KRAB or a functional variant or fragment thereof.
327. The system of any of embodiments 118-326, wherein the first expression repressor comprises an amino acid sequence of any of SEQ ID NOs: 28-33, 145-149, 151, or 152 herein, or SEQ ID NOs: 35-37 as described in International Application WO/2022/132195, which is herein incorporated by reference in its entirety, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
328. The system of any of embodiments 118-327, wherein the second expression repressor comprises an amino acid sequence of any of SEQ ID NOs: 22-27, 139-144, 150, 177-180, 183-186 herein, or SEQ ID NO: 34 as described in International Application WO/2022/132195, which is herein incorporated by reference in its entirety, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
329. The system of any of embodiments 118-328, wherein binding of the first expression repressor to the transcription regulatory element or a sequence proximal thereto decreases expression of MYC in a cell.
330. The system of embodiment 327, wherein expression is decreased by 10, 20, 30, 40, 50, 60, 70, 80, 90, or 100% compared to expression in the absence of the first expression repressor, e.g., as measured by QPCR or ELISA.
331. The system of embodiment 326 or 327, wherein binding of the first expression repressor to the transcription regulatory element appreciably decreases expression of MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
332. The system of any of embodiments 329-331, wherein binding of the first expression repressor to the transcription regulatory element appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
333. The system of any of embodiments 328-332, wherein binding of the second expression repressor to the anchor sequence or a sequence proximal thereto decreases expression of MYC in a cell.
334. The system of embodiment 333, wherein expression is decreased by 10, 20, 30, 40, 50, 60, 70, 80, 90, or 100% compared to expression in the absence of the second expression repressor, e.g., as measured by QPCR or ELISA.
335. The system of embodiment 333 or 334, wherein binding of the second expression repressor to the anchor sequence or a sequence proximal thereto appreciably decreases expression of MYC for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
336. The system of any of embodiments 334-335, wherein binding of the second expression repressor to the anchor sequence or a sequence proximal thereto appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
337. The system of any of embodiments 329-336, wherein binding of the first expression repressor to the transcription regulatory element or a sequence proximal thereto and the second expression repressor to the anchor sequence or a sequence proximal thereto decreases expression of MYC in a cell.
338. The system of any of embodiments 329-337, wherein binding of the first expression repressor to the transcription regulatory element or a sequence proximal thereto and binding of the second expression repressor to the anchor sequence or a sequence proximal thereto appreciably decreases expression of MYC at 1, 2, 3, 4, 5, 6, 7, 8, 10, 12, 16, 20, 24, 28, 32, 36, 40, 44, 48, 52, 56, 60, 64, 68, 72, 76, 80, or 96 hours post-transfection.
339. The system of any of embodiments 337 or 338, wherein expression is decreased by 10, 20, 30, 40, 50, 60, 70, 80, 90, or 100% compared to expression in the absence of the first and second expression repressors, e.g., as measured by QPCR or ELISA.
340. The system of any of embodiments 329-339, wherein binding of the first expression repressor to the transcription regulatory element or a sequence proximal thereto and the second expression repressor to the anchor sequence or a sequence proximal thereto appreciably decreases expression of MYC for a time period of at least 1, 2, 3, 4, 5, 6, 7, 8, 9, 10, 11, or 12 hours, or at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
341. The system of any of embodiments 329-340, wherein the decrease in expression resulting from the binding of the first expression repressor to the transcription regulatory element or a sequence proximal thereto and the second expression repressor to the anchor sequence or a sequence proximal thereto is greater than the decrease in expression resulting from the binding of the first expression repressor to the transcription regulatory element or a sequence proximal thereto or the binding of the second expression repressor to the anchor sequence or a sequence proximal thereto individually.
342. The system of embodiment 341, wherein the binding of the first expression repressor to the transcription regulatory element or a sequence proximal thereto and the second expression repressor to the anchor sequence or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the transcription regulatory element or a sequence proximal thereto or the binding of the second expression repressor to the anchor sequence or a sequence proximal thereto individually, e.g., as measured by QPCR or ELISA.
343. The system of any of embodiments 329-342, wherein the decrease in expression resulting from the binding of the first expression repressor to the transcription regulatory element or a sequence proximal thereto and the second expression repressor to the anchor sequence or a sequence proximal thereto persists for a longer time (e.g., more hours, days, or cell divisions) than the decrease in expression resulting from the binding of the first expression repressor to the transcription regulatory element or a sequence proximal thereto or the binding of the second expression repressor to the anchor sequence or a sequence proximal thereto individually.
344. The system of embodiment 343, wherein the binding of the first expression repressor to the transcription regulatory element or a sequence proximal thereto and the second expression repressor to the anchor sequence or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× longer (e.g., as measured in hours, days, or cell divisions) than either the binding of the first expression repressor to the transcription regulatory element or a sequence proximal thereto or the binding of the second expression repressor to the anchor sequence or a sequence proximal thereto individually, e.g., as measured by QPCR or ELISA.
345. The system of any of embodiments 329-344, wherein binding of the first expression repressor to the promoter or a sequence proximal thereto and the second expression repressor to the super-enhancer or a sequence proximal thereto appreciably decreases expression of MYC for a time period of at least 1, 2, 3, 4, 5, 6, 7, 8, 9, 10, 11, or 12 hours, or at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by QPCR or ELISA.
346. The system of any of embodiments 329-345, wherein the decrease in expression resulting from the binding of the first expression repressor to the promoter or a sequence proximal thereto and the second expression repressor to the super-enhancer or a sequence proximal thereto is greater than the decrease in expression resulting from the binding of the first expression repressor to the promoter or a sequence proximal thereto or the binding of the second expression repressor to the super-enhancer or a sequence proximal thereto individually.
347. The system of embodiment 346, wherein the binding of the first expression repressor to the promoter or a sequence proximal thereto and the second expression repressor to the super-enhancer or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the promoter or a sequence proximal thereto or the binding of the second expression repressor to the super-enhancer or a sequence proximal thereto individually, e.g., as measured by QPCR or ELISA.
348. The system of any of embodiments 329-347, wherein the decrease in expression resulting from the binding of the first expression repressor to the promoter or a sequence proximal thereto and the second expression repressor to the super-enhancer or a sequence proximal thereto persists for a longer time (e.g., more hours, days, or cell divisions) than the decrease in expression resulting from the binding of the first expression repressor to the promoter or a sequence proximal thereto or the binding of the second expression repressor to the super-enhancer or a sequence proximal thereto individually.
349. The system of embodiment 348, wherein the binding of the first expression repressor to the promoter or a sequence proximal thereto and the second expression repressor to the super-enhancer or a sequence proximal thereto decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× longer (e.g., as measured in hours, days, or cell divisions) than either the binding of the first expression repressor to the promoter or a sequence proximal thereto or the binding of the second expression repressor to the super-enhancer or a sequence proximal thereto individually, e.g., as measured by QPCR or ELISA.
350. The system of any of embodiments 329-349, wherein expression is appreciably decreased indefinitely (e.g., for a time period greater than can be experimentally measured).
351. The system of any of embodiments 329-350, wherein binding of the first expression repressor to the transcription regulatory element or a sequence proximal thereto decreases the viability of a cell comprising the transcription regulatory element or a sequence proximal thereto.
352. The system of any of embodiments 329-351, wherein contacting a plurality of cells with the first expression repressor or a nucleic acid encoding the first expression repressor decreases the viability of the plurality of cells, optionally wherein the plurality of cells comprise cancerous and non-cancerous cells and/or infected cells and uninfected cells.
353. The system of embodiment 352, wherein viability is decreased by 10, 20, 30, 40, 50, 60, 70, 80, 90, or 100% compared to viability in the absence of the first expression repressor, e.g., as measured by CellTiter Glo.
354. The system of any of embodiments 329-353, wherein, administration of the first expression repressor results in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
355. The system of any of embodiments 329-354, wherein binding of the second expression repressor to the anchor sequence or a sequence proximal thereto decreases the viability of a cell comprising the anchor sequence or a sequence proximal thereto.
356. The system of any of embodiments 329-355, wherein contacting a plurality of cells with the second expression repressor or a nucleic acid encoding the second expression repressor decreases the viability of the plurality of cells.
357. The system of any of embodiments 329-356, wherein binding of the second expression repressor to the super-enhancer or a sequence proximal thereto decreases the viability of a cell comprising the transcription regulatory element or a sequence proximal thereto.
358. The system of any of embodiments 329-357, wherein contacting a plurality of cells with the second expression repressor or a nucleic acid encoding the first expression repressor decreases the viability of the plurality of cells, optionally wherein the plurality of cells comprise cancerous and non-cancerous cells and/or infected cells and uninfected cells.
359. The system of embodiment 358, wherein viability is decreased by 10, 20, 30, 40, 50, 60, 70, 80, 90, or 100% compared to viability in the absence of the second expression repressor, e.g., as measured by CellTiter Glo.
360. The system of any of embodiments 329-359, wherein, administration of the second expression repressor results in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
361. The system of any of embodiments 329-360, wherein binding of the first expression repressor to the transcription regulatory element or a sequence proximal thereto and the second expression repressor to the anchor sequence or a sequence proximal thereto decreases the viability of a cell comprising the anchor sequence or a sequence proximal thereto.
362. The system of any of embodiments 329-361, wherein binding of the first expression repressor to the promoter or a sequence proximal thereto and the second expression repressor to the super-enhancer or a sequence proximal thereto decreases the viability of a cell
363. The system of any of embodiments 329-362, wherein contacting a plurality of cells with the system or a nucleic acid encoding the system decreases the viability of the plurality of cells.
364. The system of embodiments 329-363, wherein viability is decreased by 10, 20, 30, 40, 50, 60, 70, 80, 90, or 100% compared to viability in the absence of the system, e.g., as measured by CellTiter Glo.
365. The system of any of embodiments 329-364, wherein the decrease in viability resulting from the binding of the first expression repressor to the transcription regulatory element or a sequence proximal thereto and the second expression repressor to the anchor sequence or a sequence proximal thereto is greater than the decrease in viability resulting from the binding of the first expression repressor to the transcription regulatory element or a sequence proximal thereto or the binding of the second expression repressor to the anchor sequence or a sequence proximal thereto individually.
366. The system of any of embodiments 329-365, wherein the decrease in viability resulting from the binding of the first expression repressor to the promoter or a sequence proximal thereto and the second expression repressor to the super-enhancer or a sequence proximal thereto is greater than the decrease in viability resulting from the binding of the first expression repressor to the promoter or a sequence proximal thereto or the binding of the second expression repressor to the super-enhancer or a sequence proximal thereto individually.
367. The system of embodiment 366, wherein the binding of the first expression repressor to the transcription regulatory element or a sequence proximal thereto and the second expression repressor to the anchor sequence or a sequence proximal thereto decreases viability 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the transcription regulatory element or a sequence proximal thereto or the binding of the second expression repressor to the anchor sequence or a sequence proximal thereto individually, e.g., as measured by CellTiter Glo.
368. The system of embodiment 366 or 367, wherein the binding of the first expression repressor to the promoter or a sequence proximal thereto and the second expression repressor to the super-enhancer or a sequence proximal thereto decreases viability 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the promoter or a sequence proximal thereto or the binding of the second expression repressor to the super-enhancer or a sequence proximal thereto individually, e.g., as measured by CellTiter Glo.
369. The system of any of embodiments 329-368, wherein, administration of the first expression repressor and the second expression repressor result in apoptosis of at least 5%, 6%, 7%, 8%, 9% 10%, 12%, 15%, 17% 20%, 25% 30%, 40%, 45%, 50%, 55%, 60%, 65%, 75% of target cells (e.g., cancer cells).
370. The system of embodiments 329-369, wherein the plurality of cells comprises a plurality of cancer cells and a plurality of non-cancer cells.
371. The system of embodiment 370, wherein contacting the plurality of cells with the system or a nucleic acid encoding the system decreases the viability of the plurality of cancer cells more than it decreases the viability of the plurality of non-cancer cells.
372. The system of embodiment 370 or 371, wherein contacting the plurality of cells with the system or a nucleic acid encoding the system decreases the viability of the plurality of cancer cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of non-cancer cells.
373. The expression repressor or system of any preceding embodiments, which does not reduce viability of non-cancer cells (e.g., primary hepatocytes) by more than 5, 10, 15, or 20%, e.g., when assayed according to Example 29 as described in International Application WO/2022/132195, which is herein incorporated by reference in its entirety.
374. The expression repressor or system of embodiment 320, wherein viability is assayed 72 hours after contacting the cells with the expression repressor or system.
375. The expression repressor or system of embodiment 374, wherein the assay comprises contacting the non-cancer cells with 2.5, 2, 1.25, 1, 0.6, or 0.5 μg/ml of the expression repressor or system.
376. The system of any of embodiments 352-375, which, when contacted with a plurality of infected cells and a plurality of uninfected cells, decreases the viability of the plurality of infected cells more than it decreases the viability of the plurality of uninfected cells and/or decreases the viability of the plurality of cancerous cells more than it decreases the viability of the plurality of non-cancerous cells.
377. The system of any of embodiments 352-376, wherein the cancer is hepatocellular carcinoma (HCC), Fibrolamellar Hepatocellular Carcinoma (FHCC), Cholangiocarcinoma, Angiosarcoma, secondary liver cancer, Non-small cell lung cancer (NSCLC), Adenocarcinoma, Small cell lung cancer (SCLC), Large cell (undifferentiated) carcinoma, triple negative breast cancer, gastric adenocarcinoma, endometrial carcinoma, or pancreatic carcinoma.
378. The system of any of embodiments 352-377, wherein the cancer cells are lung cancer cells, gastric cancer cells, gastrointestinal cancer cells, colorectal cancer cells, pancreatic cancer cells, or hepatic cancer cells.
379. The system of any of embodiments 352-378, wherein the cells are human lung epithelial cells or human lung fibroblast cells
380. The system of any of embodiments 352-379, wherein the infection is viral.
381. The expression repressor of embodiment 380, wherein the viral infection is hepatitis, e.g., hepatitis B.
382. The system of any of embodiments 378-381, wherein the infected cells are human hepatocytes.
383. The system of any of embodiments 352-382, wherein the viral infection is a chronic infection.
384. A fusion protein comprising:
   a first amino acid region comprising a sequence encoding the first expression repressor of a system of any of embodiments 118-383; and
   a second amino acid region comprising a sequence encoding the second expression repressor of a system of any of embodiments 118-383.
385. The fusion protein of embodiment 384 which comprises a third amino acid region, wherein the third amino acid region is situated between the first amino acid region and the second amino acid region.
386. The fusion protein of embodiment 385, wherein the third amino acid region comprises a protease cleavage peptide sequence, e.g., a self-cleaving peptide sequence, e.g., a T2A self-cleaving peptide sequence, e.g., a sequence according to SEQ ID NO: 120.
387. The fusion protein of embodiment 386, wherein the third amino acid region comprises a protease cleavage peptide sequence, e.g., a self-cleaving peptide sequence, e.g., a tandem 2A peptide sequence, e.g., a tPT2A sequence, e.g., a sequence according to SEQ ID NO: 124.
388. The fusion protein of embodiment 385, wherein the peptide sequence comprises a T2A peptide sequence and a P2A peptide sequence.
389. The fusion protein of any of embodiments 384-388, wherein:
   the first expression repressor comprises an amino acid sequence according to SEQ ID NO: 30 or 129, or a sequence with at least 80, 85, 90, 95, or 99% identity thereto; and
   the second expression repressor comprises an amino acid sequence according to SEQ ID NO: 24 or 142, or a sequence with at least 80, 85, 90, 95, or 99% identity thereto.
390. The fusion protein of any of embodiments 384-388, wherein:
   the first expression repressor comprises an amino acid sequence according to SEQ ID NO: 30 or 129, or a sequence with at least 80, 85, 90, 95, or 99% identity thereto; and
   the second expression repressor comprises an amino acid sequence according to SEQ ID NO: 177 or 183, or a sequence with at least 80, 85, 90, 95, or 99% identity thereto.
391. The fusion protein of any of embodiments 384-388, wherein:
   the first expression repressor comprises an amino acid sequence according to SEQ ID NO: 30 or 129, or a sequence with at least 80, 85, 90, 95, or 99% identity thereto; and
   the second expression repressor comprises an amino acid sequence according to SEQ ID NO: 179 or 185, or a sequence with at least 80, 85, 90, 95, or 99% identity thereto.
392. The fusion protein of any of embodiments 384-391, which comprises an amino acid sequence of SEQ ID NO: 91, 92, 121, or 122, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
393. The fusion protein of any of embodiments 384-392, which comprises an amino acid sequence of SEQ ID NO: 181, 182, 187, or 188, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
394. A nucleic acid comprising a sequence encoding the system of any of embodiments 118-393.
395. A nucleic acid comprising a sequence encoding the system of embodiment 394.
396. The nucleic acid of embodiment 394 or 395, which comprises:
   a first region comprising a sequence encoding the first expression repressor of a system of any of embodiments 118-393; and
   a second region comprising a sequence encoding the second expression repressor of a system of any of embodiments 118-393.
397. The nucleic acid of any of embodiments 394-396, which comprises a third region, wherein the third region is situated between the first region and the second region.
398. The nucleic acid of any of embodiments 394-397, wherein the third region encodes a ribosome-skipping sequence.
399. The nucleic acid of embodiment 397 or 398, wherein the third region encodes a tPT2A peptide sequence, e.g., a sequence according to SEQ ID NO: 124.
400. The nucleic acid of any of embodiments 397-399, wherein the third region encodes a protease cleavage peptide sequence, e.g., a self-cleaving peptide sequence, e.g., a T2A self-cleaving peptide sequence, e.g., a sequence according to SEQ ID NO: 95.
401. The nucleic acid of any of embodiments 397-400, wherein the third region encodes a protease cleavage peptide sequence, e.g., a self-cleaving peptide sequence, e.g., a tandem 2A peptide sequence, e.g., a tPT2A peptide sequence, e.g., a sequence according to SEQ ID NO: 124.
402. The nucleic acid of any of embodiments 394-401, wherein
   the first expression repressor comprises an amino acid sequence according to SEQ ID NO: 30, 129 or a sequence with at least 80, 85, 90, 95, or 99% identity thereto; and
   the second expression repressor comprises an amino acid sequence according to SEQ ID NO: 24, 142, or a sequence with at least 80, 85, 90, 95, or 99% identity thereto.
403. The nucleic acid of any of embodiments 394-401, wherein
   the first expression repressor comprises an amino acid sequence according to SEQ ID NO: 30, 129 or a sequence with at least 80, 85, 90, 95, or 99% identity thereto; and
   the second expression repressor comprises an amino acid sequence according to SEQ ID NO: 177, 179, 183, or 185 or a sequence with at least 80, 85, 90, 95, or 99% identity thereto.
404. The nucleic acid of any of embodiments 394-403, which encodes an amino acid sequence of SEQ ID NO: 91, 92, 121, 122 or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
405. The nucleic acid of any of embodiments 394-404, which encodes an amino acid sequence of SEQ ID NO: 181, 182, 187, 188, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
406. The nucleic acid of any of embodiments 394-405, which comprises a nucleotide sequence of SEQ ID NO: 93, 94, 112, or 113 or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
407. The nucleic acid of any of embodiments 394-406, which comprises a nucleotide sequence of SEQ ID NO: 196, 197, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
408. A nucleic acid comprising a sequence encoding the expression repressor or the expression repressor system of any of embodiments 1-407.
409. The nucleic acid of any of embodiments 394-408, which is an RNA, e.g., an mRNA.
410. The nucleic acid of any of embodiments 394-409, which comprises an N7-methylated guanosine, e.g., linked to the 5′ end of the RNA, e.g., via a reverse 5′ to 5′ triphosphate linkage.
411. The nucleic acid of any of embodiments 394-410, which comprises a 5′ UTR.
412. The nucleic acid of any of embodiments 394-411, which comprises a Kozak sequence, e.g., between the 5′ UTR and the sequence encoding the expression repressor.
413. A system comprising:
   a first nucleic acid comprising a sequence encoding the first expression repressor of a system of any of embodiments 118-393; and
   a second nucleic acid comprising a sequence encoding a second expression repressor, e.g., the second expression repressor of a system of any of embodiments 118-393.
414. The system of embodiment 413, wherein the first nucleic acid has a nucleotide sequence of SEQ ID NO: 63, 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second nucleic acid having a nucleotide sequence of SEQ ID NO: 57, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
415. The system of embodiment 414, wherein the first nucleic acid has a nucleotide sequence of SEQ ID NO: 63, 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second nucleic acid having a nucleotide sequence of SEQ ID NO: 189, or 194, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
416. The system of embodiment 415, wherein the first nucleic acid has a nucleotide sequence of SEQ ID NO:189, 194, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto, and the second nucleic acid having a nucleotide sequence of SEQ ID NO: 63, 130, or a sequence with at least 80, 85, 90, 95, or 99% identity thereto, or a sequence with no more than 20, 19, 18, 17, 16, 15, 14, 13, 12, 11, 10, 9, 8, 7, 6, 5, 4, 3, 2, or 1 positions of difference thereto.
417. The nucleic acid or system of any of embodiments 394-416, wherein the nucleic acid comprises mRNA.
418. A vector comprising the nucleic acid encoding the system, or expression repressor of any of the preceding embodiments.
419. A lipid nanoparticle comprising the system, nucleic acid, mRNA, or vector of any of the preceding embodiments.
420. The lipid nanoparticle of embodiment 419 comprising an ionizable lipid, e.g., a cationic lipid, e.g., MC3, SSOP.
421. The lipid nanoparticle of embodiment 419 or 420, further comprising one or more of neutral lipids, ionizable amine-containing lipids, biodegradable alkyne lipids, steroids, phospholipids, polyunsaturated lipids, structural lipids (e.g., sterols), PEG, cholesterol, or polymer conjugated lipids.
422. A reaction mixture comprising the expression repressor, system, nucleic acid, vector, or lipid nanoparticle of any of the preceding embodiments.
423. The reaction mixture of embodiment 422, further comprising a cell.
424. A pharmaceutical composition comprising the expression repressor, system, nucleic acid, vector, lipid nanoparticle or the reaction mixture of any preceding embodiments.
425. A method of decreasing expression of a MYC gene in a cell, the method comprising:
   contacting the cell (e.g., a cancer cell) with an expression repressor, a system, one or more nucleic acids encoding said system or expression repressor, a vector, a lipid nanoparticle, or a pharmaceutical composition of any of embodiments 1-424,
   thereby decreasing expression of the MYC gene in the cell.
426. A method of treating cancer in a subject in need thereof, the method comprising:
   administering the expression repressor, system, nucleic acid, vector, lipid nanoparticle, or a pharmaceutical composition of any of embodiments 1-424 to the subject,
   thereby treating the cancer in the subject.
427. A method of reducing tumor growth in a subject in need thereof, the method comprising:
   administering the expression repressor, system, nucleic acid, vector, lipid nanoparticle, or a pharmaceutical composition of any of embodiments 1-424 to the subject,
   thereby reducing the tumor size in the subject.
428. The method of embodiment 427, wherein the reduction in tumor growth comprises reduction of tumor volume compared to tumor volume at the start of treatment.
429. The method of embodiment 428, wherein the reduction in tumor growth in the subject is greater compared to an untreated subject.
430. A method of increasing or restoring sensitivity of a cancer to a kinase inhibitor, e.g., a VEGFR1-3 inhibitor (e.g., lenvatinib), a tyrosine kinase inhibitor, e.g., sorafenib, an ERK inhibitor, e.g., ulixertinib, an AKT inhibitor, e.g., MK-2206, ARQ092, or TAS-117, a MEK inhibitor, e.g., trametinib or cobimetinib, or an EGFR TKI, e.g., osimertinib, erlotinib, afatinib, or gefitinib, the method comprising administering an expression repressor or system described herein to a subject having the cancer.
431. The method of embodiment 430, wherein administration of the expression repressor or system lowers the IC₅₀ of the kinase inhibitor by 10%, 20%, 30%, or 40%, e.g., in a cancer cell viability assay, e.g., an assay according to Example 38 as described in International Application WO/2022/132195, which is herein incorporated by reference in its entirety.
432. The method of embodiment 430 or 431, wherein the kinase inhibitor inhibits one or more of (e.g., all of) VEGFR, PDGFR, MEK, ERK, AKT, EGFR, or RAF kinase.
433. A method of increasing or restoring sensitivity of a cancer to a bromodomain inhibitor, e.g., a BET inhibitor, e.g., JQ1, the method comprising administering an expression repressor, system, or nucleic acid described herein (e.g., of any of embodiments 1-423) to a subject having the cancer, wherein optionally administration of the expression repressor or system lowers the IC₅₀ of the bromodomain inhibitor by 10%, 20%, 30%, 40%, 50%, 60%, 70%, 80%, 90%, or 95%, e.g., in a cancer cell viability assay, e.g., an assay according to Example 39 as described in International Application WO/2022/132195, which is herein incorporated by reference in its entirety.
434. The method of embodiment 433 wherein the bromodomain inhibitor is or comprises JQ1, BET672, or birabresib.
435. A method of increasing or restoring sensitivity of a cancer to a MEK inhibitor, e.g., Trametinib, the method comprising administering an expression repressor, system, or nucleic acid described herein (e.g., of any of embodiments 1-423) to a subject having the cancer, wherein optionally administration of the expression repressor or system lowers the IC₅₀ of the MEK inhibitor by 10%, 20%, 30%0, 40%, 50%, 60%, 70%, 80%, 90%, or 95%, e.g., in a cancer cell viability assay, e.g., an assay according to Example 51 as described in International Application WO/2022/132195, which is herein incorporated by reference in its entirety.
436. The method of any of embodiments 427-435, wherein the reduction in tumor growth in the subject is greater than or similar to a tumor size reduction when the subject is treated with a chemotherapeutic agent or small molecule MYC inhibitor.
437. The method of embodiment 436, wherein the chemotherapeutic agent is sorafenib or cisplatin.
438. The method of embodiment 437, wherein the small molecule MYC inhibitor is MYCi975.
439. The method of reducing tumor size in a subject in need thereof, the method comprising:
   administering the expression repressor, system, nucleic acid, vector, lipid nanoparticle, or a pharmaceutical composition of 1-424 to the subject, wherein the reduction in tumor size is greater than or similar to a tumor size reduction when the subject is treated with a chemotherapeutic agent.
440. The method of 439 wherein the chemotherapeutic agent is sorafenib or cisplatin.
441. The method of any of the preceding embodiments wherein the subject does not experience any significant side effects compared to when treated with a chemotherapeutic agent or a small molecule MYC inhibitor.
442. The method of any of embodiments 436-441, wherein the chemotherapeutic agent is sorafenib or cisplatin.
443. The method of embodiment 442, wherein the small molecule MYC inhibitor is MYCi975.
444. The method of any of embodiments 426-443, wherein the cancer is stage I, stage II, stage III, or stage IV cancer.
445. The method of any of preceding embodiments wherein the subject's body weight remains about the same before treatment and post-treatment.
446. The method of any of preceding embodiments, wherein the subject does not experience a decrease in body weight, or wherein the subject experiences a decrease in body weight of less than 3%, 2%, or 1% compared to at the start of treatment.
447. The method of any of the preceding embodiments wherein the subject does not experience a reduction or gain in body weight post-treatment compared to the subject's body weight before the treatment.
448. A method of treating a liver disease in a subject in need thereof, the method comprising:
   administering an expression repressor to the subject, wherein the expression repressor comprises targeting moiety that binds a MYC locus (e.g., a transcribed region of MYC, a MYC promoter, or an anchor sequence of an anchor sequence mediated conjunction (ASMC) comprising a MYC gene or to a sequence proximal to the anchor sequence), and optionally, an effector moiety, e.g., an effector moiety described herein;
   thereby treating the liver disease in the subject.
449. The method of embodiment 447, which further comprises administering to the subject a second expression repressor, the second expression repressor comprising a targeting moiety that binds to an anchor sequence of an anchor sequence mediated conjunction (ASMC) comprising a target gene, e.g., MYC, and optionally, a second effector moiety, e.g., an effector moiety described herein; e.g., KRAB;
   thereby treating the liver disease in the subject.
450. A method of treating a liver disease in a subject in need thereof, the method comprising:
   administering the expression repressor, system, nucleic acid, vector, lipid nanoparticle, or a pharmaceutical composition, of any of embodiments 1-424 to the subject,
   thereby treating the liver disease in the subject.
451. The method of embodiment 450, wherein the liver disease is a chronic liver disease.
452. The method of embodiment 450 or 451 wherein the liver disease is viral or alcohol related.
453. The method of any of embodiments 450-452, wherein the liver disease is hepatitis or hepatocellular carcinoma.
454. The method of embodiment 453, wherein the hepatocellular carcinoma is selected from HCC subtype S1, HCC subtype S2, or HCC subtype S3.
455. The method of embodiment 453 or 454, wherein the hepatocellular carcinoma is HCC S1.
456. The method of embodiment 453 or 454, wherein the hepatocellular carcinoma is HCC S2.
457. The method of any of embodiments 450-456 where the liver disease is caused by a hepatitis B virus or hepatitis C virus.
458. A method of treating a pulmonary disease in a subject in need thereof, the method comprising:
   administering an expression repressor to the subject, wherein the expression repressor comprises targeting moiety that binds a MYC locus (e.g., a transcribed region of MYC, a MYC promoter, or an anchor sequence of an anchor sequence mediated conjunction (ASMC) comprising a MYC gene or to a sequence proximal to the anchor sequence), and optionally, an effector moiety, e.g., an effector moiety described herein;
   thereby treating the pulmonary disease in the subject.
459. The method of embodiment 458, which further comprises administering to the subject a second expression repressor, the second expression repressor comprising a targeting moiety that binds a genomic locus located in a super enhancer region of a target gene, e.g., MYC, and optionally, a second effector moiety, e.g., an effector moiety described herein; e.g., KRAB;
   thereby treating the pulmonary disease in the subject
460. A method of treating a pulmonary disease in a subject in need thereof, the method comprising:
   administering the expression repressor, system, nucleic acid, vector, lipid nanoparticle, or a pharmaceutical composition, of any of embodiments 1-424 to the subject,
   thereby treating the pulmonary disease in the subject.
461. The method of embodiment 459 or 460 where the pulmonary disease is a cancer, e.g., a lung cancer, e.g., a lung carcinoma, e.g., non-small cell lung carcinoma or small cell lung carcinoma.
462. The method of any of embodiments 425-461, wherein contacting or administering comprises intravenous administration to a subject.
463. The method of any of embodiments 425-462, wherein contacting or administering comprises intra-tumoral delivery (e.g., injection).
464. The method of any of embodiments 425-463, wherein the cancer is characterized by increased MYC expression relative to a reference level (e.g., relative to a reference cell's MYC expression, e.g., an otherwise similar non-cancerous cell of the subject).
465. The method of any of embodiments 426-464, wherein the cancer is characterized by duplication of a portion of or all of a MYC gene.
466. The method of any of embodiments 426-465, wherein the cancer is selected from colorectal cancer, breast cancer, AML, prostate cancer, neuroblastoma, lung cancer, endometrial cancer, liver cancer, a lymphoma (e.g., Burkitt lymphoma), carcinoma of the cervix, or stomach cancer.
467. The method of any of embodiments 426-466, wherein the cancer is a human chorionic gonadotropin (hCG) secreting cancer.
468. The method of any of embodiments 426-467, wherein the cancer is hepatocarcinoma.
469. The method of any of embodiments 426-468, wherein the cancer is a non-responsive cancer, e.g., a non-responsive hepatocarcinoma.
470. The method of any of embodiments 426-469, wherein the cancer is non-small cell lung carcinoma or small cell lung carcinoma.
471. The method of any of embodiments 426-470, wherein the cancer over-expresses alpha-fetoprotein (AFP) (e.g., relative to a reference cell's AFP expression, e.g., an otherwise similar non-cancerous cell of the subject).
472. The method of any of embodiments 431-471, wherein cells of the cancer are characterized by the presence of a super enhancer, e.g., comprising the MYC gene or comprising the anchor-sequence mediated conjunction comprising the MYC gene, wherein optionally the cancer is selected from liver cancer, colorectal cancer, breast cancer, AML, prostate cancer, neuroblastoma, lung cancer, or endometrial cancer.
473. The method of embodiment 472, wherein the expression repressor (e.g., the second expression repressor) binds to an anchor sequence of an anchor sequence mediated conjunction (ASMC) comprising a MYC gene or to a sequence proximal to the anchor sequence.
474. The method of any of embodiments 426-473, wherein cells of the cancer are characterized by the absence of a super enhancer comprising the MYC gene or comprising the anchor-sequence mediated conjunction comprising the MYC gene.
475. The method of embodiment 474, wherein the expression repressor (e.g., the first expression repressor) binds the MYC promoter.
476. The method of any of embodiments 426-475, wherein the cancer comprises cells comprising a super enhancer comprising the MYC gene or comprising the anchor-sequence mediated conjunction comprising the MYC gene, and cells not comprising a super enhancer comprising the MYC gene or comprising the anchor-sequence mediated conjunction comprising the MYC gene.
477. The method of any of embodiments 426-476, wherein the cancer comprises cells characterized by increased MYC expression relative to a reference level (e.g., relative to a reference cell's MYC expression, e.g., an otherwise similar non-cancerous cell of the subject), and cells not characterized by increased MYC expression relative to a reference level (e.g., relative to a reference cell's MYC expression, e.g., an otherwise similar non-cancerous cell of the subject), e.g., having normal MYC expression.
478. The method of any of embodiments 426-477, wherein the expression repressor, system, nucleic acid, vector, lipid nanoparticle, or a pharmaceutical composition is administered a monotherapy.
479. The method of any of embodiments 426-478, which comprises administering a plurality of doses of the expression repressor, system, nucleic acid, vector, lipid nanoparticle, or a pharmaceutical composition to the subject, e.g., at least 2, 3, 4, 5, or 6 doses.
480. The method of any of embodiments 426-479, which comprises administering a plurality of doses of the expression repressor, system, nucleic acid, vector, lipid nanoparticle, or a pharmaceutical composition to the subject in 5 day intervals.
481. The method of any of embodiments 426-480, comprising:
   a) first, administering to the subject a first plurality of doses of an expression repressor or system described herein (e.g., of any of embodiments 1-424), wherein optionally each subsequent dose in the first plurality is administered 5 days after the previous dose in the first plurality;
   b) second, withdrawing the expression repressor or system for a period of time (a “drug holiday”), e.g., for about 2 weeks, and
   c) third, administering to the subject a second plurality of doses of the expression repressor or system, wherein optionally a subsequent dose of the second plurality is administered 5 days after the previous dose in the second plurality.
482. The method of embodiment 481, wherein the first plurality of doses comprises 4 doses.
483. The method of embodiment 480 or 481, wherein the second plurality of doses comprises 2 doses.
484. The method of any of embodiments 481-483, wherein the subject receives no therapeutic at all during the drug holiday.
485. The method of any of embodiments 481-484, wherein the subject receives a second therapeutic agent during the drug holiday.
486. The method of any of embodiments 481-485, wherein the drug holiday is at least twice as long as the time between administration of doses in the first plurality of doses.
487. The method of any of embodiments 481-487, wherein the drug holiday is at least twice as long as the time between administration of doses in the second plurality of doses.
488. The method of any of embodiments 426-487, wherein volume of the tumor declines to undetectable levels following treatment with the expression repressor or system.
489. The method of any of embodiments 426-488, tumor volume declines (e.g., to undetectable levels) after cessation of treatment with the expression repressor or system.
490. The method of any of embodiments 425-489, wherein the cancer does not become resistant to the expression repressor or system, or does not become resistant to the expression repressor or system within a period of 10, 20, 30, 40, 50, or 60 days.
491. The method of any of embodiments 425-490, wherein the cancer cells have a functional apoptotic pathway.
492. The method of any of embodiments 425-491, wherein the cancer cells have functional Caspase 3.
493. The method of embodiment 492, wherein Caspase 3 is upregulated in cancer cells upon administration of the expression repressor or system to the subject.
494. The method of any of embodiments 425-493, wherein Ki67 is downregulated in cancer cells upon administration of the expression repressor or system to the subject.
495. The method of any of embodiments 425-494, wherein cancer cell proliferation declines upon administration of the expression repressor or system to the subject.
496. The method of any of embodiments 425-495, wherein the method further comprises
   a. contacting the cell with a second therapeutic agent or
   b. administering a second therapeutic agent to the subject.
497. The method of embodiment 496, wherein the second therapeutic agent is not an expression repressor binding to MYC promoter.
498. The method of embodiment 496 or 497 wherein the second therapeutic agent is not an expression repressor, system, fusion protein, nucleic acid, vector, reaction mixture, pharmaceutical composition, or lipid nanoparticle of any of embodiments 1-424.
498. The method of any of embodiments 495-497, wherein the second therapeutic agent is the expression repressor, system, fusion protein, nucleic acid, vector, reaction mixture, pharmaceutical composition, or lipid nanoparticle, of any of embodiments 1-424.
500. The method of any of embodiments 496-498, wherein the second therapeutic agent is an immunotherapy, one or both of immune checkpoint and anti-vascular-endothelial-growth-factor therapy, systemic chemotherapy, a tyrosine kinase inhibitor, e.g., sorafenib, a mitogen-activated protein kinase kinase inhibitor (MEK inhibitor), e.g., trametinib, or a bromodomain inhibitor, e.g., a BET inhibitor, e.g., JQ1 or birabresib.
501. The method of any of embodiments 496-500, wherein the second therapeutic agent is a tyrosine kinase inhibitor, e.g., sorafenib.
502. The method of any of embodiments 496-500, wherein the second therapeutic agent is a bromodomain inhibitor, e.g., a BET inhibitor, e.g., JQ1, birabresib, or BET 672.
503. The method of any of embodiments 496-500, wherein the second therapeutic agent is a mitogen-activated protein kinase kinase inhibitor (MEK inhibitor), e.g., trametinib.
504. The method of any of embodiments 496-503, wherein the method further comprises administering an additional therapy to the subject.
505. The method of embodiment 504, wherein the additional therapy comprises surgical resection orthotopic liver transplantation, radiofrequency ablation, photodynamic therapy (PDT), laser therapy, brachytherapy, radiation therapy, trans-catheter arterial chemo- or radio-embolization, or stereotactic radiation therapy.
506. The method of any of embodiments 496-505, wherein the second therapeutic agent is selected from a checkpoint inhibitor or a small molecule.
507. The method of any of embodiments 496-506, wherein the second therapeutic agent is a chemotherapeutic agent, e.g., a kinase inhibitor or a bromodomain inhibitor, e.g., a BET inhibitor.
508. The method of embodiments 506 or 507 wherein the second therapeutic agent is selected from sorafenib, JQ1, BET672, birabresib, or trametinib.
509. The method of any of embodiments 496-507, wherein the expression repressor, system, or nucleic acid and the second therapeutic agent are administered concurrently.
510. The method of any of embodiments 496-509, wherein the expression repressor, system, or nucleic acid and the second therapeutic agent are administered sequentially.
511. The method of any of embodiments 504-509, wherein the additional therapy is administered concurrently.
512. The method of any of embodiments 504-510, wherein the additional therapy is administered sequentially.
513. The method of any of embodiments 496-509, wherein second therapeutic agent is administered simultaneously with the expression repressor, system, nucleic acid, vector, lipid nanoparticle, pharmaceutical composition, or reaction mixture of any of embodiments 1-424.
514. The method of any of embodiments 496-513, wherein second therapeutic agent is administered consecutively with the expression repressor, system, nucleic acid, vector, lipid nanoparticle, pharmaceutical composition, or reaction mixture of any of embodiments 1-424.
515. The method of any of embodiments 496-514, wherein the expression repressor, system, or nucleic acid is administered intravenously, and the second therapy is administered orally.
516. The method of any of the preceding embodiments, wherein the cancer is a resistant or refractory cancer.
517. The method of any of the preceding embodiments, wherein the cancer is resistant or refractory to a kinase inhibitor, e.g., a kinase inhibitor that inhibits one or more of VEGFR, PDGFR, or RAF kinase, e.g., sorafenib.
518. The method of any of the preceding embodiments, wherein the subject has an amplification in the MYC super-enhancer.
519. A kit comprising a container comprising a composition comprising an expression repressor, a system, one or more nucleic acids encoding said system or expression repressor, a vector, a lipid nanoparticle, reaction mixture, or a pharmaceutical composition of any of embodiments 1-424 and a set of instructions comprising at least one method for modulating, e.g., decreasing the expression of a MYC gene within a cell with said composition.
520. The method of any of embodiments 425-518, or the pharmaceutical composition of embodiment 424, wherein method comprises administering, or the pharmaceutical composition further comprises, a compound having the general structure of Formula (I):

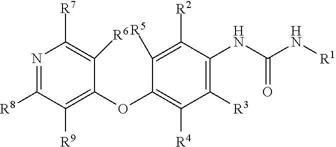

wherein:
R¹ is alkyl, cycloalkyl, or aryl;
R² is halo, alkyl, or H;
R³, R⁴, R⁵, R⁶, R⁷, are each independently H or alkyl;
R⁸ and R⁹ together form an optionally substituted aromatic ring;
or a pharmaceutically acceptable salt, tautomer or stereoisomer thereof.
521. The method or pharmaceutical composition of embodiment 520, wherein R¹ is alkyl or cycloalkyl.
522. The method or pharmaceutical composition of any of embodiment 520 or 521, wherein R¹=cyclopropane.
523. The method or pharmaceutical composition of any of embodiments 520-522, wherein R² is halo.
524. The method or pharmaceutical composition of any of embodiments 520-523, wherein R²=Cl.
525. The method or pharmaceutical composition of any of embodiments 520-524, wherein R³, R⁴, R⁵, R⁶, R⁷, are each H.
526. The method or pharmaceutical composition of any of embodiments 520-525, wherein the substituted aromatic ring is a substituted phenyl ring.
527. The method or pharmaceutical composition of any of embodiments 520-526, wherein the compound of Formula (I) comprises:

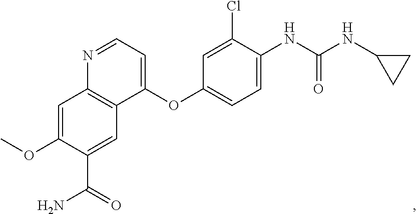 [SMILES: COc1cc2nccc(Oc3ccc(NC(=O)NC4CC4)c(Cl)c3)c2cc1C(N)=O]

or a pharmaceutically acceptable salt, tautomer or stereoisomer thereof.
528. The method or pharmaceutical composition of any of embodiments 520-527, wherein the compound of Formula (I) is administered orally.
529. The method or pharmaceutical composition of any of embodiments 520-528, wherein the compound of Formula (I) is administered at a dose of 6-10 (e.g., about 8), 10-14 (e.g., about 12) mg, or about 8-24 mg per day.
530. The method of any of embodiments 425-518, or the pharmaceutical composition of embodiment 424, wherein method comprises administering, or the pharmaceutical composition further comprises, a compound having the general structure of Formula (II):

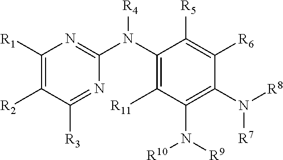

wherein R₁ is:

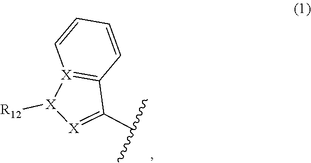

wherein X=C or N, given that one or two Xs are Ns; and
   R₁₂ is an electron pair, H or methyl;
   (2) halogen; or
   (3) methyl halogen;
R₂ is H or methyl;
R₃ is H or methyl;
R₄ is H or methyl;
R₅ is —OH or —O-methyl;
R₆ is H or methyl;
R₇ is H or methyl;
R₈ is —(CH₂)ₙ—N(R₁₃)₂, wherein n=2-6, and R₁₃=H or methyl;
R₉ is H or methyl;
R₁₀ is —(CO)—R₁₄, wherein R₁₄ is alkyl or alkene; and
R₁₁ is H or methyl,
or a pharmaceutically acceptable salt, tautomer or stereoisomer thereof.
531. The method of embodiment 530, wherein R₁ is:

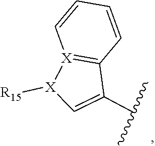

wherein X=C or N, given that one or two Xs are N; and R₁₅ is an electron pair, H or methyl.
532. The method of embodiment 530 or 531, wherein R₁ is:

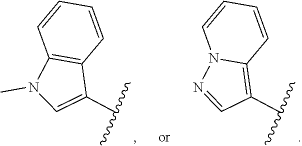 [SMILES: *c1cn(C)c2ccccc12 ; *c1cnn2ccccc12]

533. The method of any one of embodiments 530-532, wherein R₁ is:

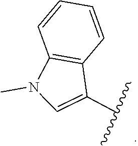 [SMILES: *c1cn(C)c2ccccc12]

534. The method of any one of embodiments 530-533, wherein:
   R₂ is H;
   R₃ is H;
   cR4 is H;
   R₅ is —O-methyl;
   R₆ is H;
   R₇ is methyl;
   R₈ is —(CH₂)₂—N(methyl)₂, wherein n=2-6, and R₁₃=H or methyl;
   R₉ is H;
   R₁₀ is —(CO)-ethylene; and
   R₁₁ is H.
535. The method of any one of embodiments 530-534, wherein Formula (II) is:

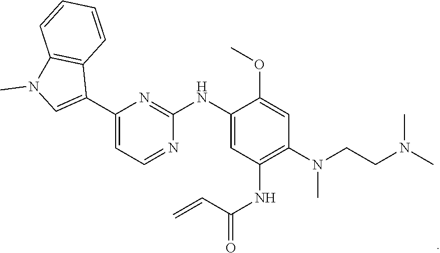

536. The method of any of embodiments 520-535, wherein the cancer is a hepatocellular carcinoma (HCC), Fibrolamellar Hepatocellular Carcinoma (FHCC), Cholangiocarcinoma, Angiosarcoma, secondary liver cancer, or non-small cell lung cancer.
537. The method of any of embodiments 520-536, wherein the nucleic acid comprises an RNA, e.g., an mRNA.
538. The method of any of embodiments 520-537, wherein:
   the first targeting moiety binds a genomic locus comprising at least 16, 17, 18, 19, or 20 nucleotides of the sequence of SEQ ID NO: 83, and
   the expression repressor comprises the first effector moiety, wherein the first effector moiety comprises a DNA methyltransferase.
539. The method of embodiment 538, wherein the first targeting moiety comprises a zinc finger domain.
540. The method of embodiment 538 or 539, wherein the first targeting moiety comprises an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
541. The method of any of embodiments 538-540, wherein the first effector moiety comprises MQ1 or a functional variant or fragment thereof.
542. The method of any of embodiments 538-541, wherein the first effector moiety comprises a sequence of SEQ ID NO: 19 or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
543. The method of any of embodiments 538-542, wherein the first effector moiety comprises a sequence of SEQ ID NO: 129, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
544. The method of any of embodiments 538-543, wherein the RNA comprises a nucleotide sequence encoding the first targeting moiety, wherein the nucleotide sequence encoding the first targeting moiety comprises a sequence according to SEQ ID NO: 131 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
545. The method of any of embodiments 538-544, wherein the RNA comprises a nucleotide sequence encoding the first effector moiety, wherein the nucleotide sequence encoding the first effector moiety comprises a sequence according to SEQ ID NO: 132, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
546. The method of any of embodiments 538-545, wherein the RNA comprises a nucleotide sequence according to SEQ ID NO: 130, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
547. The method of any of embodiments 538-546, wherein the RNA further encodes a second expression repressor, wherein the second expression repressor comprises:
   a second targeting moiety that binds a second genomic locus, and
   a second effector moiety.
548. The method of embodiment 547, wherein the second targeting moiety binds a second genomic locus comprising at least 14, 15, 16, 17, 18, 19, or 20 nucleotides of the sequence of SEQ ID NO: 77.
549. The method of embodiment 547 or 548, wherein the second targeting moiety comprises a zinc finger domain.
550. The method of any of embodiments 547-549, wherein the second targeting moiety comprises an amino acid sequence according to SEQ ID NO: 7, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
551. The method of any of embodiments 547-550, wherein the second effector moiety comprises KRAB or a functional variant or fragment thereof.
552. The method of any of embodiments 547-551, wherein the second effector moiety comprises an amino acid sequence according to SEQ ID NO:18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
553. The method of any of embodiments 547-552, wherein the second expression repressor comprises an amino acid sequence according to SEQ ID NO: 24 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
554. The method of any of embodiments 547-553, wherein the RNA comprises a nucleotide sequence according to SEQ ID NO: 113.
555. The method of any of embodiments 5209-554, wherein the nucleic acid is formulated in lipid nanoparticles (LNPs), wherein optionally the nucleic acid is encapsulated inside of the LNPs.
556. The method of any of embodiments 520-555, wherein the nucleic acid (e.g., RNA, e.g., mRNA) encoding the expression repressor is administered as an IV infusion, e.g., over 80-120 minutes, e.g., every 2 weeks.
557. The method of any of embodiments 520-556, wherein the nucleic acid is administered at a dose of about 0.001 mg/kg to 1.5 mg/kg or about 0.002 mg/kg to 1.5 mg/kg, e.g., administered every 1, 2, 3, 4, 5, 6 or 7 weeks.
558. The method of any of embodiments 520-557, wherein the nucleic acid (e.g., RNA, e.g., mRNA) encoding the expression repressor and the compound having the general structure of Formula (I) are administered on different days.
559. The method of any of embodiments 520-558, wherein the nucleic acid (e.g., RNA, e.g., mRNA) encoding the expression repressor and the compound having the general structure of Formula (I) are administered on the same day.
560. The method of embodiment 559, wherein the of the nucleic acid (e.g., RNA, e.g., mRNA) encoding the expression repressor is administered prior to administration of the compound having the general structure of Formula (I) or Formula (II).
561. The method of embodiment 559 or 560, wherein the compound having the general structure of Formula (I) or Formula (II) is administered between 1-24 hours after completion of administration of the nucleic acid (e.g., RNA, e.g., mRNA) encoding an expression repressor.

### Definitions

A, an, the: As used herein, the singular forms “a,” “an” and “the” include plural referents unless the context clearly dictates otherwise.
Agent: As used herein, the term “agent”, may be used to refer to a compound or entity of any chemical class including, for example, a polypeptide, nucleic acid, saccharide, lipid, small molecule, metal, or combination or complex thereof. As will be clear from context to those skilled in the art, in some embodiments, the term may be utilized to refer to an entity that is or comprises a cell or organism, or a fraction, extract, or component thereof. Alternatively, or additionally, as those skilled in the art will understand in light of context, in some embodiments, the term may be used to refer to a natural product in that it is found in and/or is obtained from nature. In some embodiments, again as will be understood by those skilled in the art in light of context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some embodiments, the term “agent” may refer to a compound or entity that is or comprises a polymer; in some embodiments, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.
Anchor Sequence: The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.
Anchor Sequence-Mediated Conjunction: The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences (see, e.g. FIG. 1).
Associated with: Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic or transcription complex when the nucleic acid is at least partially within the target genomic or transcription complex, and expression of a gene in the DNA sequence is affected by formation or disruption of the target genomic or transcription complex.
Biologically Active Portion of an Effector Domain: As used herein, a “biologically active portion of an effector domain” is a portion that maintains function (e.g., completely, partially, minimally) of an effector domain (e.g., a “minimal” or “core” domain).
Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).
Effector moiety: As used herein, the term “effector moiety” refers to a domain that is capable of altering the expression of a target gene when localized to an appropriate site in the nucleus of a cell. In some embodiments, an effector moiety recruits components of the transcription machinery. In some embodiments, an effector moiety inhibits recruitment of components of transcription factors or expression repressing factors. In some embodiments, an effector moiety comprises an epigenetic modifying moiety (e.g., epigenetically modifies a target DNA sequence).
Epigenetic modifying moiety: As used herein, “epigenetic modifying moiety” refers to a domain that alters: i) the structure, e.g., two dimensional structure, of chromatin; and/or ii) an epigenetic marker (e.g., one or more of DNA methylation, histone methylation, histone acetylation, histone sumoylation, histone phosphorylation, and RNA-associated silencing), when the epigenetic modifying moiety is appropriately localized to a nucleic acid (e.g., by a targeting moiety). In some embodiments, an epigenetic modifying moiety comprises an enzyme, or a functional fragment or variant thereof, that affects (e.g., increases or decreases the level of) one or more epigenetic markers. In some embodiments, an epigenetic modifying moiety comprises a DNA methyltransferase, a histone methyltransferase, CREB-binding protein (CBP), or a functional fragment of any thereof.
Expression control sequence: As used herein, the term “expression control sequence” refers to a nucleic acid sequence that increases or decreases transcription of a gene and includes (but is not limited to) a promoter and an enhancer. An “enhancing sequence” refers to a subtype of expression control sequence and increases the likelihood of gene transcription. A “silencing or repressor sequence” refers to a subtype of expression control sequence and decreases the likelihood of gene transcription.
Expression repressor: As used herein, the term “expression repressor” refers to an agent or entity with one or more functionalities that decreases expression of a target gene in a cell and that specifically binds to a DNA sequence (e.g., a DNA sequence associated with a target gene or a transcription control element operably linked to a target gene). An expression repressor comprises at least one targeting moiety and optionally one effector moiety.
Expression repression system: As used herein, the term “expression repression system” refers to a plurality of expression repressors which decrease expression of a target gene in a cell. In some embodiments, an expression repression system comprises a first expression repressor and a second expression repressor, wherein the first expression repressor and second expression repressor (or nucleic acids encoding the first expression repressor and second expression repressor) are present together in a single composition, mixture, or pharmaceutical composition. In some embodiments, an expression repression system comprises a first expression repressor and a second expression repressor, wherein the first expression repressor and second expression repressor (or nucleic acids encoding the first expression repressor and second expression repressor) are present in separate compositions or pharmaceutical compositions. In some embodiments, the first expression repressor and the second expression repressor are present in the same cell at the same time. In some embodiments, the first expression repressor and the second expression repressor are not present in the same cell at the same time, e.g., they are present sequentially. For example, the first expression repressor may be present in a cell for a first time period, and then the second expression repressor may be present in the cell for a second time period, wherein the first and second time periods may be overlapping or non-overlapping.
Fusion Molecule: As used herein, the term “fusion molecule” refers to a compound comprising two or more moieties, e.g., a targeting moiety and an effector moiety, that are covalently linked. A fusion molecule and its moieties may comprise any combination of polypeptide, nucleic acid, glycan, small molecule, or other components described herein (e.g., a targeting moiety may comprise a nucleic acid and an effector moiety may comprise a polypeptide). In some embodiments, a fusion molecule is a fusion protein, e.g., comprising one or more polypeptide domains covalently linked via peptide bonds. In some embodiments, a fusion molecule is a conjugate molecule that comprises a targeting moiety and effector moiety that are linked by a covalent bond other than a peptide bond or phosphodiester bond (e.g., a targeting moiety that comprises a nucleic acid and an effector moiety comprising a polypeptide linked by a covalent bond other than a peptide bond or phosphodiester bond). In some embodiments, an expression repressor is or comprises a fusion molecule.
Genomic complex: As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that it does not adopt when the complex is not formed.
Moiety: As used herein, the term “moiety” refers to a defined chemical group or entity with a particular structure and/or or activity, as described herein.
Modulating agent: As used herein, the term “modulating agent” refers to an agent comprising one or more targeting moieties and one or more effector moieties that is capable of altering (e.g., increasing or decreasing) expression of a target gene, e.g., MYC.
MYC: As used herein, the terms “MYC locus” refer to the portion of the human genome that encodes a MYC polypeptide (e.g., the polypeptide disclosed in NCBI Accession Number NP002458.2, or a mutant thereof), the promoter operably linked to MYC (“MYC promoter”), and the anchor sequences that form an ASMC comprising the MYC gene. In some embodiments, the MYC locus encodes a nucleic acid having NCBI Accession Number NM-002467. In some embodiments, the MYC gene is a proto-oncogene, and in some embodiments the MYC gene is an oncogene. In certain instances, a MYC gene is found on chromosome 8, at 8q24.21. In certain instances, a MYC gene begins at 128,816,862 bp from pter and ends at 128,822,856 bp from pter. In certain instances, a MYC gene is about 6 kb. In certain instances, a MYC gene encodes at least eight separate mRNA sequences—5 alternatively spliced variants and 3 un-spliced variants.
Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.
Nucleating polypeptide: As used herein, the term “nucleating polypeptide” or “conjunction nucleating polypeptide” as used herein, refers to a protein that associates with an anchor sequence directly or indirectly and may interact with one or more conjunction nucleating polypeptides (that may interact with an anchor sequence or other nucleic acids) to form a dimer (or higher order structure) comprised of two or more such conjunction nucleating polypeptides, which may or may not be identical to one another. When conjunction nucleating polypeptides associated with different anchor sequences associate with each other so that the different anchor sequences are maintained in physical proximity with one another, the structure generated thereby is an anchor-sequence-mediated conjunction. That is, the close physical proximity of a nucleating polypeptide-anchor sequence interacting with another nucleating polypeptide-anchor sequence generates an anchor sequence-mediated conjunction (e.g., in some cases, a DNA loop), that begins and ends at the anchor sequence. As those skilled in the art, reading the present specification will immediately appreciate, terms such as “nucleating polypeptide”, “nucleating molecule”, “nucleating protein”, “conjunction nucleating protein”, may sometimes be used to refer to a conjunction nucleating polypeptide. As will similarly be immediately appreciated by those skilled in the art reading the present specification, an assembles collection of two or more conjunction nucleating polypeptides (which may, in some embodiments, include multiple copies of the same agent and/or in some embodiments one or more of each of a plurality of different agents) may be referred to as a “complex”, a “dimer” a “multimer”, etc.
Operably linked: As used herein, the phrase “operably linked” refers to a juxtaposition wherein the components described are in a relationship permitting them to function in their intended manner. A transcription control element “operably linked” to a functional element, e.g., gene, is associated in such a way that expression and/or activity of the functional element, e.g., gene, is achieved under conditions compatible with the transcription control element. In some embodiments, “operably linked” transcription control elements are contiguous (e.g., covalently linked) with coding elements, e.g., genes, of interest; in some embodiments, operably linked transcription control elements act in trans to or otherwise at a distance from the functional element, e.g., gene, of interest. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other.
Peptide, Polypeptide, Protein: As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.
Pharmaceutical composition: As used herein, the term “pharmaceutical composition” refers to an active agent (e.g., a modulating agent, e.g., a disrupting agent), formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces.
Proximal: As used herein, “proximal” refers to a closeness of two sites, e.g., nucleic acid sites, such that binding of an expression repressor at the first site and/or modification of the first site by an expression repressor will produce the same or substantially the same effect as binding and/or modification of the other site. For example, a targeting moiety may bind to a first site that is proximal to an enhancer (the second site), and the effector moiety associated with said targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, or proximal to an anchor sequence is less than 5000, 4000, 3000, 2000, 1000, 900, 800, 700, 600, 500, 400, 300, 200, 100, 50, or 25 base pairs from the target gene (e.g., an exon, intron, or splice site within the target gene), transcription control element, or anchor sequence (and optionally at least 20, 25, 50, 100, 200, or 300 base pairs from the target gene (e.g., an exon, intron, or splice site within the target gene), transcription control element, or anchor sequence).
Specific: As used herein, the term “specific”, referring to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In some embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments, specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).
Specific binding: As used herein, the term “specific binding” refers to an ability to discriminate between possible binding partners in the environment in which binding is to occur. In some embodiments, a binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex. In some embodiments, specific binding is assessed by detecting or determining ability of the binding agent to compete with an alternative interaction between its partner and another entity. In some embodiments, specific binding is assessed by performing such detections or determinations across a range of concentrations.
Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.
Symptoms are reduced: As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.
Target: An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence.
Target gene: As used herein, the term “target gene” means a gene that is targeted for modulation, e.g., of expression. In some embodiments, a target gene is part of a targeted genomic complex (e.g. a gene that has at least part of its genomic sequence as part of a target genomic complex, e.g. inside an anchor sequence-mediated conjunction), which genomic complex is targeted by one or more modulating agents as described herein. In some embodiments, modulation comprises inhibition of expression of the target gene. In some embodiments, a target gene is modulated by contacting the target gene or a transcription control element operably linked to the target gene with an expression repression system, e.g., expression repressor(s), described herein. In some embodiments, a target gene is aberrantly expressed (e.g., over-expressed) in a cell, e.g., a cell in a subject (e.g., patient).
Targeting moiety: As used herein, the term “targeting moiety” means an agent or entity that specifically targets, e.g., binds, a genomic sequence element (e.g., an expression control sequence or anchor sequence). In some embodiments, the genomic sequence element is proximal to and/or operably linked to a target gene (e.g., MYC).
Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent comprises an expression repression system, e.g., an expression repressor, described herein. In some embodiments, a therapeutic agent comprises a nucleic acid encoding an expression repression system, e.g., an expression repressor, described herein. In some embodiments, a therapeutic agent comprises a pharmaceutical composition described herein.
Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) or tissue(s), etc. For example, in some embodiments, an effective amount of compound in a formulation to treat a disease, disorder, and/or condition is an amount that alleviates, ameliorates, relieves, inhibits, prevents, delays onset of, reduces seventy of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.
Halo: As used herein, the term “halo,” represents a halogen selected from bromine, chlorine, iodine, and fluorine.
Alkyl: As used herein, the term “alkyl” refers to a straight or branched hydrocarbon chain radical consisting solely of carbon and hydrogen atoms, which is saturated or unsaturated (i.e., contains one or more double and/or triple bonds), having from one to twenty-four carbon atoms (C₁-C₂₄ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, propenyl, butenyl, pentenyl, penta-1,4-dienyl, ethynyl, propynyl, butynyl, pentynyl, hexynyl, and the like. Unless stated otherwise specifically in the specification, an alkyl group is optionally substituted.
Alkylene: As used herein, the term “alkylene” or “alkylene chain” refers to a straight or branched divalent hydrocarbon chain linking the rest of the molecule to a radical group, consisting solely of carbon and hydrogen, which is saturated or unsaturated (i.e., contains one or more double (alkenylene) and/or triple bonds (alkynylene)), and having, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. The alkylene chain may be attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.
Cycloalkyl: As used herein, the term “cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals may include, for example, cyclopropyl, cyclobutyl, cyclopentyl, cyclohexyl, cycloheptyl, and cyclooctyl. Polycyclic radicals may include, for example, adamantyl, norbornyl, decalinyl, 7,7-dimethyl bicyclo[2.2.1]heptanyl, and the like. Unless otherwise stated specifically in the specification, a cycloalkyl group is optionally substituted.
Cycloalkylene: As used herein, the term “cycloalkylene” refers to a divalent cycloalkyl group. Unless otherwise stated specifically in the specification, a cycloalkylene group may be optionally substituted.
Cycloalkenylene: As used herein, the term “cycloalkenylene,” refers to a divalent carbocyclic non-aromatic group having from three to ten carbons (e.g., C₃-C₁₀ cycloalkenylene), unless otherwise specified. Non-limiting examples of the cycloalkenylene may include cycloprop-1-en-1,2-diyl; cycloprop-2-en-1,1-diyl; cycloprop-2-en-1,2-diyl; cyclobut-1-en-1,2-diyl; cyclobut-1-en-1,3-diyl; cyclobut-1-en-1,4-diyl; cyclobut-2-en-1,1-diyl; cyclobut-2-en-1,4-diyl; cyclopent-1-en-1,2-diyl; cyclopent-1-en-1,3-diyl; cyclopent-1-en-1,4-diyl; cyclopent-1-en-1,5-diyl; cyclopent-2-en-1,1-diyl; cyclopent-2-en-1,4-diyl; cyclopent-2-en-1,5-diyl; cyclopent-3-en-1,1-diyl; cyclopent-1,3-dien-1,2-diyl; cyclopent-1,3-dien-1,3-diyl; cyclopent-1,3-dien-1,4-diyl; cyclopent-1,3-dien-1,5-diyl; cyclopent-1,3-dien-5,5-diyl; norbornadien-1,2-diyl; norbornadien-1,3-diyl; norbornadien-1,4-diyl; norbornadien-1,7-diyl; norbornadien-2,3-diyl; norbornadien-2,5-diyl; norbornadien-2,6-diyl; norbornadien-2,7-diyl; and norbornadien-7,7-diyl. The cycloalkenylene may be unsubstituted or substituted (e.g., optionally substituted cycloalkenylene) as described for cycloalkyl.
Heterocyclyl: As used herein, the term “heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specifica-tion, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals may include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.
Substituted: As used herein, the term “substituted” refers to any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—OR^{a}, where R^{a} is C₁-C₁₂ alkyl or cycloalkyl); carboxyl groups (—OC(═O)R^{a} or —C(═O)OR^{a}, where R^{a} is H, C₁-C₁₂ alkyl or cycloalkyl); amine groups (—NR^{a}R^{b}, where R^{a} and R^{b} are each independently H, C₁-C₁₂ alkyl or cycloalkyl); C₁-C₁₂ alkyl groups; and cycloalkyl groups. In some embodiments the substituent is a C₁-C₁₂ alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.
Optionally: As used herein, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.
Pharmaceutically acceptable salt: As used herein, the term “pharmaceutically acceptable salt” refers to both acid and base addition salts.
Pharmaceutically acceptable acid addition salt: For example, “pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, ci1mamic acid, citric acid, cyclan1ic acid, dodecylsulfuric acid, ethane-1,2-disulfonic acid, ethanesul-fonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxoglutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.
Pharmaceutically acceptable base addition salt: The term “pharmaceutically acceptable base addition salt” refers to those salts which retain the biological effectiveness and properties of the free acids, which are not biologically or otherwise undesirable. These salts are prepared from addi-tion of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Suitable inorganic salts include the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Suitable organic bases include isopropylamine, diethylamine, ethanolamine, trimethylamine, dicyclohexylamine, choline and caffeine.
Stereoisomer: As used herein, the term “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuper-imposeable mirror images of one another.
Tautomer: As used herein, the term “tautomer” refers to a proton shift from one atom of a molecule to another atom of the same molecule. The present invention includes tautomers of any said compounds.
Cationic lipid: As used herein, the term “cationic lipid” refers to a lipid capable of being positively charged. Exemplary cationic lipids include one or more amine group(s) which bear the positive charge. Exemplary cationic lipids are ionizable such that they can exist in a positively charged or neutral form depending on pH. The ionization of the cationic lipid may affect the surface charge of the lipid nanoparticle under different pH conditions. This charge state can influence plasma protein absorption, blood clearance and tissue distribution (Semple, S. C., et al., Adv. Drug Deliv Rev 32:3-17 (1998)) as well as the ability to form endosomolytic non-bilayer structures (Hafez, I. M., et 35 al., Gene Ther 8:1188-1196 (2001)) for the intracellular delivery of nucleic acids.
Neutral lipid: As used herein, the term “neutral lipid” refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phosphatidylethanolan1ines such as 1,2-Dioleoyl-sn-glycero-3-phosphoethanolamine (DOPE), sphingomy-elins (SM), ceramides, steroids such as sterols and their derivatives. Neutral lipids may be synthetic or naturally derived.
Charged lipid: As used herein, the term “charged lipid” refers to any of a number of lipid species that exist in either a positively charged or negatively charged form independent of the pH within a useful physiological range e.g., pH −3 to pH −9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g., DOTAP, DOTMA), dialkyl dimethylaminopropanes, ethyl phosphocholines, dimethylaminoethane carbamoyl sterols (e.g. DCChol).
Lipid nanoparticle: As used herein, the term “lipid nanoparticle” refers to particles having at least one dimension on the order of nanometers (e.g., 1-1,000 nm) which include specified lipids. A lipid nanoparticle may also include a nucleic acid (e.g., mRNA). In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles comprise a nucleic acid. Such lipid nanoparticles typically comprise a nucleic acid (e.g., mRNA) and one or more excipient selected from neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The following detailed description of the embodiments of the disclosure will be better understood when read in conjunction with the appended drawings. For the purpose of illustrating the disclosure, there are shown in the drawing embodiments, which are presently exemplified. It should be understood, however, that the disclosure is not limited to the precise arrangement and instrumentalities of the embodiments shown in the drawings.

FIG. 1A depicts a schematic representation of a dual target approach based on a durable block of the MYC promoter using a DBD fused to a DNA methyltransferase, and a transient (48/72 Hours) block of CTCF/TF sites using a DBD or a DBD fused to a short-term effector.

FIG. 1B depicts guide RNA localization and chromatin context of target sites (CTCF and promoter) for the MYC gene. From top to bottom, the graphs represent, for the MYC locus in HepG2 cells, H3K4me3 (histone H3 Ky trimethylation) levels; H3K9me3 (histone H3 K9 trimethylation) levels (replicate 1); H3K9me3 (histone H3 K9 trimethylation) levels (replicate 2); H3K27me3 (histone H3 K27 trimethylation) levels; H3K27ac (histone H3 K27 acetylation) levels; GROseq_fwdStrand levels (binding of transcriptionally active RNA pol II on the forward strand); GROseq_revStrand levels (binding of transcriptionally active RNA pol II on the reverse strand); RNAseq_rep2 levels (MYC transcript levels measured using RNAseq, replicate 2); DNA methylation level as measured by WGBS (whole-genome bisulfite sequencing), and CTCF binding levels. The positions of four gRNAs are indicated with arrows. gRNAs GD-28859, GD-28616, GD-28862 target at or near the anchor site upstream of MYC, and gRNA GD-28617 targets the MYC promoter. In this disclosure, GD-28859 is also referred to as GD-59; GD-28616 is also referred to as GD-16; GD-28862 is also referred to as GD-62; and GD-28617 is also referred to as GD-17.

FIG. 1C shows a schematic diagram of an exemplary bi-cistronic construct. The 5′ end of the construct possesses a cap structure defined by an N7-methylated guanosine linked to the first nucleotide of the mRNA via a reverse 5′ to 5′ triphosphate linkage. In some embodiments, the cap structure promotes protein translation and stability. Downstream of the cap structure is an un-translated region (5′ UTR) designed to promote high levels of protein translation, followed by the canonical “Kozak” sequence that is recognized by the ribosome to start translation of the protein. Following the “Kozak” sequence is the CDS which is a single continuous sequence comprising the first expression repressor comprising a first targeting moiety and a first effector moiety and the second expression repressor comprising the second targeting moiety and the second effector moiety separated by a tPT2A “ribosome skipping” sequence (the linker). Without wishing to be bound by theory, when a ribosome reaches the tPT2A linker, it begins translating the linker into amino acids. The first 18 amino acids produced from the P2A linker remain at the C-terminal end of the first expression repressor (e.g., comprising a ZF DBD and MQ1), which the ribosome then releases. The ribosome then continues on until it reaches the T2A linker, and the first 17 residues of the T2A linker are translated and released. Next, the second polypeptide is translated, comprising a single amino acid and then the beginning of the second expression repressor (e.g., comprising a second ZF DBD and KRAB). After the CDS is a 3′ UTR which is designed to aid in high levels of translation and to also stabilize the mRNA. Finally, at the very 3′ end of the mRNA is the polyA tail. In some embodiments, the polyA tail promotes protein translation and mRNA stability.

FIGS. 2A and 2B are graphs showing the efficacy of the combination of bicistronic ZF9-MQ_ZF3-KRAB and lenvatinib. FIG. 2A is a graph showing the post treatment Hep3B cell viability relative to untreated cells. FIG. 2B is a graph showing the post treatment SK-HEP-1 cell viability relative to untreated cells. This study was performed as described in Example 1.

FIGS. 3A and 3B are graphs showing the efficacy of the combination of bicistronic ZF9-MQ_ZF3-KRAB with an AKT and ERK inhibitor. FIG. 3A is a graph showing the post treatment SK-HEP-1 cell viability relative to untreated cells for ERK inhibitor ulixertinib. FIG. 3B is a graph showing the post treatment SK-HEP-1 cell viability relative to untreated cells for AKT inhibitor MK-2206. Alone=AKT or ERK inhibitor alone, respectively; SNC=Short Non-Coding (control). This study was performed as described in Example 2.

FIGS. 4A and 4B are bar graphs showing the efficacy of the combination of bicistronic ZF9-MQ_ZF3-KRAB and osimertinib. FIG. 4A is a bar graph showing the Myc protein (normalized to untreated) concentration in PC9 cells 48 hours after treatment with PBS (untreated), osimertinib, bicistronic ZF9-MQ_ZF3-KRAB, and a combination of bicistronic ZF9-MQ_ZF3-KRAB and osimertinib. FIG. 4B is a bar graph showing the 72 hour post treatment PC9 cell viability. * denotes p<or=0.05; ** denotes p<or=0.01; *** denotes p<or=0.001. This study was performed as described in Example 3.

FIGS. 5A and 5B are bar graphs showing the efficacy of the combination of bicistronic ZF9-MQ_ZF3-KRAB and trametinib. FIG. 5A is a bar graph showing the Myc protein (normalized to untreated) concentration in H1975 cells 48 hours aftertreatment with PBS (untreated), trametinib, bicistronic ZF9-MQ_ZF3-KRAB, and a combination of bicistronic ZF9-MQ_ZF3-KRAB and trametinib. FIG. 5B is a bar graph showing the 72 hour post treatment H1975 cell viability. * denotes p<or=0.05; ** denotes p<or=0.01; *** denotes p<or=0.001. This study was performed as described in Example 3.

FIG. 6 is a graph showing the decreased survival probability with treatment on Osimertinib. Univariate Kaplan-Meier analysis of MYC over-expression and time to progression. Time to progression entries that are greater than 99th percentile and less than 30 days have been omitted.

FIG. 7A is a graph showing the cell viability of H1975 cells and two Osimertinib-resistant cell lines derived from H1975 (OsimR_1.25 and OsimR_1.5) 72 hours after treatment with Osimertinib. This study was performed as described in Example 7.

FIG. 7B is a graph showing the cell viability of H1975 cells and two Osimertinib-resistant cell lines derived from H1975 (OsimR_1.25 and OsimR_1.5) 72 hours after treatment with expression repressor. This study was performed as described in Example 7.

FIG. 8A is an image of a protein matrix gel assessing MYC protein levels after treatment with osimertinib or MR-32380. This study was performed as described in Example 8.

FIG. 8B is a graph showing MYC protein level in H1975 cells and two Osimertinib-resistant cell lines derived from H1975 (OsimR_1.25 and OsimR_1.5) after treatment with vehicle (control), osimertinib, or MR-32380. This study was performed as described in Example 8.

FIG. 9 is an image of a Western blot showing MYC protein levels in PC9 cells with a sensitizing EGFR mutation (del19) treated with osimertinib or MR-32054, alone or in combination. This study was performed as described in Example 9.

FIG. 10 is a bar graph showing MYC protein levels (normalized to Actin, untreated) in PC9 cells with a sensitizing EGFR mutation (del19) treated with osimertinib or MR-32054, alone or in combination. This study was performed as described in Example 9.

FIG. 11 is a bar graph showing cell viability in H1975 cells with a sensitizing mutation (EGFR L858R, T790M) treated with osimertinib or MR-32054, alone or in combination. This study was performed as described in Example 10.

FIG. 12 is a plot showing the Bliss synergy score for H1975 cells with a sensitizing mutation (EGFR L858R, T790M) treated with osimertinib or MR-32054, alone or in combination. All the values shown are between 0 and 40. This study was performed as described in Example 10.

FIG. 13 is a bar graph showing cell viability in PC9 cells with a sensitizing EGFR mutation (del19) treated with osimertinib or MR-32054, alone or in combination. This study was performed as described in Example 11.

FIG. 14 is a plot showing the Bliss synergy score for PC9 cells with a sensitizing EGFR mutation (del19) treated with osimertinib or MR-32054, alone or in combination. All the values shown are between 0 and 40. This study was performed as described in Example 11.

FIG. 15 is a graph showing the efficacy of the combination of bicistronic ZF9-MQ_ZF3-KRAB and osimertinib. The combination of bicistronic ZF9-MQ_ZF3-KRAB and osimertinib reduced tumor growth greater than either component alone. This study was performed as described in Example 12.

FIG. 16 is a graph showing body weight (% change from baseline) in H1975 subcutaneous tumor model with sensitizing EGFR mutations (L858R, T790M). This study was performed as described in Example 12.

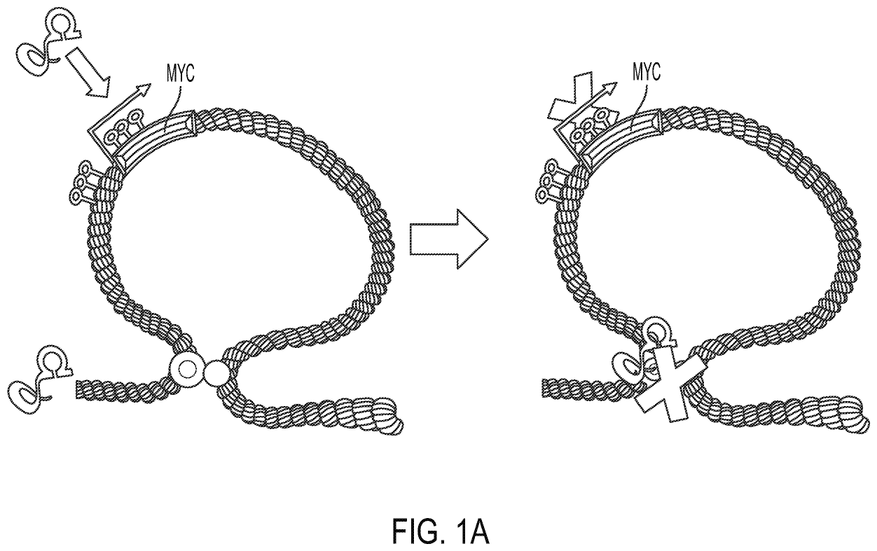

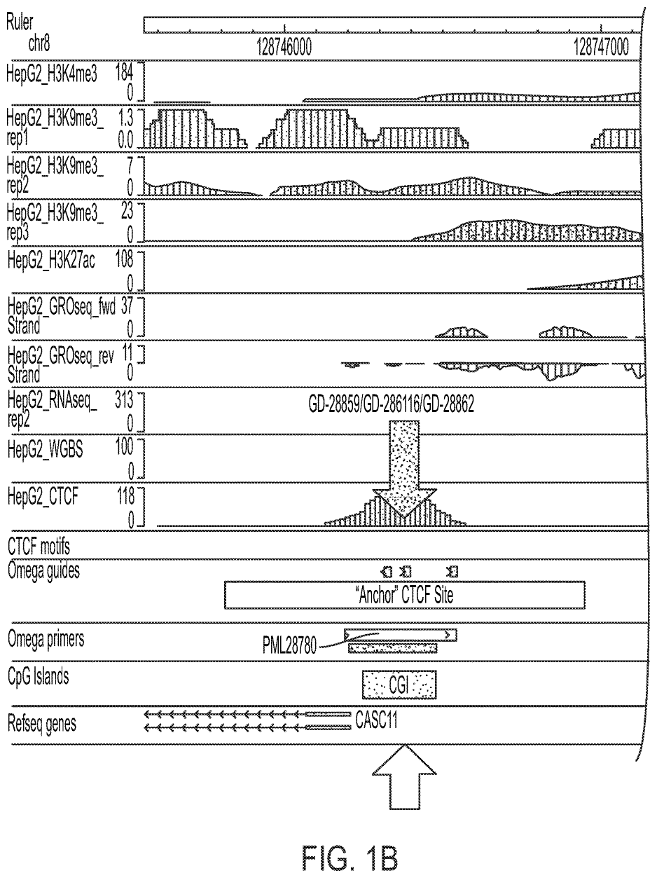

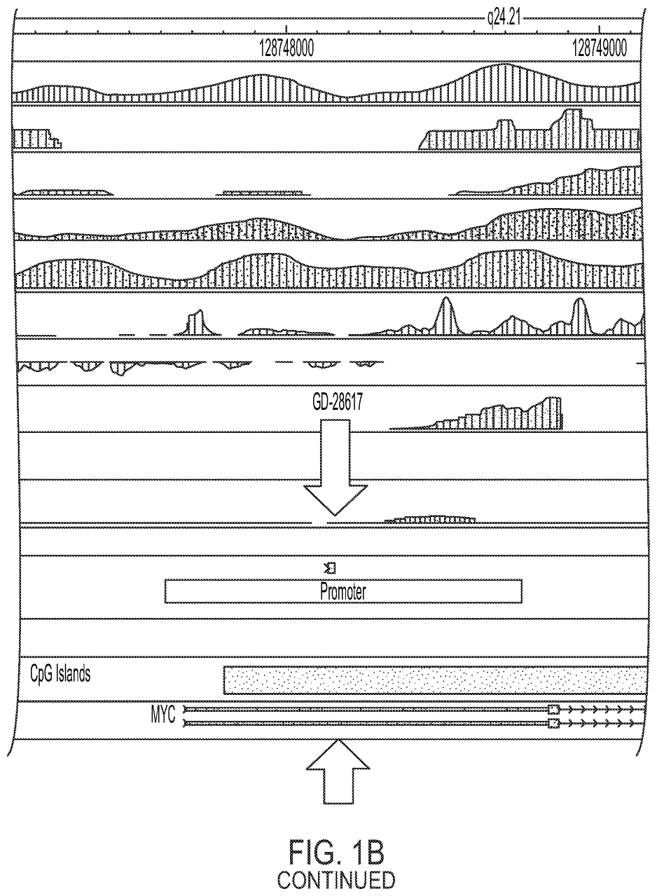

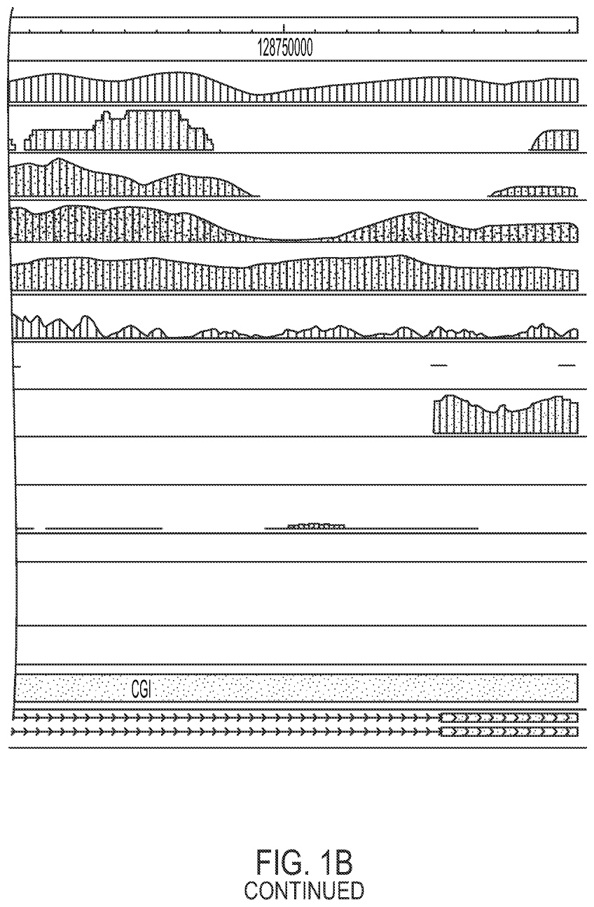

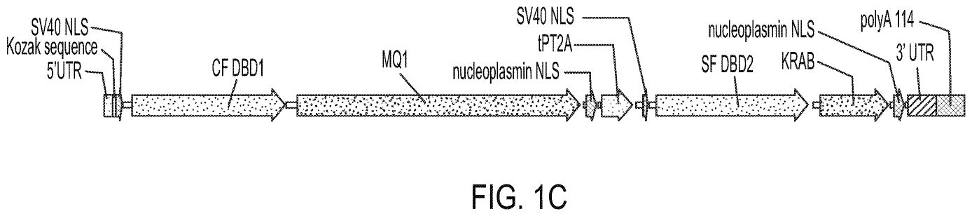

### DETAILED DESCRIPTION OF CERTAIN EMBODIMENTS

The present disclosure provides technologies for modulating, e.g., decreasing, expression of a target gene e.g., MYC in cell, e.g., in a subject or patient, through the use of an expression repressor or a system described herein.

Many different diseases and syndromes, including cancer, autoimmunity, cardiovascular disease, and obesity, can be caused by mis-regulation of gene expression. Particularly, overexpression of transcription factors has long been known to known to contribute to tumorigenesis, and recent studies indicate that overexpressed oncogenic transcription factors can alter the core autoregulatory circuitry of the cell.

MYC, a transcription factor and master cell regulator, is frequently dysregulated in over 50% of human cancer and plays a central role in nearly every aspect of the tumorigenic process. Except for early response genes, MYC typically upregulates gene expression. MYC is the most frequently amplified oncogene, and the elevated expression of its gene product is associated with tumor aggression and poor clinical outcome. Elevated levels of c-MYC can promote tumorigenesis in a wide range of tissues. Most tumor cells depend on the transcription factor c-MYC for their growth and proliferation. MYC overexpression is also associated in chronic liver disease e.g., viral and alcohol related liver disease. MYC overexpression level varies in specific cancer subtypes. Without wishing to be bound by theory, it is thought that modulating e.g., decreasing the levels of MYC in a subject (e.g., overall, or in a specific target tissue or tissues) suffering from MYC mis-regulation disorder may lessen or eliminate the symptoms of the MYC mis-regulation disorder.

### Expression Repressors

As described herein, the present disclosure in part provides expression repressors for modulating, e.g., decreasing the expression of a target gene, e.g., MYC. In some embodiments, an expression repressor may comprise a targeting moiety that binds to a target gene promoter, e.g., MYC promoter and optionally an effector moiety. In some embodiments, the targeting moiety specifically binds a target DNA sequence, e.g., MYC DNA sequence, thereby localizing the expression repressor's functionality to the DNA sequence. In some embodiments, an expression repressor comprises a targeting moiety and one effector moiety. In some embodiments an expression repressor comprises a targeting moiety and a plurality of effector moieties, e.g., as described in International Application WO/2022/132195, which is herein incorporated by reference in its entirety.

In some embodiments, an expression repressor comprises one or more targeting moieties, e.g., as described in International Application WO/2022/132195, which is herein incorporated by reference in its entirety.

In some embodiments, an expression repressor comprises a targeting moiety and an effector moiety that are covalently linked, e.g., by a peptide bond. In some embodiments, the targeting moiety and the effector moiety are situated on the same polypeptide chain, e.g., connected by one or more peptide bonds and/or a linker. In some embodiments, the expression repressor is or comprises a fusion molecule, e.g., comprising the targeting moiety and the effector moiety linked by a peptide bond and/or a linker. In some embodiments, the expression repressor comprises a targeting moiety that is disposed N-terminal of an effector moiety on the same polypeptide chain. In some embodiments, the expression repressor comprises a targeting moiety that is disposed C-terminal of an effector moiety on the same polypeptide chain. In some embodiments, an expression repressor comprises a targeting moiety and an effector moiety that are covalently linked by a non-peptide bond. In some embodiments, a targeting moiety is conjugated to an effector moiety by a non-peptide bond. In some embodiments, an expression repressor comprises a targeting moiety and a plurality of effector moieties, wherein the targeting moiety and the plurality of effector moieties are covalently linked, e.g., by peptide bonds (e.g., the targeting moiety and plurality of effector moieties are all connected by a series of covalent bonds, although each individual moiety may not share a covalent bond with every other effector moiety).

In other embodiments, an expression repressor comprises a targeting moiety and an effector moiety that are not covalently linked e.g., as described in International Application WO/2022/132195, which is herein incorporated by reference in its entirety.

In general, an expression repressor as described herein binds (e.g., via a targeting moiety) a genomic sequence element proximal to and/or operably linked to a target gene, e.g., MYC. In some embodiments, binding of the expression repressor to the genomic sequence element modulates (e.g., decreases) expression of the target gene, e.g., MYC. For example, binding of an expression repressor comprising an effector moiety that recruits or inhibits recruitment of components of the transcription machinery to the genomic sequence element may modulate (e.g., decrease) expression of the target gene, e.g., MYC. As a further example, binding of an expression repressor comprising an effector moiety with an enzymatic activity (e.g., an epigenetic modifying moiety) may modulate (e.g., decrease) expression of the target gene, e.g., MYC) through the localized enzymatic activity of the effector moiety. As a further example, both binding of an expression repressor to a genomic sequence element and the localized enzymatic activity of an expression repressor may contribute to the resulting modulation (e.g., decrease) in expression of the target gene, e.g., MYC.

In some embodiments, an expression repressor comprises an effector moiety wherein the effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB (e.g., a KRAB domain), MeCP2, HP1, RBBP4, REST, FOG1, SUZ12 or a functional variant or fragment thereof.

In some embodiments, the disclosure provides nucleic acid sequences encoding an expression repressor, an expression repressor system, a targeting moiety and/or an effector moiety as described herein. A skilled artisan is aware that the nucleic acid sequences of RNA are identical to the corresponding DNA sequences, except that typically thymine (T) is replaced by uracil (U). It will be understood that when a nucleotide sequence is represented by a DNA sequence (e.g., comprising, A, T, G, C), this disclosure also provides the corresponding RNA sequence (e.g., comprising, A, U, G, C) in which “U” replaces “T.” Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

It will be appreciated by those skilled in the art that due to the degeneracy of the genetic code, a multitude of nucleotide sequences encoding an expression repressor comprising targeting moiety and/or an effector moiety as described herein may be produced, some of which have similarity, e.g., 90%, 95%, 96%, 97%, 98%, or 99% identity to the nucleic acid sequences disclosed herein. For instance, codons AGA, AGG, CGA, CGC, CGG, and CGU all encode the amino acid arginine. Thus, at every position in the nucleic acid molecules of the invention where an arginine is specified by a codon, the codon can be altered to any of the corresponding codons described above without altering the encoded polypeptide.

In some embodiments a nucleic acid cohesion encoding an expression repressor comprising a targeting moiety and/or an effector moiety may be part or all of a codon-optimized coding region, optimized according to codon usage in mammals, e.g., humans. In some embodiments, a nucleic acid cohesion encoding a targeting moiety and/or an effector moiety is codon optimized for increasing the protein expression and/or increasing the duration of protein expression. In some embodiments, a protein produced by the codon optimized nucleic acid sequence is at least 1%, at least 2%, at least 5%, at least 10%, at least 15%, at least 20%, at least 30%, at least 40%, or at least 50% higher compared to levels of the protein when encoded by a nucleic acid sequence that is not codon optimized.

### Expression Repression Systems

Expression repression systems of the present disclosure may comprise two or more expression repressors. In some embodiments, an expression repression system comprises 2, 3, 4, 5, 6, 7, 8, 9, 10, 11, 12, or more expression repressors (and optionally no more than 15, 14, 13, 12, 11, 10, 9, 8, 7, 6, 5, 4, 3, or 2). In some embodiments, an expression repression system targets two or more different sequences (e.g., a 1^{st} and 2^{nd}, 3^{rd}, 4^{th}, 5^{th}, 6^{th}, 7^{th}, 8^{th}, 9^{th}, 10^{th}, 11^{th}, 12^{th}, and/or further DNA sequence, and optionally no more than a 20^{th}, 19^{th}, 18^{th}, 17^{th}, 16^{th}, 15^{th}, 14^{th}, 13^{th}, 12^{th}, 11^{th}, 10^{th}, 9^{th}, 8^{th}, 6^{th}, 5^{th}, 4^{th}, 3^{rd}, or 2^{nd} sequence). In some embodiments, an expression repression system comprises a plurality of expression repressors, wherein each member of the plurality of expression repressors does not detectably bind, e.g., does not bind, to another member of the plurality of expression repressors. In some embodiments, an expression repression system comprises a first expression repressor and a second expression repressor, wherein the first expression repressor does not detectably bind, e.g., does not bind, to the second expression repressor.

In some embodiments, an expression repression system of the present disclosure comprises two or more expression repressors, wherein the expression repressors are present together in a composition, pharmaceutical composition, or mixture. In some embodiments, an expression repression system of the present disclosure comprises two or more expression repressors, wherein one or more expression repressors is not admixed with at least one other expression repressor. For example, an expression repression system may comprise a first expression repressor and a second expression repressor, wherein the presence of the first expression repressor in the nucleus of a cell does not overlap with the presence of the second expression repressor in the nucleus of the same cell, wherein the expression repression system achieves a decrease in expression of a MYC gene via the non-overlapping presences of the first and second expression repressors. In some embodiments, the expression repression system achieves a greater decrease in expression of a MYC gene in comparison to the decrease in expression of a MYC gene achieved by the first or the second expression repressor alone.

In some embodiments, the expression repressors of an expression repressor system each comprise a different targeting moiety (e.g., the first, second, third, or further expression repressors each comprise different targeting moieties from one another). For example, an expression repression system may comprise a first expression repressor and a second expression repressor wherein the first expression repressor comprises a first targeting moiety (e.g., a Cas9 domain, TAL effector domain, or Zn Finger domain), and the second expression repressor comprises a second targeting moiety (e.g., a Cas9 domain, TAL effector domain, or Zn Finger domain) different from the first targeting moiety. In some embodiments, different can mean comprising distinct types of targeting moiety, e.g., the first targeting moiety comprises a Cas9 domain, and the second DNA-targeting moiety comprises a Zn finger domain. In other embodiments, different can mean comprising distinct variants of the same type of targeting moiety, e.g., the first targeting moiety comprises a first Cas9 domain (e.g., from a first species) and the second targeting moiety comprises a second Cas9 domain (e.g., from a second species). In an embodiment, when an expression repressor system comprises two or more targeting moieties of the same type, e.g., two or more Cas9 or ZF domains, the targeting moieties specifically bind two or more different sequences. For example, in an expression repressor system comprising two or more Cas9 domains, the two or more Cas9 domains may be chosen or altered such that they only appreciably bind the gRNA corresponding to their target sequence (e.g., and do not appreciably bind the gRNA corresponding to the target of another Cas9 domain). In a further example, in an expression repressor system comprising two or more effector moieties, the two or more effector moieties may be chosen or altered such that they only appreciably bind to their target sequence (e.g., and do not appreciably bind the target sequence of another effector moiety).

In some embodiments, the expression repressors of an expression repressor system each bind to a different DNA sequence (e.g., the first, second, third, or further expression repressors each bind DNA sequences that are different from one another). For example, an expression repression system may comprise a first expression repressor and a second expression repressor wherein the first expression repressor binds to a first DNA sequence, and the second expression repressor binds to a second DNA sequence. In some embodiments, different can mean that: there is at least one position that is not identical between the DNA sequence bound by one expression repressor and the DNA sequence bound by another expression repressor, or that there is at least one position present in the DNA sequence bound by one expression repressor that is not present in the DNA sequence bound by another expression repressor.

In some embodiments, the first DNA sequence may be situated on a first genomic DNA strand and the second DNA sequence may be situated on a second genomic DNA strand. In some embodiments, the first DNA sequence may be situated on the same genomic DNA strand as the second DNA sequence.

In some embodiments, an expression repression system comprises two or more (e.g., two) expression repressors and a plurality (e.g., two) of the expression repressors comprise targeting moieties that bind to different DNA sequences. In such embodiments, a first targeting moiety may bind to a first DNA sequence and a second DNA-targeting moiety may bind to a second DNA sequence, wherein the first and the second DNA sequences are different and do not overlap.

In some embodiments, the expression repressors of an expression repressor system each comprise a different effector moiety (e.g., the first, second, third, or further expression repressors each comprise a different effector moiety from one another). For example, an expression repression system may comprise a first expression repressor and a second expression repressor wherein the first expression repressor comprises a first effector moiety (e.g., comprising a DNA methyltransferase or functional fragment thereof), and the second expression repressor comprises a second effector moiety (e.g., comprising a transcription repressor (e.g., KRAB) or functional fragment thereof) different from the first effector moiety. In some embodiments, different can mean comprising distinct types of effector moiety. In other embodiments, different can mean comprising distinct variants of the same type of effector moiety, e.g., the first effector moiety comprises a first DNA methyltransferase (e.g., having a first site specificity or amino acid sequence) and the second effector moiety comprises a second DNA methyltransferase (e.g., having a second site specificity or amino acid sequence).

In some embodiments, an expression repressor system comprises a first expression repressor comprising a first effector moiety and a second expression repressor comprising a second effector moiety, wherein the first effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB, MeCP2, HP1, RBBP4, REST, FOG1, SUZ12 or a functional variant or fragment thereof, and the second effector moiety comprises a different protein chosen from MQ1, DNMT1, DNMT3A1, DNMT3A2, DNMT3B1, DNMT3B2, DNMT3B3, DNMT3B4, DNMT3B5, DNMT3B6, DNMT3L, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, KDM1A (i.e., LSD1), KDM1B (i.e., LSD2), KDM2A, KDM2B, KDM5A, KDM5B, KDM5C, KDM5D, KDM4B, NO66, SETDB1, SETDB2, EHMT2 (i.e., G9A), EHMT1 (i.e., GLP), SUV39H1, EZH2, EZH1, SUV39H2, SETD8, SUV420H1, SUV420H2, KRAB, MeCP2, HP1, RBBP4, REST, FOG1, SUZ12, or a functional variant or fragment thereof.

In some embodiments, the first or second effector moiety comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof, and the other effector moiety comprises a transcription repressor activity (e.g., KRAB, MeCP2, HP1, RBBP4, REST, FOG1, SUZ12, or a functional variant or fragment of any thereof), the first or second effector moiety comprises a histone methyltransferase activity and the other effector moiety comprises a histone deacetylase activity (e.g., HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, the first or second effector moiety comprises a histone methyltransferase activity and the other effector moiety comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof). In some embodiments, the first or second effector moiety comprises a DNA methyltransferase activity and the other effector moiety comprises a transcription repressor activity. In some embodiments the first or second effector moiety comprises a histone methyltransferase activity and the other effector moiety comprises a transcription repressor activity (e.g., KRAB, MeCP2, HP1, RBBP4, REST, FOG1, SUZ12, or a functional variant or fragment of any thereof).

In some embodiments, two or more (e.g., all) expression repressors of an expression repressor system are not covalently associated with each other, e.g., each expression repressor is not covalently associated with any other expression repressor. In another embodiment, two or more expression repressors of an expression repressor system are covalently associated with one another. In an embodiment, an expression repression system comprises a first expression repressor and a second expression repressor disposed on the same polypeptide, e.g., as a fusion molecule, e.g., connected by a peptide bond and optionally a linker. In some embodiments, the peptide is a self-cleaving peptide, e.g., a T2A self-cleaving peptide. In an embodiment, an expression repression system comprises a first expression repressor and a second expression repressor that are connected by a non-peptide bond, e.g., are conjugated to one another.

### Linkers

An expression repressor or an expression repressor system as disclosed herein may comprise one or more linkers. A linker may connect a targeting moiety to an effector moiety, an effector moiety to another effector moiety, or a targeting moiety to another targeting moiety. A linker may be a chemical bond, e.g., one or more covalent bonds or non-covalent bonds. In some embodiments, a linker is covalent. In some embodiments, a linker is non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first domain or moiety from a second domain or moiety, e.g., a DNA-targeting moiety from an effector moiety. In some embodiments, for example, a linker can be positioned between a DNA-targeting moiety and an effector moiety, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g., polypeptide) from a modulating agent, but sufficiently stable to prevent premature cleavage.

In some embodiments, one or more moieties and/or domains of an expression repressor described herein are linked with one or more linkers. In some embodiments, an expression repression may comprise a linker situated between the targeting moiety and the effector moiety. In some embodiments, the linker may have a sequence of ASGSGGGSGGARD (SEQ ID NO: 137), or ASGSGGGSGG (SEQ ID NO: 138). In some embodiments, a system comprising a first and second repressor may comprise a first linker situated between the first targeting moiety and the first effector moiety, and a second linker situated between the second targeting moiety and the second effector moiety. In some embodiments, the first and the second linker may be identical. In some embodiments, the first and the second linker may be different. In some embodiments, the first linker may comprise an amino acid sequence according to SEQ ID NO: 137 or a sequence with at least 80, 85, 90, 95, 99, or 100% identity thereto and the second linker may comprise an amino acid sequence according to SEQ ID NO: 138 or a sequence with at least 80, 85, 90, 95, 99, or 100% identity thereto.

As will be known by one of skill in the art, commonly used flexible linkers have sequences consisting primarily of stretches of Gly and Ser residues (“GS” linker). Flexible linkers may be useful for joining domains/moieties that require a certain degree of movement or interaction and may include small, non-polar (e.g., Gly) or polar (e.g., Ser or Thr) amino acids. Incorporation of Ser or Thr can also maintain the stability of a linker in aqueous solutions by forming hydrogen bonds with water molecules, and therefore reduce unfavorable interactions between a linker and moieties/domains. In some embodiments, the linker is a GS linker or a variant thereof e.g., G4S (SEQ ID NO: 207).

Rigid linkers are useful to keep a fixed distance between domains/moieties and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments. In some embodiment, the cleavable linker may be a self-cleaving linker, e.g., a T2A peptide linker. In some embodiments, the linker may comprise a “ribosome skipping” sequence, e.g., a tPT2A linker.

Examples of molecules suitable for use in linkers described herein include a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof; noncarbon linkers; carbohydrate linkers; phosphodiester linkers, or other molecule capable of covalently linking two or more components of an expression repressor. Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine, or other hydrophobic residues. Components of an expression repressor may be linked using charge-based chemistry, such that a positively charged component of an expression repressor is linked to a negative charge of another component.

### Targeting Moieties

The present disclosure provides, e.g., expression repressors comprising a targeting moiety that specifically targets, e.g., binds, a genomic sequence element (e.g., a promoter, a TSS, or an anchor sequence) in, proximal to, and/or operably linked to a target gene. Targeting moieties may specifically bind a DNA sequence, e.g., a DNA sequence associated with a target gene, e.g., MYC. Any molecule or compound that specifically binds a DNA sequence may be used as a targeting moiety.

In some embodiments, a targeting moiety targets, e.g., binds, a component of a genomic complex (e.g., ASMC). In some embodiments, a targeting moiety targets. e.g., binds, an expression control sequence (e.g., a promoter or enhancer) operably linked to a target gene. In some embodiments, a targeting moiety targets, e.g., binds, a target gene or a part of a target gene. The target of a targeting moiety may be referred to as its targeted component. A targeted component may be any genomic sequence element operably linked to a target gene, or the target gene itself, including but not limited to a promoter, enhancer, anchor sequence, exon, intron, UTR encoding sequence, a splice site, or a transcription start site. In some embodiments, a targeting moiety binds specifically to one or more target anchor sequences (e.g., within a cell) and not to non-targeted anchor sequences (e.g., within the same cell).

In some embodiments, the disclosure provides expression repressor comprising: a targeting moiety that binds a genomic locus comprising at least 14, 15, 16, 17, 18, 19, or 20 nucleotides of the sequence of SEQ ID NO: 1, 3, 97, 98, 99, 100, 101, 102, 103, 104, 105, 106, 107, 109, 110, or 75, 76, 78, 79, 80, 81, 84, 85, or 86, wherein the expression repressor is capable of decreasing expression of MYC.

In some embodiments, the disclosure provides an expression repressor comprising: a targeting moiety that bind a genomic locus comprising at least 16, 17, 18, 19, or 20 nucleotides of the sequence of SEQ ID NO: 2 or 77, 82, or 83 and wherein the expression repressor is capable of decreasing expression of target gene, e.g., MYC. In some embodiments, the expression repressor comprises an effector moiety.

In some embodiments, the disclosure provides an expression repressor comprising a targeting moiety wherein the targeting moiety binds a genomic locus that is within 1400 nt upstream or downstream of SEQ ID NO: 4.

In some embodiments, the disclosure provides an expression repressor comprising a targeting moiety wherein, the targeting moiety binds a genomic locus comprising at least 14, 15, 16, 17, 18, 19, or 20 nucleotides of the sequence of SEQ ID NO: 4, 77, 82, or 83.

In some embodiments, the disclosure provides an expression repressor comprising a targeting moiety wherein, the targeting moiety binds a genomic locus comprising at least 14, 15, 16, 17, 18, 19, or 20 nucleotides of the sequence of SEQ ID NO: 83, 96, or 108.

In some embodiments, a targeting moiety may be or comprise a CRISPR/Cas domain, a TAL effector domain, a Zn finger domain, peptide nucleic acid (PNA) or a nucleic acid molecule. In some embodiments, an expression repressor comprises one effector moiety. In some embodiments, an expression repressor comprises a plurality of targeting moieties, wherein each targeting moiety does not detectably bind, e.g., does not bind, to another targeting moiety. In some embodiments, an expression repression system comprises a plurality of expression repressors, wherein each member of the plurality of expression repressors comprises a targeting moiety, wherein each targeting moiety does not detectably bind, e.g., does not bind, to another targeting moiety. In some embodiments, an expression repression system comprises a first expression repressor comprising a first targeting moiety and a second expression repressor comprising a second targeting moiety, wherein the first targeting moiety does not detectably bind, e.g., does not bind, to the second targeting moiety. In some embodiments, an expression repression system comprises a first expression repressor comprising a first targeting moiety and a second expression repressor comprising a second targeting moiety, wherein the first targeting moiety does not detectably bind, e.g., does not bind, to another first targeting moiety, and the second targeting moiety does not detectably bind, e.g., does not bind, to another second targeting moiety. In some embodiments, a targeting moiety for use in the compositions and methods described herein is functional (e.g., binds to a DNA sequence) in a monomeric, e.g., non-dimeric, state.

In some embodiments, a targeting moiety may be or comprise a CRISPR/Cas domain, a TAL effector domain, a Zn finger domain, or a nucleic acid molecule. In some embodiments, a targeting moiety is or comprises a DNA-binding domain from a nuclease. Exemplary nucleic acid targeting moieties are described, e.g., in International Application WO/2022/132195, which is herein incorporated by reference in its entirety.

An expression repressor or a system comprising an repressor as disclosed herein, may comprise nucleic acid, e.g., one or more nucleic acids. The term “nucleic acid” refers to any compound that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is or comprises more than 50% ribonucleotides and is referred to herein as a ribonucleic acid (RNA). In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. As used herein, “recombinant” when used to describe a nucleic acid refers to any nucleic acid that does not naturally occur. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, nucleic acids may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

### CRISPR/Cas Domains

In some embodiments, a targeting moiety is or comprises a CRISPR/Cas domain, e.g., as described in International Application WO/2022/132195, which is herein incorporated by reference in its entirety. A CRISPR/Cas protein can comprise a CRISPR/Cas effector and optionally one or more other domains. A CRISPR/Cas domain typically has structural and/or functional similarity to a protein involved in the clustered regulatory interspaced short palindromic repeat (CRISPR) system, e.g., a Cas protein. The CRISPR/Cas domain optionally comprises a guide RNA, e.g., single guide RNA (sgRNA). In some embodiments, the gRNA comprised by the CRISPR/Cas domain is noncovalently bound by the CRISPR/Cas domain.

In some embodiments, the Cas protein is modified to deactivate the nuclease, e.g., nuclease-deficient Cas. In some embodiments, the Cas protein is a Cas9 protein. Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas binding to a DNA sequence may interfere with transcription at that site by steric hindrance. In some embodiments, a DNA-targeting moiety is or comprises a catalytically inactive Cas, e.g., dCas. Many catalytically inactive Cas proteins are known in the art. In some embodiments, dCas9 comprises mutations in each endonuclease domain of the Cas protein, e.g., D10A and H840A mutations.

### TAL Effector Domains

In some embodiments, a DNA-targeting moiety is or comprises a TAL effector domain, e.g., as described in International Application WO/2022/132195, which is herein incorporated by reference in its entirety. A TAL effector domain, e.g., a TAL effector domain that specifically binds a DNA sequence, comprises a plurality of TAL effector repeats or fragments thereof, and optionally one or more additional portions of naturally occurring TAL effector repeats (e.g., N- and/or C-terminal of the plurality of TAL effector domains) wherein each TAL effector repeat recognizes a nucleotide. A TAL effector protein can comprise a TAL effector domain and optionally one or more other domains. Many TAL effector domains are known to those of skill in the art and are commercially available, e.g., from Thermo Fisher Scientific.

### Zn Finger Domains

In some embodiments, a DNA-targeting moiety is or comprises a Zn finger domain. A Zn finger domain comprises a Zn finger, e.g., a naturally occurring Zn finger or engineered Zn finger, or fragment thereof. Many Zn fingers are known to those of skill in the art and are commercially available, e.g., from Sigma-Aldrich. Generally, a Zn finger domain comprises a plurality of Zn fingers, wherein each Zn finger recognizes three nucleotides. A Zn finger protein can comprise a Zn finger domain and optionally one or more other domains. In some embodiments, the Zn finger domain or Zn finger is a Zn finger domain or Zn finger as described in International Application WO/2022/132195, which is herein incorporated by reference in its entirety

In addition, as disclosed in these and other references, zinc fingers and/or multi-fingered zinc finger domains may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

In certain embodiments, the DNA-targeting moiety comprises a Zn finger domain comprising an engineered zinc finger that binds (in a sequence-specific manner) to a target DNA sequence. In some embodiments, the Zn finger domain comprises one Zn finger or fragment thereof. In some embodiments, the Zn finger domain comprises a plurality of Zn fingers (or fragments thereof), e.g., 2, 3, 4, 5, 6 or more Zn fingers (and optionally no more than 12, 11, 10, 9, 8, 7, 6, 5, 4, 3, or 2 Zn fingers). In some embodiments, the Zn finger domain comprises at least three Zn fingers. In some embodiments, the Zn finger domain comprises four, five or six Zn fingers. In some embodiments, the Zn finger domain comprises 8, 9, 10, 11 or 12 Zn fingers. In some embodiments, a Zn finger domain comprising three Zn fingers recognizes a target DNA sequence comprising 9 or 10 nucleotides. In some embodiments, a Zn finger domain comprising four Zn fingers recognizes a target DNA sequence comprising 12 to 14 nucleotides. In some embodiments, a Zn finger domain comprising six Zn fingers recognizes a target DNA sequence comprising 18 to 21 nucleotides.

In some embodiments, a targeting domain comprises a two-handed Zn finger protein. Two handed zinc finger proteins are those proteins in which two clusters of zinc fingers are separated by intervening amino acids so that the two zinc finger domains bind to two discontinuous target DNA sequences. An example of a two-handed type of zinc finger binding protein is SIP1, where a cluster of four zinc fingers is located at the amino terminus of the protein and a cluster of three Zn fingers is located at the carboxyl terminus (see Remade, et al. (1999) EMBO Journal 18(18):5073-5084). Each cluster of zinc fingers in these domains is able to bind to a unique target sequence and the spacing between the two target sequences can comprise many nucleotides.

In some embodiments, an expression repressor comprises a targeting moiety comprising an engineered DNA binding domain (DBD), e.g., a Zn finger domain comprising a Zn finger (ZFN) that binds to a target sequence, e.g., a promoter or transcription start site (TSS)) sequence operably linked to a target gene (e.g., MYC), e.g., a sequence proximal to the transcription regulatory element, e.g., an anchor sequence of an anchor sequence mediated conjunction (ASMC) comprising a target gene (e.g., MYC), e.g., a sequence proximal to the anchor sequence. In some embodiments, the ZFN can be engineered to carry epigenetic effector molecules to target sites. In some embodiments, the targeting moiety comprises a Zn Finger domain that comprises 2, 3, 4, 5, 6, 7, or 8 zinc fingers. The amino acid sequences of exemplary targeting moieties disclosed herein are listed in Table 4. The nucleotide sequences encoding exemplary targeting moieties disclosed herein are listed in Table 5. In some embodiments, an expression repressor or system described herein comprises a targeting moiety having a sequence set forth in Table 4, or a sequence with at least 70%, 75%, 80%, 85%, 90%, 95%, 98%, or 99% identity thereto. In some embodiments, a nucleic acid described herein comprises a sequence set forth in Table 5, or a sequence with at least 70%, 75%, 80%, 85%, 90%, 95%, 98%, or 99% identity thereto.

TABLE 4
Amino acid sequences of exemplary targeting moieties

| NAME | SEQ ID NO. | SEQUENCE |
|---|---|---|
| ZF1 | 5 | LEPGEKPYKCPECGKSFSRSDKLTEHQRTHTGEKPYKCPECGKSFSTKNSLTEHQRTHTGEKP |
| (aa) | | YKCPECGKSFSQSGDLRRHQRTHTGEKPYKCPECGKSFSTTGALTEHQRTHTGEKPYKCPECG |
| | | KSFSDSGNLRVHQRTHTGEKPYKCPECGKSFSQRAHLERHQRTHTGEKPYKCPECGKSFSTSG |
| | | ELVRHQRTHTGEKPTGKKTS |
| ZF2 | 6 | LEPGEKPYKCPECGKSFSSPADLTRHQRTHTGEKPYKCPECGKSESDPGHLVRHQRTHTGEKP |
| (aa) | | YKCPECGKSFSRSDNLVRHQRTHTGEKPYKCPECGKSFSQSSSLVRHQRTHTGEKPYKCPECG |
| | | KSFSTSHSLTEHQRTHTGEKPYKCPECGKSFSRNDALTEHQRTHTGEKPYKCPECGKSFSRND |
| | | ALTEHQRTHTGEKPTGKKTS |
| ZF3 | 7 | LEPGEKPYKCPECGKSFSRSDKLVRHQRTHTGEKPYKCPECGKSFSQRAHLERHQRTHTGEKP |
| (aa) | | YKCPECGKSFSDPGHLVRHQRTHTGEKPYKCPECGKSFSRSDKLVRHQRTHTGEKPYKCPECG |
| | | KSFSQLAHLRAHQRTHTGEKPYKCPECGKSFSRADNLTEHQRTHTGEKPYKCPECGKSFSDCR |
| | | DLARHQRTHTGEKPTGKKTS |
| ZF4 | 8 | LEPGEKPYKCPECGKSFSHTGHLLEHQRTHTGEKPYKCPECGKSFSTTGNLTVHQRTHTGEK |
| (aa) | | PYKCPECGKSFSDKKDLTRHQRTHTGEKPYKCPECGKSFSQSGDLRRHQRTHTGEKPYKCPE |
| | | CGKSFSQRAHLERHQRTHTGEKPYKCPECGKSFSRSDKLTEHQRTHTGEKPYKCPECGKSFS |
| | | RTDTLRDHQRTHTGEKPTGKKTS |
| ZF5 | 9 | LEPGEKPYKCPECGKSFSHTGHLLEHQRTHTGEKPYKCPECGKSFSTSGNLTEHQRTHTGEK |
| (aa) | | PYKCPECGKSESTSGNLVRHQRTHTGEKPYKCPECGKSFSQLAHLRAHQRTHTGEKPYKCPE |
| | | CGKSFSERSHLREHQRTHTGEKPYKCPECGKSFSDPGHLVRHQRTHTGEKPYKCPECGKSFS |
| | | THLDLIRHQRTHTGEKPTGKKTS |
| ZF6 | 10 | LEPGEKPYKCPECGKSFSDPGALVRHQRTHTGEKPYKCPECGKSFSERSHLREHQRTHTGEK |
| (aa) | | PYKCPECGKSFSRSDHLTNHQRTHTGEKPYKCPECGKSFSERSHLREHQRTHTGEKPYKCPE |
| | | CGKSFSRSDHLTNHQRTHTGEKPYKCPECGKSFSDPGHLVRHQRTHTGEKPYKCPECGKSFS |
| | | RSDKLVRHQRTHTGEKPTGKKTS |
| ZF7 | 11 | LEPGEKPYKCPECGKSFSRNDALTEHQRTHTGEKPYKCPECGKSFSDCRDLARHQRTHTGEKP |
| (aa) | | YKCPECGKSFSDPGHLVRHQRTHTGEKPYKCPECGKSFSQSGHLTEHQRTHTGEKPYKCPECG |
| | | KSFSREDNLHTHQRTHTGEKPYKCPECGKSFSTKNSLTEHQRTHTGEKPYKCPECGKSFSRAD |
| | | NLTEHQRTHTGEKPTGKKTS |
| ZF8 | 12 | LEPGEKPYKCPECGKSFSRSDKLTEHQRTHTGEKPYKCPECGKSESRRDELNVHQRTHTGEK |
| (aa) | | PYKCPECGKSFSRSDHLTNHQRTHTGEKPYKCPECGKSFSSPADLTRHQRTHTGEKPYKCPE |
| | | CGKSFSRSDHLTNHQRTHTGEKPYKCPECGKSFSSKKALTEHQRTHTGEKPYKCPECGKSFS |
| | | THLDLIRHQRTHTGEKPTGKKTS |
| ZF9 | 13 | LEPGEKPYKCPECGKSFSRSDDLVRHQRTHTGEKPYKCPECGKSFSREDNLHTHQRTHTGEK |
| (aa) | | PYKCPECGKSFSRSDHLTTHQRTHTGEKPYKCPECGKSFSQRANLRAHQRTHTGEKPYKCPE |
| | | CGKSFSQLAHLRAHQRTHTGEKPYKCPECGKSFSQRANLRAHQRTHTGEKPYKCPECGKSFS |
| | | ERSHLREHQRTHTGEKPTGKKTS |
| ZF10 | 14 | LEPGEKPYKCPECGKSFSQSGDLRRHQRTHTGEKPYKCPECGKSFSQSGHLTEHQRTHTGEK |
| (aa) | | PYKCPECGKSFSERSHLREHQRTHTGEKPYKCPECGKSFSDPGHLVRHQRTHTGEKPYKCPE |
| | | CGKSFSRNDTLTEHQRTHTGEKPYKCPECGKSFSRADNLTEHQRTHTGEKPYKCPECGKSFS |
| | | THLDLIRHQRTHTGEKPTGKKTS |
| ZF11 | 15 | LEPGEKPYKCPECGKSFSHTGHLLEHQRTHTGEKPYKCPECGKSFSSKKALTEHQRTHTGEKP |
| (aa) | | YKCPECGKSFSDCRDLARHQRTHTGEKPYKCPECGKSFSHTGHLLEHQRTHTGEKPYKCPECG |
| | | KSFSRNDALTEHQRTHTGEKPYKCPECGKSFSQSGDLRRHQRTHTGEKPYKCPECGKSFSDSG |
| | | NLRVHQRTHTGEKPTGKKTS |
| ZF12 | 16 | LEPGEKPYKCPECGKSFSQSSSLVRHQRTHTGEKPYKCPECGKSESRSDHLTNHQRTHTGEKP |
| (aa) | | YKCPECGKSFSQLAHLRAHQRTHTGEKPYKCPECGKSFSQSSNLVRHQRTHTGEKPYKCPECG |
| | | KSFSRSDNLVRHQRTHTGEKPYKCPECGKSFSRSDELVRHQRTHTGEKPYKCPECGKSFSQLA |
| | | HLRAHQRTHTGEKPTGKKTS |
| ZF54 | 169 | LEPGEKPYKCPECGKSFSERSHLREHQRTHTGEKPYKCPECGKSFSRSDNLVRHQRTHTGEKP |
| (aa) | | YKCPECGKSFSDCRDLARHQRTHTGEKPYKCPECGKSFSTSGELVRHQRTHTGEKPYKCPECG |
| | | KSFSTTGNLTVHQRTHTGEKPYKCPECGKSFSRSDKLVRHQRTHTGEKPYKCPECGKSFSRTD |
| | | TLRDHQRTHTGKKTS |
| ZF61 | 170 | LEPGEKPYKCPECGKSFSQKSSLIAHQRTHTGEKPYKCPECGKSFSHKNALQNHQRTHTGEKP |
| (aa) | | YKCPECGKSFSQSSNLVRHQRTHTGEKPYKCPECGKSFSRNDALTEHQRTHTGEKPYKCPECG |
| | | KSFSDKKDLTRHQRTHTGEKPYKCPECGKSFSQAGHLASHQRTHTGEKPYKCPECGKSFSDKK |
| | | DLTRHQRTHTGKKTS |
| ZF67 | 171 | LEPGEKPYKCPECGKSFSRSDNLVRHQRTHTGEKPYKCPECGKSFSDCRDLARHQRTHTGEKP |
| (aa) | | YKCPECGKSFSTSGELVRHQRTHTGEKPYKCPECGKSFSTTGNLTVHQRTHTGEKPYKCPECG |
| | | KSFSRSDKLVRHQRTHTGEKPYKCPECGKSFSRTDTLRDHQRTHTGEKPYKCPECGKSFSRED |
| | | NLHTHQRTHTGKKTS |
| ZF68 | 172 | LEPGEKPYKCPECGKSFSRSDHLTTHQRTHTGEKPYKCPECGKSFSQKSSLIAHQRTHTGEKP |
| (aa) | | YKCPECGKSFSRRDELNVHQRTHTGEKPYKCPECGKSFSQSGDLRRHQRTHTGEKPYKCPECG |
| | | KSFSQAGHLASHQRTHTGEKPYKCPECGKSFSTSGNLTEHQRTHTGEKPYKCPECGKSFSQAG |
| | | HLASHQRTHTGKKTS |

TABLE 5
Nucleotide sequences of exemplary targeting moieties

| | SEQ | |
|---|---|---|
| | ID | |
| NAME | NO. | SEQUENCE |
| ZF1 | 38 | CTGGAGCCCGGCGAGAAACCCTACAAGTGCCCCGAGTGCGGCAAATCCTTCTCTAGAAGCGACAAACTGACC |
| (nt) | | GAACATCAGAGGACCCACACCGGCGAGAAGCCTTATAAGTGTCCCGAATGCGGCAAATCCTTCAGCACCAAG |
| | | AACTCTCTGACAGAACACCAGAGAACACATACCGGAGAGAAACCTTATAAATGCCCCGAGTGCGGCAAGTCC |
| | | TTCTCCCAGTCCGGCGATCTGAGGAGACACCAAAGAACACATACCGGCGAAAAGCCTTACAAGTGCCCCGAG |
| | | TGTGGAAAGAGCTTCTCCACCACCGGCGCTCTGACCGAGCACCAGAGAACACACACCGGCGAGAAACCCTAT |
| | | AAATGTCCCGAGTGTGGCAAATCCTTCAGCGACAGCGGCAATCTGAGAGTGCACCAAAGAACCCATACCGGC |
| | | GAAAAACCCTACAAATGCCCCGAGTGCGGCAAATCCTTCAGCCAGAGGGCCCATCTGGAGAGGCACCAAAGG |
| | | ACACACACCGGAGAAAAGCCCTACAAGTGTCCCGAGTGTGGAAAAAGCTTTAGCACAAGCGGCGAGCTGGTG |
| | | AGGCATCAAAGGACCCACACCGGCGAAAAGCCCACCGGCAAAAAGACCAGC |
| ZF2 | 39 | CTGGAGCCCGGCGAGAAGCCCTACAAGTGCCCCGAGTGCGGAAAGTCCTTCAGCTCCCCCGCCGATCTGACA |
| (nt) | | AGACATCAGAGAACCCATACCGGCGAGAAACCTTACAAATGCCCCGAATGTGGCAAGTCCTTTAGCGATCCC |
| | | GGACATCTGGTGAGGCACCAGAGGACACACACCGGCGAAAAGCCCTATAAATGTCCCGAGTGTGGAAAGAGC |
| | | TTTTCTAGAAGCGACAATCTCGTGAGACACCAGAGAACCCACACCGGAGAGAAGCCTTACAAGTGCCCCGAG |
| | | TGCGGCAAATCCTTCAGCCAGAGCTCCTCTCTGGTGAGGCACCAAAGGACCCACACCGGCGAGAAACCTTAT |
| | | AAGTGTCCCGAGTGTGGCAAAAGCTTCAGCACCTCCCACTCTCTGACCGAGCATCAAAGAACCCACACCGGC |
| | | GAAAAACCTTATAAATGCCCCGAGTGTGGCAAATCCTTCAGCAGAAATGACGCTCTGACAGAGCACCAAAGA |
| | | ACACATACCGGAGAAAAGCCCTACAAATGCCCCGAGTGTGGAAAATCCTTTTCTAGAAACGATGCTCTGACC |
| | | GAACACCAAAGAACACACACCGGCGAAAAGCCTACCGGAAAAAAGACCAGC |
| ZF3 | 40 | CTGGAGCCCGGCGAAAAACCTTACAAGTGCCCCGAGTGCGGAAAGAGCTTCAGCAGAAGCGACAAACTGGTG |
| (nt) | | AGGCATCAAAGGACACATACCGGAGAGAAGCCCTATAAGTGCCCCGAATGTGGCAAATCCTTTTCCCAGAGG |
| | | GCTCATCTGGAAAGACACCAGAGGACCCATACCGGCGAAAAACCCTACAAATGTCCCGAGTGTGGAAAGAGC |
| | | TTTTCCGATCCCGGCCATCTGGTCAGACATCAGAGGACACATACCGGCGAAAAGCCTTACAAGTGTCCCGAA |
| | | TGCGGAAAATCCTTCTCCAGAAGCGACAAGCTGGTGAGGCACCAAAGAACCCACACCGGCGAAAAACCCTAT |
| | | AAATGCCCCGAGTGCGGCAAGTCCTTTAGCCAGCTGGCCCATCTGAGAGCCCACCAGAGAACACACACCGGA |
| | | GAGAAGCCTTATAAGTGTCCCGAGTGCGGAAAGTCCTTCTCTAGAGCCGACAATCTGACCGAACATCAAAGG |
| | | ACACACACCGGCGAGAAACCTTATAAATGCCCCGAGTGCGGAAAAAGCTTTTCCGACTGCAGAGATCTGGCT |
| | | AGACACCAGAGAACCCACACCGGCGAGAAACCCACCGGCAAAAAGACCAGC |
| ZF4 | 41 | CTGGAGCCCGGCGAAAAGCCTTATAAATGTCCCGAATGCGGCAAGAGCTTTAGCCACACCGGCCATCTGCTG |
| (nt) | | GAACACCAAAGGACCCATACCGGCGAAAAGCCCTATAAGTGCCCCGAGTGTGGCAAGAGCTTCAGCACCACC |
| | | GGCAATCTGACAGTCCATCAGAGGACCCACACCGGAGAGAAACCCTATAAATGCCCCGAGTGTGGAAAGTCC |
| | | TTCTCCGACAAGAAGGATCTGACAAGACACCAGAGGACCCATACCGGCGAGAAACCCTACAAATGCCCCGAG |
| | | TGCGGCAAATCCTTCTCCCAGAGCGGCGATCTGAGGAGACATCAAAGAACACATACCGGCGAAAAACCCTAT |
| | | AAGTGCCCCGAATGCGGCAAGTCCTTCAGCCAGAGGGCCCATCTGGAAAGGCATCAGAGGACACACACCGGC |
| | | GAGAAGCCTTACAAATGTCCCGAGTGCGGAAAGAGCTTCTCTAGAAGCGACAAGCTGACCGAGCATCAGAGG |
| | | ACCCACACCGGAGAAAAACCTTACAAGTGCCCCGAGTGCGGCAAAAGCTTCAGCAGAACCGACACACTGAGA |
| | | GATCACCAAAGGACACACACCGGCGAGAAACCCACCGGCAAAAAGACCAGC |
| ZF5 | 42 | CTGGAGCCCGGCGAGAAGCCTTATAAGTGCCCCGAGTGTGGCAAGAGCTTTAGCCACACCGGCCATCTGCTG |
| (nt) | | GAGCATCAAAGGACACACACCGGAGAAAAGCCCTATAAGTGCCCCGAGTGTGGCAAATCCTTCAGCACCTCCG |
| | | GCAATCTCACCGAACACCAGAGAACACACACCGGAGAAAAACCTTACAAATGTCCCGAGTGTGGAAAGAGCTT |
| | | TTCCACCAGCGGCAATCTGGTGAGACATCAAAGAACACATACCGGCGAAAAACCCTATAAATGCCCCGAGTG |
| | | TGGAAAATCCTTCTCCCAACTGGCCCATCTGAGGGCCCACCAGAGGACACATACCGGAGAAAAACCCTACAAA |
| | | TGCCCCGAATGCGGAAAAAGCTTCTCCGAGAGAAGCCATCTGAGAGAGCACCAAAGGACCCATACCGGAGAAA |
| | | AGCCTTACAAGTGTCCCGAGTGCGGAAAAAGCTTTAGCGATCCCGGACATCTGGTGAGACATCAGAGAACCC |
| | | ACACCGGCGAAAAGCCTTATAAATGTCCCGAATGTGGCAAGTCCTTTAGCACCCATCTGGATCTGATTAGAC |
| | | ACCAAAGAACCCACACCGGCGAGAAACCCACCGGAAAAAAGACCAGC |
| ZF6 | 43 | CTGGAGCCCGGCGAAAAGCCTTACAAATGTCCCGAGTGCGGAAAGTCCTTCAGCGACCCCGGCGCTCTGGTG |
| (nt) | | AGACATCAAAGAACACATACCGGCGAGAAACCTTATAAATGCCCCGAATGTGGAAAATCCTTCAGCGAAAGA |
| | | AGCCATCTGAGGGAACACCAGAGGACCCACACCGGCGAAAAACCTTATAAGTGCCCCGAATGCGGAAAAAGC |
| | | TTTTCTAGAAGCGATCATCTGACCAACCATCAGAGAACACACACCGGCGAAAAGCCCTATAAATGTCCCGAG |
| | | TGTGGCAAATCCTTTAGCGAGAGGTCCCATCTGAGAGAGCACCAGAGGACACATACCGGAGAGAAGCCCTAC |
| | | AAGTGCCCCGAGTGTGGCAAGAGCTTTAGCAGAAGCGACCATCTGACCAATCATCAAAGGACCCACACCGGA |
| | | GAGAAGCCTTACAAGTGTCCCGAGTGCGGAAAGTCCTTTTCCGATCCCGGCCACCTCGTGAGGCACCAAAGA |
| | | ACCCATACCGGCGAGAAACCCTACAAATGCCCCGAGTGTGGAAAGAGCTTCTCCAGAAGCGACAAGCTGGTG |
| | | AGGCATCAGAGGACACACACCGGCGAAAAACCCACCGGCAAGAAAACCAGC |
| ZF7 | 44 | CTGGAGCCCGGAGAGAAGCCCTACAAATGCCCCGAGTGTGGAAAGAGCTTCTCTAGAAATGACGCTCTGACA |
| (nt) | | GAACACCAGAGGACCCATACCGGCGAGAAACCTTACAAATGCCCCGAGTGCGGAAAAAGCTTTAGCGATTGC |
| | | AGAGATCTGGCTAGACATCAGAGAACACACACCGGCGAGAAGCCCTATAAGTGCCCCGAATGCGGCAAGAGC |
| | | TTTAGCGACCCCGGCCATCTGGTGAGACATCAAAGGACACATACCGGAGAAAAACCTTACAAGTGCCCCGAG |
| | | TGCGGAAAGTCCTTCTCCCAGAGCGGCCATCTCACCGAGCATCAAAGGACCCACACCGGCGAAAAGCCTTAT |
| | | AAATGTCCCGAATGTGGCAAGTCCTTCTCTAGAGAGGATAATCTGCACACCCATCAGAGGACCCACACCGGC |
| | | GAAAAGCCTTATAAATGCCCCGAATGTGGAAAGTCCTTTTCCACCAAGAACTCTCTGACCGAGCATCAGAGG |
| | | ACACACACCGGAGAGAAACCCTATAAATGTCCCGAGTGTGGCAAGAGCTTCAGCAGAGCCGACAATCTGACA |
| | | GAGCACCAAAGAACACATACCGGCGAAAAGCCCACCGGCAAAAAGACCAGC |
| ZF8 | 45 | CTGGAGCCCGGCGAGAAACCCTACAAGTGCCCCGAGTGTGGCAAATCCTTCTCTAGATCCGACAAACTGACC |
| (nt) | | GAACATCAGAGGACCCATACCGGCGAAAAACCTTATAAATGTCCCGAGTGCGGAAAGTCCTTCTCTAGAAGG |
| | | GACGAGCTGAACGTGCATCAGAGAACACATACCGGCGAGAAGCCCTATAAATGCCCCGAATGCGGCAAAAGC |
| | | TTCTCTAGAAGCGATCATCTGACCAACCACCAGAGAACCCATACCGGAGAAAAGCCTTACAAGTGTCCCGAA |
| | | TGTGGAAAATCCTTCAGCTCCCCCGCCGATCTGACCAGACACCAAAGGACCCACACCGGCGAGAAGCCCTAT |
| | | AAATGCCCCGAGTGCGGCAAGAGCTTTTCCAGATCCGACCATCTGACCAATCATCAAAGAACCCACACCGGC |
| | | GAAAAGCCTTATAAATGTCCCGAGTGCGGCAAATCCTTTTCCAGCAAGAAGGCTCTGACCGAGCATCAAAGG |
| | | ACCCATACCGGCGAGAAGCCTTACAAATGCCCCGAGTGTGGAAAGTCCTTTAGCACCCATCTGGATCTGATT |
| | | AGACACCAGAGGACACACACCGGAGAGAAACCCACCGGCAAAAAGACCAGC |
| ZF9 | 46 | CTGGAGCCCGGCGAGAAACCTTACAAATGCCCCGAGTGCGGCAAGAGCTTCAGCAGAAGCGACGATCTGGTG |
| (nt) | | AGGCACCAAAGAACCCACACCGGCGAAAAACCTTACAAGTGTCCCGAATGCGGAAAGTCCTTCAGCAGAGAG |
| | | GACAATCTGCACACCCACCAGAGAACACACACCGGAGAAAAGCCTTACAAGTGCCCCGAATGCGGCAAATCC |
| | | TTTTCTAGAAGCGATCATCTGACCACCCACCAAAGAACACATACCGGCGAGAAGCCTTACAAATGTCCCGAG |
| | | TGCGGAAAGTCCTTCTCCCAGAGAGCCAATCTGAGGGCTCATCAAAGGACCCATACCGGCGAAAAGCCCTAC |
| | | AAATGCCCCGAGTGCGGAAAATCCTTCAGCCAGCTGGCCCATCTGAGAGCCCACCAAAGGACACACACCGGA |
| | | GAGAAACCCTATAAGTGCCCCGAGTGTGGAAAAAGCTTTTCCCAGAGGGCCAATCTGAGGGCCCATCAGAGG |
| | | ACCCATACCGGAGAGAAGCCTTATAAATGTCCCGAGTGCGGAAAAAGCTTCAGCGAGAGGAGCCATCTGAGG |
| | | GAACATCAAAGAACCCACACCGGCGAAAAACCCACCGGAAAAAAGACCAGC |
| ZF10 | 47 | CTGGAGCCCGGCGAGAAACCCTACAAGTGCCCCGAGTGTGGAAAAAGCTTTAGCCAAAGCGGCGATCTGAGG |
| (nt) | | AGACACCAAAGAACACACACCGGCGAGAAGCCCTACAAATGTCCCGAGTGCGGAAAGAGCTTCAGCCAGAGC |
| | | GGCCATCTGACCGAGCATCAGAGAACCCATACCGGCGAAAAACCTTATAAGTGCCCCGAGTGTGGAAAGTCC |
| | | TTCTCCGAGAGATCCCATCTGAGAGAACACCAGAGGACACACACCGGCGAAAAACCTTATAAGTGTCCCGAG |
| | | TGCGGAAAGTCCTTCAGCGATCCCGGCCATCTGGTGAGACATCAAAGGACACATACCGGCGAAAAACCTTAT |
| | | AAGTGTCCCGAATGCGGCAAGAGCTTTAGCAGAAACGACACACTCACCGAACACCAGAGGACCCACACCGGC |
| | | GAGAAACCCTACAAATGCCCCGAGTGCGGCAAATCCTTTTCTAGAGCCGACAATCTGACCGAACACCAGAGG |
| | | ACCCATACCGGAGAAAAGCCTTACAAATGTCCCGAGTGTGGCAAATCCTTCTCCACCCATCTGGATCTGATT |
| | | AGACACCAAAGAACACATACCGGAGAAAAGCCCACCGGAAAAAAGACCAGC |
| ZF11 | 48 | CTGGAGCCCGGCGAAAAACCCTATAAGTGCCCCGAATGTGGAAAGAGCTTCAGCCATACCGGCCATCTGCTG |
| (nt) | | GAACACCAAAGGACACACACCGGCGAGAAACCTTACAAGTGTCCCGAGTGCGGAAAAAGCTTCTCCTCCAAA |
| | | AAGGCTCTCACCGAGCACCAGAGAACACATACCGGCGAAAAGCCTTATAAGTGCCCCGAGTGTGGCAAATCC |
| | | TTTTCCGACTGTAGAGATCTGGCCAGACATCAAAGAACCCACACCGGAGAGAAACCTTATAAATGCCCCGAG |
| | | TGCGGCAAGTCCTTTAGCCATACCGGCCATCTGCTGGAGCACCAGAGGACCCATACCGGCGAGAAGCCTTAC |
| | | AAATGCCCCGAGTGCGGCAAAAGCTTCAGCAGAAATGACGCTCTGACCGAGCATCAAAGGACCCATACCGGC |
| | | GAAAAGCCCTACAAGTGTCCCGAGTGTGGAAAGTCCTTCTCCCAGAGCGGCGATCTGAGGAGACACCAGAGA |
| | | ACACACACCGGCGAGAAACCCTATAAATGTCCCGAGTGCGGAAAGAGCTTTAGCGACAGCGGCAATCTGAGG |
| | | GTGCATCAAAGAACACACACCGGCGAAAAACCCACCGGAAAAAAGACAAGC |
| ZF12 | 49 | CACCGGCGAAAAGCCTTATAAGTGCCCCGAGTGCGGCAAGTCCTTCTCTAGAAGCGATCACCTCACCAATCA |
| (nt) | | TCAGAGGACACATACCGGAGAGAAGCCCTATAAGTGCCCCGAGTGCGGCAAGAGCTTTAGCCAGCTGGCTCA |
| | | TCTGAGAGCTCACCAAAGAACCCATACCGGCGAGAAGCCTTACAAATGCCCCGAGTGTGGAAAATCCTTTTC |
| | | CCAGTCCAGCAACCTCGTCAGACATCAAAGGACCCATACCGGCGAAAAGCCTTACAAGTGTCCCGAGTGCGG |
| | | AAAGTCCTTCTCTAGATCCGACAACCTCGTGAGGCACCAGAGAACCCACACCGGCGAGAAACCTTACAAATG |
| | | TCCCGAGTGTGGCAAAAGCTTTTCTAGAAGCGACGAGCTGGTGAGACATCAAAGAACCCATACCGGCGAAAA |
| | | ACCTTATAAGTGTCCCGAGTGCGGCAAATCCTTTAGCCAGCTGGCCCATCTGAGGGCCCACCAGAGAACACA |
| | | TACCGGCGAAAAACCCACCGGCAAAAAGACAAGC |
| ZF12 | 115 | CTGGAGCCCGGCGAGAAACCCTATAAATGCCCCGAATGCGGAAAAAGCTTCAGCCAGTCCAGCTCTCTGGTG |
| (nt) | | AGACATCAGAGGACACACACCGGCGAAAAGCCTTATAAGTGCCCCGAGTGCGGCAAGTCCTTCTCTAGAAGC |
| Full | | GATCACCTCACCAATCATCAGAGGACACATACCGGAGAGAAGCCCTATAAGTGCCCCGAGTGCGGCAAGAGC |
| length | | TTTAGCCAGCTGGCTCATCTGAGAGCTCACCAAAGAACCCATACCGGCGAGAAGCCTTACAAATGCCCCGAG |
| | | TGTGGAAAATCCTTTTCCCAGTCCAGCAACCTCGTCAGACATCAAAGGACCCATACCGGCGAAAAGCCTTAC |
| | | AAGTGTCCCGAGTGCGGAAAGTCCTTCTCTAGATCCGACAACCTCGTGAGGCACCAGAGAACCCACACCGGC |
| | | GAGAAACCTTACAAATGTCCCGAGTGTGGCAAAAGCTTTTCTAGAAGCGACGAGCTGGTGAGACATCAAAGA |
| | | ACCCATACCGGCGAAAAACCTTATAAGTGTCCCGAGTGCGGCAAATCCTTTAGCCAGCTGGCCCATCTGAGG |
| | | GCCCACCAGAGAACACATACCGGCGAAAAACCCACCGGCAAAAAGACAAGC |
| ZF9 | 131 | CUGGAGCCCGGCGAGAAACCUUACAAAJGCCCCGAGUGCGGCAAGAGCUUCAGCAGAAGCGACGAUCUGGUG |
| | | AGGCACCAAAGAACCCACACCGGCGAAAAACCUUACAAGUGUCCCGAAUGCGGAAAGUCCUUCAGCAGAGAG |
| | | GACAAUCUGCACACCCACCAGAGAACACACACCGGAGAAAAGCCUUACAAGUGCCCCGAAUGCGGCAAAUCC |
| | | UUUUCUAGAAGCGAUCAUCUGACCACCCACCAAAGAACACAUACCGGCGAGAAGCCUUACAAAUGUCCCGAG |
| | | UGCGGAAAGUCCUUCUCCCAGAGAGCCAAUCUGAGGGCUCAUCAAAGGACCCAUACCGGCGAAAAGCCCUAC |
| | | AAAUGCCCCGAGUGCGGAAAAUCCUUCAGCCAGCUGGCCCAUCUGAGAGCCCACCAAAGGACACACACCGGA |
| | | GAGAAACCCUAUAAGUGCCCCGAGUGUGGAAAAAGCUUUUCCCAGAGGGCCAAUCUGAGGGCCCAUCAGAGG |
| | | ACCCAUACCGGAGAGAAGCCUUAUAAAJGUCCCGAGUGCGGAAAAAGCUUCAGCGAGAGGAGCCAUCUGAGG |
| | | GAACAUCAAAGAACCCACACCGGCGAAAAACCCACCGGAAAAAAGACCAGC |
| ZF54 | 173 | CTGGAGCCTGGAGAGAAACCCTACAAATGCCCGGAATGCGGGAAGTCCTTTTCCGAACGCTCGCACCTGAGG |
| | | GAACACCAGAGAACTCACACCGGCGAAAAACCCTATAAGTGCCCAGAATGCGGAAAGAGCTTTTCACGGTCG |
| | | GACAACCTCGTGCGGCACCAACGCACTCATACCGGAGAGAAGCCGTACAAGTGTCCTGAGTGCGGAAAGTCA |
| | | TTCTCCGACTGCCGGGATTTGGCCCGCCACCAAAGAACACACACTGGCGAAAAGCCCTACAAGTGCCCGGAG |
| | | TGTGGAAAGTCCTTCAGCACTTCCGGAGAGCTGGTCCGGCACCAGAGGACCCACACCGGGGAGAAGCCTTAC |
| | | AAATGTCCAGAGTGCGGTAAAAGCTTCTCCACCACCGGCAACCTCACCGTGCACCAGCGGACCCACACTGGA |
| | | GAAAAGCCGTATAAATGCCCCGAATGCGGCAAGAGCTTCTCGCGATCCGATAAGCTTGTGCGGCATCAGAGA |
| | | ACGCACACTGGGGAAAAGCCTTATAAGTGTCCGGAGTGCGGCAAATCCTTCTCCCGCACTGACACCCTGCGG |
| | | GACCATCAGCGCACCCATACCGGCAAAAAGACCTCT |
| ZF61 | 174 | CTTGAACCCGGGGAGAAGCCCTACAAGTGCCCGGAATGCGGAAAGAGCTTCAGCCAGAAGTCCTCGCTGATC |
| | | GCGCACCAGAGGACTCACACCGGCGAAAAGCCATACAAGTGTCCTGAGTGTGGCAAATCCTTCTCGCACAAG |
| | | AACGCACTGCAGAACCACCAGAGAACCCACACCGGGGAAAAGCCGTATAAGTGCCCCGAATGTGGAAAGTCG |
| | | TTCAGCCAGTCATCCAACCTCGTGCGCCATCAAAGGACTCATACCGGAGAGAAACCTTACAAATGCCCTGAA |
| | | TGCGGCAAATCTTTCTCCCGGAATGATGCCCTGACCGAGCACCAGCGCACTCACACGGGAGAGAAGCCGTAC |
| | | AAATGTCCGGAGTGCGGAAAGTCCTTCTCCGACAAGAAGGACTTGACCAGACACCAGCGGACCCATACTGGC |
| | | GAAAAACCCTATAAGTGTCCAGAGTGCGGGAAGTCCTTTAGCCAAGCCGGTCACCTCGCTTCCCACCAACGG |
| | | ACCCACACAGGAGAAAAGCCTTATAAATGCCCCGAGTGCGGCAAAAGCTTCTCCGATAAGAAGGACCTGACT |
| | | CGGCATCAGCGCACCCATACCGGAAAGAAAACCTCA |
| ZF67 | 175 | CTGGAGCCTGGCGAAAAACCCTATAAGTGCCCAGAATGCGGAAAGAGCTTTTCACGGTCGGACAACCTCGTG |
| | | CGGCACCAACGCACTCATACCGGAGAGAAGCCGTACAAGTGTCCTGAGTGCGGAAAGTCATTCTCCGACTGC |
| | | CGGGATTTGGCCCGCCACCAAAGAACACACACTGGCGAAAAGCCCTACAAGTGCCCGGAGTGTGGAAAGTCC |
| | | TTCAGCACTTCCGGAGAGCTGGTCCGGCACCAGAGGACCCACACCGGGGAGAAGCCTTACAAATGTCCAGAG |
| | | TGCGGTAAAAGCTTCTCCACCACCGGCAACCTCACCGTGCACCAGCGGACCCACACTGGAGAAAAGCCGTAT |
| | | AAATGCCCCGAATGCGGCAAGAGCTTCTCGCGATCCGATAAGCTTGTGCGGCATCAGAGAACGCACACTGGG |
| | | GAAAAGCCTTATAAGTGTCCGGAGTGCGGCAAATCCTTCTCCCGCACTGACACCCTGCGGGACCACCAGAGA |
| | | ACCCATACTGGCGAGAAGCCATACAAATGCCCGGAATGTGGAAAGAGTTTCTCGCGCGAGGACAACCTCCAC |
| | | ACCCATCAGCGCACCCATACCGGCAAAAAGACCTCT |
| ZF68 | 176 | CTGGAACCCGGAGAGAAACCCTACAAATGCCCAGAGTGCGGCAAATCGTTCTCACGGTCCGATCACCTCACC |
| | | ACCCACCAGAGGACCCATACCGGGGAGAAGCCTTACAAGTGTCCTGAGTGTGGAAAGTCCTTCAGCCAAAAG |
| | | TCCTCGCTGATCGCACACCAGCGCACGCACACTGGGGAAAAGCCATATAAATGCCCGGAGTGTGGCAAATCC |
| | | TTCTCCCGCCGCGACGAACTGAACGTGCACCAGAGAACCCACACTGGAGAGAAGCCGTATAAGTGTCCGGAG |
| | | TGCGGAAAGAGCTTCTCGCAATCCGGGGACCTTCGGAGACATCAGAGGACACACACTGGCGAAAAGCCCTAT |
| | | AAGTGCCCTGAGTGCGGGAAGTCCTTTAGCCAAGCCGGTCACCTGGCCTCCCACCAACGGACTCACACCGGC |
| | | GAAAAACCGTACAAGTGCCCCGAATGCGGAAAGTCGTTCTCTACCTCCGGAAACTTGACCGAACACCAGCGG |
| | | ACCCACACCGGAGAAAAGCCGTACAAATGTCCTGAATGCGGCAAAAGCTTCAGCCAGGCCGGTCATCTCGCG |
| | | AGCCATCAGCGGACTCATACTGGCAAAAAGACCTCA |

In some embodiments, an expression repression comprises a targeting moiety comprising an engineered DNA binding domain (DBD), e.g., a Zn finger domain comprising a Zn finger (ZFN) that binds to a target sequence, e.g., a promoter or transcription start site (TSS)) sequence operably linked to a target gene (e.g., MYC), e.g., a sequence proximal to the transcription regulatory element, e.g., an anchor sequence of an anchor sequence mediated conjunction (ASMC) comprising a target gene (e.g., MYC), e.g., a sequence proximal to the anchor sequence in mouse genome. In some embodiments, the ZFN can be engineered to carry epigenetic effector molecules to target sites. In some embodiments, the targeting moiety comprises a Zn Finger domain that comprises 2, 3, 4, 5, 6, 7, or 8 zinc fingers. The amino acid sequences of exemplary targeting moieties disclosed herein are listed in Table 14. The nucleotide sequences encoding exemplary targeting moieties disclosed herein are listed in Table 15. In some embodiments, an expression repressor or system described herein comprises a targeting moiety having a sequence set forth in Table 14, or a sequence with at least 70%, 75%, 80%, 85%, 90%, 95%, 98%, or 99% identity thereto. In some embodiments, a nucleic acid described herein comprises a sequence set forth in Table 15, or a sequence with at least 70%, 75%, 80%, 85%, 90%, 95%, 98%, or 99% identity thereto.

TABLE 14
Amino acid sequences of exemplary mouse-specific targeting moieties

| | SEQ | |
|---|---|---|
| | ID | |
| Name | NO. | SEQUENCE |
| ZF15 | 154 | LEPGEKPYKCPECGKSFSTSGELVRHQRTHTGEKPYKCPECGKSFSERSHLREHQRTHTGEKPYKCPECGK |
| (aa) | | SFSRNDTLTEHQRTHTGEKPYKCPECGKSFSRADNLTEHQRTHTGEKPYKCPECGKSFSTSGSLVRHQRTH |
| | | TGEKPYKCPECGKSFSSPADLTRHQRTHTGEKPYKCPECGKSFSDSGNLRVHQRTHTGEKPTGKKTS |
| ZF16 | 155 | LEPGEKPYKCPECGKSFSRSDKLVRHQRTHTGEKPYKCPECGKSFSQRANLRAHQRTHTGEKPYKCPECG |
| (aa) | | KSFSRADNLTEHQRTHTGEKPYKCPECGKSFSQSSSLVRHQRTHTGEKPYKCPECGKSFSDKKDLTRHQR |
| | | THTGEKPYKCPECGKSFSSPADLTRHQRTHTGEKPYKCPECGKSFSQSGHLTEHQRTHTGEKPTGKKTS |
| ZF17 | 156 | LEPGEKPYKCPECGKSFSQSGDLRRHQRTHTGEKPYKCPECGKSFSQSGDLRRHQRTHTGEKPYKCPECG |
| (aa) | | KSFSERSHLREHQRTHTGEKPYKCPECGKSFSTTGNLTVHQRTHTGEKPYKCPECGKSFSHRTTLTNHQR |
| | | THTGEKPYKCPECGKSFSDSGNLRVHQRTHTGEKPYKCPECGKSFSQSSSLVRHQRTHTGEKPTGKKTS |

TABLE 15
Nucleotide sequences of exemplary mouse-specific targeting moieties

| | SEQ | |
|---|---|---|
| | ID | |
| Name | NO. | SEQUENCE |
| ZF15 | 157 | CTTGAGCCCGGAGAAAAGCCATACAAATGTCCTGAATGCGGAAAGTCATTTTCTACGAGCGGCGAACT |
| nt | | CGTGCGGCACCAAAGGACTCATACCGGCGAAAAGCCTTACAAATGCCCGGAGTGCGGAAAAAGCTTCT |
| | | CCGAGCGCTCGCACTTGCGGGAACACCAGCGAACCCACACAGGGGAGAAACCGTATAAGTGCCCAGAG |
| | | TGCGGCAAATCGTTCTCCCGGAACGACACCCTGACCGAACACCAACGCACTCATACTGGCGAAAAACC |
| | | TTACAAGTGCCCTGAGTGTGGAAAGAGCTTCTCCCGCGCCGATAACCTGACCGAGCACCAGCGGACCC |
| | | ATACCGGGGAAAAGCCGTACAAGTGTCCGGAATGCGGCAAAAGCTTCAGCACCTCGGGTTCCCTGGTC |
| | | CGGCATCAGAGAACTCACACCGGAGAGAAACCCTATAAGTGTCCTGAGTGCGGGAAGTCCTTTTCATC |
| | | GCCCGCGGACCTGACTAGACACCAGAGGACCCACACCGGGGAGAAGCCCTACAAGTGCCCCGAATGTG |
| | | GAAAGTCCTTCTCCGACTCCGGCAACCTCCGGGTGCACCAGCGCACCCACACTGGAGAGAAGCCGACC |
| | | GGAAAGAAAACTTCC |
| ZF16 | 158 | CTGGAACCCGGAGAAAAACCTTATAAGTGCCCTGAATGCGGAAAGTCATTCTCGAGGTCGGACAAGCT |
| nt | | CGTGCGGCACCAGAGGACACACACCGGGGAAAAGCCATACAAGTGTCCCGAATGTGGAAAGTCCTTCA |
| | | GCCAACGCGCCAACCTGAGAGCTCATCAGCGGACTCACACTGGCGAAAAACCGTACAAATGCCCCGAA |
| | | TGCGGCAAAAGCTTCTCCCGCGCCGACAACTTGACCGAGCACCAGCGGACCCATACCGGCGAAAAGCC |
| | | GTACAAGTGCCCGGAGTGTGGGAAGTCGTTCAGCCAGTCCTCTTCCCTCGTGCGCCACCAACGCACCC |
| | | ATACTGGGGAGAAGCCCTATAAGTGTCCTGAGTGTGGCAAATCATTCAGCGATAAGAAGGATCTTACC |
| | | CGGCACCAACGGACTCATACCGGAGAGAAGCCTTACAAGTGCCCCGAGTGCGGAAAGAGCTTCTCGTC |
| | | CCCGGCGGACCTGACTAGACACCAGCGCACCCACACCGGAGAAAAGCCCTACAAGTGCCCAGAGTGCG |
| | | GGAAGTCCTTTTCCCAATCCGGTCACCTGACTGAGCACCAGAGAACCCACACGGGAGAGAAACCGACC |
| | | GGAAAGAAAACCTCC |
| ZF17 | 159 | TTGGAACCCGGAGAAAAGCCATACAAATGCCCCGAATGCGGAAAGTCGTTCAGCCAGTCCGGCGACCT |
| nt | | CAGACGGCACCAACGGACTCACACCGGCGAAAAACCGTACAAGTGCCCAGAGTGCGGCAAAAGCTTTA |
| | | GCCAGTCGGGCGATCTGCGGAGACATCAGCGCACTCACACTGGTGAAAAGCCCTACAAGTGTCCTGAG |
| | | TGCGGGAAGTCCTTCAGCGAGCGCTCCCATCTTCGCGAGCACCAGAGAACCCACACTGGAGAAAAACC |
| | | TTATAAGTGCCCTGAGTGTGGCAAATCCTTCTCAACCACCGGCAACCTGACTGTGCACCAGCGGACCC |
| | | ACACAGGGGAGAAGCCTTACAAGTGCCCGGAGTGTGGGAAGTCATTCTCCCATCGGACGACCCTGACC |
| | | AACCACCAGAGGACCCATACTGGCGAAAAGCCGTATAAGTGTCCGGAGTGCGGAAAGAGCTTCTCCGA |
| | | CTCCGGAAACCTCAGGGTGCACCAACGCACCCACACCGGAGAGAAGCCGTACAAATGTCCCGAATGTG |
| | | GAAAGTCCTTCTCCCAATCCTCTTCGCTGGTCCGGCACCAGCGAACTCATACCGGGGAAAAGCCCACC |
| | | GGAAAGAAAACCTCG |

### Effector Moieties

In some embodiments, expression repressors of the present disclosure comprise one or more effector moieties. In some embodiments, an effector moiety, when used as part of an expression repressor or an expression repression system described herein, decreases expression of a target gene in a cell.

In some embodiments, the effector moiety has functionality unrelated to the binding of the targeting moiety. For example, effector moieties may target, e.g., bind, a genomic sequence element or genomic complex component proximal to the genomic sequence element targeted by the targeting moiety or recruit a transcription factor. As a further example, an effector moiety may comprise an enzymatic activity, e.g., a genetic modification functionality.

In some embodiments, an effector moiety comprises an epigenetic modifying moiety. In some embodiments, an effector moiety comprises a DNA modifying functionality, e.g., a DNA methyltransferase. In some embodiments, an effector moiety is or comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.

In some embodiments, an effector moiety comprises a transcription repressor. In some embodiments the transcription repressor blocks recruitment of a factor that stimulates or promotes transcription, e.g., of the target gene. In some embodiments, the transcription repressor recruits a factor that inhibits transcription, e.g., of the target gene. In some embodiments, an effector moiety, e.g., transcription repressor, is or comprises a protein chosen from KRAB, MeCP2, HP1, RBBP4, REST, FOG1, SUZ12, or a functional variant or fragment of any thereof.

In some embodiments an effector moiety promotes epigenetic modification, e.g., directly or indirectly. For example, an effector moiety can indirectly promote epigenetic modification by recruiting an endogenous protein that epigenetically modifies the chromatin. An effector moiety can directly promote epigenetic modification by catalyzing epigenetic modification, wherein the effector moiety comprises enzymatic activity and directly places an epigenetic mark on the chromatin.

In some embodiments, an effector moiety comprises a histone modifying functionality, e.g., a histone methyltransferase, histone demethylase, or histone deacetylase activity. In some embodiments, a effector moiety is or comprises a protein chosen from KDM1A (i.e., LSD1), KDM1B (i.e., LSD2), KDM2A, KDM2B, KDM5A, KDM5B, KDM5C, KDM5D, KDM4B, NO66, or a functional variant or fragment of any thereof. In some embodiments, a effector moiety is or comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

In some embodiments, an effector moiety comprises a protein having a functionality described herein. In some embodiments, an effector moiety is or comprises a protein selected from: KRAB (e.g., as according to NP_056209.2 or the protein encoded by NM_015394.5); a SET domain (e.g., the SET domain of: SETDB1 (e.g., as according to NP_001353347.1 or the protein encoded by NM_001366418.1); EZH2 (e.g., as according to NP-004447.2 or the protein encoded by NM_004456.5); G9A (e.g., as according to NP_001350618.1 or the protein encoded by NM_001363689.1); or SUV39H1 (e.g., as according to NP_003164.1 or the protein encoded by NM_003173.4)); histone demethylase LSD1 (e.g., as according to NP_055828.2 or the protein encoded by NM_015013.4); FOG1 (e.g., the N-terminal residues of FOG1) (e.g., as according to NP_722520.2 or the protein encoded by NM_153813.3); or KAP1 (e.g., as according to NP_005753.1 or the protein encoded by NM_005762.3); a functional fragment or variant of any thereof, or a polypeptide with a sequence that has at least 80, 85, 90, 91, 92, 93, 94, 95, 96, 97, 98, or 99% identity to any of the above-referenced sequences.

In some embodiments, a effector moiety is or comprises a protein selected from: DNMT3A (e.g., human DNMT3A) (e.g., as according to NP_072046.2 or the protein encoded by NM_022552.4); DNMT3B (e.g., as according to NP_008823.1 or the protein encoded by NM_006892.4); DNMT3L (e.g., as according to NP_787063.1 or the protein encoded by NM_175867.3); DNMT3A/3L complex, bacterial MQ1 (e.g., as according to CAA35058.1 or P15840.3); a functional fragment of any thereof, or a polypeptide with a sequence that has at least 80, 85, 90, 91, 92, 93, 94, 95, 96, 97, 98, or 99% identity to any of the above-referenced sequences.

In another aspect, the disclosure is directed to an expression repressor or a polypeptide comprising one or more (e.g., one) targeting moiety and one or more effector moiety, wherein the one or more effector moiety is or comprises Kruppel-associated box (KRAB) e.g., as according to NP_056209.2 or the protein encoded by NM_015394.5 or a functional variant or fragment thereof. In some embodiments, KRAB is a synthetic KRAB construct. In some embodiments, KRAB comprises an amino acid sequence of SEQ ID NO: 18:

(SEQ ID NO: 18)
DAKSLTAWSRTLVTFKDVFVDFTREEWKLLDTAQQILYRNVMLEN
YKNLVSLGYQLTKPDVILRLEKGEEPWLVEREIHQETHPDSETAF
EIKSSV

In some embodiments, the KRAB effector moiety is encoded by a nucleotide sequence of SEQ ID NO: 51. In some embodiments, a nucleotide sequence described herein comprises a sequence of SEQ ID NO: 51 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 51)
GACGCCAAGAGCCTGACCGCCTGGAGCCGGACCCTGGTGACCTTC
AAGGACGTGTTCGTGGACTTCACCCGGGAGGAGTGGAAGCTGCTG
GACACCGCCCAGCAGATCCTGTACCGGAACGTGATGCTGGAGAAC
TACAAGAACCTGGTGAGCCTGGGCTACCAGCTGACCAAGCCCGAC
GTGATCCTGCGGCTGGAGAAGGGCGAGGAGCCCTGGCTGGTGGAG
CGGGAGATCCACCAGGAGACCCACCCCGACAGCGAGACCGCCTTC
GAGATCAAGAGCAGCGTG

In some embodiments, KRAB for use in a polypeptide or an expression repressor described herein is a variant, e.g., comprising one or more mutations, relative to the KRAB sequence of SEQ ID NO: 18. In some embodiments, an KRAB variant comprises one or more amino acid substitutions, deletions, or insertions relative to SEQ ID NO: 18.

In some embodiments, the polypeptide or the expression repressor is a fusion protein comprising a effector moiety that is or comprises KRAB and a DNA-targeting moiety.

In another aspect, the disclosure is directed to an expression repressor or a polypeptide comprising one or more (e.g., one) targeting moiety and one or more effector moiety, wherein the one or more effector moiety is or comprises MQ1, e.g., bacterial MQ1, or a functional variant or fragment thereof. In some embodiments, MQ1 is Mollicutes spiroplasma MQ1. In some embodiments, MQ1 is Spiroplasma monobiae MQ1. In some embodiments, MQ1 is MQ1 from strain ATCC 33825 and/or corresponding to Uniprot ID P15840. In some embodiments, MQ1 comprises an amino acid sequence of SEQ ID NO: 19. In some embodiments, MQ1 comprises an amino acid sequence of SEQ ID NO: 87. In some embodiments, an effector domain described herein comprises SEQ ID NO: 19 or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 19)
SKVENKTKKLRVFEAFAGIGAQRKALEKVRKDEYEIVGLAEWYVP
AIVMYQAIHNNFHTKLEYKSVSREEMIDYLENKTLSWNSKNPVSN
GYWKRKKDDELKIIYNAIKLSEKEGNIFDIRDLYKRTLKNIDLLT
YSFPCQDLSQQGIQKGMKRGSGTRSGLLWEIERALDSTEKNDLPK
YLLMENVGALLHKKNEEELNQWKQKLESLGYQNSIEVLNAADFGS
SQARRRVFMISTLNEFVELPKGDKKPKSIKKVLNKIVSEKDILNN
LLKYNLTEFKKTKSNINKASLIGYSKFNSEGYVYDPEFTGPTLTA
SGANSRIKIKDGSNIRKMNSDETFLYIGFDSQDGKRVNEIEFLTE
NQKIFVCGNSISVEVLEAIIDKIGG
(SEQ ID NO: 87)
MSKVENKTKKLRVFEAFAGIGAQRKALEKVRKDEYEIVGLAEWYV
PAIVMYQAIHNNFHTKLEYKSVSREEMIDYLENKTLSWNSKNPVS
NGYWKRKKDDELKIIYNAIKLSEKEGNIFDIRDLYKRTLKNIDLL
TYSFPCQDLSQQGIQKGMKRGSGTRSGLLWEIERALDSTEKNDLP
KYLLMENVGALLHKKNEEELNQWKQKLESLGYQNSIEVLNAADFG
SSQARRRVFMISTLNEFVELPKGDKKPKSIKKVLNKIVSEKDILN
NLLKYNLTEFKKTKSNINKASLIGYSKENSEGYVYDPEFTGPTLT
ASGANSRIKIKDGSNIRKMNSDETFLYIGFDSQDGKRVNEIEFLT
ENQKIFVCGNSISVEVLEAIIDKIGG

In some embodiments, MQ1 is encoded by a nucleotide sequence of SEQ ID NO: 52 or 132. In some embodiments, a nucleic acid described herein comprises a sequence of SEQ ID NO: 52, 132 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 52)
AGCAAGGTGGAGAACAAGACCAAGAAGCTGCGGGTGTTCGAGGCC
TTCGCCGGCATCGGCGCCCAGCGGAAGGCCCTGGAGAAGGTGCGG
AAGGACGAGTACGAGATCGTGGGCCTGGCCGAGTGGTACGTGCCC
GCCATCGTGATGTACCAGGCCATCCACAACAACTTCCACACCAAG
CTGGAGTACAAGAGCGTGAGCCGGGAGGAGATGATCGACTACCTG
GAGAACAAGACCCTGAGCTGGAACAGCAAGAACCCCGTGAGCAAC
GGCTACTGGAAGCGGAAGAAGGACGACGAGCTGAAGATCATCTAC
AACGCCATCAAGCTGAGCGAGAAGGAGGGCAACATCTTCGACATC
CGGGACCTGTACAAGCGGACCCTGAAGAACATCGACCTGCTGACC
TACAGCTTCCCCTGCCAGGACCTGAGCCAGCAGGGCATCCAGAAG
GGCATGAAGCGGGGCAGCGGCACCCGGAGCGGCCTGCTGTGGGAG
ATCGAGCGGGCCCTGGACAGCACCGAGAAGAACGACCTGCCCAAG
TACCTGCTGATGGAGAACGTGGGCGCCCTGCTGCACAAGAAGAAC
GAGGAGGAGCTGAACCAGTGGAAGCAGAAGCTGGAGAGCCTGGGC
TACCAGAACAGCATCGAGGTGCTGAACGCCGCCGACTTCGGCAGC
AGCCAGGCCCGGCGGCGGGTGTTCATGATCAGCACCCTGAACGAG
TTCGTGGAGCTGCCCAAGGGCGACAAGAAGCCCAAGAGCATCAAG
AAGGTGCTGAACAAGATCGTGAGCGAGAAGGACATCCTGAACAAC
CTGCTGAAGTACAACCTGACCGAGTTCAAGAAAACCAAGAGCAAC
ATCAACAAGGCCAGCCTGATCGGCTACAGCAAGTTCAACAGCGAG
GGCTACGTGTACGACCCCGAGTTCACCGGCCCCACCCTGACCGCC
AGCGGCGCCAACAGCCGGATCAAGATCAAGGACGGCAGCAACATC
CGGAAGATGAACAGCGACGAGACCTTCCTGTACATCGGCTTCGAC
AGCCAGGACGGCAAGCGGGTGAACGAGATCGAGTTCCTGACCGAG
AACCAGAAGATCTTCGTGTGCGGCAACAGCATCAGCGTGGAGGTG
CTGGAGGCCATCATCGACAAGATCGGCGGC
(SEQ ID NO: 132)
AGCAAGGUGGAGAACAAGACCAAGAAGCUGCGGGUGUUCGAGGCC
UUCGCCGGCAUCGGCGCCCAGCGGAAGGCCCUGGAGAAGGUGCGG
AAGGACGAGUACGAGAUCGUGGGCCUGGCCGAGUGGUACGUGCCC
GCCAUCGUGAUGUACCAGGCCAUCCACAACAACUUCCACACCAAG
CUGGAGUACAAGAGCGUGAGCCGGGAGGAGAUGAUCGACUACCUG
GAGAACAAGACCCUGAGCUGGAACAGCAAGAACCCCGUGAGCAAC
GGCUACUGGAAGCGGAAGAAGGACGACGAGCUGAAGAUCAUCUAC
AACGCCAUCAAGCUGAGCGAGAAGGAGGGCAACAUCUUCGACAUC
CGGGACCUGUACAAGCGGACCCUGAAGAACAUCGACCUGCUGACC
UACAGCUUCCCCUGCCAGGACCUGAGCCAGCAGGGCAUCCAGAAG
GGCAUGAAGCGGGGCAGCGGCACCCGGAGCGGCCUGCUGUGGGAG
AUCGAGCGGGCCCUGGACAGCACCGAGAAGAACGACCUGCCCAAG
UACCUGCUGAUGGAGAACGUGGGCGCCCUGCUGCACAAGAAGAAC
GAGGAGGAGCUGAACCAGUGGAAGCAGAAGCUGGAGAGCCUGGGC
UACCAGAACAGCAUCGAGGUGCUGAACGCCGCCGACUUCGGCAGC
AGCCAGGCCCGGCGGCGGGUGUUCAUGAUCAGCACCCUGAACGAG
UUCGUGGAGCUGCCCAAGGGCGACAAGAAGCCCAAGAGCAUCAAG
AAGGUGCUGAACAAGAUCGUGAGCGAGAAGGACAUCCUGAACAAC
CUGCUGAAGUACAACCUGACCGAGUUCAAGAAaACCAAGAGCAAC
AUCAACAAGGCCAGCCUGAUCGGCUACAGCAAGUUCAACAGCGAG
GGCUACGUGUACGACCCCGAGUUCACCGGCCCCACCCUGACCGCC
AGCGGCGCCAACAGCCGGAUCAAGAUCAAGGACGGCAGCAACAUC
CGGAAGAUGAACAGCGACGAGACCUUCCUGUACAUCGGCUUCGAC
AGCCAGGACGGCAAGCGGGUGAACGAGAUCGAGUUCCUGACCGAG
AACCAGAAGAUCUUCGUGUGCGGCAACAGCAUCAGCGUGGAGGUG
CUGGAGGCCAUCAUCGACAAGAUCGGCGGC

In some embodiments, MQ1 for use in a polypeptide or an expression repressor described herein is a variant, e.g., comprising one or more mutations, relative to a wildtype MQ1 (e.g., SEQ ID NO: 19). In some embodiments, an MQ1 variant comprises one or more amino acid substitutions, deletions, or insertions relative to a wildtype MQ1, e.g., the MQ1 of SEQ ID NO: 19. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1).

In some embodiments, the one or more effector moiety is or comprises DNMT1, e.g., human DNMT1, or a functional variant or fragment thereof. In some embodiments, DNMT1 is human DNMT1, e.g., corresponding to Gene ID 1786, e.g., corresponding to UniProt ID P26358.2. In some embodiments, DNMT1 comprises an amino acid sequence of SEQ ID NO: 20 as described in International Application WO/2022/132195, which is herein incorporated by reference in its entirety. In some embodiments, an effector domain described herein comprises a sequence according to, e.g., SEQ ID NO: 20 as described in International Application WO/2022/132195, which is herein incorporated by reference in its entirety, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, DNMT1 is encoded by a nucleotide sequence according to, e.g., SEQ ID NO: 53 as described in International Application WO/2022/132195, which is herein incorporated by reference in its entirety, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

An exemplary effector moiety may include, but is not limited to any effector moiety as described in International Application WO/2022/132195, which is herein incorporated by reference in its entirety.

### Additional Moieties

An expression repressor may further comprise one or more additional moieties (e.g., in addition to one or more targeting moieties and one or more effector moieties). In some embodiments, an additional moiety is selected from a tagging or monitoring moiety, a cleavable moiety (e.g., a cleavable moiety positioned between a DNA-targeting moiety and an effector moiety or at the N- or C-terminal end of a polypeptide), a small molecule, a membrane translocating polypeptide, or a pharmaco-agent moiety.

### Exemplary Expression Repressors

The following exemplary expression repressors are presented for illustration purposes only and are not intended to be limiting.

In some embodiments, an expression repressor comprises a targeting moiety comprising a Zn Finger domain, and an effector moiety comprising KRAB, e.g., a KRAB domain. In some embodiments, the expression repressors are encoded by a nucleic acid sequence of any of SEQ ID NOs: 55, 56, 57, 58, 59, 60, 189, 194, 195, and 196 (e.g., a nucleic acid (e.g., cDNA) encoding the expression repressor). The nucleic acid sequences of these exemplary expression repressors are disclosed in Table 6. In some embodiments, a nucleic acid described herein comprises a nucleic acid sequence of any of SEQ ID NOs: 55-60, 189, 194-196, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the nucleic acid sequence comprises a poly-A sequence, and in other embodiments, the nucleic acid lacks the poly-A sequence.

TABLE 6
Nucleotide sequences of exemplary ZF-KRAB effectors

| | SEQ | |
|---|---|---|
| | ID | |
| NAME | NO. | SEQUENCE |
| ZFI- | 55 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| (nt) | | CCGGCGAGAAACCCTACAAGTGCCCCGAGTGCGGCAAATCCTTCTCTAGAAGCGACAAAC |
| | | TGACCGAACATCAGAGGACCCACACCGGCGAGAAGCCTTATAAGTGTCCCGAATGCGGCA |
| | | AATCCTTCAGCACCAAGAACTCTCTGACAGAACACCAGAGAACACATACCGGAGAGAAAC |
| | | CTTATAAATGCCCCGAGTGCGGCAAGTCCTTCTCCCAGTCCGGCGATCTGAGGAGACACC |
| | | AAAGAACACATACCGGCGAAAAGCCTTACAAGTGCCCCGAGTGTGGAAAGAGCTTCTCCA |
| | | CCACCGGCGCTCTGACCGAGCACCAGAGAACACACACCGGCGAGAAACCCTATAAATGTC |
| | | CCGAGTGTGGCAAATCCTTCAGCGACAGCGGCAATCTGAGAGTGCACCAAAGAACCCATA |
| | | CCGGCGAAAAACCCTACAAATGCCCCGAGTGCGGCAAATCCTTCAGCCAGAGGGCCCATC |
| | | TGGAGAGGCACCAAAGGACACACACCGGAGAAAAGCCCTACAAGTGTCCCGAGTGTGGAA |
| | | AAAGCTTTAGCACAAGCGGCGAGCTGGTGAGGCATCAAAGGACCCACACCGGCGAAAAGC |
| | | CCACCGGCAAAAAGACCAGCGCTAGCGGCAGCGGCGGCGGCAGCGGCGGCGACGCCAAGA |
| | | GCCTGACCGCCTGGAGCCGGACCCTGGTGACCTTCAAGGACGTGTTCGTGGACTTCACCC |
| | | GGGAGGAGTGGAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGACGTGATCCTGC |
| | | GGCTGGAGAAGGGCGAGGAGCCCTGGCTGGTGGAGCGGGAGATCCACCAGGAGACCCACC |
| | | CCGACAGCGAGACCGCCTTCGAGATCAAGAGCAGCGTGAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAA |
| ZF2- | 56 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| (nt) | | CCGGCGAGAAGCCCTACAAGTGCCCCGAGTGCGGAAAGTCCTTCAGCTCCCCCGCCGATC |
| | | TGACAAGACATCAGAGAACCCATACCGGCGAGAAACCTTACAAATGCCCCGAATGTGGCA |
| | | AGTCCTTTAGCGATCCCGGACATCTGGTGAGGCACCAGAGGACACACACCGGCGAAAAGC |
| | | CCTATAAATGTCCCGAGTGTGGAAAGAGCTTTTCTAGAAGCGACAATCTCGTGAGACACC |
| | | AGAGAACCCACACCGGAGAGAAGCCTTACAAGTGCCCCGAGTGCGGCAAATCCTTCAGCC |
| | | AGAGCTCCTCTCTGGTGAGGCACCAAAGGACCCACACCGGCGAGAAACCTTATAAGTGTC |
| | | CCGAGTGTGGCAAAAGCTTCAGCACCTCCCACTCTCTGACCGAGCATCAAAGAACCCACA |
| | | CCGGCGAAAAACCTTATAAATGCCCCGAGTGTGGCAAATCCTTCAGCAGAAATGACGCTC |
| | | TGACAGAGCACCAAAGAACACATACCGGAGAAAAGCCCTACAAATGCCCCGAGTGTGGAA |
| | | AATCCTTTTCTAGAAACGATGCTCTGACCGAACACCAAAGAACACACACCGGCGAAAAGC |
| | | CTACCGGAAAAAAGACCAGCGCTAGCGGCAGCGGCGGCGGCAGCGGCGGCGACGCCAAGA |
| | | GCCTGACCGCCTGGAGCCGGACCCTGGTGACCTTCAAGGACGTGTTCGTGGACTTCACCC |
| | | GGGAGGAGTGGAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGACGTGATCCTGC |
| | | GGCTGGAGAAGGGCGAGGAGCCCTGGCTGGTGGAGCGGGAGATCCACCAGGAGACCCACC |
| | | CCGACAGCGAGACCGCCTTCGAGATCAAGAGCAGCGTGAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAA |
| ZF3- | 57 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| (nt) | | CCGGCGAAAAACCTTACAAGTGCCCCGAGTGCGGAAAGAGCTTCAGCAGAAGCGACAAAC |
| | | TGGTGAGGCATCAAAGGACACATACCGGAGAGAAGCCCTATAAGTGCCCCGAATGTGGCA |
| | | AATCCTTTTCCCAGAGGGCTCATCTGGAAAGACACCAGAGGACCCATACCGGCGAAAAAC |
| | | CCTACAAATGTCCCGAGTGTGGAAAGAGCTTTTCCGATCCCGGCCATCTGGTCAGACATC |
| | | AGAGGACACATACCGGCGAAAAGCCTTACAAGTGTCCCGAATGCGGAAAATCCTTCTCCA |
| | | GAAGCGACAAGCTGGTGAGGCACCAAAGAACCCACACCGGCGAAAAACCCTATAAATGCC |
| | | CCGAGTGCGGCAAGTCCTTTAGCCAGCTGGCCCATCTGAGAGCCCACCAGAGAACACACA |
| | | CCGGAGAGAAGCCTTATAAGTGTCCCGAGTGCGGAAAGTCCTTCTCTAGAGCCGACAATC |
| | | TGACCGAACATCAAAGGACACACACCGGCGAGAAACCTTATAAATGCCCCGAGTGCGGAA |
| | | AAAGCTTTTCCGACTGCAGAGATCTGGCTAGACACCAGAGAACCCACACCGGCGAGAAAC |
| | | CCACCGGCAAAAAGACCAGCGCTAGCGGCAGCGGCGGCGGCAGCGGCGGCGACGCCAAGA |
| | | GCCTGACCGCCTGGAGCCGGACCCTGGTGACCTTCAAGGACGTGTTCGTGGACTTCACCC |
| | | GGGAGGAGTGGAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGACGTGATCCTGC |
| | | GGCTGGAGAAGGGCGAGGAGCCCTGGCTGGTGGAGCGGGAGATCCACCAGGAGACCCACC |
| | | CCGACAGCGAGACCGCCTTCGAGATCAAGAGCAGCGTGAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAA |
| ZF4- | 58 | AGGAAATAAGAGAGAAAAGAAGAGTAAGAAGAAATATAAGAGCCACCATGGCCCCCAAGAA |
| KRAB | | GAAGCGGAAGGTGGGCATCCACGGCGTGCCCGCCGCCGGCAGCAGCGGATCCCTGGAGCCC |
| (nt) | | GGCGAAAAGCCTTATAAATGTCCCGAATGCGGCAAGAGCTTTAGCCACACCGGCCATCTGC |
| | | TGGAACACCAAAGGACCCATACCGGCGAAAAGCCCTATAAGTGCCCCGAGTGTGGCAAGAG |
| | | CTTCAGCACCACCGGCAATCTGACAGTCCATCAGAGGACCCACACCGGAGAGAAACCCTAT |
| | | AAATGCCCCGAGTGTGGAAAGTCCTTCTCCGACAAGAAGGATCTGACAAGACACCAGAGGA |
| | | CCCATACCGGCGAGAAACCCTACAAATGCCCCGAGTGCGGCAAATCCTTCTCCCAGAGCGG |
| | | CGATCTGAGGAGACATCAAAGAACACATACCGGCGAAAAACCCTATAAGTGCCCCGAATGC |
| | | GGCAAGTCCTTCAGCCAGAGGGCCCATCTGGAAAGGCATCAGAGGACACACACCGGCGAGA |
| | | AGCCTTACAAATGTCCCGAGTGCGGAAAGAGCTTCTCTAGAAGCGACAAGCTGACCGAGCA |
| | | TCAGAGGACCCACACCGGAGAAAAACCTTACAAGTGCCCCGAGTGCGGCAAAAGCTTCAGC |
| | | AGAACCGACACACTGAGAGATCACCAAAGGACACACACCGGCGAGAAACCCACCGGCAAAA |
| | | AGACCAGCGCTAGCGGCAGCGGCGGCGGCAGCGGCGGCGACGCCAAGAGCCTGACCGCCTG |
| | | GAGCCGGACCCTGGTGACCTTCAAGGACGTGTTCGTGGACTTCACCCGGGAGGAGTGGAAG |
| | | CTGCTGGACACCGCCCAGCAGATCCTGTACCGGAACGTGATGCTGGAGAACTACAAGAACC |
| | | TGGTGAGCCTGGGCTACCAGCTGACCAAGCCCGACGTGATCCTGCGGCTGGAGAAGGGCGA |
| | | GGAGCCCTGGCTGGTGGAGCGGGAGATCCACCAGGAGACCCACCCCGACAGCGAGACCGCC |
| | | TTCGAGATCAAGAGCAGCGTGAGCGGCGGCAAGCGGCCCGCCGCCACCAAGAAGGCCGGCC |
| | | AGGCCAAGAAGAAGAAGGGCAGCTACCCCTACGACGTGCCCGACTACGCCTGAGCGGCCGC |
| | | TTAATTAAGCTGCCTTCTGCGGGGCTTGCCTTCTGGCCATGCCCTTCTTCTCTCCCTTGCA |
| | | CCTGTACCTCTTGGTCTTTGAATAAAGCCTGAGTAGGAAGTCTAGAAAAAAAAAAAAAAAA |
| | | AAAAAAAAAAAAAAAAAAAAAAAAAAAAAAAAAAAAAAAAAAAAAAAAAAAAAAAAAAAAA |
| | | AAA |
| ZF5- | 59 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| (nt) | | CCGGCGAGAAGCCTTATAAGTGCCCCGAGTGTGGCAAGAGCTTTAGCCACACCGGCCATC |
| | | TGCTGGAGCATCAAAGGACACACACCGGAGAAAAGCCCTATAAGTGCCCCGAGTGTGGCA |
| | | AATCCTTCAGCACCTCCGGCAATCTCACCGAACACCAGAGAACACACACCGGAGAAAAAC |
| | | CTTACAAATGTCCCGAGTGTGGAAAGAGCTTTTCCACCAGCGGCAATCTGGTGAGACATC |
| | | AAAGAACACATACCGGCGAAAAACCCTATAAATGCCCCGAGTGTGGAAAATCCTTCTCCC |
| | | AACTGGCCCATCTGAGGGCCCACCAGAGGACACATACCGGAGAAAAACCCTACAAATGCC |
| | | CCGAATGCGGAAAAAGCTTCTCCGAGAGAAGCCATCTGAGAGAGCACCAAAGGACCCATA |
| | | CCGGAGAAAAGCCTTACAAGTGTCCCGAGTGCGGAAAAAGCTTTAGCGATCCCGGACATC |
| | | TGGTGAGACATCAGAGAACCCACACCGGCGAAAAGCCTTATAAATGTCCCGAATGTGGCA |
| | | AGTCCTTTAGCACCCATCTGGATCTGATTAGACACCAAAGAACCCACACCGGCGAGAAAC |
| | | CCACCGGAAAAAAGACCAGCGCTAGCGGCAGCGGCGGCGGCAGCGGCGGCGACGCCAAGA |
| | | GCCTGACCGCCTGGAGCCGGACCCTGGTGACCTTCAAGGACGTGTTCGTGGACTTCACCC |
| | | GGGAGGAGTGGAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGACGTGATCCTGC |
| | | GGCTGGAGAAGGGCGAGGAGCCCTGGCTGGTGGAGCGGGAGATCCACCAGGAGACCCACC |
| | | CCGACAGCGAGACCGCCTTCGAGATCAAGAGCAGCGTGAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAA |
| ZF6- | 60 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| (nt) | | CCGGCGAAAAGCCTTACAAATGTCCCGAGTGCGGAAAGTCCTTCAGCGACCCCGGCGCTC |
| | | TGGTGAGACATCAAAGAACACATACCGGCGAGAAACCTTATAAATGCCCCGAATGTGGAA |
| | | AATCCTTCAGCGAAAGAAGCCATCTGAGGGAACACCAGAGGACCCACACCGGCGAAAAAC |
| | | CTTATAAGTGCCCCGAATGCGGAAAAAGCTTTTCTAGAAGCGATCATCTGACCAACCATC |
| | | AGAGAACACACACCGGCGAAAAGCCCTATAAATGTCCCGAGTGTGGCAAATCCTTTAGCG |
| | | AGAGGTCCCATCTGAGAGAGCACCAGAGGACACATACCGGAGAGAAGCCCTACAAGTGCC |
| | | CCGAGTGTGGCAAGAGCTTTAGCAGAAGCGACCATCTGACCAATCATCAAAGGACCCACA |
| | | CCGGAGAGAAGCCTTACAAGTGTCCCGAGTGCGGAAAGTCCTTTTCCGATCCCGGCCACC |
| | | TCGTGAGGCACCAAAGAACCCATACCGGCGAGAAACCCTACAAATGCCCCGAGTGTGGAA |
| | | AGAGCTTCTCCAGAAGCGACAAGCTGGTGAGGCATCAGAGGACACACACCGGCGAAAAAC |
| | | CCACCGGCAAGAAAACCAGCGCTAGCGGCAGCGGCGGCGGCAGCGGCGGCGACGCCAAGA |
| | | GCCTGACCGCCTGGAGCCGGACCCTGGTGACCTTCAAGGACGTGTTCGTGGACTTCACCC |
| | | GGGAGGAGTGGAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGACGTGATCCTGC |
| | | GGCTGGAGAAGGGCGAGGAGCCCTGGCTGGTGGAGCGGGAGATCCACCAGGAGACCCACC |
| | | CCGACAGCGAGACCGCCTTCGAGATCAAGAGCAGCGTGAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAA |
| ZF54- | 189 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| | | CTGGAGAGAAACCCTACAAATGCCCGGAATGCGGGAAGTCCTTTTCCGAACGCTCGCACC |
| | | TGAGGGAACACCAGAGAACTCACACCGGCGAAAAACCCTATAAGTGCCCAGAATGCGGAA |
| | | AGAGCTTTTCACGGTCGGACAACCTCGTGCGGCACCAACGCACTCATACCGGAGAGAAGC |
| | | CGTACAAGTGTCCTGAGTGCGGAAAGTCATTCTCCGACTGCCGGGATTTGGCCCGCCACC |
| | | AAAGAACACACACTGGCGAAAAGCCCTACAAGTGCCCGGAGTGTGGAAAGTCCTTCAGCA |
| | | CTTCCGGAGAGCTGGTCCGGCACCAGAGGACCCACACCGGGGAGAAGCCTTACAAATGTC |
| | | CAGAGTGCGGTAAAAGCTTCTCCACCACCGGCAACCTCACCGTGCACCAGCGGACCCACA |
| | | CTGGAGAAAAGCCGTATAAATGCCCCGAATGCGGCAAGAGCTTCTCGCGATCCGATAAGC |
| | | TTGTGCGGCATCAGAGAACGCACACTGGGGAAAAGCCTTATAAGTGTCCGGAGTGCGGCA |
| | | AATCCTTCTCCCGCACTGACACCCTGCGGGACCATCAGCGCACCCATACCGGCAAAAAGA |
| | | CCTCTGCTAGCGGCAGCGGCGGCGGCAGCGGCGGCGCCCGGGACGACGCCAAGAGCCTGA |
| | | CCGCCTGGAGCCGGACCCTGGTGACCTTCAAGGACGTGTTCGTGGACTTCACCCGGGAGG |
| | | AGTGGAAGCTGCTGGACACCGCCCAGCAGATCCTGTACCGGAACGTGATGCTGGAGAACT |
| | | ACAAGAACCTGGTGAGCCTGGGCTACCAGCTGACCAAGCCCGACGTGATCCTGCGGCTGG |
| | | AGAAGGGCGAGGAGCCCTGGCTGGTGGAGCGGGAGATCCACCAGGAAACCCACCCCGACA |
| | | GCGAAACCGCCTTCGAGATCAAGAGCAGCGTGCCCAGCAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAA |
| ZF61- | 193 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTTGAAC |
| | | CCGGGGAGAAGCCCTACAAGTGCCCGGAATGCGGAAAGAGCTTCAGCCAGAAGTCCTCGC |
| | | TGATCGCGCACCAGAGGACTCACACCGGCGAAAAGCCATACAAGTGTCCTGAGTGTGGCA |
| | | AATCCTTCTCGCACAAGAACGCACTGCAGAACCACCAGAGAACCCACACCGGGGAAAAGC |
| | | CGTATAAGTGCCCCGAATGTGGAAAGTCGTTCAGCCAGTCATCCAACCTCGTGCGCCATC |
| | | AAAGGACTCATACCGGAGAGAAACCTTACAAATGCCCTGAATGCGGCAAATCTTTCTCCC |
| | | GGAATGATGCCCTGACCGAGCACCAGCGCACTCACACGGGAGAGAAGCCGTACAAATGTC |
| | | CGGAGTGCGGAAAGTCCTTCTCCGACAAGAAGGACTTGACCAGACACCAGCGGACCCATA |
| | | CTGGCGAAAAACCCTATAAGTGTCCAGAGTGCGGGAAGTCCTTTAGCCAAGCCGGTCACC |
| | | TCGCTTCCCACCAACGGACCCACACAGGAGAAAAGCCTTATAAATGCCCCGAGTGCGGCA |
| | | AAAGCTTCTCCGATAAGAAGGACCTGACTCGGCATCAGCGCACCCATACCGGAAAGAAAA |
| | | CCTCAGCTAGCGGCAGCGGCGGCGGCAGCGGCGGCGCCCGGGACGACGCCAAGAGCCTGA |
| | | CCGCCTGGAGCCGGACCCTGGTGACCTTCAAGGACGTGTTCGTGGACTTCACCCGGGAGG |
| | | AGTGGAAGCTGCTGGACACCGCCCAGCAGATCCTGTACCGGAACGTGATGCTGGAGAACT |
| | | ACAAGAACCTGGTGAGCCTGGGCTACCAGCTGACCAAGCCCGACGTGATCCTGCGGCTGG |
| | | AGAAGGGCGAGGAGCCCTGGCTGGTGGAGCGGGAGATCCACCAGGAAACCCACCCCGACA |
| | | GCGAAACCGCCTTCGAGATCAAGAGCAGCGTGCCCAGCAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAA |
| ZF67- | 194 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| | | CTGGCGAAAAACCCTATAAGTGCCCAGAATGCGGAAAGAGCTTTTCACGGTCGGACAACC |
| | | TCGTGCGGCACCAACGCACTCATACCGGAGAGAAGCCGTACAAGTGTCCTGAGTGCGGAA |
| | | AGTCATTCTCCGACTGCCGGGATTTGGCCCGCCACCAAAGAACACACACTGGCGAAAAGC |
| | | CCTACAAGTGCCCGGAGTGTGGAAAGTCCTTCAGCACTTCCGGAGAGCTGGTCCGGCACC |
| | | AGAGGACCCACACCGGGGAGAAGCCTTACAAATGTCCAGAGTGCGGTAAAAGCTTCTCCA |
| | | CCACCGGCAACCTCACCGTGCACCAGCGGACCCACACTGGAGAAAAGCCGTATAAATGCC |
| | | CCGAATGCGGCAAGAGCTTCTCGCGATCCGATAAGCTTGTGCGGCATCAGAGAACGCACA |
| | | CTGGGGAAAAGCCTTATAAGTGTCCGGAGTGCGGCAAATCCTTCTCCCGCACTGACACCC |
| | | TGCGGGACCACCAGAGAACCCATACTGGCGAGAAGCCATACAAATGCCCGGAATGTGGAA |
| | | AGAGTTTCTCGCGCGAGGACAACCTCCACACCCATCAGCGCACCCATACCGGCAAAAAGA |
| | | CCTCTGCTAGCGGCAGCGGCGGCGGCAGCGGCGGCGCCCGGGACGACGCCAAGAGCCTGA |
| | | CCGCCTGGAGCCGGACCCTGGTGACCTTCAAGGACGTGTTCGTGGACTTCACCCGGGAGG |
| | | AGTGGAAGCTGCTGGACACCGCCCAGCAGATCCTGTACCGGAACGTGATGCTGGAGAACT |
| | | ACAAGAACCTGGTGAGCCTGGGCTACCAGCTGACCAAGCCCGACGTGATCCTGCGGCTGG |
| | | AGAAGGGCGAGGAGCCCTGGCTGGTGGAGCGGGAGATCCACCAGGAAACCCACCCCGACA |
| | | GCGAAACCGCCTTCGAGATCAAGAGCAGCGTGCCCAGCAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAA |
| ZF68- | 195 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAAC |
| | | CCGGAGAGAAACCCTACAAATGCCCAGAGTGCGGCAAATCGTTCTCACGGTCCGATCACC |
| | | TCACCACCCACCAGAGGACCCATACCGGGGAGAAGCCTTACAAGTGTCCTGAGTGTGGAA |
| | | AGTCCTTCAGCCAAAAGTCCTCGCTGATCGCACACCAGCGCACGCACACTGGGGAAAAGC |
| | | CATATAAATGCCCGGAGTGTGGCAAATCCTTCTCCCGCCGCGACGAACTGAACGTGCACC |
| | | AGAGAACCCACACTGGAGAGAAGCCGTATAAGTGTCCGGAGTGCGGAAAGAGCTTCTCGC |
| | | AATCCGGGGACCTTCGGAGACATCAGAGGACACACACTGGCGAAAAGCCCTATAAGTGCC |
| | | CTGAGTGCGGGAAGTCCTTTAGCCAAGCCGGTCACCTGGCCTCCCACCAACGGACTCACA |
| | | CCGGCGAAAAACCGTACAAGTGCCCCGAATGCGGAAAGTCGTTCTCTACCTCCGGAAACT |
| | | TGACCGAACACCAGCGGACCCACACCGGAGAAAAGCCGTACAAATGTCCTGAATGCGGCA |
| | | AAAGCTTCAGCCAGGCCGGTCATCTCGCGAGCCATCAGCGGACTCATACTGGCAAAAAGA |
| | | CCTCAGCTAGCGGCAGCGGCGGCGGCAGCGGCGGCGCCCGGGACGACGCCAAGAGCCTGA |
| | | CCGCCTGGAGCCGGACCCTGGTGACCTTCAAGGACGTGTTCGTGGACTTCACCCGGGAGG |
| | | AGTGGAAGCTGCTGGACACCGCCCAGCAGATCCTGTACCGGAACGTGATGCTGGAGAACT |
| | | ACAAGAACCTGGTGAGCCTGGGCTACCAGCTGACCAAGCCCGACGTGATCCTGCGGCTGG |
| | | AGAAGGGCGAGGAGCCCTGGCTGGTGGAGCGGGAGATCCACCAGGAAACCCACCCCGACA |
| | | GCGAAACCGCCTTCGAGATCAAGAGCAGCGTGCCCAGCAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCTGAGCGGCCGCTTAATTAAGCTGCCTTCTGCGGGGCTTGCCTTCTGGCCATG |
| | | CCCTTCTTCTCTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAA |

In some embodiments, an expression repressor comprises a targeting moiety comprising a Zn Finger domain (e.g., having an amino acid sequence according to any of SEQ ID NO: 5-10 or 169-172), and an effector moiety comprising KRAB (e.g., an amino acid sequence SEQ ID NO: 18), e.g., a KRAB domain. In some embodiments, an expression repressor described herein comprises an amino sequence of any of SEQ ID NOs: 22, 23, 24, 25, 26, 27, 139-144, 177-180, or 183-186. The protein sequence of these exemplary expression repressors are disclosed in Table 7. In some embodiments, an expression repressor described herein comprises an amino acid sequence of any of SEQ ID NOs: 22-27, 139-144, 177-180, 183-186 or a sequence with at least 80, 85, 90, 95, 99, or 10000 identity thereto, or having no more than 20, 19, 18, 17, 16, 15, 14, 13, 12, 11, 10, 9, 8, 7, 6, 5, 4, 3, 2, or 1 positions of difference thereto.

TABLE 7
Amino Acid sequences of exemplary Zinc Finger-KRAB effectors

| | SEQ | |
|---|---|---|
| | ID | |
| NAME | NO. | SEQUENCE |
| ZF1- | 22 | MAPKKKRKVGIHGVPAAGSSGSLEPGEKPYKCPECGKSFSRSDKLTEHQRTHTGEKPYKCPECGKSF |
| KRAB | | STKNSLTEHQRTHTGEKPYKCPECGKSFSQSGDLRRHQRTHTGEKPYKCPECGKSFSTTGALTEHQR |
| (aa) | | THTGEKPYKCPECGKSFSDSGNLRVHQRTHTGEKPYKCPECGKSFSQRAHLERHQRTHTGEKPYKCP |
| | | ECGKSFSTSGELVRHQRTHTGEKPTGKKTSASGSGGGSGGDAKSLTAWSRTLVTFKDVFVDFTREEW |
| | | KLLDTAQQILYRNVMLENYKNLVSLGYQLTKPDVILRLEKGEEPWLVEREIHQETHPDSETAFEIKS |
| | | SVSGGKRPAATKKAGQAKKKKGSYPYDVPDYA |
| ZF2- | 23 | MAPKKKRKVGIHGVPAAGSSGSLEPGEKPYKCPECGKSFSSPADLTRHQRTHTGEKPYKCPECGKSF |
| KRAB | | SDPGHLVRHQRTHTGEKPYKCPECGKSFSRSDNLVRHQRTHTGEKPYKCPECGKSFSQSSSLVRHQR |
| (aa) | | THTGEKPYKCPECGKSFSTSHSLTEHQRTHTGEKPYKCPECGKSFSRNDALTEHQRTHTGEKPYKCP |
| | | ECGKSFSRNDALTEHQRTHTGEKPTGKKTSASGSGGGSGGDAKSLTAWSRTLVTFKDVFVDFTREEW |
| | | KLLDTAQQILYRNVMLENYKNLVSLGYQLTKPDVILRLEKGEEPWLVEREIHQETHPDSETAFEIKS |
| | | SVSGGKRPAATKKAGQAKKKKGSYPYDVPDYA |
| ZF3- | 24 | MAPKKKRKVGIHGVPAAGSSGSLEPGEKPYKCPECGKSFSRSDKLVRHQRTHTGEKPYKCPECGKSF |
| KRAB | | SQRAHLERHQRTHTGEKPYKCPECGKSFSDPGHLVRHQRTHTGEKPYKCPECGKSFSRSDKLVRHQR |
| (aa) | | THTGEKPYKCPECGKSFSQLAHLRAHQRTHTGEKPYKCPECGKSFSRADNLTEHQRTHTGEKPYKCP |
| | | ECGKSFSDCRDLARHQRTHTGEKPTGKKTSASGSGGGSGGDAKSLTAWSRTLVTFKDVFVDFTREEW |
| | | KLLDTAQQILYRNVMLENYKNLVSLGYQLTKPDVILRLEKGEEPWLVEREIHQETHPDSETAFEIKS |
| | | SVSGGKRPAATKKAGQAKKKKGSYPYDVPDYA |
| ZF4- | 25 | MAPKKKRKVGIHGVPAAGSSGSLEPGEKPYKCPECGKSFSHTGHLLEHQRTHTGEKPYKCPECGKSF |
| KRAB | | STTGNLTVHQRTHTGEKPYKCPECGKSFSDKKDLTRHQRTHTGEKPYKCPECGKSFSQSGDLRRHQR |
| (aa) | | THTGEKPYKCPECGKSFSQRAHLERHQRTHTGEKPYKCPECGKSFSRSDKLTEHQRTHTGEKPYKCP |
| | | ECGKSFSRTDTLRDHQRTHTGEKPTGKKTSASGSGGGSGGDAKSLTAWSRTLVTFKDVFVDFTREEW |
| | | KLLDTAQQILYRNVMLENYKNLVSLGYQLTKPDVILRLEKGEEPWLVEREIHQETHPDSETAFEIKS |
| | | SVSGGKRPAATKKAGQAKKKKGSYPYDVPDYA |
| ZF5- | 26 | MAPKKKRKVGIHGVPAAGSSGSLEPGEKPYKCPECGKSFSHTGHLLEHQRTHTGEKPYKCPECGKSF |
| KRAB | | STSGNLTEHQRTHTGEKPYKCPECGKSESTSGNLVRHQRTHTGEKPYKCPECGKSFSQLAHLRAHQR |
| (aa) | | THTGEKPYKCPECGKSFSERSHLREHQRTHTGEKPYKCPECGKSFSDPGHLVRHQRTHTGEKPYKCP |
| | | ECGKSFSTHLDLIRHQRTHTGEKPTGKKTSASGSGGGSGGDAKSLTAWSRTLVTFKDVFVDFTREEW |
| | | KLLDTAQQILYRNVMLENYKNLVSLGYQLTKPDVILRLEKGEEPWLVEREIHQETHPDSETAFEIKS |
| | | SVSGGKRPAATKKAGQAKKKKGSYPYDVPDYA |
| ZF6- | 27 | MAPKKKRKVGIHGVPAAGSSGSLEPGEKPYKCPECGKSFSDPGALVRHQRTHTGEKPYKCPECGKS |
| KRAB | | FSERSHLREHQRTHTGEKPYKCPECGKSFSRSDHLTNHQRTHTGEKPYKCPECGKSFSERSHLREH |
| (aa) | | QRTHTGEKPYKCPECGKSFSRSDHLTNHQRTHTGEKPYKCPECGKSFSDPGHLVRHQRTHTGEKPY |
| | | KCPECGKSFSRSDKLVRHQRTHTGEKPTGKKTSASGSGGGSGGDAKSLTAWSRTLVTFKDVFVDFT |
| | | REEWKLLDTAQQILYRNVMLENYKNLVSLGYQLTKPDVILRLEKGEEPWLVEREIHQETHPDSETA |
| | | FEIKSSVSGGKRPAATKKAGQAKKKKGSYPYDVPDYA |
| tPT2A | 134 | PLEGSSGSGSPKKKRKVGIHGVPAAGSSGSLEPGEKPYKCPECGKSFSRSDKLVRHQRTHTGEKPY |
| fragment | | KCPECGKSFSQRAHLERHQRTHTGEKPYKCPECGKSFSDPGHLVRHQRTHTGEKPYKCPECGKSFS |
| 3+ZF3- | | RSDKLVRHQRTHTGEKPYKCPECGKSFSQLAHLRAHQRTHTGEKPYKCPECGKSFSRADNLTEHQR |
| KRAB | | THTGEKPYKCPECGKSFSDCRDLARHQRTHTGEKPTGKKTSASGSGGGSGGDAKSLTAWSRTLVTF |
| | | KDVFVDFTREEWKLLDTAQQILYRNVMLENYKNLVSLGYQLTKPDVILRLEKGEEPWLVEREIHQE |
| | | THPDSETAFEIKSSVSGGKRPAATKKAGQAKKKKGS |
| ZF1- | 139 | MAPKKKRKVGIHGVPAAGSSGSLEPGEKPYKCPECGKSFSRSDKLTEHQRTHTGEKPYKCPECGKS |
| KRAB | | FSTKNSLTEHQRTHTGEKPYKCPECGKSFSQSGDLRRHQRTHTGEKPYKCPECGKSFSTTGALTEH |
| without | | QRTHTGEKPYKCPECGKSFSDSGNLRVHQRTHTGEKPYKCPECGKSFSQRAHLERHQRTHTGEKPY |
| HA | | KCPECGKSFSTSGELVRHQRTHTGEKPTGKKTSASGSGGGSGGDAKSLTAWSRTLVTFKDVFVDFT |
| (aa) | | REEWKLLDTAQQILYRNVMLENYKNLVSLGYQLTKPDVILRLEKGEEPWLVEREIHQETHPDSETA |
| | | FEIKSSVSGGKRPAATKKAGQAKKKKGS |
| ZF2- | 140 | MAPKKKRKVGIHGVPAAGSSGSLEPGEKPYKCPECGKSFSSPADLTRHQRTHTGEKPYKCPECGKS |
| KRAB | | FSDPGHLVRHQRTHTGEKPYKCPECGKSFSRSDNLVRHQRTHTGEKPYKCPECGKSFSQSSSLVRH |
| without | | QRTHTGEKPYKCPECGKSFSTSHSLTEHQRTHTGEKPYKCPECGKSFSRNDALTEHQRTHTGEKPY |
| HA | | KCPECGKSFSRNDALTEHQRTHTGEKPTGKKTSASGSGGGSGGDAKSLTAWSRTLVTFKDVFVDFT |
| (aa) | | REEWKLLDTAQQILYRNVMLENYKNLVSLGYQLTKPDVILRLEKGEEPWLVEREIHQETHPDSETA |
| | | FEIKSSVSGGKRPAATKKAGQAKKKKGS |
| ZF3- | 141 | MAPKKKRKVGIHGVPAAGSSGSLEPGEKPYKCPECGKSFSRSDKLVRHQRTHTGEKPYKCPECGKS |
| KRAB | | FSQRAHLERHQRTHTGEKPYKCPECGKSFSDPGHLVRHQRTHTGEKPYKCPECGKSFSRSDKLVRH |
| without | | QRTHTGEKPYKCPECGKSFSQLAHLRAHQRTHTGEKPYKCPECGKSFSRADNLTEHQRTHTGEKPY |
| HA | | KCPECGKSFSDCRDLARHQRTHTGEKPTGKKTSASGSGGGSGGDAKSLTAWSRTLVTFKDVFVDFT |
| (aa) | | REEWKLLDTAQQILYRNVMLENYKNLVSLGYQLTKPDVILRLEKGEEPWLVEREIHQETHPDSETA |
| | | FEIKSSVSGGKRPAATKKAGQAKKKKGS |
| ZF4- | 142 | MAPKKKRKVGIHGVPAAGSSGSLEPGEKPYKCPECGKSFSHTGHLLEHQRTHTGEKPYKCPECGKS |
| KRAB | | FSTTGNLTVHQRTHTGEKPYKCPECGKSFSDKKDLTRHQRTHTGEKPYKCPECGKSFSQSGDLRRH |
| without | | QRTHTGEKPYKCPECGKSFSQRAHLERHQRTHTGEKPYKCPECGKSFSRSDKLTEHQRTHTGEKPY |
| HA | | KCPECGKSFSRTDTLRDHQRTHTGEKPTGKKTSASGSGGGSGGDAKSLTAWSRTLVTFKDVFVDFT |
| (aa) | | REEWKLLDTAQQILYRNVMLENYKNLVSLGYQLTKPDVILRLEKGEEPWLVEREIHQETHPDSETA |
| | | FEIKSSVSGGKRPAATKKAGQAKKKKGS |
| ZF5- | 143 | MAPKKKRKVGIHGVPAAGSSGSLEPGEKPYKCPECGKSFSHTGHLLEHQRTHTGEKPYKCPECGKS |
| KRAB | | FSTSGNLTEHQRTHTGEKPYKCPECGKSFSTSGNLVRHQRTHTGEKPYKCPECGKSFSQLAHLRAH |
| without | | QRTHTGEKPYKCPECGKSFSERSHLREHQRTHTGEKPYKCPECGKSFSDPGHLVRHQRTHTGEKPY |
| HA | | KCPECGKSFSTHLDLIRHQRTHTGEKPTGKKTSASGSGGGSGGDAKSLTAWSRTLVTFKDVFVDFT |
| (aa) | | REEWKLLDTAQQILYRNVMLENYKNLVSLGYQLTKPDVILRLEKGEEPWLVEREIHQETHPDSETA |
| | | FEIKSSVSGGKRPAATKKAGQAKKKKGS |
| ZF6- | 144 | MAPKKKRKVGIHGVPAAGSSGSLEPGEKPYKCPECGKSFSDPGALVRHQRTHTGEKPYKCPECGKS |
| KRAB | | FSERSHLREHQRTHTGEKPYKCPECGKSFSRSDHLTNHQRTHTGEKPYKCPECGKSFSERSHLREH |
| without | | QRTHTGEKPYKCPECGKSFSRSDHLTNHQRTHTGEKPYKCPECGKSFSDPGHLVRHQRTHTGEKPY |
| HA (aa) | | KCPECGKSFSRSDKLVRHQRTHTGEKPTGKKTSASGSGGGSGGDAKSLTAWSRTLVTFKDVFVDFT |
| | | REEWKLLDTAQQILYRNVMLENYKNLVSLGYQLTKPDVILRLEKGEEPWLVEREIHQETHPDSETA |
| | | FEIKSSVSGGKRPAATKKAGQAKKKKGS |
| ZF54- | 177 | MAPKKKRKVGIHGVPAAGSSGSLEPGEKPYKCPECGKSFSERSHLREHQRTHTGEKPYKCPECGKS |
| KRAB aa | | FSRSDNLVRHQRTHTGEKPYKCPECGKSFSDCRDLARHQRTHTGEKPYKCPECGKSFSTSGELVRH |
| | | QRTHTGEKPYKCPECGKSFSTTGNLTVHQRTHTGEKPYKCPECGKSFSRSDKLVRHQRTHTGEKPY |
| | | KCPECGKSFSRTDTLRDHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGSYPYDVPDYA |
| ZF61- | 178 | MAPKKKRKVGIHGVPAAGSSGSLEPGEKPYKCPECGKSFSQKSSLIAHQRTHTGEKPYKCPECGKS |
| KRAB aa | | FSHKNALQNHQRTHTGEKPYKCPECGKSFSQSSNLVRHQRTHTGEKPYKCPECGKSFSRNDALTEH |
| | | QRTHTGEKPYKCPECGKSFSDKKDLTRHQRTHTGEKPYKCPECGKSFSQAGHLASHQRTHTGEKPY |
| | | KCPECGKSFSDKKDLTRHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGSYPYDVPDYA |
| ZF67- | 179 | MAPKKKRKVGIHGVPAAGSSGSLEPGEKPYKCPECGKSFSRSDNLVRHQRTHTGEKPYKCPECGKS |
| KRAB aa | | FSDCRDLARHQRTHTGEKPYKCPECGKSFSTSGELVRHQRTHTGEKPYKCPECGKSFSTTGNLTVH |
| | | QRTHTGEKPYKCPECGKSFSRSDKLVRHQRTHTGEKPYKCPECGKSFSRTDTLRDHQRTHTGEKPY |
| | | KCPECGKSFSREDNLHTHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGSYPYDVPDYA |
| ZF68- | 180 | MAPKKKRKVGIHGVPAAGSSGSLEPGEKPYKCPECGKSFSRSDHLTTHQRTHTGEKPYKCPECGKS |
| KRAB aa | | FSQKSSLIAHQRTHTGEKPYKCPECGKSFSRRDELNVHQRTHTGEKPYKCPECGKSFSQSGDLRRH |
| | | QRTHTGEKPYKCPECGKSFSQAGHLASHQRTHTGEKPYKCPECGKSFSTSGNLTEHQRTHTGEKPY |
| | | KCPECGKSFSQAGHLASHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGSYPYDVPDYA |
| ZF54- | 183 | MAPKKKRKVGIHGVPAAGSSGSLEPGEKPYKCPECGKSFSERSHLREHQRTHTGEKPYKCPECGKS |
| KRAB aa | | FSRSDNLVRHQRTHTGEKPYKCPECGKSFSDCRDLARHQRTHTGEKPYKCPECGKSFSTSGELVRH |
| without | | QRTHTGEKPYKCPECGKSFSTTGNLTVHQRTHTGEKPYKCPECGKSFSRSDKLVRHQRTHTGEKPY |
| HA tag | | KCPECGKSFSRTDTLRDHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGS |
| ZF61- | 184 | MAPKKKRKVGIHGVPAAGSSGSLEPGEKPYKCPECGKSFSQKSSLIAHQRTHTGEKPYKCPECGKS |
| KRAB aa | | FSHKNALQNHQRTHTGEKPYKCPECGKSFSQSSNLVRHQRTHTGEKPYKCPECGKSFSRNDALTEH |
| without | | QRTHTGEKPYKCPECGKSFSDKKDLTRHQRTHTGEKPYKCPECGKSFSQAGHLASHQRTHTGEKPY |
| HA tag | | KCPECGKSFSDKKDLTRHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGS |
| ZF67- | 185 | MAPKKKRKVGIHGVPAAGSSGSLEPGEKPYKCPECGKSFSRSDNLVRHQRTHTGEKPYKCPECGKS |
| KRAB aa | | FSDCRDLARHQRTHTGEKPYKCPECGKSFSTSGELVRHQRTHTGEKPYKCPECGKSFSTTGNLTVH |
| without | | QRTHTGEKPYKCPECGKSFSRSDKLVRHQRTHTGEKPYKCPECGKSFSRTDTLRDHQRTHTGEKPY |
| HA tag | | KCPECGKSFSREDNLHTHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGS |
| ZF68- | 186 | MAPKKKRKVGIHGVPAAGSSGSLEPGEKPYKCPECGKSFSRSDHLTTHQRTHTGEKPYKCPECGKS |
| KRAB aa | | FSQKSSLIAHQRTHTGEKPYKCPECGKSFSRRDELNVHQRTHTGEKPYKCPECGKSFSQSGDLRRH |
| without | | QRTHTGEKPYKCPECGKSFSQAGHLASHQRTHTGEKPYKCPECGKSFSTSGNLTEHQRTHTGEKPY |
| HA tag | | KCPECGKSFSQAGHLASHQRTHTGKKTSASGSGGGSGGARDDAKSLTAWSRTLVTFKDVFVDFTRE |
| | | EWKLLDTAQQILYRNVMLENYKNLVSLGYQLTKPDVILRLEKGEEPWLVEREIHQETHPDSETAFE |
| | | IKSSVPSSGGKRPAATKKAGQAKKKKGS |

In some embodiments, an expression repressor comprises a targeting moiety comprising a Zn Finger domain (e.g., one encoded by a nucleotide sequence of any of SEQ ID NO: 44-49 or 115), and an effector moiety comprising MQ1, e.g., a bacterial MQ1 (e.g., one encoded by a nucleotide sequence of SEQ ID NO: 52). In some embodiments, the expression repressors are encoded by the nucleic sequence of SEQ ID NOs: 61, 62, 63, 64, 65, 66, 116, 117, 118, or 130. The nucleic acid sequence of these exemplary expression repressors are disclosed in Table 8. In some embodiments, a nucleic acid described herein comprises a nucleic acid sequence of any of SEQ ID NO: 61-66, 116-118, 130 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the nucleic acid sequence comprises a poly-A sequence, and in other embodiments, the nucleic acid lacks the poly-A sequence. For example, in some embodiments, a nucleic acid described herein comprises a sequence according to any of SEQ ID NO: 61-66, 116-118, or 130 (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the 3′ poly-A sequence, or comprising a 3′ poly-A sequence of a shorter length.

TABLE 8
Nucleotide sequences of exemplary ZF-MQ1 effectors

| | SEQ | |
|---|---|---|
| | ID | |
| NAME | NO | SEQUENCE |
| ZF7- | 61 | AGGAAATAAGAGAGAAAAGAAGAGTAAGAAGAAATATAAGAGCCACCATGGCCCCCAAGA |
| MQ1 | | AGAAGCGGAAGGTGGGCATCCACGGCGTGCCCGCCGCCGGCAGCAGCGGATCCCTGGAGC |
| (nt) | | CCGGAGAGAAGCCCTACAAATGCCCCGAGTGTGGAAAGAGCTTCTCTAGAAATGACGCTC |
| | | TGACAGAACACCAGAGGACCCATACCGGCGAGAAACCTTACAAATGCCCCGAGTGCGGAA |
| | | AAAGCTTTAGCGATTGCAGAGATCTGGCTAGACATCAGAGAACACACACCGGCGAGAAGC |
| | | CCTATAAGTGCCCCGAATGCGGCAAGAGCTTTAGCGACCCCGGCCATCTGGTGAGACATC |
| | | AAAGGACACATACCGGAGAAAAACCTTACAAGTGCCCCGAGTGCGGAAAGTCCTTCTCCC |
| | | AGAGCGGCCATCTCACCGAGCATCAAAGGACCCACACCGGCGAAAAGCCTTATAAATGTC |
| | | CCGAATGTGGCAAGTCCTTCTCTAGAGAGGATAATCTGCACACCCATCAGAGGACCCACA |
| | | CCGGCGAAAAGCCTTATAAATGCCCCGAATGTGGAAAGTCCTTTTCCACCAAGAACTCTC |
| | | TGACCGAGCATCAGAGGACACACACCGGAGAGAAACCCTATAAATGTCCCGAGTGTGGCA |
| | | AGAGCTTCAGCAGAGCCGACAATCTGACAGAGCACCAAAGAACACATACCGGCGAAAAGC |
| | | CCACCGGCAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCCTGAGCGGCCG |
| | | CTTAATTAAGCTGCCTTCTGCGGGGCTTGCCTTCTGGCCATGCCCTTCTTCTCTCCCTTG |
| | | CACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAA |
| | | AAAAAAA |
| ZF8- | 62 | AGGAAATAAGAGAGAAAAGAAGAGTAAGAAGAAATATAAGAGCCACCATGGCCCCCAAGA |
| MQ1 | | AGAAGCGGAAGGTGGGCATCCACGGCGTGCCCGCCGCCGGCAGCAGCGGATCCCTGGAGC |
| (nt) | | CCGGCGAGAAACCCTACAAGTGCCCCGAGTGTGGCAAATCCTTCTCTAGATCCGACAAAC |
| | | TGACCGAACATCAGAGGACCCATACCGGCGAAAAACCTTATAAATGTCCCGAGTGCGGAA |
| | | AGTCCTTCTCTAGAAGGGACGAGCTGAACGTGCATCAGAGAACACATACCGGCGAGAAGC |
| | | CCTATAAATGCCCCGAATGCGGCAAAAGCTTCTCTAGAAGCGATCATCTGACCAACCACC |
| | | AGAGAACCCATACCGGAGAAAAGCCTTACAAGTGTCCCGAATGTGGAAAATCCTTCAGCT |
| | | CCCCCGCCGATCTGACCAGACACCAAAGGACCCACACCGGCGAGAAGCCCTATAAATGCC |
| | | CCGAGTGCGGCAAGAGCTTTTCCAGATCCGACCATCTGACCAATCATCAAAGAACCCACA |
| | | CCGGCGAAAAGCCTTATAAATGTCCCGAGTGCGGCAAATCCTTTTCCAGCAAGAAGGCTC |
| | | TGACCGAGCATCAAAGGACCCATACCGGCGAGAAGCCTTACAAATGCCCCGAGTGTGGAA |
| | | AGTCCTTTAGCACCCATCTGGATCTGATTAGACACCAGAGGACACACACCGGAGAGAAAC |
| | | CCACCGGCAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCCTGAGCGGCCG |
| | | CTTAATTAAGCTGCCTTCTGCGGGGCTTGCCTTCTGGCCATGCCCTTCTTCTCTCCCTTG |
| | | CACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAA |
| | | AAAAAAA |
| ZF9- | 63 | AGGAAATAAGAGAGAAAAGAAGAGTAAGAAGAAATATAAGAGCCACCATGGCCCCCAAGA |
| 63 | | AGAAGCGGAAGGTGGGCATCCACGGCGTGCCCGCCGCCGGCAGCAGCGGATCCCTGGAGC |
| MQ1 | | CCGGCGAGAAACCTTACAAATGCCCCGAGTGCGGCAAGAGCTTCAGCAGAAGCGACGATC |
| (nt) | | AGTCCTTCAGCAGAGAGGACAATCTGCACACCCACCAGAGAACACACACCGGAGAAAAGC |
| | | TGGTGAGGCACCAAAGAACCCACACCGGCGAAAAACCTTACAAGTGTCCCGAATGCGGAA |
| | | CTTACAAGTGCCCCGAATGCGGCAAATCCTTTTCTAGAAGCGATCATCTGACCACCCACC |
| | | AAAGAACACATACCGGCGAGAAGCCTTACAAATGTCCCGAGTGCGGAAAGTCCTTCTCCC |
| | | AGAGAGCCAATCTGAGGGCTCATCAAAGGACCCATACCGGCGAAAAGCCCTACAAATGCC |
| | | CCGAGTGCGGAAAATCCTTCAGCCAGCTGGCCCATCTGAGAGCCCACCAAAGGACACACA |
| | | CCGGAGAGAAACCCTATAAGTGCCCCGAGTGTGGAAAAAGCTTTTCCCAGAGGGCCAATC |
| | | TGAGGGCCCATCAGAGGACCCATACCGGAGAGAAGCCTTATAAATGTCCCGAGTGCGGAA |
| | | AAAGCTTCAGCGAGAGGAGCCATCTGAGGGAACATCAAAGAACCCACACCGGCGAAAAAC |
| | | CCACCGGAAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCCTGAGCGGCCG |
| | | CTTAATTAAGCTGCCTTCTGCGGGGCTTGCCTTCTGGCCATGCCCTTCTTCTCTCCCTTG |
| | | CACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAA |
| | | AAAAAAA |
| ZF10- | 64 | AGTGTGGAAAAAGCTTTAGCCAAAGCGGCGATCTGAGGAGACACCAAAGAACACACACCGG |
| MQ1 | | CGAGAAGCCCTACAAATGTCCCGAGTGCGGAAAGAGCTTCAGCCAGAGCGGCCATCTGACC |
| (nt) | | GAGCATCAGAGAACCCATACCGGCGAAAAACCTTATAAGTGCCCCGAGTGTGGAAAGTCCT |
| | | TCTCCGAGAGATCCCATCTGAGAGAACACCAGAGGACACACACCGGCGAAAAACCTTATAA |
| | | GTGTCCCGAGTGCGGAAAGTCCTTCAGCGATCCCGGCCATCTGGTGAGACATCAAAGGACA |
| | | CATACCGGCGAAAAACCTTATAAGTGTCCCGAATGCGGCAAGAGCTTTAGCAGAAACGACA |
| | | CACTCACCGAACACCAGAGGACCCACACCGGCGAGAAACCCTACAAATGCCCCGAGTGCGG |
| | | CAAATCCTTTTCTAGAGCCGACAATCTGACCGAACACCAGAGGACCCATACCGGAGAAAAG |
| | | CCTTACAAATGTCCCGAGTGTGGCAAATCCTTCTCCACCCATCTGGATCTGATTAGACACC |
| | | AAAGAACACATACCGGAGAAAAGCCCACCGGAAAAAAGACCAGCGCTAGCGGCAGCGGCGG |
| | | CGGCAGCGGCGGCGCCCGGGACAGCAAGGTGGAGAACAAGACCAAGAAGCTGCGGGTGTTC |
| | | GAGGCCTTCGCCGGCATCGGCGCCCAGCGGAAGGCCCTGGAGAAGGTGCGGAAGGACGAGT |
| | | ACGAGATCGTGGGCCTGGCCGAGTGGTACGTGCCCGCCATCGTGATGTACCAGGCCATCCA |
| | | CAACAACTTCCACACCAAGCTGGAGTACAAGAGCGTGAGCCGGGAGGAGATGATCGACTAC |
| | | CTGGAGAACAAGACCCTGAGCTGGAACAGCAAGAACCCCGTGAGCAACGGCTACTGGAAGC |
| | | GGAAGAAGGACGACGAGCTGAAGATCATCTACAACGCCATCAAGCTGAGCGAGAAGGAGGG |
| | | CAACATCTTCGACATCCGGGACCTGTACAAGCGGACCCTGAAGAACATCGACCTGCTGACC |
| | | TACAGCTTCCCCTGCCAGGACCTGAGCCAGCAGGGCATCCAGAAGGGCATGAAGCGGGGCA |
| | | GCGGCACCCGGAGCGGCCTGCTGTGGGAGATCGAGCGGGCCCTGGACAGCACCGAGAAGAA |
| | | CGACCTGCCCAAGTACCTGCTGATGGAGAACGTGGGCGCCCTGCTGCACAAGAAGAACGAG |
| | | GAGGAGCTGAACCAGTGGAAGCAGAAGCTGGAGAGCCTGGGCTACCAGAACAGCATCGAGG |
| | | TGCTGAACGCCGCCGACTTCGGCAGCAGCCAGGCCCGGCGGCGGGTGTTCATGATCAGCAC |
| | | CCTGAACGAGTTCGTGGAGCTGCCCAAGGGCGACAAGAAGCCCAAGAGCATCAAGAAGGTG |
| | | CTGAACAAGATCGTGAGCGAGAAGGACATCCTGAACAACCTGCTGAAGTACAACCTGACCG |
| | | AGTTCAAGAAAACCAAGAGCAACATCAACAAGGCCAGCCTGATCGGCTACAGCAAGTTCAA |
| | | CAGCGAGGGCTACGTGTACGACCCCGAGTTCACCGGCCCCACCCTGACCGCCAGCGGCGCC |
| | | AACAGCCGGATCAAGATCAAGGACGGCAGCAACATCCGGAAGATGAACAGCGACGAGACCT |
| | | TCCTGTACATCGGCTTCGACAGCCAGGACGGCAAGCGGGTGAACGAGATCGAGTTCCTGAC |
| | | CGAGAACCAGAAGATCTTCGTGTGCGGCAACAGCATCAGCGTGGAGGTGCTGGAGGCCATC |
| | | ATCGACAAGATCGGCGGCCCCAGCAGCGGCGGCAAGCGGCCCGCCGCCACCAAGAAGGCCG |
| | | GCCAGGCCAAGAAGAAGAAGGGCAGCTACCCCTACGACGTGCCCGACTACGCCTGAGCGGC |
| | | CGCTTAATTAAGCTGCCTTCTGCGGGGCTTGCCTTCTGGCCATGCCCTTCTTCTCTCCCTT |
| | | GCACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAAA |
| | | AAAAAA |
| ZF11- | 65 | GCCTTATAAGTGCCCCGAGTGTGGCAAATCCTTTTCCGACTGTAGAGATCTGGCCAGACA |
| MQ1 | | TCAAAGAACCCACACCGGAGAGAAACCTTATAAATGCCCCGAGTGCGGCAAGTCCTTTAG |
| (nt) | | CCATACCGGCCATCTGCTGGAGCACCAGAGGACCCATACCGGCGAGAAGCCTTACAAATG |
| | | CCCCGAGTGCGGCAAAAGCTTCAGCAGAAATGACGCTCTGACCGAGCATCAAAGGACCCA |
| | | TACCGGCGAAAAGCCCTACAAGTGTCCCGAGTGTGGAAAGTCCTTCTCCCAGAGCGGCGA |
| | | TCTGAGGAGACACCAGAGAACACACACCGGCGAGAAACCCTATAAATGTCCCGAGTGCGG |
| | | AAAGAGCTTTAGCGACAGCGGCAATCTGAGGGTGCATCAAAGAACACACACCGGCGAAAA |
| | | ACCCACCGGAAAAAAGACAAGCGCTAGCGGCAGCGGCGGCGGCAGCGGCGGCGCCCGGGA |
| | | CAGCAAGGTGGAGAACAAGACCAAGAAGCTGCGGGTGTTCGAGGCCTTCGCCGGCATCGG |
| | | CGCCCAGCGGAAGGCCCTGGAGAAGGTGCGGAAGGACGAGTACGAGATCGTGGGCCTGGC |
| | | CGAGTGGTACGTGCCCGCCATCGTGATGTACCAGGCCATCCACAACAACTTCCACACCAA |
| | | GCTGGAGTACAAGAGCGTGAGCCGGGAGGAGATGATCGACTACCTGGAGAACAAGACCCT |
| | | GAGCTGGAACAGCAAGAACCCCGTGAGCAACGGCTACTGGAAGCGGAAGAAGGACGACGA |
| | | GCTGAAGATCATCTACAACGCCATCAAGCTGAGCGAGAAGGAGGGCAACATCTTCGACAT |
| | | CCGGGACCTGTACAAGCGGACCCTGAAGAACATCGACCTGCTGACCTACAGCTTCCCCTG |
| | | CCAGGACCTGAGCCAGCAGGGCATCCAGAAGGGCATGAAGCGGGGCAGCGGCACCCGGAG |
| | | CGGCCTGCTGTGGGAGATCGAGCGGGCCCTGGACAGCACCGAGAAGAACGACCTGCCCAA |
| | | GTACCTGCTGATGGAGAACGTGGGCGCCCTGCTGCACAAGAAGAACGAGGAGGAGCTGAA |
| | | CCAGTGGAAGCAGAAGCTGGAGAGCCTGGGCTACCAGAACAGCATCGAGGTGCTGAACGC |
| | | CGCCGACTTCGGCAGCAGCCAGGCCCGGCGGCGGGTGTTCATGATCAGCACCCTGAACGA |
| | | GTTCGTGGAGCTGCCCAAGGGCGACAAGAAGCCCAAGAGCATCAAGAAGGTGCTGAACAA |
| | | GATCGTGAGCGAGAAGGACATCCTGAACAACCTGCTGAAGTACAACCTGACCGAGTTCAA |
| | | GAAAACCAAGAGCAACATCAACAAGGCCAGCCTGATCGGCTACAGCAAGTTCAACAGCGA |
| | | GGGCTACGTGTACGACCCCGAGTTCACCGGCCCCACCCTGACCGCCAGCGGCGCCAACAG |
| | | CCGGATCAAGATCAAGGACGGCAGCAACATCCGGAAGATGAACAGCGACGAGACCTTCCT |
| | | GTACATCGGCTTCGACAGCCAGGACGGCAAGCGGGTGAACGAGATCGAGTTCCTGACCGA |
| | | GAACCAGAAGATCTTCGTGTGCGGCAACAGCATCAGCGTGGAGGTGCTGGAGGCCATCAT |
| | | CGACAAGATCGGCGGCCCCAGCAGCGGCGGCAAGCGGCCCGCCGCCACCAAGAAGGCCGG |
| | | CCAGGCCAAGAAGAAGAAGGGCAGCTACCCCTACGACGTGCCCGACTACGCCTGAGCGGC |
| | | CGCTTAATTAAGCTGCCTTCTGCGGGGCTTGCCTTCTGGCCATGCCCTTCTTCTCTCCCT |
| | | TGCACCTGTACCTCTTGGTCTTTGAATAAAGCCTGAGTAGGAAGTCTAGAAAAAAAAAAA |
| | | AAAAAAAAAAAAAAAAAAAAAAAAAAAAAAAAAAAAAAAAAAAAAAAAAAAAAAAAAAAA |
| | | AAAAAAAAA |
| ZF12- | 66 | GACCCATACCGGCGAAAAGCCTTACAAGTGTCCCGAGTGCGGAAAGTCCTTCTCTAGATC |
| MQ1 | | CGACAACCTCGTGAGGCACCAGAGAACCCACACCGGCGAGAAACCTTACAAATGTCCCGA |
| (nt) | | GTGTGGCAAAAGCTTTTCTAGAAGCGACGAGCTGGTGAGACATCAAAGAACCCATACCGG |
| | | CGAAAAACCTTATAAGTGTCCCGAGTGCGGCAAATCCTTTAGCCAGCTGGCCCATCTGAG |
| | | GGCCCACCAGAGAACACATACCGGCGAAAAACCCACCGGCAAAAAGACAAGCGCTAGCGG |
| | | CAGCGGCGGCGGCAGCGGCGGCGCCCGGGACAGCAAGGTGGAGAACAAGACCAAGAAGCT |
| | | GCGGGTGTTCGAGGCCTTCGCCGGCATCGGCGCCCAGCGGAAGGCCCTGGAGAAGGTGCG |
| | | GAAGGACGAGTACGAGATCGTGGGCCTGGCCGAGTGGTACGTGCCCGCCATCGTGATGTA |
| | | CCAGGCCATCCACAACAACTTCCACACCAAGCTGGAGTACAAGAGCGTGAGCCGGGAGGA |
| | | GATGATCGACTACCTGGAGAACAAGACCCTGAGCTGGAACAGCAAGAACCCCGTGAGCAA |
| | | CGGCTACTGGAAGCGGAAGAAGGACGACGAGCTGAAGATCATCTACAACGCCATCAAGCT |
| | | GAGCGAGAAGGAGGGCAACATCTTCGACATCCGGGACCTGTACAAGCGGACCCTGAAGAA |
| | | CATCGACCTGCTGACCTACAGCTTCCCCTGCCAGGACCTGAGCCAGCAGGGCATCCAGAA |
| | | GGGCATGAAGCGGGGCAGCGGCACCCGGAGCGGCCTGCTGTGGGAGATCGAGCGGGCCCT |
| | | GGACAGCACCGAGAAGAACGACCTGCCCAAGTACCTGCTGATGGAGAACGTGGGCGCCCT |
| | | GCTGCACAAGAAGAACGAGGAGGAGCTGAACCAGTGGAAGCAGAAGCTGGAGAGCCTGGG |
| | | CTACCAGAACAGCATCGAGGTGCTGAACGCCGCCGACTTCGGCAGCAGCCAGGCCCGGCG |
| | | GCGGGTGTTCATGATCAGCACCCTGAACGAGTTCGTGGAGCTGCCCAAGGGCGACAAGAA |
| | | GCCCAAGAGCATCAAGAAGGTGCTGAACAAGATCGTGAGCGAGAAGGACATCCTGAACAA |
| | | CCTGCTGAAGTACAACCTGACCGAGTTCAAGAAAACCAAGAGCAACATCAACAAGGCCAG |
| | | CCTGATCGGCTACAGCAAGTTCAACAGCGAGGGCTACGTGTACGACCCCGAGTTCACCGG |
| | | CCCCACCCTGACCGCCAGCGGCGCCAACAGCCGGATCAAGATCAAGGACGGCAGCAACAT |
| | | CCGGAAGATGAACAGCGACGAGACCTTCCTGTACATCGGCTTCGACAGCCAGGACGGCAA |
| | | GCGGGTGAACGAGATCGAGTTCCTGACCGAGAACCAGAAGATCTTCGTGTGCGGCAACAG |
| | | CATCAGCGTGGAGGTGCTGGAGGCCATCATCGACAAGATCGGCGGCCCCAGCAGCGGCGG |
| | | CAAGCGGCCCGCCGCCACCAAGAAGGCCGGCCAGGCCAAGAAGAAGAAGGGCAGCTACCC |
| | | CTACGACGTGCCCGACTACGCCTGAGCGGCCGCTTAATTAAGCTGCCTTCTGCGGGGCTT |
| | | GCCTTCTGGCCATGCCCTTCTTCTCTCCCTTGCACCTGTACCTCTTGGTCTTTGAATAAA |
| | | GCCTGAGTAGGAAGTCTAGAAAAAAAAAAAAAAAAAAAAAAAAAAAAAAAAAAAAAAAAA |
| | | AAAAAAAAAAAAAAAAAAAAAAAAAAAAAAAAAAAAAAA |
| ZF10- | 116 | AGGAAATAAGAGAGAAAAGAAGAGTAAGAAGAAATATAAGAGCCACCATGGCCCCCAAGA |
| MQ1 | | AGAAGCGGAAGGTGGGCATCCACGGCGTGCCCGCCGCCGGCAGCAGCGGATCCCTGGAGC |
| (nt) full | | CCGGCGAGAAACCCTACAAGTGCCCCGAGTGTGGAAAAAGCTTTAGCCAAAGCGGCGATC |
| length | | TGAGGAGACACCAAAGAACACACACCGGCGAGAAGCCCTACAAATGTCCCGAGTGCGGAA |
| | | AGAGCTTCAGCCAGAGCGGCCATCTGACCGAGCATCAGAGAACCCATACCGGCGAAAAAC |
| | | CTTATAAGTGCCCCGAGTGTGGAAAGTCCTTCTCCGAGAGATCCCATCTGAGAGAACACC |
| | | AGAGGACACACACCGGCGAAAAACCTTATAAGTGTCCCGAGTGCGGAAAGTCCTTCAGCG |
| | | ATCCCGGCCATCTGGTGAGACATCAAAGGACACATACCGGCGAAAAACCTTATAAGTGTC |
| | | CCGAATGCGGCAAGAGCTTTAGCAGAAACGACACACTCACCGAACACCAGAGGACCCACA |
| | | CCGGCGAGAAACCCTACAAATGCCCCGAGTGCGGCAAATCCTTTTCTAGAGCCGACAATC |
| | | TGACCGAACACCAGAGGACCCATACCGGAGAAAAGCCTTACAAATGTCCCGAGTGTGGCA |
| | | AATCCTTCTCCACCCATCTGGATCTGATTAGACACCAAAGAACACATACCGGAGAAAAGC |
| | | CCACCGGAAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCCTGAGCGGCCG |
| | | CTTAATTAAGCTGCCTTCTGCGGGGCTTGCCTTCTGGCCATGCCCTTCTTCTCTCCCTTG |
| | | CACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAA |
| | | AAAAAAA |
| ZF11- | 117 | AGGAAATAAGAGAGAAAAGAAGAGTAAGAAGAAATATAAGAGCCACCATGGCCCCCAAGA |
| 117 | | AGAAGCGGAAGGTGGGCATCCACGGCGTGCCCGCCGCCGGCAGCAGCGGATCCCTGGAGC |
| MQ1 | | CCGGCGAAAAACCCTATAAGTGCCCCGAATGTGGAAAGAGCTTCAGCCATACCGGCCATC |
| (nt) full | | AAAGCTTCTCCTCCAAAAAGGCTCTCACCGAGCACCAGAGAACACATACCGGCGAAAAGC |
| length | | TGCTGGAACACCAAAGGACACACACCGGCGAGAAACCTTACAAGTGTCCCGAGTGCGGAA |
| | | CTTATAAGTGCCCCGAGTGTGGCAAATCCTTTTCCGACTGTAGAGATCTGGCCAGACATC |
| | | AAAGAACCCACACCGGAGAGAAACCTTATAAATGCCCCGAGTGCGGCAAGTCCTTTAGCC |
| | | ATACCGGCCATCTGCTGGAGCACCAGAGGACCCATACCGGCGAGAAGCCTTACAAATGCC |
| | | CCGAGTGCGGCAAAAGCTTCAGCAGAAATGACGCTCTGACCGAGCATCAAAGGACCCATA |
| | | CCGGCGAAAAGCCCTACAAGTGTCCCGAGTGTGGAAAGTCCTTCTCCCAGAGCGGCGATC |
| | | TGAGGAGACACCAGAGAACACACACCGGCGAGAAACCCTATAAATGTCCCGAGTGCGGAA |
| | | AGAGCTTTAGCGACAGCGGCAATCTGAGGGTGCATCAAAGAACACACACCGGCGAAAAAC |
| | | CCACCGGAAAAAAGACAAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCCTGAGCGGCCG |
| | | CTTAATTAAGCTGCCTTCTGCGGGGCTTGCCTTCTGGCCATGCCCTTCTTCTCTCCCTTG |
| | | CACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAA |
| | | AAAAAAA |
| ZF12- | 118 | AGGAAATAAGAGAGAAAAGAAGAGTAAGAAGAAATATAAGAGCCACCATGGCCCCCAAGA |
| MQ1 | | AGAAGCGGAAGGTGGGCATCCACGGCGTGCCCGCCGCCGGCAGCAGCGGATCCCTGGAGC |
| (nt) full | | CCGGCGAGAAACCCTATAAATGCCCCGAATGCGGAAAAAGCTTCAGCCAGTCCAGCTCTC |
| length | | TGGTGAGACATCAGAGGACACACACCGGCGAAAAGCCTTATAAGTGCCCCGAGTGCGGCA |
| | | AGTCCTTCTCTAGAAGCGATCACCTCACCAATCATCAGAGGACACATACCGGAGAGAAGC |
| | | CCTATAAGTGCCCCGAGTGCGGCAAGAGCTTTAGCCAGCTGGCTCATCTGAGAGCTCACC |
| | | AAAGAACCCATACCGGCGAGAAGCCTTACAAATGCCCCGAGTGTGGAAAATCCTTTTCCC |
| | | AGTCCAGCAACCTCGTCAGACATCAAAGGACCCATACCGGCGAAAAGCCTTACAAGTGTC |
| | | CCGAGTGCGGAAAGTCCTTCTCTAGATCCGACAACCTCGTGAGGCACCAGAGAACCCACA |
| | | CCGGCGAGAAACCTTACAAATGTCCCGAGTGTGGCAAAAGCTTTTCTAGAAGCGACGAGC |
| | | TGGTGAGACATCAAAGAACCCATACCGGCGAAAAACCTTATAAGTGTCCCGAGTGCGGCA |
| | | AATCCTTTAGCCAGCTGGCCCATCTGAGGGCCCACCAGAGAACACATACCGGCGAAAAAC |
| | | CCACCGGCAAAAAGACAAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCCTGAGCGGCCG |
| | | CTTAATTAAGCTGCCTTCTGCGGGGCTTGCCTTCTGGCCATGCCCTTCTTCTCTCCCTTG |
| | | CACCTGTACCTCTTGGTCTTTGAATAAAGCCTGAGTAGGAAGTCTAGAAAAAAAAAAAAA |
| | | AAAAAAAAAAAAAAAAAAAAAAAAAAAAAAAAAAAAAAAAAAAAAAAAAAAAAAAAAAAA |
| | | AAAAAAA |
| ZF9- | 130 | GGGAAATAAGAGAGAAAAGAAGAGTAAGAAGAAATATAAGAGCCACCATGGCCCCCAAGA |
| MQ1 | | AGAAGCGGAAGGTGGGCATCCACGGCGTGCCCGCCGCCGGCAGCAGCGGATCCCTGGAGC |
| without | | CCGGCGAGAAACCTTACAAATGCCCCGAGTGCGGCAAGAGCTTCAGCAGAAGCGACGATC |
| HA | | TGGTGAGGCACCAAAGAACCCACACCGGCGAAAAACCTTACAAGTGTCCCGAATGCGGAA |
| | | AGTCCTTCAGCAGAGAGGACAATCTGCACACCCACCAGAGAACACACACCGGAGAAAAGC |
| | | CTTACAAGTGCCCCGAATGCGGCAAATCCTTTTCTAGAAGCGATCATCTGACCACCCACC |
| | | AAAGAACACATACCGGCGAGAAGCCTTACAAATGTCCCGAGTGCGGAAAGTCCTTCTCCC |
| | | AGAGAGCCAATCTGAGGGCTCATCAAAGGACCCATACCGGCGAAAAGCCCTACAAATGCC |
| | | CCGAGTGCGGAAAATCCTTCAGCCAGCTGGCCCATCTGAGAGCCCACCAAAGGACACACA |
| | | CCGGAGAGAAACCCTATAAGTGCCCCGAGTGTGGAAAAAGCTTTTCCCAGAGGGCCAATC |
| | | TGAGGGCCCATCAGAGGACCCATACCGGAGAGAAGCCTTATAAATGTCCCGAGTGCGGAA |
| | | AAAGCTTCAGCGAGAGGAGCCATCTGAGGGAACATCAAAGAACCCACACCGGCGAAAAAC |
| | | CCACCGGAAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTGAGCGGCCGCTTAATTAAGCTGCCTTCTGCGGGGCT |
| | | TGCCTTCTGGCCATGCCCTTCTTCTCTCCCTTGCACCTGTACCTCTTGGTCTTTGAATAA |
| | | AGCCTGAGTAGGAAGTCTAGAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAA |
| | | AAAAAAAAAAAAAA |

In some embodiments, an expression repressor comprises a targeting moiety comprising a Zn Finger domain (e.g., comprising an amino acid sequence of any of SEQ ID NO:11-14), and an effector moiety comprising MQ1, e.g., a bacterial MQ1 (e.g., SEQ ID NO: 19). In some embodiments, the expression repressor comprises an amino sequence of any of SEQ ID NOs: 28, 29, 30, 31, 32, 33, 129, and 145-149. The protein sequence of these exemplary expression repressors are disclosed in Table 9. In some embodiments, an expression repressor described herein comprises an amino acid sequence of any of SEQ ID NOs: 28-33, 129 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

TABLE 9
Amino acid sequences of exemplary ZF-MQ1 effectors

| | SEQ | |
|---|---|---|
| NAME | ID NO. | SEQUENCE |
| ZF7- | 28 | MAPKKKRKVGIHGVPAAGSSGSLEPGEKPYKCPECGKSFSRNDALTEHQRTHTGEKP |
| MQ1 | | YKCPECGKSFSDCRDLARHQRTHTGEKPYKCPECGKSFSDPGHLVRHQRTHTGEKPY |
| (aa) | | KCPECGKSFSQSGHLTEHQRTHTGEKPYKCPECGKSFSREDNLHTHQRTHTGEKPYK |
| | | CPECGKSFSTKNSLTEHQRTHTGEKPYKCPECGKSFSRADNLTEHQRTHTGEKPTGK |
| | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGSYPYDVPDYA |
| ZF8- | 29 | MAPKKKRKVGIHGVPAAGSSGSLEPGEKPYKCPECGKSFSRSDKLTEHQRTHTGEKP |
| MQ1 | | YKCPECGKSFSRRDELNVHQRTHTGEKPYKCPECGKSFSRSDHLTNHQRTHTGEKPY |
| (aa) | | KCPECGKSFSSPADLTRHQRTHTGEKPYKCPECGKSFSRSDHLTNHQRTHTGEKPYK |
| | | CPECGKSFSSKKALTEHQRTHTGEKPYKCPECGKSFSTHLDLIRHQRTHTGEKPTGK |
| | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGSYPYDVPDYA |
| ZF9- | 30 | MAPKKKRKVGIHGVPAAGSSGSLEPGEKPYKCPECGKSFSRSDDLVRHQRTHTGEKP |
| MQ1 | | YKCPECGKSFSREDNLHTHQRTHTGEKPYKCPECGKSFSRSDHLTTHQRTHTGEKPY |
| (aa) | | KCPECGKSFSQRANLRAHQRTHTGEKPYKCPECGKSFSQLAHLRAHQRTHTGEKPYK |
| | | CPECGKSFSQRANLRAHQRTHTGEKPYKCPECGKSFSERSHLREHQRTHTGEKPTGK |
| | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGSYPYDVPDYA |
| ZF10- | 31 | MAPKKKRKVGIHGVPAAGSSGSLEPGEKPYKCPECGKSFSQSGDLRRHQRTHTGEKPY |
| MQ1 | | KCPECGKSFSQSGHLTEHQRTHTGEKPYKCPECGKSFSERSHLREHQRTHTGEKPYKC |
| (aa) | | PECGKSFSDPGHLVRHQRTHTGEKPYKCPECGKSFSRNDTLTEHQRTHTGEKPYKCPE |
| | | CGKSFSRADNLTEHQRTHTGEKPYKCPECGKSFSTHLDLIRHQRTHTGEKPTGKKTSA |
| | | SGSGGGSGGARDSKVENKTKKLRVFEAFAGIGAQRKALEKVRKDEYEIVGLAEWYVPA |
| | | IVMYQAIHNNFHTKLEYKSVSREEMIDYLENKTLSWNSKNPVSNGYWKRKKDDELKII |
| | | YNAIKLSEKEGNIFDIRDLYKRTLKNIDLLTYSFPCQDLSQQGIQKGMKRGSGTRSGL |
| | | LWEIERALDSTEKNDLPKYLLMENVGALLHKKNEEELNQWKQKLESLGYQNSIEVLNA |
| | | ADFGSSQARRRVFMISTLNEFVELPKGDKKPKSIKKVLNKIVSEKDILNNLLKYNLTE |
| | | FKKTKSNINKASLIGYSKENSEGYVYDPEFTGPTLTASGANSRIKIKDGSNIRKMNSD |
| | | ETFLYIGFDSQDGKRVNEIEFLTENQKIFVCGNSISVEVLEAIIDKIGGPSSGGKRPA |
| | | ATKKAGQAKKKKGSYPYDVPDYA |
| ZF11- | 32 | MAPKKKRKVGIHGVPAAGSSGSLEPGEKPYKCPECGKSFSHTGHLLEHQRTHTGEKP |
| MQ1 | | YKCPECGKSFSSKKALTEHQRTHTGEKPYKCPECGKSFSDCRDLARHQRTHTGEKPY |
| (aa) | | KCPECGKSFSHTGHLLEHQRTHTGEKPYKCPECGKSFSRNDALTEHQRTHTGEKPYK |
| | | CPECGKSFSQSGDLRRHQRTHTGEKPYKCPECGKSFSDSGNLRVHQRTHTGEKPTGK |
| | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGSYPYDVPDYA |
| ZF12- | 33 | MAPKKKRKVGIHGVPAAGSSGSLEPGEKPYKCPECGKSFSQSSSLVRHQRTHTGEKP |
| MQ1 | | YKCPECGKSFSRSDHLTNHQRTHTGEKPYKCPECGKSFSQLAHLRAHQRTHTGEKPY |
| (aa) | | KCPECGKSFSQSSNLVRHQRTHTGEKPYKCPECGKSFSRSDNLVRHQRTHTGEKPYK |
| | | CPECGKSFSRSDELVRHQRTHTGEKPYKCPECGKSFSQLAHLRAHQRTHTGEKPTGK |
| | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGSYPYDVPDYA |
| ZF9- | 129 | MAPKKKRKVGIHGVPAAGSSGSLEPGEKPYKCPECGKSFSRSDDLVRHQRTHTGEKP |
| MQ1 | | YKCPECGKSFSREDNLHTHQRTHTGEKPYKCPECGKSFSRSDHLTTHQRTHTGEKPY |
| without | | KCPECGKSFSQRANLRAHQRTHTGEKPYKCPECGKSFSQLAHLRAHQRTHTGEKPYK |
| HA | | CPECGKSFSQRANLRAHQRTHTGEKPYKCPECGKSFSERSHLREHQRTHTGEKPTGK |
| | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVEMISTLNEFVELPKGDKKPKSIKKVLNKIVSEKDILN |
| | | NLLKYNLTEFKKTKSNINKASLIGYSKFNSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGS |
| ZF9- | 133 | MAPKKKRKVGIHGVPAAGSSGSLEPGEKPYKCPECGKSFSRSDDLVRHQRTHTGEKP |
| MQ1+ | | YKCPECGKSFSREDNLHTHQRTHTGEKPYKCPECGKSFSRSDHLTTHQRTHTGEKPY |
| fragment1 | | KCPECGKSFSQRANLRAHQRTHTGEKPYKCPECGKSFSQLAHLRAHQRTHTGEKPYK |
| of | | CPECGKSFSQRANLRAHQRTHTGEKPYKCPECGKSFSERSHLREHQRTHTGEKPTGK |
| tPT2A | | KTSASGSGGGSGGARDSKVENKTKKLRVFEAFAGIGAQRKALEKVRKDEYEIVGLAE |
| (aa) | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGSYPYDVPDYAGSYPYDVPDYAATNFSLLKQAGD |
| | | VEENPG |
| ZF7- | 145 | MAPKKKRKVGIHGVPAAGSSGSLEPGEKPYKCPECGKSFSRNDALTEHQRTHTGEKP |
| MQ1 | | YKCPECGKSFSDCRDLARHQRTHTGEKPYKCPECGKSFSDPGHLVRHQRTHTGEKPY |
| without | | KCPECGKSFSQSGHLTEHQRTHTGEKPYKCPECGKSFSREDNLHTHQRTHTGEKPYK |
| HA | | CPECGKSFSTKNSLTEHQRTHTGEKPYKCPECGKSFSRADNLTEHQRTHTGEKPTGK |
| (aa) | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKFNSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGS |
| ZF8- | 146 | MAPKKKRKVGIHGVPAAGSSGSLEPGEKPYKCPECGKSFSRSDKLTEHQRTHTGEKP |
| MQ1 | | YKCPECGKSFSRRDELNVHQRTHTGEKPYKCPECGKSFSRSDHLTNHQRTHTGEKPY |
| without | | KCPECGKSFSSPADLTRHQRTHTGEKPYKCPECGKSFSRSDHLTNHQRTHTGEKPYK |
| HA | | CPECGKSFSSKKALTEHQRTHTGEKPYKCPECGKSFSTHLDLIRHQRTHTGEKPTGK |
| (aa) | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVEMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGS |
| ZF10- | 147 | MAPKKKRKVGIHGVPAAGSSGSLEPGEKPYKCPECGKSFSQSGDLRRHQRTHTGEKPY |
| MQ1 | | KCPECGKSFSQSGHLTEHQRTHTGEKPYKCPECGKSFSERSHLREHQRTHTGEKPYKC |
| without | | PECGKSFSDPGHLVRHQRTHTGEKPYKCPECGKSFSRNDTLTEHQRTHTGEKPYKCPE |
| HA | | CGKSFSRADNLTEHQRTHTGEKPYKCPECGKSFSTHLDLIRHQRTHTGEKPTGKKTSA |
| (aa) | | SGSGGGSGGARDSKVENKTKKLRVFEAFAGIGAQRKALEKVRKDEYEIVGLAEWYVPA |
| | | IVMYQAIHNNFHTKLEYKSVSREEMIDYLENKTLSWNSKNPVSNGYWKRKKDDELKII |
| | | YNAIKLSEKEGNIFDIRDLYKRTLKNIDLLTYSFPCQDLSQQGIQKGMKRGSGTRSGL |
| | | LWEIERALDSTEKNDLPKYLLMENVGALLHKKNEEELNQWKQKLESLGYQNSIEVLNA |
| | | ADFGSSQARRRVFMISTLNEFVELPKGDKKPKSIKKVLNKIVSEKDILNNLLKYNLTE |
| | | FKKTKSNINKASLIGYSKFNSEGYVYDPEFTGPTLTASGANSRIKIKDGSNIRKMNSD |
| | | ETFLYIGFDSQDGKRVNEIEFLTENQKIFVCGNSISVEVLEAIIDKIGGPSSGGKRPA |
| | | ATKKAGQAKKKKGS |
| ZF11- | 148 | MAPKKKRKVGIHGVPAAGSSGSLEPGEKPYKCPECGKSFSHTGHLLEHQRTHTGEKP |
| MQ1 | | YKCPECGKSFSSKKALTEHQRTHTGEKPYKCPECGKSFSDCRDLARHQRTHTGEKPY |
| without | | KCPECGKSFSHTGHLLEHQRTHTGEKPYKCPECGKSFSRNDALTEHQRTHTGEKPYK |
| HA | | CPECGKSFSQSGDLRRHQRTHTGEKPYKCPECGKSFSDSGNLRVHQRTHTGEKPTGK |
| (aa) | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGS |
| ZF12- | 149 | MAPKKKRKVGIHGVPAAGSSGSLEPGEKPYKCPECGKSFSQSSSLVRHQRTHTGEKP |
| MQ1 | | YKCPECGKSFSRSDHLTNHQRTHTGEKPYKCPECGKSFSQLAHLRAHQRTHTGEKPY |
| without | | KCPECGKSFSQSSNLVRHQRTHTGEKPYKCPECGKSFSRSDNLVRHQRTHTGEKPYK |
| HA | | CPECGKSFSRSDELVRHQRTHTGEKPYKCPECGKSFSQLAHLRAHQRTHTGEKPTGK |
| (aa) | | KTSASGSGGGSGGARDSKVENKTKKLRVFEAFAGIGAQRKALEKVRKDEYEIVGLAE |
| | | WYVPAIVMYQAIHNNFHTKLEYKSVSREEMIDYLENKTLSWNSKNPVSNGYWKRKKD |
| | | DELKIIYNAIKLSEKEGNIFDIRDLYKRTLKNIDLLTYSFPCQDLSQQGIQKGMKRG |
| | | SGTRSGLLWEIERALDSTEKNDLPKYLLMENVGALLHKKNEEELNQWKQKLESLGYQ |
| | | NSIEVLNAADFGSSQARRRVFMISTLNEFVELPKGDKKPKSIKKVLNKIVSEKDILN |
| | | NLLKYNLTEFKKTKSNINKASLIGYSKENSEGYVYDPEFTGPTLTASGANSRIKIKD |
| | | GSNIRKMNSDETFLYIGFDSQDGKRVNEIEFLTENQKIFVCGNSISVEVLEAIIDKI |
| | | GGPSSGGKRPAATKKAGQAKKKKGS |

In some embodiments an expression repressor comprises a targeting moiety comprising a Zn Finger domain (e.g., having an amino acid sequence of any of SEQ ID NO: 11-14), and an effector moiety comprising MQ1, e.g., a bacterial MQ1 (e.g., SEQ ID NO: 87).

In some embodiments, an expression repressor comprises a targeting moiety comprising a Zn Finger domain (e.g., one encoded by a nucleotide sequence of any of SEQ ID NO: 166-168), and an effector moiety comprising MQ1, e.g., a bacterial MQ1 (e.g., one encoded by a nucleotide sequence of SEQ ID NO: 52). In some embodiments, the expression repressors are encoded by the nucleic sequence of SEQ ID NOs: 157, 158, or 159. The nucleic acid sequence of these exemplary expression repressors are disclosed in Table 16. In some embodiments, a nucleic acid described herein comprises a nucleic acid sequence of any of SEQ ID NO: 166-168 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the nucleic acid sequence comprises a poly-A sequence, and in other embodiments, the nucleic acid lacks the poly-A sequence. For example, in some embodiments, a nucleic acid described herein comprises a sequence according to any of SEQ ID NO: 166-168 (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the 3′ poly-A sequence, or comprising a 3′ poly-A sequence of a shorter length.

TABLE 16
Nucleotide sequences of exemplary mouse-specific ZF-MQ1 effectors

| | SEQ | |
|---|---|---|
| Name | ID NO. | SEQUENCE |
| ZF15- | 166 | AGGAAATAAGAGAGAAAAGAAGAGTAAGAAGAAATATAAGAGCCACCATGGCCCCCAAGAAGAAGCGGAA |
| MQ1 nt | | GGTGGGCATCCACGGCGTGCCCGCCGCCGGCAGCAGCGGATCCCTTGAGCCCGGAGAAAAGCCATACAAA |
| | | TGTCCTGAATGCGGAAAGTCATTTTCTACGAGCGGCGAACTCGTGCGGCACCAAAGGACTCATACCGGCG |
| | | AAAAGCCTTACAAATGCCCGGAGTGCGGAAAAAGCTTCTCCGAGCGCTCGCACTTGCGGGAACACCAGCG |
| | | AACCCACACAGGGGAGAAACCGTATAAGTGCCCAGAGTGCGGCAAATCGTTCTCCCGGAACGACACCCTG |
| | | ACCGAACACCAACGCACTCATACTGGCGAAAAACCTTACAAGTGCCCTGAGTGTGGAAAGAGCTTCTCCC |
| | | GCGCCGATAACCTGACCGAGCACCAGCGGACCCATACCGGGGAAAAGCCGTACAAGTGTCCGGAATGCGG |
| | | CAAAAGCTTCAGCACCTCGGGTTCCCTGGTCCGGCATCAGAGAACTCACACCGGAGAGAAACCCTATAAG |
| | | TGTCCTGAGTGCGGGAAGTCCTTTTCATCGCCCGCGGACCTGACTAGACACCAGAGGACCCACACCGGGG |
| | | AGAAGCCCTACAAGTGCCCCGAATGTGGAAAGTCCTTCTCCGACTCCGGCAACCTCCGGGTGCACCAGCG |
| | | CACCCACACTGGAGAGAAGCCGACCGGAAAGAAAACTTCCGCCTCCGGTTCGGGAGGAGGCTCAGGAGGA |
| | | GCGAGAGATTCCAAGGTCGAGAACAAGACCAAGAAGCTGCGGGTGTTCGAGGCCTTTGCTGGCATCGGAG |
| | | CCCAGAGGAAGGCCCTCGAGAAGGTCCGCAAGGATGAGTACGAGATCGTGGGACTCGCGGAGTGGTACGT |
| | | GCCCGCCATTGTGATGTACCAGGCCATCCATAACAACTTCCACACTAAGCTGGAGTACAAGTCCGTGTCC |
| | | CGGGAGGAAATGATTGACTACCTGGAGAATAAGACCCTGTCATGGAACTCTAAGAACCCCGTGTCGAACG |
| | | GTTACTGGAAGAGAAAGAAGGATGACGAACTGAAGATTATCTACAACGCGATCAAGCTGAGCGAGAAGGA |
| | | GGGCAACATCTTCGACATCCGGGACCTCTACAAGCGCACCTTGAAGAACATCGATCTGCTGACCTACTCC |
| | | TTCCCGTGCCAAGACCTGAGCCAGCAGGGCATCCAGAAGGGGATGAAACGGGGCTCCGGTACTCGCAGCG |
| | | GCTTGCTGTGGGAAATTGAGCGGGCCCTGGATAGCACCGAGAAGAACGACTTGCCGAAGTATCTTCTCAT |
| | | GGAAAACGTCGGGGCTCTCCTTCACAAGAAGAACGAGGAAGAACTGAACCAGTGGAAGCAAAAGCTGGAA |
| | | TCCCTCGGATACCAGAACTCCATTGAGGTCCTGAACGCCGCCGACTTCGGATCGTCGCAAGCCAGACGGA |
| | | GGGTGTTCATGATTAGCACTCTGAACGAATTCGTGGAACTGCCGAAGGGCGACAAGAAGCCTAAGTCCAT |
| | | CAAGAAGGTGCTGAACAAGATCGTGTCCGAGAAGGACATTCTCAACAATCTGCTGAAGTACAACCTGACA |
| | | GAGTTCAAGAAAACCAAGTCCAACATCAACAAGGCCTCCTTGATTGGTTACTCAAAGTTCAACAGCGAGG |
| | | GATACGTGTACGACCCCGAATTCACTGGACCCACTCTGACCGCCTCCGGAGCAAACTCTAGGATTAAGAT |
| | | CAAGGACGGCTCCAACATCCGGAAGATGAACTCCGACGAAACCTTTCTGTACATCGGCTTCGACTCGCAA |
| | | GACGGAAAGCGCGTGAACGAGATCGAATTTCTTACCGAAAACCAGAAGATCTTCGTGTGCGGCAATTCAA |
| | | TCTCCGTGGAAGTCCTGGAAGCGATTATCGACAAGATCGGAGGCAGTGGTGGAAAGCGCCCAGCAGCCAC |
| | | TAAGAAGGCCGGACAGGCCAAGAAGAAGAAGGGATCCTACCCTTACGATGTGCCGGATTACGCTTGAGCG |
| | | GCCGCTTAATTAAGCTGCCTTCTGCGGGGCTTGCCTTCTGGCCATGCCCTTCTTCTCTCCCTTGCACCTG |
| | | TACCTCTTGGTCTTTGAATAAAGCCTGAGTAGGAAGTCTAGAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAA |
| | | AAAAAAAAAAAAAAA |
| ZF16- | 167 | AGGAAATAAGAGAGAAAAGAAGAGTAAGAAGAAATATAAGAGCCACCATGGCCCCCAAGAAGAAGCGGAA |
| MQ1 nt | | GGTGGGCATCCACGGCGTGCCCGCCGCCGGCAGCAGCGGATCCCTGGAACCCGGAGAAAAACCTTATAAG |
| | | TGCCCTGAATGCGGAAAGTCATTCTCGAGGTCGGACAAGCTCGTGCGGCACCAGAGGACACACACCGGGG |
| | | AAAAGCCATACAAGTGTCCCGAATGTGGAAAGTCCTTCAGCCAACGCGCCAACCTGAGAGCTCATCAGCG |
| | | GACTCACACTGGCGAAAAACCGTACAAATGCCCCGAATGCGGCAAAAGCTTCTCCCGCGCCGACAACTTG |
| | | ACCGAGCACCAGCGGACCCATACCGGCGAAAAGCCGTACAAGTGCCCGGAGTGTGGGAAGTCGTTCAGCC |
| | | AGTCCTCTTCCCTCGTGCGCCACCAACGCACCCATACTGGGGAGAAGCCCTATAAGTGTCCTGAGTGTGG |
| | | CAAATCATTCAGCGATAAGAAGGATCTTACCCGGCACCAACGGACTCATACCGGAGAGAAGCCTTACAAG |
| | | TGCCCCGAGTGCGGAAAGAGCTTCTCGTCCCCGGCGGACCTGACTAGACACCAGCGCACCCACACCGGAG |
| | | AAAAGCCCTACAAGTGCCCAGAGTGCGGGAAGTCCTTTTCCCAATCCGGTCACCTGACTGAGCACCAGAG |
| | | AACCCACACGGGAGAGAAACCGACCGGAAAGAAAACCTCCGCCTCCGGTTCGGGAGGAGGCTCAGGAGGA |
| | | GCGAGAGATTCCAAGGTCGAGAACAAGACCAAGAAGCTGCGGGTGTTCGAGGCCTTTGCTGGCATCGGAG |
| | | CCCAGAGGAAGGCCCTCGAGAAGGTCCGCAAGGATGAGTACGAGATCGTGGGACTCGCGGAGTGGTACGT |
| | | GCCCGCCATTGTGATGTACCAGGCCATCCATAACAACTTCCACACTAAGCTGGAGTACAAGTCCGTGTCC |
| | | CGGGAGGAAATGATTGACTACCTGGAGAATAAGACCCTGTCATGGAACTCTAAGAACCCCGTGTCGAACG |
| | | GTTACTGGAAGAGAAAGAAGGATGACGAACTGAAGATTATCTACAACGCGATCAAGCTGAGCGAGAAGGA |
| | | GGGCAACATCTTCGACATCCGGGACCTCTACAAGCGCACCTTGAAGAACATCGATCTGCTGACCTACTCC |
| | | TTCCCGTGCCAAGACCTGAGCCAGCAGGGCATCCAGAAGGGGATGAAACGGGGCTCCGGTACTCGCAGCG |
| | | GCTTGCTGTGGGAAATTGAGCGGGCCCTGGATAGCACCGAGAAGAACGACTTGCCGAAGTATCTTCTCAT |
| | | GGAAAACGTCGGGGCTCTCCTTCACAAGAAGAACGAGGAAGAACTGAACCAGTGGAAGCAAAAGCTGGAA |
| | | TCCCTCGGATACCAGAACTCCATTGAGGTCCTGAACGCCGCCGACTTCGGATCGTCGCAAGCCAGACGGA |
| | | GGGTGTTCATGATTAGCACTCTGAACGAATTCGTGGAACTGCCGAAGGGCGACAAGAAGCCTAAGTCCAT |
| | | CAAGAAGGTGCTGAACAAGATCGTGTCCGAGAAGGACATTCTCAACAATCTGCTGAAGTACAACCTGACA |
| | | GAGTTCAAGAAAACCAAGTCCAACATCAACAAGGCCTCCTTGATTGGTTACTCAAAGTTCAACAGCGAGG |
| | | GATACGTGTACGACCCCGAATTCACTGGACCCACTCTGACCGCCTCCGGAGCAAACTCTAGGATTAAGAT |
| | | CAAGGACGGCTCCAACATCCGGAAGATGAACTCCGACGAAACCTTTCTGTACATCGGCTTCGACTCGCAA |
| | | GACGGAAAGCGCGTGAACGAGATCGAATTTCTTACCGAAAACCAGAAGATCTTCGTGTGCGGCAATTCAA |
| | | TCTCCGTGGAAGTCCTGGAAGCGATTATCGACAAGATCGGAGGCAGTGGTGGAAAGCGCCCAGCAGCCAC |
| | | TAAGAAGGCCGGACAGGCCAAGAAGAAGAAGGGATCCTACCCTTACGATGTGCCGGATTACGCTTGAGCG |
| | | GCCGCTTAATTAAGCTGCCTTCTGCGGGGCTTGCCTTCTGGCCATGCCCTTCTTCTCTCCCTTGCACCTG |
| | | TACCTCTTGGTCTTTGAATAAAGCCTGAGTAGGAAGTCTAGAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAA |
| | | AAAAAAAAAAAAAAA |
| ZF17- | 168 | AGGAAATAAGAGAGAAAAGAAGAGTAAGAAGAAATATAAGAGCCACCATGGCCCCCAAGAAGAAGCGGAA |
| MQ1 nt | | GGTGGGCATCCACGGCGTGCCCGCCGCCGGCAGCAGCGGATCCTTGGAACCCGGAGAAAAGCCATACAAA |
| | | TGCCCCGAATGCGGAAAGTCGTTCAGCCAGTCCGGCGACCTCAGACGGCACCAACGGACTCACACCGGCG |
| | | AAAAACCGTACAAGTGCCCAGAGTGCGGCAAAAGCTTTAGCCAGTCGGGCGATCTGCGGAGACATCAGCG |
| | | CACTCACACTGGTGAAAAGCCCTACAAGTGTCCTGAGTGCGGGAAGTCCTTCAGCGAGCGCTCCCATCTT |
| | | CGCGAGCACCAGAGAACCCACACTGGAGAAAAACCTTATAAGTGCCCTGAGTGTGGCAAATCCTTCTCAA |
| | | CCACCGGCAACCTGACTGTGCACCAGCGGACCCACACAGGGGAGAAGCCTTACAAGTGCCCGGAGTGTGG |
| | | GAAGTCATTCTCCCATCGGACGACCCTGACCAACCACCAGAGGACCCATACTGGCGAAAAGCCGTATAAG |
| | | TGTCCGGAGTGCGGAAAGAGCTTCTCCGACTCCGGAAACCTCAGGGTGCACCAACGCACCCACACCGGAG |
| | | AGAAGCCGTACAAATGTCCCGAATGTGGAAAGTCCTTCTCCCAATCCTCTTCGCTGGTCCGGCACCAGCG |
| | | AACTCATACCGGGGAAAAGCCCACCGGAAAGAAAACCTCGGCCTCCGGTTCGGGAGGAGGCTCAGGAGGA |
| | | GCGAGAGATTCCAAGGTCGAGAACAAGACCAAGAAGCTGCGGGTGTTCGAGGCCTTTGCTGGCATCGGAG |
| | | CCCAGAGGAAGGCCCTCGAGAAGGTCCGCAAGGATGAGTACGAGATCGTGGGACTCGCGGAGTGGTACGT |
| | | GCCCGCCATTGTGATGTACCAGGCCATCCATAACAACTTCCACACTAAGCTGGAGTACAAGTCCGTGTCC |
| | | CGGGAGGAAATGATTGACTACCTGGAGAATAAGACCCTGTCATGGAACTCTAAGAACCCCGTGTCGAACG |
| | | GTTACTGGAAGAGAAAGAAGGATGACGAACTGAAGATTATCTACAACGCGATCAAGCTGAGCGAGAAGGA |
| | | GGGCAACATCTTCGACATCCGGGACCTCTACAAGCGCACCTTGAAGAACATCGATCTGCTGACCTACTCC |
| | | TTCCCGTGCCAAGACCTGAGCCAGCAGGGCATCCAGAAGGGGATGAAACGGGGCTCCGGTACTCGCAGCG |
| | | GCTTGCTGTGGGAAATTGAGCGGGCCCTGGATAGCACCGAGAAGAACGACTTGCCGAAGTATCTTCTCAT |
| | | GGAAAACGTCGGGGCTCTCCTTCACAAGAAGAACGAGGAAGAACTGAACCAGTGGAAGCAAAAGCTGGAA |
| | | TCCCTCGGATACCAGAACTCCATTGAGGTCCTGAACGCCGCCGACTTCGGATCGTCGCAAGCCAGACGGA |
| | | GGGTGTTCATGATTAGCACTCTGAACGAATTCGTGGAACTGCCGAAGGGCGACAAGAAGCCTAAGTCCAT |
| | | CAAGAAGGTGCTGAACAAGATCGTGTCCGAGAAGGACATTCTCAACAATCTGCTGAAGTACAACCTGACA |
| | | GAGTTCAAGAAAACCAAGTCCAACATCAACAAGGCCTCCTTGATTGGTTACTCAAAGTTCAACAGCGAGG |
| | | GATACGTGTACGACCCCGAATTCACTGGACCCACTCTGACCGCCTCCGGAGCAAACTCTAGGATTAAGAT |
| | | CAAGGACGGCTCCAACATCCGGAAGATGAACTCCGACGAAACCTTTCTGTACATCGGCTTCGACTCGCAA |
| | | GACGGAAAGCGCGTGAACGAGATCGAATTTCTTACCGAAAACCAGAAGATCTTCGTGTGCGGCAATTCAA |
| | | TCTCCGTGGAAGTCCTGGAAGCGATTATCGACAAGATCGGAGGCAGTGGTGGAAAGCGCCCAGCAGCCAC |
| | | TAAGAAGGCCGGACAGGCCAAGAAGAAGAAGGGATCCTACCCTTACGATGTGCCGGATTACGCTTGAGCG |
| | | GCCGCTTAATTAAGCTGCCTTCTGCGGGGCTTGCCTTCTGGCCATGCCCTTCTTCTCTCCCTTGCACCTG |
| | | TACCTCTTGGTCTTTGAATAAAGCCTGAGTAGGAAGTCTAGAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAAAAAAAAAAAA |
| | | AAAAAAAAAAAAAAA |

In some embodiments, an expression repressor comprises a targeting moiety comprising a Zn Finger domain (e.g., comprising an amino acid sequence of any of SEQ ID NO:154-156), and an effector moiety comprising MQ1, e.g., a bacterial MQ1 (e.g., SEQ ID NO: 19). In some embodiments, the expression repressor comprises an amino sequence of any of SEQ ID NOs: 160-165. The protein sequences of these exemplary expression repressors are disclosed in Table 17. In some embodiments, an expression repressor described herein comprises an amino acid sequence of any of SEQ ID NOs: 160-165 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

TABLE 17
Amino acid sequences of exemplary ZF-MQ1 effectors

| | SEQ | |
|---|---|---|
| | ID | |
| Name | NO. | SEQUENCE |
| ZF15- | 160 | MAPKKKRKVGIHGVPAAGSSGSLEPGEKPYKCPECGKSFSTSGELVRHQRTHTGEKPYKCP |
| MQ1 | | ECGKSFSERSHLREHQRTHTGEKPYKCPECGKSFSRNDTLTEHQRTHTGEKPYKCPECGKS |
| aa | | FSRADNLTEHQRTHTGEKPYKCPECGKSFSTSGSLVRHQRTHTGEKPYKCPECGKSFSSPA |
| | | DLTRHQRTHTGEKPYKCPECGKSFSDSGNLRVHQRTHTGEKPTGKKTSASGSGGGSGGARD |
| | | SKVENKTKKLRVFEAFAGIGAQRKALEKVRKDEYEIVGLAEWYVPAIVMYQAIHNNFHTKL |
| | | EYKSVSREEMIDYLENKTLSWNSKNPVSNGYWKRKKDDELKIIYNAIKLSEKEGNIFDIRD |
| | | LYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVEL |
| | | PKGDKKPKSIKKVLNKIVSEKDILNNLLKYNLTEFKKTKSNINKASLIGYSKENSEGYVYD |
| | | PEFTGPTLTASGANSRIKIKDGSNIRKMNSDETFLYIGFDSQDGKRVNEIEFLTENQKIFV |
| | | CGNSISVEVLEAIIDKIGGSGGKRPAATKKAGQAKKKKGSYPYDVPDYA |
| ZF16- | 161 | MAPKKKRKVGIHGVPAAGSSGSLEPGEKPYKCPECGKSFSRSDKLVRHQRTHTGEKPYKCP |
| MQ1 | | ECGKSFSQRANLRAHQRTHTGEKPYKCPECGKSFSRADNLTEHQRTHTGEKPYKCPECGKS |
| aa | | FSQSSSLVRHQRTHTGEKPYKCPECGKSFSDKKDLTRHQRTHTGEKPYKCPECGKSFSSPA |
| | | DLTRHQRTHTGEKPYKCPECGKSFSQSGHLTEHQRTHTGEKPTGKKTSASGSGGGSGGARD |
| | | SKVENKTKKLRVFEAFAGIGAQRKALEKVRKDEYEIVGLAEWYVPAIVMYQAIHNNFHTKL |
| | | EYKSVSREEMIDYLENKTLSWNSKNPVSNGYWKRKKDDELKIIYNAIKLSEKEGNIFDIRD |
| | | LYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVEL |
| | | PKGDKKPKSIKKVLNKIVSEKDILNNLLKYNLTEFKKTKSNINKASLIGYSKENSEGYVYD |
| | | PEFTGPTLTASGANSRIKIKDGSNIRKMNSDETFLYIGFDSQDGKRVNEIEFLTENQKIFV |
| | | CGNSISVEVLEAIIDKIGGSGGKRPAATKKAGQAKKKKGSYPYDVPDYA |
| ZF17- | 162 | MAPKKKRKVGIHGVPAAGSSGSLEPGEKPYKCPECGKSFSQSGDLRRHQRTHTGEKPYKCP |
| MQ1 | | ECGKSFSQSGDLRRHQRTHTGEKPYKCPECGKSFSERSHLREHQRTHTGEKPYKCPECGKS |
| aa | | FSTTGNLTVHQRTHTGEKPYKCPECGKSFSHRTTLTNHQRTHTGEKPYKCPECGKSFSDSG |
| | | NLRVHQRTHTGEKPYKCPECGKSFSQSSSLVRHQRTHTGEKPTGKKTSASGSGGGSGGARD |
| | | SKVENKTKKLRVFEAFAGIGAQRKALEKVRKDEYEIVGLAEWYVPAIVMYQAIHNNFHTKL |
| | | EYKSVSREEMIDYLENKTLSWNSKNPVSNGYWKRKKDDELKIIYNAIKLSEKEGNIFDIRD |
| | | LYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVEL |
| | | PKGDKKPKSIKKVLNKIVSEKDILNNLLKYNLTEFKKTKSNINKASLIGYSKENSEGYVYD |
| | | PEFTGPTLTASGANSRIKIKDGSNIRKMNSDETFLYIGFDSQDGKRVNEIEFLTENQKIFV |
| | | CGNSISVEVLEAIIDKIGGSGGKRPAATKKAGQAKKKKGSYPYDVPDYA |
| ZF15- | 163 | MAPKKKRKVGIHGVPAAGSSGSLEPGEKPYKCPECGKSFSTSGELVRHQRTHTGEKPYKCP |
| MQ1 | | ECGKSFSERSHLREHQRTHTGEKPYKCPECGKSFSRNDTLTEHQRTHTGEKPYKCPECGKS |
| aa | | FSRADNLTEHQRTHTGEKPYKCPECGKSFSTSGSLVRHQRTHTGEKPYKCPECGKSFSSPA |
| without | | DLTRHQRTHTGEKPYKCPECGKSFSDSGNLRVHQRTHTGEKPTGKKTSASGSGGGSGGARD |
| HA tag | | SKVENKTKKLRVFEAFAGIGAQRKALEKVRKDEYEIVGLAEWYVPAIVMYQAIHNNFHTKL |
| | | EYKSVSREEMIDYLENKTLSWNSKNPVSNGYWKRKKDDELKIIYNAIKLSEKEGNIFDIRD |
| | | LYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVEL |
| | | PKGDKKPKSIKKVLNKIVSEKDILNNLLKYNLTEFKKTKSNINKASLIGYSKENSEGYVYD |
| | | PEFTGPTLTASGANSRIKIKDGSNIRKMNSDETFLYIGFDSQDGKRVNEIEFLTENQKIFV |
| | | CGNSISVEVLEAIIDKIGGSGGKRPAATKKAGQAKKKKGS |
| ZF16- | 164 | MAPKKKRKVGIHGVPAAGSSGSLEPGEKPYKCPECGKSFSRSDKLVRHQRTHTGEKPYKCP |
| MQ1 | | ECGKSFSQRANLRAHQRTHTGEKPYKCPECGKSFSRADNLTEHQRTHTGEKPYKCPECGKS |
| aa | | FSQSSSLVRHQRTHTGEKPYKCPECGKSFSDKKDLTRHQRTHTGEKPYKCPECGKSFSSPA |
| without | | DLTRHQRTHTGEKPYKCPECGKSFSQSGHLTEHQRTHTGEKPTGKKTSASGSGGGSGGARD |
| HA tag | | SKVENKTKKLRVFEAFAGIGAQRKALEKVRKDEYEIVGLAEWYVPAIVMYQAIHNNFHTKL |
| | | EYKSVSREEMIDYLENKTLSWNSKNPVSNGYWKRKKDDELKIIYNAIKLSEKEGNIFDIRD |
| | | LYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVEL |
| | | PKGDKKPKSIKKVLNKIVSEKDILNNLLKYNLTEFKKTKSNINKASLIGYSKENSEGYVYD |
| | | PEFTGPTLTASGANSRIKIKDGSNIRKMNSDETFLYIGFDSQDGKRVNEIEFLTENQKIFV |
| | | CGNSISVEVLEAIIDKIGGSGGKRPAATKKAGQAKKKKGS |
| ZF17- | 165 | MAPKKKRKVGIHGVPAAGSSGSLEPGEKPYKCPECGKSFSQSGDLRRHQRTHTGEKPYKCP |
| MQ1 | | ECGKSFSQSGDLRRHQRTHTGEKPYKCPECGKSFSERSHLREHQRTHTGEKPYKCPECGKS |
| aa | | FSTTGNLTVHQRTHTGEKPYKCPECGKSFSHRTTLTNHQRTHTGEKPYKCPECGKSFSDSG |
| without | | NLRVHQRTHTGEKPYKCPECGKSFSQSSSLVRHQRTHTGEKPTGKKTSASGSGGGSGGARD |
| HA tag | | SKVENKTKKLRVFEAFAGIGAQRKALEKVRKDEYEIVGLAEWYVPAIVMYQAIHNNFHTKL |
| | | EYKSVSREEMIDYLENKTLSWNSKNPVSNGYWKRKKDDELKIIYNAIKLSEKEGNIFDIRD |
| | | LYKRTLKNIDLLTYSFPCQDLSQQGIQKGMKRGSGTRSGLLWEIERALDSTEKNDLPKYLL |
| | | MENVGALLHKKNEEELNQWKQKLESLGYQNSIEVLNAADFGSSQARRRVFMISTLNEFVEL |
| | | PKGDKKPKSIKKVLNKIVSEKDILNNLLKYNLTEFKKTKSNINKASLIGYSKENSEGYVYD |
| | | PEFTGPTLTASGANSRIKIKDGSNIRKMNSDETFLYIGFDSQDGKRVNEIEFLTENQKIFV |
| | | CGNSISVEVLEAIIDKIGGSGGKRPAATKKAGQAKKKKGS |

In some embodiments, the present disclosure provides an expression repressor system comprising a first targeting moiety comprising a first ZF, a first effector moiety comprising a DNA methyltransferase, e.g., MQ1 or a functional fragment thereof, a second targeting moiety comprising a second ZF, and a second effector moiety comprising KRAB, e.g., a KRAB domain. In some embodiments, the expression repressor system is encoded by a first nucleic acid encoding the first targeting moiety and first effector moiety, wherein expression is driven by a first promoter or IRES, and a second nucleic acid encoding the second targeting moiety and second effector moiety, wherein expression is driven by a second promoter or IRES. In some embodiments, mono-cistronic sequences are used. In some embodiments, the nucleic acid encoding the expression repressor system is a multi-cistronic sequence. In some embodiments, the multi-cistronic sequence is a bi-cistronic sequence. In some embodiments, the multi-cistronic sequence comprises a sequence encoding the first expression repressor and a sequence encoding the second expression repressor. In some embodiments, the multi-cistronic sequence encodes a self-cleavable peptide sequence, e.g., a 2A peptide sequence, e.g., a T2A peptide sequence, a P2A sequence. In some embodiments, the multi-cistronic sequence encodes a T2A peptide sequence and a P2A peptide sequence. In some embodiments, the multi-cistronic sequence encodes a tandem 2A sequence, e.g., a tPT2A sequence. In some embodiments, the multi-cistronic construct encodes, from 5′ to 3′, (i) a first nuclear localization signal, e.g., a SV40 NLS, (ii) a first targeting moiety, e.g., a DNA binding domain, e.g., a zinc finger binding domain, e.g., ZF-9, (iii) a first effector moiety, e.g., a DNA methyltransferase, e.g., MQ1, (iv) a second nuclear localization signal, e.g., a nucleoplasmin NLS, (v) a linker, e.g., a tPT2A linker, (vi) a third nuclear localization signal, e.g., a SV40NLS, (vii) a second targeting moiety, e.g., a DNA binding domain, e.g., a zinc finger binding domain, e.g., ZF-3, (viii) a second effector moiety, e.g., a transcription repressor moiety, e.g., KRAB, and (ix) a fourth nuclear localization signal, e.g., a nucleoplasmin NLS. In some embodiments, the bi-cistronic construct further comprises a polyA tail. In some embodiments, upon transcription of the bi-cistronic gene construct, a single mRNA transcript encoding the first expression repressor, and the second expression repressor are produced, which upon translation gets cleaved, e.g., after the glycine residue within the 2A peptide, to yield the first expression repressor and the second expression repressor as two separate proteins. In some embodiments, the first and the second expression repressor are separated by “ribosome-skipping”. In some embodiments the first expression repressor and/or the second expression repressor retains a fragment of the 2A peptide after ribosome skipping. In some embodiments, the expression level of the first and second expression repressor are equal. In some embodiments, the expression level of the first and the second expression repressor are different. In some embodiments, the protein level of the first expression repressor is within 1%, 2%, 5%, or 10% of (greater than or less than) the protein level of the second expression repressor.

In some embodiments, a system encoded by a bi-cistronic nucleic acid decreases expression of a target gene (e.g., MYC) at least 1%, at least 2%, at least 3%, at least 4%, at least 5%, at least 10%, at least 20%, at least 30%, at least 40%, at least 50%, in a cell, than an otherwise similar system wherein the first and second expression repressor are encoded by mono-cistronic nucleic acids.

In some embodiments, the bi-cistronic sequence encodes an amino acid of SEQ ID NO: 91, 92, 121, 122, 181, 182, 187, 188, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, an expression repressor system comprises a targeting moiety comprising a Zn Finger domain (e.g., comprising an amino acid sequence of any of SEQ ID NO:7 or 13), and an effector moiety comprising MQ1, e.g., a bacterial MQ1 (e.g., SEQ ID NO: 19) or KRAB, e.g., a KRAB domain (e.g., SEQ ID NO: 18). In some embodiments, the expression repressor comprises an amino sequence of any of SEQ ID NOs: 91, 92, 121, 122, 181, 182, 187, 188. The protein sequence of these exemplary expression repressor systems are disclosed in Table 10. In some embodiments, an expression repressor system described herein comprises an amino acid sequence of any of SEQ ID NOs: 91-92, 121-122, 181, 182, 187, 188, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, the bi-cistronic sequence comprises nucleic acid sequence of SEQ ID NO: 93 or 112 (e.g., a nucleic acid (e.g., cDNA) encoding the expression repressor) or SEQ ID NO: 94 or 113 (e.g., a nucleic acid (e.g., cDNA) encoding the expression repressor). In some embodiments, the bi-cistronic sequence comprises nucleic acid sequence of SEQ ID NO: 196 (e.g., a nucleic acid (e.g., cDNA) encoding the expression repressor) or SEQ ID NO: 197 (e.g., a nucleic acid (e.g., cDNA) encoding the expression repressor). In some embodiments, a nucleic acid described herein comprises a nucleic acid sequence of SEQ ID NO: 93, 94, 112, 113, 196, or 197, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. The nucleic acid sequence encoding these exemplary expression repressor systems are disclosed in Table 10. In some embodiments, the nucleic acid sequence comprises a poly-A sequence, and in other embodiments, the nucleic acid lacks the poly-A sequence.

TABLE 10
Amino acid sequences of, and Nucleic acid sequences encoding,
exemplary expression repressor systems

| | SEQ | |
|---|---|---|
| | ID | |
| Name | NO: | SEQUENCE |
| ZF9- | 91 | MAPKKKRKVGIHGVPAAGSSGSLEPGEKPYKCPECGKSFSRSDDLVRHQRTHTGEKPYKC |
| MQ1+ | | PECGKSFSREDNLHTHQRTHTGEKPYKCPECGKSFSRSDHLTTHQRTHTGEKPYKCPECG |
| ZF3- | | KSFSQRANLRAHQRTHTGEKPYKCPECGKSFSQLAHLRAHQRTHTGEKPYKCPECGKSFS |
| KRAB | | QRANLRAHQRTHTGEKPYKCPECGKSFSERSHLREHQRTHTGEKPTGKKTSASGSGGGSG |
| (aa) | | GARDSKVENKTKKLRVFEAFAGIGAQRKALEKVRKDEYEIVGLAEWYVPAIVMYQAIHNN |
| | | FHTKLEYKSVSREEMIDYLENKTLSWNSKNPVSNGYWKRKKDDELKIIYNAIKLSEKEGN |
| | | IFDIRDLYKRTLKNIDLLTYSFPCQDLSQQGIQKGMKRGSGTRSGLLWEIERALDSTEKN |
| | | DLPKYLLMENVGALLHKKNEEELNQWKQKLESLGYQNSIEVLNAADFGSSQARRRVEMIS |
| | | TLNEFVELPKGDKKPKSIKKVLNKIVSEKDILNNLLKYNLTEFKKTKSNINKASLIGYSK |
| | | FNSEGYVYDPEFTGPTLTASGANSRIKIKDGSNIRKMNSDETFLYIGFDSQDGKRVNEIE |
| | | FLTENQKIFVCGNSISVEVLEAIIDKIGGPSSGGKRPAATKKAGQAKKKKGSYPYDVPDY |
| | | AGSYPYDVPDYAATNFSLLKQAGDVEENPGPTSAGKLGSGEGRGSLLTCGDVEENPGPLE |
| | | GSSGSGSPKKKRKVGIHGVPAAGSSGSLEPGEKPYKCPECGKSFSRSDKLVRHQRTHTGE |
| | | KPYKCPECGKSFSQRAHLERHQRTHTGEKPYKCPECGKSFSDPGHLVRHQRTHTGEKPYK |
| | | CPECGKSFSRSDKLVRHQRTHTGEKPYKCPECGKSFSQLAHLRAHQRTHTGEKPYKCPEC |
| | | GKSFSRADNLTEHQRTHTGEKPYKCPECGKSFSDCRDLARHQRTHTGEKPTGKKTSASGS |
| | | GGGSGGDAKSLTAWSRTLVTFKDVFVDFTREEWKLLDTAQQILYRNVMLENYKNLVSLGY |
| | | QLTKPDVILRLEKGEEPWLVEREIHQETHPDSETAFEIKSSVSGGKRPAATKKAGQAKKK |
| | | KGSYPYDVPDYA*SSGGKRPAATKKAGQAKKKKGSYPYDVPDYA |
| ZF3- | 92 | MAPKKKRKVGIHGVPAAGSSGSLEPGEKPYKCPECGKSFSRSDKLVRHQRTHTGEKPYKC |
| KRAB | | PECGKSFSQRAHLERHQRTHTGEKPYKCPECGKSFSDPGHLVRHQRTHTGEKPYKCPECG |
| +ZF9- | | KSFSRSDKLVRHQRTHTGEKPYKCPECGKSFSQLAHLRAHQRTHTGEKPYKCPECGKSFS |
| MQ1 | | RADNLTEHQRTHTGEKPYKCPECGKSFSDCRDLARHQRTHTGEKPTGKKTSASGSGGGSG |
| (aa) | | GDAKSLTAWSRTLVTFKDVFVDFTREEWKLLDTAQQILYRNVMLENYKNLVSLGYQLTKP |
| | | DVILRLEKGEEPWLVEREIHQETHPDSETAFEIKSSVSSGGKRPAATKKAGQAKKKKGSY |
| | | PYDVPDYAGSYPYDVPDYAATNFSLLKQAGDVEENPGPTSAGKLGSGEGRGSLLTCGDVE |
| | | ENPGPLEGSSGSGSPKKKRKVGIHGVPAAGSSGSLEPGEKPYKCPECGKSFSRSDDLVRH |
| | | QRTHTGEKPYKCPECGKSFSREDNLHTHQRTHTGEKPYKCPECGKSFSRSDHLTTHQRTH |
| | | TGEKPYKCPECGKSFSQRANLRAHQRTHTGEKPYKCPECGKSFSQLAHLRAHQRTHTGEK |
| | | PYKCPECGKSFSQRANLRAHQRTHTGEKPYKCPECGKSFSERSHLREHQRTHTGEKPTGK |
| | | KTSASGSGGGSGGARDSKVENKTKKLRVFEAFAGIGAQRKALEKVRKDEYEIVGLAEWYV |
| | | PAIVMYQAIHNNFHTKLEYKSVSREEMIDYLENKTLSWNSKNPVSNGYWKRKKDDELKII |
| | | YNAIKLSEKEGNIFDIRDLYKRTLKNIDLLTYSFPCQDLSQQGIQKGMKRGSGTRSGLLW |
| | | EIERALDSTEKNDLPKYLLMENVGALLHKKNEEELNQWKQKLESLGYQNSIEVLNAADFG |
| | | SSQARRRVFMISTLNEFVELPKGDKKPKSIKKVLNKIVSEKDILNNLLKYNLTEFKKTKS |
| | | NINKASLIGYSKFNSEGYVYDPEFTGPTLTASGANSRIKIKDGSNIRKMNSDETFLYIGF |
| | | DSQDGKRVNEIEFLTENQKIFVCGNSISVEVLEAIIDKIGGPSGGKRPAATKKAGQAKKK |
| | | KGSYPYDVPDYA*SSGGKRPAATKKAGQAKKKKGSYPYDVPDYA |
| ZF9- | 93 | GCGGCAAATCCTTTTCTAGAAGCGATCATCTGACCACCCACCAAAGAACACATACCGGCG |
| MQ1+ | | AGAAGCCTTACAAATGTCCCGAGTGCGGAAAGTCCTTCTCCCAGAGAGCCAATCTGAGGG |
| ZF3- | | CTCATCAAAGGACCCATACCGGCGAAAAGCCCTACAAATGCCCCGAGTGCGGAAAATCCT |
| KRAB | | TCAGCCAGCTGGCCCATCTGAGAGCCCACCAAAGGACACACACCGGAGAGAAACCCTATA |
| (nt) | | AGTGCCCCGAGTGTGGAAAAAGCTTTTCCCAGAGGGCCAATCTGAGGGCCCATCAGAGGA |
| | | CCCATACCGGAGAGAAGCCTTATAAATGTCCCGAGTGCGGAAAAAGCTTCAGCGAGAGGA |
| | | GCCATCTGAGGGAACATCAAAGAACCCACACCGGCGAAAAACCCACCGGAAAAAAGACCA |
| | | GCGCTAGCGGCAGCGGCGGCGGCAGCGGCGGCGCCCGGGACAGCAAGGTGGAGAACAAGA |
| | | CCAAGAAGCTGCGGGTGTTCGAGGCCTTCGCCGGCATCGGCGCCCAGCGGAAGGCCCTGG |
| | | AGAAGGTGCGGAAGGACGAGTACGAGATCGTGGGCCTGGCCGAGTGGTACGTGCCCGCCA |
| | | TCGTGATGTACCAGGCCATCCACAACAACTTCCACACCAAGCTGGAGTACAAGAGCGTGA |
| | | GCCGGGAGGAGATGATCGACTACCTGGAGAACAAGACCCTGAGCTGGAACAGCAAGAACC |
| | | CCGTGAGCAACGGCTACTGGAAGCGGAAGAAGGACGACGAGCTGAAGATCATCTACAACG |
| | | CCATCAAGCTGAGCGAGAAGGAGGGCAACATCTTCGACATCCGGGACCTGTACAAGCGGA |
| | | CCCTGAAGAACATCGACCTGCTGACCTACAGCTTCCCCTGCCAGGACCTGAGCCAGCAGG |
| | | GCATCCAGAAGGGCATGAAGCGGGGCAGCGGCACCCGGAGCGGCCTGCTGTGGGAGATCG |
| | | AGCGGGCCCTGGACAGCACCGAGAAGAACGACCTGCCCAAGTACCTGCTGATGGAGAACG |
| | | TGGGCGCCCTGCTGCACAAGAAGAACGAGGAGGAGCTGAACCAGTGGAAGCAGAAGCTGG |
| | | AGAGCCTGGGCTACCAGAACAGCATCGAGGTGCTGAACGCCGCCGACTTCGGCAGCAGCC |
| | | AGGCCCGGCGGCGGGTGTTCATGATCAGCACCCTGAACGAGTTCGTGGAGCTGCCCAAGG |
| | | GCGACAAGAAGCCCAAGAGCATCAAGAAGGTGCTGAACAAGATCGTGAGCGAGAAGGACA |
| | | TCCTGAACAACCTGCTGAAGTACAACCTGACCGAGTTCAAGAAaACCAAGAGCAACATCA |
| | | ACAAGGCCAGCCTGATCGGCTACAGCAAGTTCAACAGCGAGGGCTACGTGTACGACCCCG |
| | | AGTTCACCGGCCCCACCCTGACCGCCAGCGGCGCCAACAGCCGGATCAAGATCAAGGACG |
| | | GCAGCAACATCCGGAAGATGAACAGCGACGAGACCTTCCTGTACATCGGCTTCGACAGCC |
| | | AGGACGGCAAGCGGGTGAACGAGATCGAGTTCCTGACCGAGAACCAGAAGATCTTCGTGT |
| | | GCGGCAACAGCATCAGCGTGGAGGTGCTGGAGGCCATCATCGACAAGATCGGCGGCCCCA |
| | | GCAGCGGCGGCAAGCGGCCCGCCGCCACCAAGAAGGCCGGCCAGGCCAAGAAGAAGAAGG |
| | | GCAGCTACCCCTACGACGTGCCCGACTACGCCGGGTCCTACCCGTACGACGTGCCCGATT |
| | | ACGCCGCCACCAACTTCTCGCTGCTGAAGCAGGCCGGAGATGTGGAAGAAAACCCTGGAC |
| | | CTACCAGTGCCGGAAAGCTCGGTAGCGGAGAGGGTCGGGGAAGCCTGCTTACTTGCGGCG |
| | | ACGTGGAAGAGAACCCCGGTCCGCTGGAGGGTTCGTCCGGCTCCGGATCCCCCAAGAAGA |
| | | AGCGGAAGGTGGGCATCCACGGCGTGCCCGCCGCCGGCAGCAGCGGATCCCTGGAGCCCG |
| | | GCGAAAAACCTTACAAGTGCCCCGAGTGCGGAAAGAGCTTCAGCAGAAGCGACAAACTGG |
| | | TGAGGCATCAAAGGACACATACCGGAGAGAAGCCCTATAAGTGCCCCGAATGTGGCAAAT |
| | | CCTTTTCCCAGAGGGCTCATCTGGAAAGACACCAGAGGACCCATACCGGCGAAAAACCCT |
| | | ACAAATGTCCCGAGTGTGGAAAGAGCTTTTCCGATCCCGGCCATCTGGTCAGACATCAGA |
| | | GGACACATACCGGCGAAAAGCCTTACAAGTGTCCCGAATGCGGAAAATCCTTCTCCAGAA |
| | | GCGACAAGCTGGTGAGGCACCAAAGAACCCACACCGGCGAAAAACCCTATAAATGCCCCG |
| | | AGTGCGGCAAGTCCTTTAGCCAGCTGGCCCATCTGAGAGCCCACCAGAGAACACACACCG |
| | | GAGAGAAGCCTTATAAGTGTCCCGAGTGCGGAAAGTCCTTCTCTAGAGCCGACAATCTGA |
| | | CCGAACATCAAAGGACACACACCGGCGAGAAACCTTATAAATGCCCCGAGTGCGGAAAAA |
| | | GCTTTTCCGACTGCAGAGATCTGGCTAGACACCAGAGAACCCACACCGGCGAGAAACCCA |
| | | CCGGCAAAAAGACCAGCGCTAGCGGCAGCGGCGGCGGCAGCGGCGGCGACGCCAAGAGCC |
| | | TGACCGCCTGGAGCCGGACCCTGGTGACCTTCAAGGACGTGTTCGTGGACTTCACCCGGG |
| | | AGGAGTGGAAGCTGCTGGACACCGCCCAGCAGATCCTGTACCGGAACGTGATGCTGGAGA |
| | | ACTACAAGAACCTGGTGAGCCTGGGCTACCAGCTGACCAAGCCCGACGTGATCCTGCGGC |
| | | TGGAGAAGGGCGAGGAGCCCTGGCTGGTGGAGCGGGAGATCCACCAGGAGACCCACCCCG |
| | | ACAGCGAGACCGCCTTCGAGATCAAGAGCAGCGTGAGCGGCGGCAAGCGGCCCGCCGCCA |
| | | CCAAGAAGGCCGGCCAGGCCAAGAAGAAGAAGGGCAGCTACCCCTACGACGTGCCCGACT |
| | | ACGCCTGAAGCAGCGGCGGCAAGCGGCCCGCCGCCACCAAGAAGGCCGGCCAGGCCAAGA |
| | | AGAAGAAGGGCAGCTACCCCTACGACGTGCCCGACTACGCCTGAGCGGCCGCTTAATTAA |
| | | GCTGCCTTCTGCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAAAAAAAAAAAA |
| | | AAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAA |
| ZF3- | 94 | TATAAATGCCCCGAGTGCGGAAAAAGCTTTTCCGACTGCAGAGATCTGGCTAGACACCAG |
| KRAB | | AGAACCCACACCGGCGAGAAACCCACCGGCAAAAAGACCAGCGCTAGCGGCAGCGGCGGC |
| +ZF9- | | GGCAGCGGCGGCGACGCCAAGAGCCTGACCGCCTGGAGCCGGACCCTGGTGACCTTCAAG |
| MQ1 | | GACGTGTTCGTGGACTTCACCCGGGAGGAGTGGAAGCTGCTGGACACCGCCCAGCAGATC |
| (nt) | | CTGTACCGGAACGTGATGCTGGAGAACTACAAGAACCTGGTGAGCCTGGGCTACCAGCTG |
| | | ACCAAGCCCGACGTGATCCTGCGGCTGGAGAAGGGCGAGGAGCCCTGGCTGGTGGAGCGG |
| | | GAGATCCACCAGGAGACCCACCCCGACAGCGAGACCGCCTTCGAGATCAAGAGCAGCGTG |
| | | AGCAGCGGCGGCAAGCGGCCCGCCGCCACCAAGAAGGCCGGCCAGGCCAAGAAGAAGAAG |
| | | GGCAGCTACCCCTACGACGTGCCCGACTACGCCGGGTCCTACCCGTACGACGTGCCCGAT |
| | | TACGCCGCCACCAACTTCTCGCTGCTGAAGCAGGCCGGAGATGTGGAAGAAAACCCTGGA |
| | | CCTACCAGTGCCGGAAAGCTCGGTAGCGGAGAGGGTCGGGGAAGCCTGCTTACTTGCGGC |
| | | GACGTGGAAGAGAACCCCGGTCCGCTGGAGGGTTCGTCCGGCTCCGGATCCCCCAAGAAG |
| | | AAGCGGAAGGTGGGCATCCACGGCGTGCCCGCCGCCGGCAGCAGCGGATCCCTGGAGCCC |
| | | GGCGAGAAACCTTACAAATGCCCCGAGTGCGGCAAGAGCTTCAGCAGAAGCGACGATCTG |
| | | GTGAGGCACCAAAGAACCCACACCGGCGAAAAACCTTACAAGTGTCCCGAATGCGGAAAG |
| | | TCCTTCAGCAGAGAGGACAATCTGCACACCCACCAGAGAACACACACCGGAGAAAAGCCT |
| | | TACAAGTGCCCCGAATGCGGCAAATCCTTTTCTAGAAGCGATCATCTGACCACCCACCAA |
| | | AGAACACATACCGGCGAGAAGCCTTACAAATGTCCCGAGTGCGGAAAGTCCTTCTCCCAG |
| | | AGAGCCAATCTGAGGGCTCATCAAAGGACCCATACCGGCGAAAAGCCCTACAAATGCCCC |
| | | GAGTGCGGAAAATCCTTCAGCCAGCTGGCCCATCTGAGAGCCCACCAAAGGACACACACC |
| | | GGAGAGAAACCCTATAAGTGCCCCGAGTGTGGAAAAAGCTTTTCCCAGAGGGCCAATCTG |
| | | AGGGCCCATCAGAGGACCCATACCGGAGAGAAGCCTTATAAATGTCCCGAGTGCGGAAAA |
| | | AGCTTCAGCGAGAGGAGCCATCTGAGGGAACATCAAAGAACCCACACCGGCGAAAAACCC |
| | | ACCGGAAAAAAGACCAGCGCTAGCGGCAGCGGCGGCGGCAGCGGCGGCGCCCGGGACAGC |
| | | AAGGTGGAGAACAAGACCAAGAAGCTGCGGGTGTTCGAGGCCTTCGCCGGCATCGGCGCC |
| | | CAGCGGAAGGCCCTGGAGAAGGTGCGGAAGGACGAGTACGAGATCGTGGGCCTGGCCGAG |
| | | TGGTACGTGCCCGCCATCGTGATGTACCAGGCCATCCACAACAACTTCCACACCAAGCTG |
| | | GAGTACAAGAGCGTGAGCCGGGAGGAGATGATCGACTACCTGGAGAACAAGACCCTGAGC |
| | | TGGAACAGCAAGAACCCCGTGAGCAACGGCTACTGGAAGCGGAAGAAGGACGACGAGCTG |
| | | AAGATCATCTACAACGCCATCAAGCTGAGCGAGAAGGAGGGCAACATCTTCGACATCCGG |
| | | GACCTGTACAAGCGGACCCTGAAGAACATCGACCTGCTGACCTACAGCTTCCCCTGCCAG |
| | | GACCTGAGCCAGCAGGGCATCCAGAAGGGCATGAAGCGGGGCAGCGGCACCCGGAGCGGC |
| | | CTGCTGTGGGAGATCGAGCGGGCCCTGGACAGCACCGAGAAGAACGACCTGCCCAAGTAC |
| | | CTGCTGATGGAGAACGTGGGCGCCCTGCTGCACAAGAAGAACGAGGAGGAGCTGAACCAG |
| | | TGGAAGCAGAAGCTGGAGAGCCTGGGCTACCAGAACAGCATCGAGGTGCTGAACGCCGCC |
| | | GACTTCGGCAGCAGCCAGGCCCGGCGGCGGGTGTTCATGATCAGCACCCTGAACGAGTTC |
| | | GTGGAGCTGCCCAAGGGCGACAAGAAGCCCAAGAGCATCAAGAAGGTGCTGAACAAGATC |
| | | GTGAGCGAGAAGGACATCCTGAACAACCTGCTGAAGTACAACCTGACCGAGTTCAAGAAA |
| | | ACCAAGAGCAACATCAACAAGGCCAGCCTGATCGGCTACAGCAAGTTCAACAGCGAGGGC |
| | | TACGTGTACGACCCCGAGTTCACCGGCCCCACCCTGACCGCCAGCGGCGCCAACAGCCGG |
| | | ATCAAGATCAAGGACGGCAGCAACATCCGGAAGATGAACAGCGACGAGACCTTCCTGTAC |
| | | ATCGGCTTCGACAGCCAGGACGGCAAGCGGGTGAACGAGATCGAGTTCCTGACCGAGAAC |
| | | CAGAAGATCTTCGTGTGCGGCAACAGCATCAGCGTGGAGGTGCTGGAGGCCATCATCGAC |
| | | AAGATCGGCGGCCCCAGCGGCGGCAAGCGGCCCGCCGCCACCAAGAAGGCCGGCCAGGCC |
| | | AAGAAGAAGAAGGGCAGCTACCCCTACGACGTGCCCGACTACGCCTGAAGCAGCGGCGGC |
| | | AAGCGGCCCGCCGCCACCAAGAAGGCCGGCCAGGCCAAGAAGAAGAAGGGCAGCTACCCC |
| | | TACGACGTGCCCGACTACGCCTGAGCGGCCGCTTAATTAAGCTGCCTTCTGCGGGGCTTG |
| | | CCTTCTGGCCATGCCCTTCTTCTCTCCCTTGCACCTGTACCTCTTGGTCTTTGAATAAAG |
| | | CCTGAGTAGGAAGTCTAGAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAAAAAAA |
| | | AAAAAAAAAAAA |
| ZF9- | 112 | AGGAAATAAGAGAGAAAAGAAGAGTAAGAAGAAATATAAGAGCCACCATGGCCCCCAAGA |
| MQ1+ | | AGAAGCGGAAGGTGGGCATCCACGGCGTGCCCGCCGCCGGCAGCAGCGGATCCCTGGAGC |
| ZF3- | | CCGGCGAGAAACCTTACAAATGCCCCGAGTGCGGCAAGAGCTTCAGCAGAAGCGACGATC |
| KRAB | | TGGTGAGGCACCAAAGAACCCACACCGGCGAAAAACCTTACAAGTGTCCCGAATGCGGAA |
| full nt | | AGTCCTTCAGCAGAGAGGACAATCTGCACACCCACCAGAGAACACACACCGGAGAAAAGC |
| sequence | | CTTACAAGTGCCCCGAATGCGGCAAATCCTTTTCTAGAAGCGATCATCTGACCACCCACC |
| | | AAAGAACACATACCGGCGAGAAGCCTTACAAATGTCCCGAGTGCGGAAAGTCCTTCTCCC |
| | | AGAGAGCCAATCTGAGGGCTCATCAAAGGACCCATACCGGCGAAAAGCCCTACAAATGCC |
| | | CCGAGTGCGGAAAATCCTTCAGCCAGCTGGCCCATCTGAGAGCCCACCAAAGGACACACA |
| | | CCGGAGAGAAACCCTATAAGTGCCCCGAGTGTGGAAAAAGCTTTTCCCAGAGGGCCAATC |
| | | TGAGGGCCCATCAGAGGACCCATACCGGAGAGAAGCCTTATAAATGTCCCGAGTGCGGAA |
| | | AAAGCTTCAGCGAGAGGAGCCATCTGAGGGAACATCAAAGAACCCACACCGGCGAAAAAC |
| | | CCACCGGAAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AaACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCCGGGTCCTACC |
| | | CGTACGACGTGCCCGATTACGCCGCCACCAACTTCTCGCTGCTGAAGCAGGCCGGAGATG |
| | | TGGAAGAAAACCCTGGACCTACCAGTGCCGGAAAGCTCGGTAGCGGAGAGGGTCGGGGAA |
| | | GCCTGCTTACTTGCGGCGACGTGGAAGAGAACCCCGGTCCGCTGGAGGGTTCGTCCGGCT |
| | | CCGGATCCCCCAAGAAGAAGCGGAAGGTGGGCATCCACGGCGTGCCCGCCGCCGGCAGCA |
| | | GCGGATCCCTGGAGCCCGGCGAAAAACCTTACAAGTGCCCCGAGTGCGGAAAGAGCTTCA |
| | | GCAGAAGCGACAAACTGGTGAGGCATCAAAGGACACATACCGGAGAGAAGCCCTATAAGT |
| | | GCCCCGAATGTGGCAAATCCTTTTCCCAGAGGGCTCATCTGGAAAGACACCAGAGGACCC |
| | | ATACCGGCGAAAAACCCTACAAATGTCCCGAGTGTGGAAAGAGCTTTTCCGATCCCGGCC |
| | | ATCTGGTCAGACATCAGAGGACACATACCGGCGAAAAGCCTTACAAGTGTCCCGAATGCG |
| | | GAAAATCCTTCTCCAGAAGCGACAAGCTGGTGAGGCACCAAAGAACCCACACCGGCGAAA |
| | | AACCCTATAAATGCCCCGAGTGCGGCAAGTCCTTTAGCCAGCTGGCCCATCTGAGAGCCC |
| | | ACCAGAGAACACACACCGGAGAGAAGCCTTATAAGTGTCCCGAGTGCGGAAAGTCCTTCT |
| | | CTAGAGCCGACAATCTGACCGAACATCAAAGGACACACACCGGCGAGAAACCTTATAAAT |
| | | GCCCCGAGTGCGGAAAAAGCTTTTCCGACTGCAGAGATCTGGCTAGACACCAGAGAACCC |
| | | ACACCGGCGAGAAACCCACCGGCAAAAAGACCAGCGCTAGCGGCAGCGGCGGCGGCAGCG |
| | | GCGGCGACGCCAAGAGCCTGACCGCCTGGAGCCGGACCCTGGTGACCTTCAAGGACGTGT |
| | | TCGTGGACTTCACCCGGGAGGAGTGGAAGCTGCTGGACACCGCCCAGCAGATCCTGTACC |
| | | GGAACGTGATGCTGGAGAACTACAAGAACCTGGTGAGCCTGGGCTACCAGCTGACCAAGC |
| | | CCGACGTGATCCTGCGGCTGGAGAAGGGCGAGGAGCCCTGGCTGGTGGAGCGGGAGATCC |
| | | ACCAGGAGACCCACCCCGACAGCGAGACCGCCTTCGAGATCAAGAGCAGCGTGAGCGGCG |
| | | GCAAGCGGCCCGCCGCCACCAAGAAGGCCGGCCAGGCCAAGAAGAAGAAGGGCAGCTACC |
| | | CCTACGACGTGCCCGACTACGCCTGAAGCAGCGGCGGCAAGCGGCCCGCCGCCACCAAGA |
| | | AGGCCGGCCAGGCCAAGAAGAAGAAGGGCAGCTACCCCTACGACGTGCCCGACTACGCCT |
| | | GAGCGGCCGCTTAATTAAGCTGCCTTCTGCGGGGCTTGCCTTCTGGCCATGCCCTTCTTC |
| | | TCTCCCTTGCACCTGTACCTCTTGGTCTTTGAATAAAGCCTGAGTAGGAAGTCTAGAAAA |
| | | AAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAA |
| ZF3- | 113 | AGGAAATAAGAGAGAAAAGAAGAGTAAGAAGAAATATAAGAGCCACCATGGCCCCCAAGA |
| KRAB | | AGAAGCGGAAGGTGGGCATCCACGGCGTGCCCGCCGCCGGCAGCAGCGGATCCCTGGAGC |
| +ZF9- | | CCGGCGAAAAACCTTACAAGTGCCCCGAGTGCGGAAAGAGCTTCAGCAGAAGCGACAAAC |
| MQ1 | | TGGTGAGGCATCAAAGGACACATACCGGAGAGAAGCCCTATAAGTGCCCCGAATGTGGCA |
| full nt | | AATCCTTTTCCCAGAGGGCTCATCTGGAAAGACACCAGAGGACCCATACCGGCGAAAAAC |
| sequence | | CCTACAAATGTCCCGAGTGTGGAAAGAGCTTTTCCGATCCCGGCCATCTGGTCAGACATC |
| | | AGAGGACACATACCGGCGAAAAGCCTTACAAGTGTCCCGAATGCGGAAAATCCTTCTCCA |
| | | GAAGCGACAAGCTGGTGAGGCACCAAAGAACCCACACCGGCGAAAAACCCTATAAATGCC |
| | | CCGAGTGCGGCAAGTCCTTTAGCCAGCTGGCCCATCTGAGAGCCCACCAGAGAACACACA |
| | | CCGGAGAGAAGCCTTATAAGTGTCCCGAGTGCGGAAAGTCCTTCTCTAGAGCCGACAATC |
| | | TGACCGAACATCAAAGGACACACACCGGCGAGAAACCTTATAAATGCCCCGAGTGCGGAA |
| | | AAAGCTTTTCCGACTGCAGAGATCTGGCTAGACACCAGAGAACCCACACCGGCGAGAAAC |
| | | CCACCGGCAAAAAGACCAGCGCTAGCGGCAGCGGCGGCGGCAGCGGCGGCGACGCCAAGA |
| | | GCCTGACCGCCTGGAGCCGGACCCTGGTGACCTTCAAGGACGTGTTCGTGGACTTCACCC |
| | | GGGAGGAGTGGAAGCTGCTGGACACCGCCCAGCAGATCCTGTACCGGAACGTGATGCTGG |
| | | AGAACTACAAGAACCTGGTGAGCCTGGGCTACCAGCTGACCAAGCCCGACGTGATCCTGC |
| | | GGCTGGAGAAGGGCGAGGAGCCCTGGCTGGTGGAGCGGGAGATCCACCAGGAGACCCACC |
| | | CCGACAGCGAGACCGCCTTCGAGATCAAGAGCAGCGTGAGCAGCGGCGGCAAGCGGCCCG |
| | | CCGCCACCAAGAAGGCCGGCCAGGCCAAGAAGAAGAAGGGCAGCTACCCCTACGACGTGC |
| | | CCGACTACGCCGGGTCCTACCCGTACGACGTGCCCGATTACGCCGCCACCAACTTCTCGC |
| | | TGCTGAAGCAGGCCGGAGATGTGGAAGAAAACCCTGGACCTACCAGTGCCGGAAAGCTCG |
| | | GTAGCGGAGAGGGTCGGGGAAGCCTGCTTACTTGCGGCGACGTGGAAGAGAACCCCGGTC |
| | | CGCTGGAGGGTTCGTCCGGCTCCGGATCCCCCAAGAAGAAGCGGAAGGTGGGCATCCACG |
| | | GCGTGCCCGCCGCCGGCAGCAGCGGATCCCTGGAGCCCGGCGAGAAACCTTACAAATGCC |
| | | CCGAGTGCGGCAAGAGCTTCAGCAGAAGCGACGATCTGGTGAGGCACCAAAGAACCCACA |
| | | CCGGCGAAAAACCTTACAAGTGTCCCGAATGCGGAAAGTCCTTCAGCAGAGAGGACAATC |
| | | TGCACACCCACCAGAGAACACACACCGGAGAAAAGCCTTACAAGTGCCCCGAATGCGGCA |
| | | AATCCTTTTCTAGAAGCGATCATCTGACCACCCACCAAAGAACACATACCGGCGAGAAGC |
| | | CTTACAAATGTCCCGAGTGCGGAAAGTCCTTCTCCCAGAGAGCCAATCTGAGGGCTCATC |
| | | AAAGGACCCATACCGGCGAAAAGCCCTACAAATGCCCCGAGTGCGGAAAATCCTTCAGCC |
| | | AGCTGGCCCATCTGAGAGCCCACCAAAGGACACACACCGGAGAGAAACCCTATAAGTGCC |
| | | CCGAGTGTGGAAAAAGCTTTTCCCAGAGGGCCAATCTGAGGGCCCATCAGAGGACCCATA |
| | | CCGGAGAGAAGCCTTATAAATGTCCCGAGTGCGGAAAAAGCTTCAGCGAGAGGAGCCATC |
| | | TGAGGGAACATCAAAGAACCCACACCGGCGAAAAACCCACCGGAAAAAAGACCAGCGCTA |
| | | GCGGCAGCGGCGGCGGCAGCGGCGGCGCCCGGGACAGCAAGGTGGAGAACAAGACCAAGA |
| | | AGCTGCGGGTGTTCGAGGCCTTCGCCGGCATCGGCGCCCAGCGGAAGGCCCTGGAGAAGG |
| | | TGCGGAAGGACGAGTACGAGATCGTGGGCCTGGCCGAGTGGTACGTGCCCGCCATCGTGA |
| | | TGTACCAGGCCATCCACAACAACTTCCACACCAAGCTGGAGTACAAGAGCGTGAGCCGGG |
| | | AGGAGATGATCGACTACCTGGAGAACAAGACCCTGAGCTGGAACAGCAAGAACCCCGTGA |
| | | GCAACGGCTACTGGAAGCGGAAGAAGGACGACGAGCTGAAGATCATCTACAACGCCATCA |
| | | AGCTGAGCGAGAAGGAGGGCAACATCTTCGACATCCGGGACCTGTACAAGCGGACCCTGA |
| | | AGAACATCGACCTGCTGACCTACAGCTTCCCCTGCCAGGACCTGAGCCAGCAGGGCATCC |
| | | AGAAGGGCATGAAGCGGGGCAGCGGCACCCGGAGCGGCCTGCTGTGGGAGATCGAGCGGG |
| | | CCCTGGACAGCACCGAGAAGAACGACCTGCCCAAGTACCTGCTGATGGAGAACGTGGGCG |
| | | CCCTGCTGCACAAGAAGAACGAGGAGGAGCTGAACCAGTGGAAGCAGAAGCTGGAGAGCC |
| | | TGGGCTACCAGAACAGCATCGAGGTGCTGAACGCCGCCGACTTCGGCAGCAGCCAGGCCC |
| | | GGCGGCGGGTGTTCATGATCAGCACCCTGAACGAGTTCGTGGAGCTGCCCAAGGGCGACA |
| | | AGAAGCCCAAGAGCATCAAGAAGGTGCTGAACAAGATCGTGAGCGAGAAGGACATCCTGA |
| | | ACAACCTGCTGAAGTACAACCTGACCGAGTTCAAGAAAACCAAGAGCAACATCAACAAGG |
| | | CCAGCCTGATCGGCTACAGCAAGTTCAACAGCGAGGGCTACGTGTACGACCCCGAGTTCA |
| | | CCGGCCCCACCCTGACCGCCAGCGGCGCCAACAGCCGGATCAAGATCAAGGACGGCAGCA |
| | | ACATCCGGAAGATGAACAGCGACGAGACCTTCCTGTACATCGGCTTCGACAGCCAGGACG |
| | | GCAAGCGGGTGAACGAGATCGAGTTCCTGACCGAGAACCAGAAGATCTTCGTGTGCGGCA |
| | | ACAGCATCAGCGTGGAGGTGCTGGAGGCCATCATCGACAAGATCGGCGGCCCCAGCGGCG |
| | | GCAAGCGGCCCGCCGCCACCAAGAAGGCCGGCCAGGCCAAGAAGAAGAAGGGCAGCTACC |
| | | CCTACGACGTGCCCGACTACGCCTGAAGCAGCGGCGGCAAGCGGCCCGCCGCCACCAAGA |
| | | AGGCCGGCCAGGCCAAGAAGAAGAAGGGCAGCTACCCCTACGACGTGCCCGACTACGCCT |
| | | GAGCGGCCGCTTAATTAAGCTGCCTTCTGCGGGGCTTGCCTTCTGGCCATGCCCTTCTTC |
| | | TCTCCCTTGCACCTGTACCTCTTGGTCTTTGAATAAAGCCTGAGTAGGAAGTCTAGAAAA |
| | | AAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAA |
| ZF09- | 196 | GGGAAATAAGAGAGAAAAGAAGAGTAAGAAGAAATATAAGAGCCACCATGGCCCCAAAGA |
| MQ1- | | AGAAGAGAAAGGTCGGAATTCATGGCGTGCCCGCAGCCGGCAGCAGCGGTTCCCTGGAGC |
| tPT2A- | | CCGGCGAGAAACCTTACAAATGCCCCGAGTGCGGCAAGAGCTTCAGCAGAAGCGACGATC |
| ZF54- | | TGGTGAGGCACCAAAGAACCCACACCGGCGAAAAACCTTACAAGTGTCCCGAATGCGGAA |
| KRAB | | AGTCCTTCAGCAGAGAGGACAATCTGCACACCCACCAGAGAACACACACCGGAGAAAAGC |
| nt | | CTTACAAGTGCCCCGAATGCGGCAAATCCTTTTCTAGAAGCGATCATCTGACCACCCACC |
| | | AAAGAACACATACCGGCGAGAAGCCTTACAAATGTCCCGAGTGCGGAAAGTCCTTCTCCC |
| | | AGAGAGCCAATCTGAGGGCTCATCAAAGGACCCATACCGGCGAAAAGCCCTACAAATGCC |
| | | CCGAGTGCGGAAAATCCTTCAGCCAGCTGGCCCATCTGAGAGCCCACCAAAGGACACACA |
| | | CCGGAGAGAAACCCTATAAGTGCCCCGAGTGTGGAAAAAGCTTTTCCCAGAGGGCCAATC |
| | | TGAGGGCCCATCAGAGGACCCATACCGGAGAGAAGCCTTATAAATGTCCCGAGTGCGGAA |
| | | AAAGCTTCAGCGAGAGGAGCCATCTGAGGGAACATCAAAGAACCCACACCGGCGAAAAAC |
| | | CCACCGGAAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCGGCGGCAAGCGGCCCGCCGCCACCAAGAAGGCCGGCCAGG |
| | | CCAAGAAGAAGAAGGGCAGCTACCCCTACGACGTGCCCGACTACGCCGGGTCCGCCACCA |
| | | ACTTCTCGCTGCTGAAGCAGGCCGGAGACGTGGAAGAAAACCCTGGACCTACCAGTGCCG |
| | | GAAAGCTCGGTAGCGGAGAGGGTCGGGGAAGCCTGCTTACTTGCGGCGACGTGGAAGAGA |
| | | ACCCCGGTCCGCTGGAGGGTTCGTCCGGCTCCGGATCCCCCAAGAAGAAGCGGAAGGTGG |
| | | GCATCCACGGCGTGCCCGCCGCCGGCAGCAGCGGATCCCTGGAGCCTGGAGAGAAACCCT |
| | | ACAAATGCCCGGAATGCGGGAAGTCCTTTTCCGAACGCTCGCACCTGAGGGAACACCAGA |
| | | GAACTCACACCGGCGAAAAACCCTATAAGTGCCCAGAATGCGGAAAGAGCTTTTCACGGT |
| | | CGGACAACCTCGTGCGGCACCAACGCACTCATACCGGAGAGAAGCCGTACAAGTGTCCTG |
| | | AGTGCGGAAAGTCATTCTCCGACTGCCGGGATTTGGCCCGCCACCAAAGAACACACACTG |
| | | GCGAAAAGCCCTACAAGTGCCCGGAGTGTGGAAAGTCCTTCAGCACTTCCGGAGAGCTGG |
| | | TCCGGCACCAGAGGACCCACACCGGGGAGAAGCCTTACAAATGTCCAGAGTGCGGTAAAA |
| | | GCTTCTCCACCACCGGCAACCTCACCGTGCACCAGCGGACCCACACTGGAGAAAAGCCGT |
| | | ATAAATGCCCCGAATGCGGCAAGAGCTTCTCGCGATCCGATAAGCTTGTGCGGCATCAGA |
| | | GAACGCACACTGGGGAAAAGCCTTATAAGTGTCCGGAGTGCGGCAAATCCTTCTCCCGCA |
| | | CTGACACCCTGCGGGACCATCAGCGCACCCATACCGGCAAAAAGACCTCTGCTAGCGGCA |
| | | GCGGCGGCGGCAGCGGCGGCGCCCGGGACGACGCCAAGAGCCTGACCGCCTGGAGCCGGA |
| | | CCCTGGTGACCTTCAAGGACGTGTTCGTGGACTTCACCCGGGAGGAGTGGAAGCTGCTGG |
| | | ACACCGCCCAGCAGATCCTGTACCGGAACGTGATGCTGGAGAACTACAAGAACCTGGTGA |
| | | GCCTGGGCTACCAGCTGACCAAGCCCGACGTGATCCTGCGGCTGGAGAAGGGCGAGGAGC |
| | | CCTGGCTGGTGGAGCGGGAGATCCACCAGGAAACCCACCCCGACAGCGAAACCGCCTTCG |
| | | AGATCAAGAGCAGCGTGCCCAGCAGCGGCGGCAAGCGGCCCGCCGCCACCAAGAAGGCCG |
| | | GCCAGGCCAAGAAGAAGAAGGGCAGCTACCCCTACGACGTGCCCGACTACGCCTGAGCGG |
| | | CCGCTTAATTAAGCTGCCTTCTGCGGGGCTTGCCTTCTGGCCATGCCCTTCTTCTCTCCC |
| | | TTGCACCTGTACCTCTTGGTCTTTGAATAAAGCCTGAGTAGGAAGTCTAGAAAAAAAAAA |
| | | AAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAA |
| ZF54- | 197 | GGGAAATAAGAGAGAAAAGAAGAGTAAGAAGAAATATAAGAGCCACCATGGCCCCAAAGA |
| KRAB- | | AGAAGAGAAAGGTCGGAATTCATGGCGTGCCCGCAGCCGGCAGCAGCGGTTCCCTGGAGC |
| tPT2A- | | CTGGAGAGAAACCCTACAAATGCCCGGAATGCGGGAAGTCCTTTTCCGAACGCTCGCACC |
| ZF09- | | TGAGGGAACACCAGAGAACTCACACCGGCGAAAAACCCTATAAGTGCCCAGAATGCGGAA |
| MQ1 | | AGAGCTTTTCACGGTCGGACAACCTCGTGCGGCACCAACGCACTCATACCGGAGAGAAGC |
| nt | | CGTACAAGTGTCCTGAGTGCGGAAAGTCATTCTCCGACTGCCGGGATTTGGCCCGCCACC |
| | | AAAGAACACACACTGGCGAAAAGCCCTACAAGTGCCCGGAGTGTGGAAAGTCCTTCAGCA |
| | | CTTCCGGAGAGCTGGTCCGGCACCAGAGGACCCACACCGGGGAGAAGCCTTACAAATGTC |
| | | CAGAGTGCGGTAAAAGCTTCTCCACCACCGGCAACCTCACCGTGCACCAGCGGACCCACA |
| | | CTGGAGAAAAGCCGTATAAATGCCCCGAATGCGGCAAGAGCTTCTCGCGATCCGATAAGC |
| | | TTGTGCGGCATCAGAGAACGCACACTGGGGAAAAGCCTTATAAGTGTCCGGAGTGCGGCA |
| | | AATCCTTCTCCCGCACTGACACCCTGCGGGACCATCAGCGCACCCATACCGGCAAAAAGA |
| | | CCTCTGCTAGCGGCAGCGGCGGCGGCAGCGGCGGCGCCCGGGACGACGCCAAGAGCCTGA |
| | | CCGCCTGGAGCCGGACCCTGGTGACCTTCAAGGACGTGTTCGTGGACTTCACCCGGGAGG |
| | | AGTGGAAGCTGCTGGACACCGCCCAGCAGATCCTGTACCGGAACGTGATGCTGGAGAACT |
| | | ACAAGAACCTGGTGAGCCTGGGCTACCAGCTGACCAAGCCCGACGTGATCCTGCGGCTGG |
| | | AGAAGGGCGAGGAGCCCTGGCTGGTGGAGCGGGAGATCCACCAGGAAACCCACCCCGACA |
| | | GCGAAACCGCCTTCGAGATCAAGAGCAGCGTGCCCAGCAGCGGCGGCAAGCGGCCCGCCG |
| | | CCACCAAGAAGGCCGGCCAGGCCAAGAAGAAGAAGGGCAGCTACCCCTACGACGTGCCCG |
| | | ACTACGCCGGGTCCGCCACCAACTTCTCGCTGCTGAAGCAGGCCGGAGACGTGGAAGAAA |
| | | ACCCTGGACCTACCAGTGCCGGAAAGCTCGGTAGCGGAGAGGGTCGGGGAAGCCTGCTTA |
| | | CTTGCGGCGACGTGGAAGAGAACCCCGGTCCGCTGGAGGGTTCGTCCGGCTCCGGATCCC |
| | | CCAAGAAGAAGCGGAAGGTGGGCATCCACGGCGTGCCCGCCGCCGGCAGCAGCGGATCCC |
| | | TGGAGCCCGGCGAGAAACCTTACAAATGCCCCGAGTGCGGCAAGAGCTTCAGCAGAAGCG |
| | | ACGATCTGGTGAGGCACCAAAGAACCCACACCGGCGAAAAACCTTACAAGTGTCCCGAAT |
| | | GCGGAAAGTCCTTCAGCAGAGAGGACAATCTGCACACCCACCAGAGAACACACACCGGAG |
| | | AAAAGCCTTACAAGTGCCCCGAATGCGGCAAATCCTTTTCTAGAAGCGATCATCTGACCA |
| | | CCCACCAAAGAACACATACCGGCGAGAAGCCTTACAAATGTCCCGAGTGCGGAAAGTCCT |
| | | TCTCCCAGAGAGCCAATCTGAGGGCTCATCAAAGGACCCATACCGGCGAAAAGCCCTACA |
| | | AATGCCCCGAGTGCGGAAAATCCTTCAGCCAGCTGGCCCATCTGAGAGCCCACCAAAGGA |
| | | CACACACCGGAGAGAAACCCTATAAGTGCCCCGAGTGTGGAAAAAGCTTTTCCCAGAGGG |
| | | CCAATCTGAGGGCCCATCAGAGGACCCATACCGGAGAGAAGCCTTATAAATGTCCCGAGT |
| | | GCGGAAAAAGCTTCAGCGAGAGGAGCCATCTGAGGGAACATCAAAGAACCCACACCGGCG |
| | | AAAAACCCACCGGAAAAAAGACCAGCGCTAGCGGCAGCGGCGGCGGCAGCGGCGGCGCCC |
| | | GGGACAGCAAGGTGGAGAACAAGACCAAGAAGCTGCGGGTGTTCGAGGCCTTCGCCGGCA |
| | | TCGGCGCCCAGCGGAAGGCCCTGGAGAAGGTGCGGAAGGACGAGTACGAGATCGTGGGCC |
| | | TGGCCGAGTGGTACGTGCCCGCCATCGTGATGTACCAGGCCATCCACAACAACTTCCACA |
| | | CCAAGCTGGAGTACAAGAGCGTGAGCCGGGAGGAGATGATCGACTACCTGGAGAACAAGA |
| | | CCCTGAGCTGGAACAGCAAGAACCCCGTGAGCAACGGCTACTGGAAGCGGAAGAAGGACG |
| | | ACGAGCTGAAGATCATCTACAACGCCATCAAGCTGAGCGAGAAGGAGGGCAACATCTTCG |
| | | ACATCCGGGACCTGTACAAGCGGACCCTGAAGAACATCGACCTGCTGACCTACAGCTTCC |
| | | CCTGCCAGGACCTGAGCCAGCAGGGCATCCAGAAGGGCATGAAGCGGGGCAGCGGCACCC |
| | | GGAGCGGCCTGCTGTGGGAGATCGAGCGGGCCCTGGACAGCACCGAGAAGAACGACCTGC |
| | | CCAAGTACCTGCTGATGGAGAACGTGGGCGCCCTGCTGCACAAGAAGAACGAGGAGGAGC |
| | | TGAACCAGTGGAAGCAGAAGCTGGAGAGCCTGGGCTACCAGAACAGCATCGAGGTGCTGA |
| | | ACGCCGCCGACTTCGGCAGCAGCCAGGCCCGGCGGCGGGTGTTCATGATCAGCACCCTGA |
| | | ACGAGTTCGTGGAGCTGCCCAAGGGCGACAAGAAGCCCAAGAGCATCAAGAAGGTGCTGA |
| | | ACAAGATCGTGAGCGAGAAGGACATCCTGAACAACCTGCTGAAGTACAACCTGACCGAGT |
| | | TCAAGAAAACCAAGAGCAACATCAACAAGGCCAGCCTGATCGGCTACAGCAAGTTCAACA |
| | | GCGAGGGCTACGTGTACGACCCCGAGTTCACCGGCCCCACCCTGACCGCCAGCGGCGCCA |
| | | ACAGCCGGATCAAGATCAAGGACGGCAGCAACATCCGGAAGATGAACAGCGACGAGACCT |
| | | TCCTGTACATCGGCTTCGACAGCCAGGACGGCAAGCGGGTGAACGAGATCGAGTTCCTGA |
| | | CCGAGAACCAGAAGATCTTCGTGTGCGGCAACAGCATCAGCGTGGAGGTGCTGGAGGCCA |
| | | TCATCGACAAGATCGGCGGCCCCAGCGGCGGCAAGCGGCCCGCCGCCACCAAGAAGGCCG |
| | | GCCAGGCCAAGAAGAAGAAGGGCAGCTACCCCTACGACGTGCCCGACTACGCCTGAGCGG |
| | | CCGCTTAATTAAGCTGCCTTCTGCGGGGCTTGCCTTCTGGCCATGCCCTTCTTCTCTCCC |
| | | TTGCACCTGTACCTCTTGGTCTTTGAATAAAGCCTGAGTAGGAAGTCTAGAAAAAAAAAA |
| | | AAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAA |
| ZF09- | 208 | GGGAAATAAGAGAGAAAAGAAGAGTAAGAAGAAATATAAGAGCCACCATGGCCCCAAAGA |
| MQ1- | | AGAAGAGAAAGGTCGGAATTCATGGCGTGCCCGCAGCCGGCAGCAGCGGTTCCCTGGAGC |
| tPT2A- | | CCGGCGAGAAACCTTACAAATGCCCCGAGTGCGGCAAGAGCTTCAGCAGAAGCGACGATC |
| ZF54- | | TGGTGAGGCACCAAAGAACCCACACCGGCGAAAAACCTTACAAGTGTCCCGAATGCGGAA |
| KRAB | | AGTCCTTCAGCAGAGAGGACAATCTGCACACCCACCAGAGAACACACACCGGAGAAAAGC |
| nt (no | | CTTACAAGTGCCCCGAATGCGGCAAATCCTTTTCTAGAAGCGATCATCTGACCACCCACC |
| HA | | AAAGAACACATACCGGCGAGAAGCCTTACAAATGTCCCGAGTGCGGAAAGTCCTTCTCCC |
| tags) | | AGAGAGCCAATCTGAGGGCTCATCAAAGGACCCATACCGGCGAAAAGCCCTACAAATGCC |
| (MR- | | CCGAGTGCGGAAAATCCTTCAGCCAGCTGGCCCATCTGAGAGCCCACCAAAGGACACACA |
| 32380) | | CCGGAGAGAAACCCTATAAGTGCCCCGAGTGTGGAAAAAGCTTTTCCCAGAGGGCCAATC |
| | | TGAGGGCCCATCAGAGGACCCATACCGGAGAGAAGCCTTATAAATGTCCCGAGTGCGGAA |
| | | AAAGCTTCAGCGAGAGGAGCCATCTGAGGGAACATCAAAGAACCCACACCGGCGAAAAAC |
| | | CCACCGGAAAAAAGACCAGCGCTAGCGGCAGCGGCGGCGGCAGCGGCGGCGCCCGGGACA |
| | | GCAAGGTGGAGAACAAGACCAAGAAGCTGCGGGTGTTCGAGGCCTTCGCCGGCATCGGCG |
| | | CCCAGCGGAAGGCCCTGGAGAAGGTGCGGAAGGACGAGTACGAGATCGTGGGCCTGGCCG |
| | | AGTGGTACGTGCCCGCCATCGTGATGTACCAGGCCATCCACAACAACTTCCACACCAAGC |
| | | TGGAGTACAAGAGCGTGAGCCGGGAGGAGATGATCGACTACCTGGAGAACAAGACCCTGA |
| | | GCTGGAACAGCAAGAACCCCGTGAGCAACGGCTACTGGAAGCGGAAGAAGGACGACGAGC |
| | | TGAAGATCATCTACAACGCCATCAAGCTGAGCGAGAAGGAGGGCAACATCTTCGACATCC |
| | | GGGACCTGTACAAGCGGACCCTGAAGAACATCGACCTGCTGACCTACAGCTTCCCCTGCC |
| | | AGGACCTGAGCCAGCAGGGCATCCAGAAGGGCATGAAGCGGGGCAGCGGCACCCGGAGCG |
| | | GCCTGCTGTGGGAGATCGAGCGGGCCCTGGACAGCACCGAGAAGAACGACCTGCCCAAGT |
| | | ACCTGCTGATGGAGAACGTGGGCGCCCTGCTGCACAAGAAGAACGAGGAGGAGCTGAACC |
| | | AGTGGAAGCAGAAGCTGGAGAGCCTGGGCTACCAGAACAGCATCGAGGTGCTGAACGCCG |
| | | CCGACTTCGGCAGCAGCCAGGCCCGGCGGCGGGTGTTCATGATCAGCACCCTGAACGAGT |
| | | TCGTGGAGCTGCCCAAGGGCGACAAGAAGCCCAAGAGCATCAAGAAGGTGCTGAACAAGA |
| | | TCGTGAGCGAGAAGGACATCCTGAACAACCTGCTGAAGTACAACCTGACCGAGTTCAAGA |
| | | AAACCAAGAGCAACATCAACAAGGCCAGCCTGATCGGCTACAGCAAGTTCAACAGCGAGG |
| | | GCTACGTGTACGACCCCGAGTTCACCGGCCCCACCCTGACCGCCAGCGGCGCCAACAGCC |
| | | GGATCAAGATCAAGGACGGCAGCAACATCCGGAAGATGAACAGCGACGAGACCTTCCTGT |
| | | ACATCGGCTTCGACAGCCAGGACGGCAAGCGGGTGAACGAGATCGAGTTCCTGACCGAGA |
| | | ACCAGAAGATCTTCGTGTGCGGCAACAGCATCAGCGTGGAGGTGCTGGAGGCCATCATCG |
| | | ACAAGATCGGCGGCCCCAGCGGCGGCAAGCGGCCCGCCGCCACCAAGAAGGCCGGCCAGG |
| | | CCAAGAAGAAGAAGGGCAGCGGGTCCGCCACCAACTTCTCGCTGCTGAAGCAGGCCGGAG |
| | | ACGTGGAAGAAAACCCTGGACCTACCAGTGCCGGAAAGCTCGGTAGCGGAGAGGGTCGGG |
| | | GAAGCCTGCTTACTTGCGGCGACGTGGAAGAGAACCCCGGTCCGCTGGAGGGTTCGTCCG |
| | | GCTCCGGATCCCCCAAGAAGAAGCGGAAGGTGGGCATCCACGGCGTGCCCGCCGCCGGCA |
| | | GCAGCGGATCCCTGGAGCCTGGAGAGAAACCCTACAAATGCCCGGAATGCGGGAAGTCCT |
| | | TTTCCGAACGCTCGCACCTGAGGGAACACCAGAGAACTCACACCGGCGAAAAACCCTATA |
| | | AGTGCCCAGAATGCGGAAAGAGCTTTTCACGGTCGGACAACCTCGTGCGGCACCAACGCA |
| | | CTCATACCGGAGAGAAGCCGTACAAGTGTCCTGAGTGCGGAAAGTCATTCTCCGACTGCC |
| | | GGGATTTGGCCCGCCACCAAAGAACACACACTGGCGAAAAGCCCTACAAGTGCCCGGAGT |
| | | GTGGAAAGTCCTTCAGCACTTCCGGAGAGCTGGTCCGGCACCAGAGGACCCACACCGGGG |
| | | AGAAGCCTTACAAATGTCCAGAGTGCGGTAAAAGCTTCTCCACCACCGGCAACCTCACCG |
| | | TGCACCAGCGGACCCACACTGGAGAAAAGCCGTATAAATGCCCCGAATGCGGCAAGAGCT |
| | | TCTCGCGATCCGATAAGCTTGTGCGGCATCAGAGAACGCACACTGGGGAAAAGCCTTATA |
| | | AGTGTCCGGAGTGCGGCAAATCCTTCTCCCGCACTGACACCCTGCGGGACCATCAGCGCA |
| | | CCCATACCGGCAAAAAGACCTCTGCTAGCGGCAGCGGCGGCGGCAGCGGCGGCGCCCGGG |
| | | ACGACGCCAAGAGCCTGACCGCCTGGAGCCGGACCCTGGTGACCTTCAAGGACGTGTTCG |
| | | TGGACTTCACCCGGGAGGAGTGGAAGCTGCTGGACACCGCCCAGCAGATCCTGTACCGGA |
| | | ACGTGATGCTGGAGAACTACAAGAACCTGGTGAGCCTGGGCTACCAGCTGACCAAGCCCG |
| | | ACGTGATCCTGCGGCTGGAGAAGGGCGAGGAGCCCTGGCTGGTGGAGCGGGAGATCCACC |
| | | AGGAAACCCACCCCGACAGCGAAACCGCCTTCGAGATCAAGAGCAGCGTGCCCAGCAGCG |
| | | GCGGCAAGCGGCCCGCCGCCACCAAGAAGGCCGGCCAGGCCAAGAAGAAGAAGGGCAGCT |
| | | GAGCGGCCGCTTAATTAAGCTGCCTTCTGCGGGGCTTGCCTTCTGGCCATGCCCTTCTTC |
| | | TCTCCCTTGCACCTGTACCTCTTGGTCTTTGAATAAAGCCTGAGTAGGAAGTCTAGAAAA |
| | | AAAAAAAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAA |
| ZF9- | 121 | MAPKKKRKVGIHGVPAAGSSGSLEPGEKPYKCPECGKSFSRSDDLVRHQRTHTGEKPYKC |
| MQ1+ | | PECGKSFSREDNLHTHQRTHTGEKPYKCPECGKSFSRSDHLTTHQRTHTGEKPYKCPECG |
| ZF3- | | KSFSQRANLRAHQRTHTGEKPYKCPECGKSFSQLAHLRAHQRTHTGEKPYKCPECGKSES |
| KRAB | | QRANLRAHQRTHTGEKPYKCPECGKSFSERSHLREHQRTHTGEKPTGKKTSASGSGGGSG |
| (aa) | | GARDSKVENKTKKLRVFEAFAGIGAQRKALEKVRKDEYEIVGLAEWYVPAIVMYQAIHNN |
| | | FHTKLEYKSVSREEMIDYLENKTLSWNSKNPVSNGYWKRKKDDELKIIYNAIKLSEKEGN |
| | | IFDIRDLYKRTLKNIDLLTYSFPCQDLSQQGIQKGMKRGSGTRSGLLWEIERALDSTEKN |
| | | DLPKYLLMENVGALLHKKNEEELNQWKQKLESLGYQNSIEVLNAADFGSSQARRRVFMIS |
| | | TLNEFVELPKGDKKPKSIKKVLNKIVSEKDILNNLLKYNLTEFKKTKSNINKASLIGYSK |
| | | FNSEGYVYDPEFTGPTLTASGANSRIKIKDGSNIRKMNSDETFLYIGFDSQDGKRVNEIE |
| | | FLTENQKIFVCGNSISVEVLEAIIDKIGGPSSGGKRPAATKKAGQAKKKKGSYPYDVPDY |
| | | AGSYPYDVPDYAATNFSLLKQAGDVEENPGPTSAGKLGSGEGRGSLLTCGDVEENPGPLE |
| | | GSSGSGSPKKKRKVGIHGVPAAGSSGSLEPGEKPYKCPECGKSFSRSDKLVRHQRTHTGE |
| | | KPYKCPECGKSFSQRAHLERHQRTHTGEKPYKCPECGKSFSDPGHLVRHQRTHTGEKPYK |
| | | CPECGKSFSRSDKLVRHQRTHTGEKPYKCPECGKSFSQLAHLRAHQRTHTGEKPYKCPEC |
| | | GKSFSRADNLTEHQRTHTGEKPYKCPECGKSFSDCRDLARHQRTHTGEKPTGKKTSASGS |
| | | GGGSGGDAKSLTAWSRTLVTFKDVFVDFTREEWKLLDTAQQILYRNVMLENYKNLVSLGY |
| | | QLTKPDVILRLEKGEEPWLVEREIHQETHPDSETAFEIKSSVSGGKRPAATKKAGQAKKK |
| | | KGSYPYDVPDYA |
| ZF3- | 122 | MAPKKKRKVGIHGVPAAGSSGSLEPGEKPYKCPECGKSFSRSDKLVRHQRTHTGEKPYKC |
| KRAB | | PECGKSFSQRAHLERHQRTHTGEKPYKCPECGKSFSDPGHLVRHQRTHTGEKPYKCPECG |
| +ZF9- | | KSFSRSDKLVRHQRTHTGEKPYKCPECGKSFSQLAHLRAHQRTHTGEKPYKCPECGKSFS |
| MQ1 | | RADNLTEHQRTHTGEKPYKCPECGKSFSDCRDLARHQRTHTGEKPTGKKTSASGSGGGSG |
| (aa) | | GDAKSLTAWSRTLVTFKDVFVDFTREEWKLLDTAQQILYRNVMLENYKNLVSLGYQLTKP |
| | | DVILRLEKGEEPWLVEREIHQETHPDSETAFEIKSSVSSGGKRPAATKKAGQAKKKKGSY |
| | | PYDVPDYAGSYPYDVPDYAATNFSLLKQAGDVEENPGPTSAGKLGSGEGRGSLLTCGDVE |
| | | ENPGPLEGSSGSGSPKKKRKVGIHGVPAAGSSGSLEPGEKPYKCPECGKSFSRSDDLVRH |
| | | QRTHTGEKPYKCPECGKSFSREDNLHTHQRTHTGEKPYKCPECGKSFSRSDHLTTHQRTH |
| | | TGEKPYKCPECGKSFSQRANLRAHQRTHTGEKPYKCPECGKSFSQLAHLRAHQRTHTGEK |
| | | PYKCPECGKSFSQRANLRAHQRTHTGEKPYKCPECGKSFSERSHLREHQRTHTGEKPTGK |
| | | KTSASGSGGGSGGARDSKVENKTKKLRVFEAFAGIGAQRKALEKVRKDEYEIVGLAEWYV |
| | | PAIVMYQAIHNNFHTKLEYKSVSREEMIDYLENKTLSWNSKNPVSNGYWKRKKDDELKII |
| | | YNAIKLSEKEGNIFDIRDLYKRTLKNIDLLTYSFPCQDLSQQGIQKGMKRGSGTRSGLLW |
| | | EIERALDSTEKNDLPKYLLMENVGALLHKKNEEELNQWKQKLESLGYQNSIEVLNAADFG |
| | | SSQARRRVFMISTLNEFVELPKGDKKPKSIKKVLNKIVSEKDILNNLLKYNLTEFKKTKS |
| | | NINKASLIGYSKFNSEGYVYDPEFTGPTLTASGANSRIKIKDGSNIRKMNSDETFLYIGF |
| | | DSQDGKRVNEIEFLTENQKIFVCGNSISVEVLEAIIDKIGGPSGGKRPAATKKAGQAKKK |
| | | KGSYPYDVPDYA |
| ZF09- | 181 | MAPKKKRKVGIHGVPAAGSSGSLEPGEKPYKCPECGKSFSRSDDLVRHQRTHTGEKPYKC |
| MQ1- | | PECGKSFSREDNLHTHQRTHTGEKPYKCPECGKSFSRSDHLTTHQRTHTGEKPYKCPECG |
| tPT2A- | | KSFSQRANLRAHQRTHTGEKPYKCPECGKSFSQLAHLRAHQRTHTGEKPYKCPECGKSES |
| ZF54- | | QRANLRAHQRTHTGEKPYKCPECGKSFSERSHLREHQRTHTGEKPTGKKTSASGSGGGSG |
| KRAB | | GARDSKVENKTKKLRVFEAFAGIGAQRKALEKVRKDEYEIVGLAEWYVPAIVMYQAIHNN |
| aa | | FHTKLEYKSVSREEMIDYLENKTLSWNSKNPVSNGYWKRKKDDELKIIYNAIKLSEKEGN |
| | | IFDIRDLYKRTLKNIDLLTYSFPCQDLSQQGIQKGMKRGSGTRSGLLWEIERALDSTEKN |
| | | DLPKYLLMENVGALLHKKNEEELNQWKQKLESLGYQNSIEVLNAADFGSSQARRRVEMIS |
| | | TLNEFVELPKGDKKPKSIKKVLNKIVSEKDILNNLLKYNLTEFKKTKSNINKASLIGYSK |
| | | FNSEGYVYDPEFTGPTLTASGANSRIKIKDGSNIRKMNSDETFLYIGFDSQDGKRVNEIE |
| | | FLTENQKIFVCGNSISVEVLEAIIDKIGGPSGGKRPAATKKAGQAKKKKGSYPYDVPDYA |
| | | GSATNFSLLKQAGDVEENPGPTSAGKLGSGEGRGSLLTCGDVEENPGPLEGSSGSGSPKK |
| | | KRKVGIHGVPAAGSSGSLEPGEKPYKCPECGKSFSERSHLREHQRTHTGEKPYKCPECGK |
| | | SFSRSDNLVRHQRTHTGEKPYKCPECGKSFSDCRDLARHQRTHTGEKPYKCPECGKSEST |
| | | SGELVRHQRTHTGEKPYKCPECGKSFSTTGNLTVHQRTHTGEKPYKCPECGKSFSRSDKL |
| | | VRHQRTHTGEKPYKCPECGKSFSRTDTLRDHQRTHTGKKTSASGSGGGSGGARDDAKSLT |
| | | AWSRTLVTFKDVFVDFTREEWKLLDTAQQILYRNVMLENYKNLVSLGYQLTKPDVILRLE |
| | | KGEEPWLVEREIHQETHPDSETAFEIKSSVPSSGGKRPAATKKAGQAKKKKGSYPYDVPD |
| | | YA |
| ZF54- | 182 | MAPKKKRKVGIHGVPAAGSSGSLEPGEKPYKCPECGKSFSERSHLREHQRTHTGEKPYKC |
| KRAB- | | PECGKSFSRSDNLVRHQRTHTGEKPYKCPECGKSFSDCRDLARHQRTHTGEKPYKCPECG |
| tPT2A- | | KSFSTSGELVRHQRTHTGEKPYKCPECGKSFSTTGNLTVHQRTHTGEKPYKCPECGKSES |
| ZF09- | | RSDKLVRHQRTHTGEKPYKCPECGKSFSRTDTLRDHQRTHTGKKTSASGSGGGSGGARDD |
| MQ1 | | AKSLTAWSRTLVTFKDVFVDFTREEWKLLDTAQQILYRNVMLENYKNLVSLGYQLTKPDV |
| aa | | ILRLEKGEEPWLVEREIHQETHPDSETAFEIKSSVPSSGGKRPAATKKAGQAKKKKGSYP |
| | | YDVPDYAGSATNFSLLKQAGDVEENPGPTSAGKLGSGEGRGSLLTCGDVEENPGPLEGSS |
| | | GSGSPKKKRKVGIHGVPAAGSSGSLEPGEKPYKCPECGKSFSRSDDLVRHQRTHTGEKPY |
| | | KCPECGKSFSREDNLHTHQRTHTGEKPYKCPECGKSFSRSDHLTTHQRTHTGEKPYKCPE |
| | | CGKSFSQRANLRAHQRTHTGEKPYKCPECGKSFSQLAHLRAHQRTHTGEKPYKCPECGKS |
| | | FSQRANLRAHQRTHTGEKPYKCPECGKSFSERSHLREHQRTHTGEKPTGKKTSASGSGGG |
| | | SGGARDSKVENKTKKLRVFEAFAGIGAQRKALEKVRKDEYEIVGLAEWYVPAIVMYQAIH |
| | | NNFHTKLEYKSVSREEMIDYLENKTLSWNSKNPVSNGYWKRKKDDELKIIYNAIKLSEKE |
| | | GNIFDIRDLYKRTLKNIDLLTYSFPCQDLSQQGIQKGMKRGSGTRSGLLWEIERALDSTE |
| | | KNDLPKYLLMENVGALLHKKNEEELNQWKQKLESLGYQNSIEVLNAADFGSSQARRRVEM |
| | | ISTLNEFVELPKGDKKPKSIKKVLNKIVSEKDILNNLLKYNLTEFKKTKSNINKASLIGY |
| | | SKFNSEGYVYDPEFTGPTLTASGANSRIKIKDGSNIRKMNSDETFLYIGFDSQDGKRVNE |
| | | IEFLTENQKIFVCGNSISVEVLEAIIDKIGGPSGGKRPAATKKAGQAKKKKGSYPYDVPD |
| | | YA |
| ZF09- | 187 | MAPKKKRKVGIHGVPAAGSSGSLEPGEKPYKCPECGKSFSRSDDLVRHQRTHTGEKPYKC |
| MQ1- | | PECGKSFSREDNLHTHQRTHTGEKPYKCPECGKSFSRSDHLTTHQRTHTGEKPYKCPECG |
| tPT2A- | | KSFSQRANLRAHQRTHTGEKPYKCPECGKSFSQLAHLRAHQRTHTGEKPYKCPECGKSFS |
| ZF54- | | QRANLRAHQRTHTGEKPYKCPECGKSFSERSHLREHQRTHTGEKPTGKKTSASGSGGGSG |
| KRAB | | GARDSKVENKTKKLRVFEAFAGIGAQRKALEKVRKDEYEIVGLAEWYVPAIVMYQAIHNN |
| aa | | FHTKLEYKSVSREEMIDYLENKTLSWNSKNPVSNGYWKRKKDDELKIIYNAIKLSEKEGN |
| without | | IFDIRDLYKRTLKNIDLLTYSFPCQDLSQQGIQKGMKRGSGTRSGLLWEIERALDSTEKN |
| HA tag | | DLPKYLLMENVGALLHKKNEEELNQWKQKLESLGYQNSIEVLNAADFGSSQARRRVFMIS |
| | | TLNEFVELPKGDKKPKSIKKVLNKIVSEKDILNNLLKYNLTEFKKTKSNINKASLIGYSK |
| | | FNSEGYVYDPEFTGPTLTASGANSRIKIKDGSNIRKMNSDETFLYIGEDSQDGKRVNEIE |
| | | FLTENQKIFVCGNSISVEVLEAIIDKIGGPSGGKRPAATKKAGQAKKKKGSYPYDVPDYA |
| | | GSATNFSLLKQAGDVEENPGPTSAGKLGSGEGRGSLLTCGDVEENPGPLEGSSGSGSPKK |
| | | KRKVGIHGVPAAGSSGSLEPGEKPYKCPECGKSFSERSHLREHQRTHTGEKPYKCPECGK |
| | | SFSRSDNLVRHQRTHTGEKPYKCPECGKSFSDCRDLARHQRTHTGEKPYKCPECGKSEST |
| | | SGELVRHQRTHTGEKPYKCPECGKSFSTTGNLTVHQRTHTGEKPYKCPECGKSFSRSDKL |
| | | VRHQRTHTGEKPYKCPECGKSFSRTDTLRDHQRTHTGKKTSASGSGGGSGGARDDAKSLT |
| | | AWSRTLVTFKDVFVDFTREEWKLLDTAQQILYRNVMLENYKNLVSLGYQLTKPDVILRLE |
| | | KGEEPWLVEREIHQETHPDSETAFEIKSSVPSSGGKRPAATKKAGQAKKKKGS |
| ZF54- | 188 | MAPKKKRKVGIHGVPAAGSSGSLEPGEKPYKCPECGKSFSERSHLREHQRTHTGEKPYKC |
| KRAB- | | PECGKSFSRSDNLVRHQRTHTGEKPYKCPECGKSFSDCRDLARHQRTHTGEKPYKCPECG |
| tPT2A- | | KSFSTSGELVRHQRTHTGEKPYKCPECGKSFSTTGNLTVHQRTHTGEKPYKCPECGKSES |
| ZF09- | | RSDKLVRHQRTHTGEKPYKCPECGKSFSRTDTLRDHQRTHTGKKTSASGSGGGSGGARDD |
| MQ1 | | AKSLTAWSRTLVTFKDVFVDFTREEWKLLDTAQQILYRNVMLENYKNLVSLGYQLTKPDV |
| aa | | ILRLEKGEEPWLVEREIHQETHPDSETAFEIKSSVPSSGGKRPAATKKAGQAKKKKGSYP |
| without | | YDVPDYAGSATNFSLLKQAGDVEENPGPTSAGKLGSGEGRGSLLTCGDVEENPGPLEGSS |
| HA tag | | GSGSPKKKRKVGIHGVPAAGSSGSLEPGEKPYKCPECGKSFSRSDDLVRHQRTHTGEKPY |
| | | KCPECGKSFSREDNLHTHQRTHTGEKPYKCPECGKSFSRSDHLTTHQRTHTGEKPYKCPE |
| | | CGKSFSQRANLRAHQRTHTGEKPYKCPECGKSFSQLAHLRAHQRTHTGEKPYKCPECGKS |
| | | FSQRANLRAHQRTHTGEKPYKCPECGKSFSERSHLREHQRTHTGEKPTGKKTSASGSGGG |
| | | SGGARDSKVENKTKKLRVFEAFAGIGAQRKALEKVRKDEYEIVGLAEWYVPAIVMYQAIH |
| | | NNFHTKLEYKSVSREEMIDYLENKTLSWNSKNPVSNGYWKRKKDDELKIIYNAIKLSEKE |
| | | GNIFDIRDLYKRTLKNIDLLTYSFPCQDLSQQGIQKGMKRGSGTRSGLLWEIERALDSTE |
| | | KNDLPKYLLMENVGALLHKKNEEELNQWKQKLESLGYQNSIEVLNAADFGSSQARRRVEM |
| | | ISTLNEFVELPKGDKKPKSIKKVLNKIVSEKDILNNLLKYNLTEFKKTKSNINKASLIGY |
| | | SKFNSEGYVYDPEFTGPTLTASGANSRIKIKDGSNIRKMNSDETFLYIGFDSQDGKRVNE |
| | | IEFLTENQKIFVCGNSISVEVLEAIIDKIGGPSGGKRPAATKKAGQAKKKKGS |

In some embodiments, an expression repressor comprises a nuclear localization sequence (NLS). In some embodiments, the expression repressor comprises an NLS, e.g., an SV40 NLS at the N-terminus. In some embodiments, the expression repressor comprises an NLS, e.g., a nucleoplasmin NLS at the C-terminus. In some embodiments, the expression repressor comprises a first NLS at the N-terminus and a second NLS at the C-terminus. In some embodiments the first and the second NLS have the same sequence. In some embodiments, the first and the second NLS have different sequences. In some embodiments, the expression repression repressor comprises an SV40 NLS, e.g., the expression repressor comprises a sequence according to PKKKRK (SEQ ID NO: 135). In some embodiments, the N-terminal sequence comprises an NLS and a spacer, e.g., having a sequence according to: MAPKKKRKVGIHGVPAAGSSGS (SEQ ID NO: 88). In some embodiments, the expression repressor comprises a C-terminal sequence comprising one or more of, e.g., any two or all three of: a spacer, a nucleoplasmin nuclear localization sequence and an HA-tag: e.g., SGGKRPAATKKAGQAKKKGSYPYDVPDYA (SEQ ID NO: 89). In some embodiments, the expression repressor comprises an epitope tag, e.g., an HA tag: YPYDVPDYA (SEQ ID NO: 90). For example, the expression repressor may comprise two copies of the epitope tag.

While an epitope tag is useful in many research contexts, it is sometimes desirable to omit an epitope tag in a therapeutic context. Accordingly, in some embodiments, the expression repressor lacks an epitope tag. In some embodiments, an expression repressor described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the HA tag of SEQ ID NO: 90. In some embodiments, a nucleic acid described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking a region encoding the HA tag of SEQ ID NO: 90. In some embodiments, the expression repressor comprises a nucleoplasmin NLS, e.g., the expression repressor comprises a sequence of KRPAATKKAGQAKKK (SEQ ID NO: 136). In some embodiments, the expression repressor does not comprise an NLS. In some embodiments, the expression repressor does not comprise an epitope tag. In some embodiments the expression repressor does not comprise an HA tag. In some embodiments, the expression repressor does not comprise an HA tag sequence according to SEQ ID NO: 90.

In some embodiments, the present disclosure provides an expression repressor system comprises a self-cleaving peptide. Self-cleaving peptides, first discovered in picornaviruses, are peptides of between 19 to 22 amino acids in length and are usually found between two proteins in some members of the picornavirus family. Using self-cleaving proteins, picornaviruses are capable of producing equimolar levels of multiple genes from the same mRNA. Such self-cleaving proteins are known to be found in other species of viruses and a person skilled in the art, based on the information provided herein, will be readily able to determine a suitable substitution for the self-cleaving protein disclosed herein, if required. In some embodiments, an expression repressor system comprises a self-cleaving peptide, e.g., a 2A self-cleaving peptide. In some embodiments, the 2A peptide comprises a single cleavage site, e.g., a 2A peptide, e.g., a P2A, a T2A, a E2A, or a F2A peptide. In some embodiments the self-cleaving peptide, e.g., a 2A peptide, comprises two cleavage sites, e.g., pPT2A, or P2A-T2A-E2A. In some embodiments, an expression repressor system comprises a self-cleaving peptide comprising a plurality of cleavage sites, e.g., a T2A self-cleaving peptide and a P2A self-cleaving peptide. In some embodiments, the 2A peptide gets cleaved after translation. In some embodiments, the self-cleaving peptide produces two or more fragments after cleaving. In some embodiments, the 2A peptide fragments comprise the sequences of SEQ ID NO: 126-127. In some embodiments, the 2A self-cleaving peptide comprises a sequence of SEQ ID NO: 120, 124, 125 or derivative thereof. In some embodiments, SEQ ID NO: 95 comprises a sequence of a self-cleaving peptide.

(SEQ ID NO: 95)
PSSGGKRPAATKKAGQAKKKKGSYPYDVPDYAGSYPYDVPDYA
ATNFSLLKQAGDVEENPGPTSAGKLGSGEGRGSLLTCGDVEEN
PGPLEGSSGSGSPKKKRKVGIHGVPAAGSSGS
(SEQ ID NO: 120)
EGRGSLLTCGDVEENPGP
(SEQ ID NO: 124)
ATNFSLLKQAGDVEENPGPTSAGKLGSGEGRGSLLTCGDVEENPGP
(SEQ ID NO: 125)
ATNFSLLKQAGDVEENPGP
(SEQ ID NO: 126)
ATNFSLLKQAGDVEENPG
(SEQ ID NO: 127)
PTSAGKLGSGEGRGSLLTCGDVEENPG
P

It is of course understood that although a 2A sequence, e.g., tPT2A sequence (e.g., according to SEQ ID NO: 124), may be referred to in the scientific literature and herein as a self-cleaving peptide, this is according to a non-limiting theory. According to another non-limiting theory, in some embodiments, a 2A sequence acts via ribosome-skipping. For instance, an mRNA encoding a 2A sequence may induce ribosome skipping, wherein the ribosome fails to form a peptide bond while translating the 2A region, resulting in a release of the first part of the translation product. The ribosome then produces the second part of the translation product. Overall, it is well established that a 2A sequence placed between a first sequence and a second sequence will lead to the production of a first protein comprising the first sequence and a separate, second protein comprising the second sequence. This disclosure is not bound by any particular theory as to the molecular mechanism by which this is achieved.

### Functional Characteristics

An expression repressor or a system of the present disclosure can be used to decrease expression of a target gene, e.g., MYC, in a cell. In general, an expression repressor or a system as described herein binds (e.g., via a targeting moiety) a genomic sequence element proximal to and/or operably linked to a target gene, e.g., MYC. In some embodiments, binding of the expression repressor or the system to the genomic sequence element modulates (e.g., decreases) expression of the target gene, e.g., MYC. For example, binding of an expression repressor or a system comprising an effector moiety that inhibits recruitment of components of the transcription machinery to the genomic sequence element may modulate (e.g., decrease) expression of the target gene, e.g., MYC. As a further example, binding of an expression repressor or a system comprising an effector moiety with an enzymatic activity (e.g., an epigenetic modifying moiety) may modulate (e.g., decrease) expression of the target gene, e.g., MYC) through the localized enzymatic activity of the effector moiety. As a further example, both binding of an expression repressor or a system to a genomic sequence element and the localized enzymatic activity of an expression repressor or a system may contribute to the resulting modulation (e.g., decrease) in expression of the target gene, e.g., MYC.

In some embodiments, decreasing expression comprises decreasing the level of RNA, e.g., mRNA, encoded by the target gene e.g., MYC. In some embodiments, decreasing expression comprises decreasing the level of a protein encoded by the target gene e.g., MYC. In some embodiments, decreasing expression comprises both decreasing the level of mRNA and protein encoded by the target gene e.g., MYC. In some embodiments, the expression of a target gene in a cell contacted by or comprising the expression repressor or the expression repression system disclosed herein is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of expression of the target gene in a cell not contacted by or comprising the expression repressor or the expression repression system disclosed herein. Expression of a target gene e.g., MYC may be assayed by methods known to those of skill in the art, including RT-PCR, ELISA, Western blot, and the methods of Examples 2-9 as described in International Application WO/2022/132195, which is herein incorporated by reference in its entirety. Expression level of a target gene, e.g., MYC in a subject, e.g., a patient, e.g., a patient having a MYC mis-regulation disorder, e.g., a patient having a hepatic disease, a patient having a neoplasia and/or viral or alcohol related hepatic disease, e.g., a patient having a hepatocarcinoma, e.g., a patient having a hepatocarcinoma subtype S1 or hepatocarcinoma subtype S2, may be assessed by evaluating blood (e.g., whole blood) levels of the target gene, e.g., MYC, e.g., by the method of either Oglesbee et al. Clin Chem. 2013 October; 59(10):1461-9. Doi: 10.1373/clinchem.2013.207472 or Deutsch et al. J Neurol Neurosurg Psychiatry. 2014 September; 85(9):994-1002. Doi: 10.1136/jnnp-2013-306788, the contents of which are hereby incorporated by reference in their entirety.

An expression repressor or a system of the present disclosure can be used to decrease expression of a target gene e.g., MYC in a cell for a time period. In some embodiments, the expression of a target gene e.g., MYC in a cell contacted by or comprising the expression repressor or the system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, 14, or 15 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the expression of a target gene, e.g., MYC in a cell contacted by or comprising the expression repressor or the system is appreciably decreased for no more than 10, 9, 8, 7, 6, 5, 4, 3, 2, or 1 years. In some embodiments, the expression of a target gene e.g., MYC in a cell contacted by or comprising the expression repressor or the system is appreciably decreased for at least 1, 2, 3, 4, 5, 6, 7, 8, 9, or 10 cell divisions. An expression repressor or a system of the present disclosure can be used to methylate CpG nucleotides in a target promoter, e.g., MYC promoter. In some embodiments, the transcriptional changes in MYC expression correlates to percentage of CpG methylation. In some embodiments, the methylation persists for at least 1 days, at least 2 days, at least 5 days, at least 7 days, at least 10 days, at least 15 days, or at least 20 days post-treatment with an expression repressor or a system disclosed herein.

An expression repressor or a system of the present disclosure can be used to decrease the viability of a cell comprising the target locus, e.g., MYC locus. In some embodiments, expression repressor or a system of the present disclosure can be used to decrease the viability of a plurality of cells comprising the target locus, e.g., MYC locus. In some embodiments, the number of viable cells contacted by or comprising the expression repressor, or the system is appreciably decreased by 10, 20, 30, 40, 50, 60, 70, 80, 90, or 100% compared to number of viable cells in a control population of cells that is not contacted by or does not comprise the expression repressor or the system.

In some embodiments, an expression repressor or a system of the present disclosure can be used to decrease the viability of a plurality of cells comprising cancer cells and non-cancer cells. In some embodiments, an expression repressor or a system of the present disclosure can be used to decrease the viability of the plurality of cancer cells more than it decreases the viability of the plurality of non-cancer cells. In some embodiments, an expression repressor or a system of the present disclosure can be used to decrease the viability of the plurality of cancer cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of non-cancer cells.

In some embodiments, an expression repressor or a system of the present disclosure can be used to decrease the viability of a plurality of cells comprising infected cells and uninfected cells. In some embodiments, an expression repressor or a system of the present disclosure can be used to decrease the viability of the plurality of infected cells more than it decreases the viability of the plurality of uninfected cells. In some embodiments, an expression repressor or a system of the present disclosure can be used to decrease the viability of the plurality of infected cells 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than it decreases the viability of the plurality of uninfected cells.

An expression repression system may comprise a plurality of expression repressors, where each expression repressor comprises an effector moiety with a different functionality than the effector moiety of another expression repressor. For example, an expression repression system may comprise two expression repressors, where the first expression repressor comprises a first effector moiety comprising an epigenetic modifying moiety e.g., DNA methyltransferase, e.g., MQ1 and the second expression repressor comprises a second effector moiety comprising a transcription repressor, e.g., KRAB. In some embodiments, the second expression repressor does not comprise a second effector moiety. In some embodiments, an expression repression system comprises expression repressors comprising a combination of effector moieties whose functionalities are complementary to one another with regard to inhibiting expression of a target gene, e.g., MYC, where the functionalities together enable inhibition of expression and, optionally, do not inhibit or negligibly inhibit expression when present individually. In some embodiments, an expression repression system comprises a plurality of expression repressors, wherein each expression repressor comprises an effector moiety that complements the effector moieties of each other expression repressor, e.g., each effector moiety decreases expression of a target gene, e.g., MYC.

In some embodiments, an expression repression system comprises expression repressors comprising a combination of effector moieties whose functionalities synergize with one another with regards to inhibiting expression of a target gene, e.g., MYC. Without wishing to be bound by theory, epigenetic modifications to a genomic locus may be cumulative, in that multiple repressive epigenetic markers (e.g., multiple different types of epigenetic markers and/or more extensive marking of a given type) individually together reduce expression more effectively than individual modifications alone (e.g., producing a greater decrease in expression and/or a longer-lasting decrease in expression). In some embodiments, an expression repression system comprises a plurality of expression repressors, wherein each expression repressor comprises an effector moiety that synergizes with the effector moieties of each other expression repressor, e.g., each effector moiety decreases expression of a target gene, e.g., MYC.

In some embodiments, an expression repressor or a system modulates (e.g., decreases) expression of a target gene, e.g., MYC by altering one or more epigenetic markers associated with the target gene, e.g., MYC or an expression control sequence operably linked thereto. In some embodiments, altering comprises decreasing the level of an epigenetic marker associated with the target gene, e.g., MYC or an expression control sequence operably linked thereto. Epigenetic markers include, but are not limited to, DNA methylation, histone methylation, and histone deacetylation.

In some embodiments, altering the level of an epigenetic marker decreases the level of the epigenetic marker associated with the target gene, e.g., MYC or an expression control sequence operably linked thereto by at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of the epigenetic marker associated with the target gene, e.g., MYC or an expression control sequence operably linked thereto in a cell not contacted by or comprising the expression repressor or the system. The level of an epigenetic marker may be assayed by methods known to those of skill in the art, including whole genome bisulfite sequencing, reduced representation bisulfite sequencing, bisulfite amplicon sequencing, methylation arrays, pyrosequencing, ChIP-seq, or ChIP-qPCR. In some embodiments, the changes (e.g., increase or decrease) in epigenetic marker e.g., DNA methylation may be assayed using bisulfite genomic sequencing at precise genomic coordinates according to hg19 reference genome, e.g., in between chr8:129188693-129189048 according to hg19 reference genome. In some embodiments, the changes (e.g., increase or decrease) in epigenetic marker e.g., DNA methylation may be assayed using bisulfite genomic sequencing at a genomic location according to SEQ ID NO: 123.

(SEQ ID NO: 123)
CAGAGAAGGAGGAAGTTAATTCACATTCTTAATTTTTTCTAAGGGC
AAAAAAAAAAAAAAAATGCACCAGCTCATTTTCCATCTCTGCTTG
GGTCATCAGTGTGCATTGTGAGCCTGTACAAAGGCCTTAGACGGG
GAATGCTGCCGAGAGCATCACCTTTTATGTCTTCTTTTATATGAA
ATGTGCCACTTCCCCACTAACCCTGGCTCTGGGCTCTGCCTCTGC
TCTCCTGATGGTGTGTTTATGGTGGATTCAGCATTCTGGGCCACA
CAAGGAAGCTGCAGGGGGTGTCCAAGTTCACATGTCCCCGCATTC
CAGGCGAATGTTTCTGACATTGAGCAATGATATGGCTCT

An expression repressor or the system of the present disclosure can be used to alter the level of an epigenetic marker associated with the target gene, e.g., MYC or an expression control sequence operably linked thereto in a cell for a time period. In some embodiments, the level of the epigenetic marker associated with the target gene or an expression control sequence operably linked thereto in a cell contacted by or comprising the expression repressor or the system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the level of an epigenetic marker associated with the target gene, e.g., MYC or an expression control sequence operably linked thereto in a cell contacted by or comprising the expression repressor or the system is appreciably decreased for no more than 10, 9, 8, 7, 6, 5, 4, 3, 2, or 1 years.

### Combinations of Repressors

In some embodiments, an expression repression system comprises a first expression repressor comprising a first effector moiety and a second expression repressor comprising a second effector moiety wherein the first effector moiety and second effector moiety are different from one another. In some embodiments, the first effector moiety is or comprises a first epigenetic modifying moiety (e.g., that increases or decreases a first epigenetic marker) or functional fragment thereof and the second effector moiety is or comprises a second epigenetic modifying moiety (e.g., that increases or decreases a second epigenetic marker) or functional fragment thereof. In some embodiments, the first effector moiety is or comprises a DNA methyltransferase or functional fragment thereof and the second effector moiety is or comprises a KRAB or functional fragment thereof.

### Target Sites

Expression repressors or expression repressor systems disclosed herein are useful for modulating, e.g., decreasing, expression of a target gene, e.g., MYC in cell, e.g., in a subject or patient. A target gene, e.g., MYC may be any gene known to those of skill in the art. In some embodiments, a target gene, e.g., MYC is associated with a disease or condition in a subject, e.g., a mammal, e.g., a human, bovine, horse, sheep, chicken, rat, mouse, cat, or dog. A target gene may include coding sequences, e.g., exons, and/or non-coding sequences, e.g., introns, 3′UTR, or 5′UTR. In some embodiments, a target gene is operably linked to a transcription control element.

A targeting moiety suitable for use in an expression repressor or an expression repressor of system described herein may bind, e.g., specifically bind, to any site within a target gene, e.g., MYC, transcription control element operably linked to a target gene, e.g., MYC to an anchor sequence (e.g., an anchor sequence proximal to a target gene or associated with an anchor sequence-mediated conjunction operably linked to a target gene, e.g., MYC (e.g., an anchor sequence-mediated conjunction is operably linked to a target gene if disruption of the conjunction alters expression of the target gene, e.g., MYC)), or to a regulatory element located in a super enhancer region (e.g., a regulatory element located in a super enhancer region of MYC).

In some embodiments, an expression repressor described herein binds at a site or at a location that is proximal to the site. For example, a targeting moiety may bind to a first site that is proximal to a repressor (the second site), and the effector moiety associated with said targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, e.g., MYC, or proximal to an anchor sequence is less than 5000, 4000, 3000, 2000, 1000, 900, 800, 700, 600, 500, 400, 300, 200, 100, 50, or 25 base pairs from the target gene, e.g., MYC (e.g., an exon, intron, or splice site within the target gene, e.g., MYC), transcription control element, or anchor sequence (and optionally at least 20, 25, 50, 100, 200, or 300 base pairs from the target gene, e.g., MYC (e.g., an exon, intron, or splice site within the target gene), transcription control element, or anchor sequence).

In some embodiments, a targeting moiety binds to a transcription control element operably linked to a target gene (e.g., MYC), e.g., a promoter or enhancer. In some embodiments, a targeting moiety binds to a portion of or a site within a promoter operably linked to a target gene, e.g., MYC. In some embodiments, a targeting moiety binds to the transcription start site of a target gene, e.g., MYC. In some embodiments, a targeting moiety binds to a portion of or a site within an enhancer operably linked to a target gene, e.g., MYC. In some embodiments, a genomic complex (e.g., ASMC) co-localizes two or more genomic sequence elements, wherein the two or more genomic sequence elements include a promoter. A promoter is, typically, a sequence element that initiates transcription of an associated gene. Promoters are typically near the 5′ end of a gene, not far from its transcription start site. As those of ordinary skill are aware, transcription of protein-coding genes in eukaryotic cells is typically initiated by binding of general transcription factors (e.g., TFIID, TFIIE, TFIIH, FUSE, CT-element etc.) and Mediator to core promoter sequences as a preinitiation complex that directs RNA polymerase II to the transcription start site, and in many instances remains bound to the core promoter sequences even after RNA polymerase escapes and elongation of the primary transcript is initiated. In some embodiments, a promoter includes a sequence element such as TATA, Inr, DPE, or BRE, but those skilled in the art are well aware that such sequences are not necessarily required to define a promoter. Those skilled in the art are familiar with a variety of positive (e.g., enhancers) or negative (e.g., repressors or silencers) transcription control elements that are associated with genes. In some embodiments, a transcription control element is a transcription factor binding site. Typically, when a cognate regulatory protein is bound to such a transcription control element, transcription from the associated gene(s) is altered (e.g., increased or decreased). In some embodiments, a targeting moiety binds to a genomic sequence located within a genomic coordinate GRCh37: chr8:129162465-129212140.

In some embodiments, a targeting moiety binds to a target sequence comprised by or partially comprised by a genomic sequence element. In some embodiments, the genomic sequence element is or comprises an expression control sequence. In some embodiments, the genomic sequence element is or comprises the target gene, e.g., MYC or a part of the target gene, e.g., MYC. In some embodiments, a targeting moiety binds to a target sequence that is at least 10, 11, 12, 13, 14, 15, 16, 17, 18, 19, 20, 21, 22, 23, 24, 25, 26, 27, 28, 29, 30, 31, 32, 33, 34, or 35 bases long (and optionally no more than 40, 39, 38, 37, 36, 35, 34, 33, 32, 31, 30, 29, 28, 27, 26, 25, 24, 23, 22, 21, or 20 bases long). In some embodiments, a targeting moiety binds to a target sequence that is 10-30, 15-30, 15-25, 18-24, 19-23, 20-23, 21-23, or 22-23 bases long. In some embodiments, the target sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases long. In some embodiments, the genomic sequence element is or comprises an anchor sequence.

Each ASMC comprises one or more anchor sequences, e.g., a plurality. In some embodiments, anchor sequences can be manipulated or altered to modulate (e.g., disrupt) a naturally occurring genomic complex (e.g., ASMC) or to form a new genomic complex (e.g., ASMC) (e.g., to form a non-naturally occurring genomic complex (e.g., ASMC) with an exogenous or altered anchor sequence). In some embodiments, an anchor sequence-mediated conjunction can be disrupted to alter, e.g., inhibit, e.g., decrease expression of a target gene. Such disruptions may modulate gene expression by, e.g., changing topological structure of DNA, e.g., by modulating the ability of a target gene to interact with a transcription control element (e.g., enhancing and silencing/repressive sequences).

In some embodiments, a targeting moiety binds to an anchor sequence, e.g., an anchor sequence proximal to a target gene, e.g., MYC or associated with an anchor sequence-mediated conjunction (ASMC) operably linked to a target gene, e.g., MYC (e.g., an anchor sequence-mediated conjunction is operably linked to a target gene, e.g., MYC if disruption of the conjunction alters expression of the target gene, e.g., MYC). In general, an anchor sequence is a genomic sequence element to which a genomic complex component, e.g., nucleating polypeptide binds specifically. In some embodiments, binding of a genomic complex component to an anchor sequence nucleates complex formation, e.g., ASMC formation. In some embodiments, a targeting moiety binds to a target gene, e.g., MYC locus. A locus is generally defined to encompass transcribed region, promoter, and anchor sites of an ASMC comprising a target gene, e.g., MYC. In some embodiments, a targeting moiety binds to a sequence comprising any one of SEQ ID NOs: 75-86 or 199-206. In some embodiments, the first targeting moiety binds to a sequence comprising any one of SEQ ID NOs: 75-86 and the second targeting moiety binds to a sequence comprising any one of SEQ ID NOs: 75-86, wherein the first and the second targeting moiety binds to the same sequence. In some embodiments, the first targeting moiety binds to a sequence comprising any one of SEQ ID NOs: 75-86 and the second targeting moiety binds to a sequence comprising any one of SEQ ID NOs: 75-86 wherein the first and the second targeting moiety binds to different sequences. In some embodiments, the first targeting moiety binds to a sequence comprising any of SEQ ID NOs: 83, 203, or 206 and the second targeting moiety binds to a sequence comprising SEQ ID NO:77. In some embodiments, the first targeting moiety binds to a sequence comprising SEQ ID NO: 77 and the second targeting moiety binds to a sequence comprising any of SEQ ID NOs:83, 203, or 206. In some embodiments, the first targeting moiety binds to a sequence comprising any of SEQ ID NOs: 83, 203, or 206 and the second targeting moiety binds to a sequence comprising any of SEQ ID NOs:199, 204, or 205. In some embodiments, the first targeting moiety binds to a sequence comprising any of SEQ ID NOs: 199, 204, or 205 and the second targeting moiety binds to a sequence comprising any of SEQ ID NOs:83, 203, or 206. In some embodiments, the first targeting moiety binds to a sequence comprising any of SEQ ID NOs: 83, 203, or 206 and the second targeting moiety binds to a sequence comprising SEQ ID NO: 201. In some embodiments, a nucleic acid encoding the first and second expression repressors comprises a first region that encodes the first expression repressor, wherein the first region is upstream of a second region that encodes the second expression repressor. In some embodiments, a nucleic acid encoding the first and second expression repressors comprises a first region that encodes the first expression repressor, wherein the first region is downstream of a second region that encodes the second expression repressor. In some embodiments, the first targeting moiety binds to a sequence comprising any one of SEQ ID NOs: 75-86 or 199-206, and the second targeting moiety (e.g., a CRISPR/Cas domain comprising a gRNA) binds to a sequence comprising any one of SEQ ID NOs: 1-4. In some embodiments, a targeting moiety binds to a sequence comprising any one of SEQ ID NOs: 96-110. In some embodiments, the first targeting moiety binds to a sequence comprising any one of SEQ ID NOs: 96-110 and the second targeting moiety binds to a sequence comprising any one of SEQ ID NOs: 96-110, wherein the first and the second targeting moiety binds to the same sequence. In some embodiments, the first targeting moiety binds to a sequence comprising any one of SEQ ID NOs: 96-110 and the second targeting moiety binds to a sequence comprising any one of SEQ ID NOs: 96-110 wherein the first and the second targeting moiety binds to different sequences. In some embodiments, the first targeting moiety binds to a sequence comprising any one of SEQ ID NOs: 96-110, and the second targeting moiety (e.g., a CRISPR/Cas domain comprising a gRNA) binds to a sequence comprising any one of SEQ ID NOs: 1-4. In some embodiments, the first targeting moiety binds to a sequence comprising any one of the SEQ ID Nos. disclosed in tables 2, 12, or 13, and the second targeting moiety (e.g., a CRISPR/Cas domain comprising a gRNA) binds to a sequence comprising any one of the SEQ ID Nos. disclosed in tables 2, 12, or 13.

TABLE 2
Exemplary target sequences

| Target | | Genomic | |
|---|---|---|---|
| Site | Target Sequence | Coordinates | Strand |
| CTCF | ATGATCTCTGCTGCCAGTAG | chr8: 128746342- | + |
| | (SEQ ID NO: 1) | 128746364 | |
| CTCF | ATCGCGCCTGGATGTCAACG | chr8: 128746321- | − |
| | (SEQ ID NO: 2) | 128746343 | |
| CTCF | ATTGTGCAGTGCATCGGATT | chr8: 128746525- | + |
| | (SEQ ID NO: 3) | 128746547 | |
| Promoter | GTCAAACAGTACTGCTACGG | chr8: 128748014- | + |
| | (SEQ ID NO: 4) | 128748036 | |

Exemplary target sequences are disclosed in Table 12.

TABLE 12
Exemplary target sequences

| | SEQ ID | | GENOMIC |
|---|---|---|---|
| NAME | NO: | SEQUENCE | COORDINATES |
| ZF1-KRAB | 75 | GCTGGAAACCTTGCACCTCGG | GRCh37: |
| | | | chr8: 128746267- |
| | | | 128746287 |
| ZF2-KRAB | 76 | CTGCTGCCAGTAGAGGGCACA | GRCh37: |
| | | | chr8: 128746349- |
| | | | 128746369 |
| ZF3-KRAB | 77 | GCCCAGAGAGGGGGCGGAGGG | GRCh37: |
| | | | chr8: 128746405- |
| | | | 128746425 |
| ZF4-KRAB | 78 | ACGCGGGGAGCAACCAATCGC | GRCh37: |
| | | | chr8: 128746455- |
| | | | 128746475 |
| ZF5-KRAB | 79 | ACTGGCAGCAGAGATCATCGC | GRCh37: |
| | | | chr8: 128746339- |
| | | | 128746359 |
| ZF6-KRAB | 80 | GGGGGCAGGAGCAGGAGCGTC | GRCh37: |
| | | | chr8: 128746287- |
| | | | 128746307 |
| ZF7-MQ1 | 81 | CAGCCTTAGCGAGGCGCCCTG | GRCh37: |
| | | | chr8: 128747885- |
| | | | 128747905 |
| ZF8-MQ1 | 82 | ACTCACAGGACAAGGATGCGG | GRCh37: |
| | | | chr8: 128747990- |
| | | | 128748010 |
| ZF9-MQ1 | 83 | AGCAAAAGAAAATGGTAGGCG | GRCh37: |
| | | | chr8: 128748069- |
| | | | 128748089 |
| ZF10-MQ1 | 84 | ACTCAGCCGGGCAGCCGAGCA | GRCh37: |
| | | | chr8: 128748143- |
| | | | 128748163 |
| ZF11-MQ1 | 85 | CGTACCAGGCTGCAGGGCGCC | GRCh37: |
| | | | chr8: 128747897- |
| | | | 128747917 |
| ZF12-MQI | 86 | AGAGTGGAGGAAAGAAGGGTA | GRCh37: |
| | | | chr8: 128747829- |
| | | | 128747849 |
| ZF54-KRAB | 199 | ACGGGGAATGCTGCCGAGAGC | GRCh37: |
| | | | chr8: 129188825- |
| | | | 129188845 |
| ZF61-KRAB | 200 | ACCTGAACCCTGGAAATTATA | GRCh37: |
| | | | chr8: 129209866- |
| | | | 129209888 |
| ZF67-KRAB | 201 | TAGACGGGGAATGCTGCCGAG | GRCh37: |
| | | | chr8: 129188822- |
| | | | 129188842 |
| ZF68-KRAB | 202 | TGACATTGAGCAATGATATGG | GRCh37: |
| | | | chr8: 129189024- |
| | | | 129189044 |
| ZF09-MQ1-tPT2A- | 203 | AGCAAAAGAAAATGGTAGGCG | GRCh37: |
| ZF54-KRAB target | | | chr8: 128748069- |
| sequence 1 | | | 128748089 |
| ZF09-MQ1-tPT2A- | 204 | ACGGGGAATGCTGCCGAGAGC | GRCh37: |
| ZF54-KRAB target | | | chr8: 129188825- |
| sequence 2 | | | 129188845 |
| ZF54-KRAB- | 205 | ACGGGGAATGCTGCCGAGAGC | GRCh37: |
| tPT2A-ZF09-MQ1 | | | chr8: 129188825- |
| target sequence 1 | | | 129188845 |
| ZF54-KRAB- | 206 | AGCAAAAGAAAATGGTAGGCG | GRCh37: |
| tPT2A-ZF09-MQ1 | | | chr8: 128748069- |
| target sequence 2 | | | 128748089 |

TABLE 13
Exemplary Target sequences

| Target Sequence | Genomic Coordinates |
|---|---|
| TGCCACTTCCCCACTAACCC | GRCh37: chr8: 129188878- |
| (SEQ ID NO: 96) | 129188900 |
| GGCCACACAAGGAAGCTGCA | GRCh37: chr8: 129188958- |
| (SEQ ID NO: 97) | 129188980 |
| CCACACAAGGAAGCTGCAGG | GRCh37: chr8: 129188960- |
| (SEQ ID NO: 98) | 129188982 |
| TGATTGGAATGCAACCCGAA | GRCh37: chr8: 129189067- |
| (SEQ ID NO: 99) | 129189089 |
| TTTTGCCCTTGCTACCCCAA | GRCh37: chr8: 129189457- |
| (SEQ ID NO: 100) | 129189479 |
| AGCTGATGGTATCCACTAGG | GRCh37: chr8: 129189554- |
| (SEQ ID NO: 101) | 129189576 |
| CACATCCAAGAATGTAGTGG | GRCh37: chr8: 129189679- |
| (SEQ ID NO: 102) | 129189701 |
| GATACAGCCACAAAGCTCAC | GRCh37: chr8: 129209511- |
| (SEQ ID NO: 103) | 129209533 |
| ATTACATAACAGAATCCAGG | GRCh37: chr8: 129209643- |
| (SEQ ID NO: 104) | 129209665 |
| CCCTTGACTGTGCTGCCACC | GRCh37: chr8: 129209658- |
| (SEQ ID NO: 105) | 129209680 |
| CAGACGAGGAACCTGAACCC | GRCh37: chr8: 129209856- |
| (SEQ ID NO: 106) | 129209878 |
| AGAATCCCTTGGGGTAGCAA | GRCh37: chr8: 129189452- |
| (SEQ ID NO: 107) | 129189474 |
| CAGCACTCTCGCTGACCGCA | GRCh37: chr8: 129189190- |
| (SEQ ID NO: 108) | 129189212 |
| GTTGAGTCATGTGTACTCTG | GRCh37: chr8: 129189274- |
| (SEQ ID NO: 109) | 129189296 |
| AGGAACAGGATGTTACAACT | GRCh37: chr8: 129189421- |
| (SEQ ID NO: 110) | 129189443 |
| GGGGCCACTAGGGACAGGAT | GRCh37: chr19: 55627120- |
| (SEQ ID NO: 111) | 55627139 |

In some embodiments, an expression repressor binds a genomic locus having a sequence set forth herein, e.g., any one of SEQ ID NOs: 1-4, 75-86, 96-110, or 199-206. It is understood that, in many cases, the genomic locus being bound comprises double stranded DNA, and this locus can be described by giving the sequence of its sense strand or its antisense strand. Thus, a gRNA having a given spacer sequence may cause expression repressor to bind to a particular genomic locus, wherein one strand of the genomic locus has a sequence similar or identical to the spacer sequence, and the other strand of the genomic locus has the complementary sequence. Typically, gRNA binding to the genomic locus will involve some unwinding of the genomic locus and pairing of the gRNA spacer with the strand to which it the spacer complementary.

In some embodiments, a targeting moiety binds to an anchor sequence, e.g., an anchor sequence proximal to a target gene, e.g., MYC or associated with an anchor sequence-mediated conjunction (ASMC) operably linked to a target gene, e.g., MYC (e.g., an anchor sequence-mediated conjunction is operably linked to a target gene, e.g., MYC if disruption of the conjunction alters expression of the target gene, e.g., MYC) in mouse genome. In general, an anchor sequence is a genomic sequence element to which a genomic complex component, e.g., nucleating polypeptide binds specifically. In some embodiments, binding of a genomic complex component to an anchor sequence nucleates complex formation, e.g., ASMC formation. In some embodiments, a targeting moiety binds to a target gene, e.g., MYC locus. A locus is generally defined to encompass transcribed region, promoter, and anchor sites of an ASMC comprising a target gene, e.g., MYC. In some embodiments, a targeting moiety binds to a sequence comprising any one of SEQ ID NOs: 190-192. In some embodiments, the targeting moiety binds to a sequence comprising any one of the SEQ ID Nos. disclosed in Table 18. Exemplary target sequences in mouse genome are disclosed in Table 18.

TABLE 18
Exemplary target sequences in mouse genome

| | SEQ | | |
|---|---|---|---|
| | ID | | GENOMIC |
| NAME | NO: | SEQUENCE | COORDINATES |
| ZF15- | 190 | AACACAGTTCA | GRCm38: |
| MQ1 | | GCCGAGCGCT | chr15: 61985053- |
| | | | 61985073 |
| ZF16- | 191 | CGAACAACCGT | GRCm38: |
| MQ1 | | ACAGAAAGGG | chr15: 61985079- |
| | | | 61985099 |
| ZF17- | 192 | GTAAACAGTAA | GRCm38: |
| MQ1 | | TAGCGCAGCA | chr15: 61985151- |
| | | | 61985171 |

In some embodiments, an expression repressor binds a genomic locus having a sequence set forth herein, e.g., any one of SEQ ID NOs: 190-192. It is understood that, in many cases, the genomic locus being bound comprises double stranded DNA, and this locus can be described by giving the sequence of its sense strand or its antisense strand.

In some embodiments, an expression repressor or system described herein acts at an enhancing sequence. In some embodiments, the enhancing sequence is an enhancer, a stretch enhancer, a shadow enhancer, a locus control region (LCR), or a super enhancer. In some embodiments, the super enhancer comprises a cluster of enhancers and other regulatory elements. In some embodiments, these sequences are located in a region 0.2-2 Mb upstream or downstream of a transcription start site. In some embodiments, the region is a non-coding region. In some embodiments, the region contains at least one SNP associated with higher risk of developing cancer. In some embodiments, the region is associated with long-range regulation of a target gene, e.g., MYC. In some embodiments, the regions are cell-type specific. In some embodiments, a super-enhancer modifies (e.g., increases or decreases) target gene expression, e.g., MYC expression, by recruiting the target gene promoter, e.g., MYC promoter. In some embodiments, the super enhancer interacts with a target gene promoter, e.g., MYC promoter, through an enhancer docking site. In some embodiments, the enhancer docking site is an anchor sequence. In some embodiments, the enhancer docking site is located at least 100 bp, 200 bp, 500 bp, 1000 bp, 1500 bp, 2000 bp, or 3000 bp away from the target gene promoter, e.g., MYC promoter. In some embodiments, a super enhancer region is at least 100 bp, at least 200 bp, at least 300 bp, at least 500 bp, at least 1 kb, at least 2 kb, at least 3 kb, at least 5 kb, at least 10 kb, at least 15 kb, at least 20 kb, or at least 25 kb long.

In one embodiment, the anchor sequence-mediated conjunction comprises a loop, such as an intra-chromosomal loop. In certain embodiments, the anchor sequence-mediated conjunction has a plurality of loops. One or more loops may include a first anchor sequence, a nucleic acid sequence, a transcriptional control sequence, and a second anchor sequence. In another embodiment, at least one loop includes, in order, a first anchor sequence, a transcriptional control sequence, and a second anchor sequence, or a first anchor sequence, a nucleic acid sequence, and a second anchor sequence. In yet another embodiment, either one or both of the nucleic acid sequences and the transcriptional control sequence is located within or outside the loop. In still another embodiment, one or more of the loops comprises a transcriptional control sequence.

In some embodiments, the anchor sequence-mediated conjunction includes a TATA box, a CAAT box, a GC box, or a CAP site. In some embodiments, the anchor sequence-mediated conjunction comprises a plurality of loops, and where the anchor sequence-mediated conjunction comprises at least one of an anchor sequence, a nucleic acid sequence, and a transcriptional control sequence in one or more of the loops.

In some embodiments, transcription is inhibited by inclusion of an activating loop or exclusion of a repressive loop. In one such embodiment, the anchor sequence-mediated conjunction excludes a transcriptional control sequence that decreases transcription of the nucleic acid sequence. In some embodiments, transcription is repressed by inclusion of a repressive loop or exclusion of an activating loop. In one such embodiment, the anchor sequence-mediated conjunction includes a transcriptional control sequence that decreases transcription of the nucleic acid sequence.

The anchor sequences may be non-contiguous with one another. In embodiments with noncontiguous anchor sequences, the first anchor sequence may be separated from the second anchor sequence by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 15 500 kb, or about 175 kb to about 500 kb. In some embodiments, the first anchor sequence is separated from the second anchor sequence by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 20 100 Mb, 200 Mb, 300 Mb, 400 Mb, 500 Mb, or any size therebetween.

In some embodiments, an anchor sequence comprises a nucleating polypeptide binding motif, e.g., a CTCF-binding motif: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO: 71), where N is any nucleotide.

A CTCF-binding motif may also be in an opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO: 72). Where N is any nucleotide

In some embodiments, an anchor sequence comprises SEQ ID NO: 71 or SEQ ID NO: 72 or a sequence at least 75%, at least 80%, at least 90%, at least 95%, at least 96%, at least 97%, at least 98%, at least 99% identical to either SEQ ID NO: 71 or SEQ ID NO: 72.

In some embodiments, an anchor sequence-mediated conjunction comprises at least a first anchor sequence and a second anchor sequence. For example, in some embodiments, a first anchor sequence and a second anchor sequence may each comprise a nucleating polypeptide binding motif, e.g., each comprises a CTCF binding motif.

In some embodiments, a first anchor sequence and second anchor sequence comprise different sequences, e.g., a first anchor sequence comprises a CTCF binding motif, and a second anchor sequence comprises an anchor sequence other than a CTCF binding motif. In some embodiments, each anchor sequence comprises a nucleating polypeptide binding motif and one or more flanking nucleotides on one or both sides of a nucleating polypeptide binding motif.

Two CTCF-binding motifs (e.g., contiguous or non-contiguous CTCF binding motifs) that can form an ASMC may be present in a genome in any orientation, e.g., in the same orientation (tandem) either 5′-3′ (left tandem, e.g., the two CTCF-binding motifs that comprise SEQ ID NO:71) or 3′-5′ (right tandem, e.g., the two CTCF-binding motifs comprise SEQ ID NO: 72), or convergent orientation, where one CTCF-binding motif comprises SEQ ID NO:71 and another other comprises SEQ ID NO:72.

In some embodiments, an anchor sequence comprises a CTCF binding motif associated with a target gene (e.g., MYC), wherein the target gene is associated with a disease, disorder and/or condition, e.g., MYC mis-regulating disorder, e.g., hepatic disorder, (e.g., hepatocarcinoma) or lung cancer.

In some embodiments, before administration of an expression repressor or system described herein, the target gene, e.g., MYC has a defined state of expression, e.g., in a diseased state. For example, the target gene, e.g., MYC may have a high level of expression in a disease cell. By disrupting the anchor sequence-mediated conjunction, expression of the target gene, e.g., MYC may be decreased.

A targeting moiety suitable for use in an expression repressor of an expression repression system described herein may bind, e.g., specifically bind, to a site comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs (and optionally no more 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 nucleotides or base pairs). In some embodiments, a DNA-targeting moiety binds to a site comprising 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs.

In some embodiments, the target gene is MYC. In some embodiments, MYC is located on human chromosome 8. In some embodiments, the expression repressor or the expression repressor system as described herein binds to the transcription start site (TSS) of MYC.

### Other Compositions

### Nucleic Acids and Vectors

The present disclosure is further directed, in part, to nucleic acids encoding expression repressors or expression repression systems described herein. In some embodiments, an expression repressor may be provided via a composition comprising a nucleic acid encoding the expression repressor, wherein the nucleic acid is associated with sufficient other sequences to achieve expression of the expression repressor, in a system of interest (e.g., in a particular cell, tissue, organism, etc.). In some embodiments, an expression repression system may be provided via a composition comprising a nucleic acid encoding the expression repression system, e.g., expression repressor(s) of the expression repression system, wherein the nucleic acid is associated with sufficient other sequences to achieve expression of the expression repression system, e.g., expression repressor(s) of the expression repression system, in a system of interest (e.g., in a particular cell, tissue, organism, etc.).

In some particular embodiments, the present disclosure provides compositions of nucleic acids that encode an expression repressor or polypeptide portion thereof. In some such embodiments, provided nucleic acids may be or include DNA, RNA, or any other nucleic acid moiety or entity as described herein, and may be prepared by any technology described herein or otherwise available (e.g., synthesis, cloning, amplification, in vitro or in vivo transcription, etc.). In some embodiments, provided nucleic acids that encode an expression repressor or polypeptide portion thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, a composition for delivering an expression repressor described herein is or comprises RNA, e.g., mRNA, comprising one or more nucleic acids encoding an expression repressor or one or more components of an expression repressor, as described herein.

In some embodiments, a composition for delivering an expression repression system described herein is or comprises RNA, e.g., mRNA, comprising one or more nucleic acids encoding one or more components of an expression repression system, e.g., expression repressor(s) of the expression repression system as described herein.

Nucleic acids as described herein or nucleic acids encoding a protein described herein, may be incorporated into a vector. In some embodiments, a composition for delivering an expression repression system described herein is or comprises a vector, e.g., a viral vector, comprising one or more nucleic acids encoding one or more components of an expression repression system, e.g., expression repressor(s) of the expression repression system as described herein. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

### Cells

The present disclosure is further directed, in part, to cells comprising an expression repressor or an expression repression system described herein. Any cell, e.g., cell line, e.g., a cell line suitable for expression of a recombinant polypeptide, known to one of skill in the art is suitable to comprise an expression repressor or an expression repression system described herein. In some embodiments, a cell, e.g., cell line, may be used to express an expression repressor or an expression repression system, e.g., expression repressor(s), described herein. In some embodiments, a cell, e.g., cell line, may be used to express or amplify a nucleic acid, e.g., a vector, encoding an expression repressor or an expression repression system, e.g., expression repressor(s), described herein. In some embodiments, a cell comprises a nucleic acid encoding an expression repressor or an expression repression system, e.g., expression repressor(s), described herein.

In some embodiments, a cell comprises a first nucleic acid encoding a first component of an expression repression system, e.g., a first expression repressor, and a second nucleic acid encoding a second component of the expression repression system, e.g., a second expression repressor. In some embodiments, wherein a cell comprises nucleic acid encoding an expression repression system comprising two or more expression repressors, the sequences encoding each expression repressor are disposed on separate nucleic acid molecules, e.g., on different vectors, e.g., a first vector encoding a first expression repressor and a second vector encoding a second expression repressor. In some embodiments, the sequences encoding each expression repressor are disposed on the same nucleic acid molecule, e.g., on the same vector. In some embodiments, some or all of the nucleic acid encoding the expression repression system is integrated into the genomic DNA of the cell. In some embodiments, the nucleic acid encoding a first expression repressor of an expression repression system is integrated into the genomic DNA of a cell, and the nucleic acid encoding a second expression repressor of an expression repression system is not integrated into the genomic DNA of a cell (e.g., is situated on a vector). In some embodiments, the nucleic acid(s) encoding a first and a second expression repressor of an expression repression system are integrated into the genomic DNA of a cell, e.g., at the same (e.g., adjacent or colocalized) or different sites in the genomic DNA.

Examples of cells that may comprise and/or express an expression repression system or expression repressor described herein include, but are not limited to, hepatocytes, neuronal cells, endothelial cells, myocytes, and lymphocytes.

The present disclosure is further directed, in part, to a cell made by a method or process described herein. In some embodiments, the disclosure provides a cell produced by: providing an expression repressor or an expression repression system described herein, providing the cell, and contacting the cell with the expression repressor (or a nucleic acid encoding the expression repressor, or a composition comprising said expression repressor or nucleic acid) or the expression repression system (or a nucleic acid encoding the expression repression system, or a composition comprising said expression repression system or nucleic acid). In some embodiments, contacting a cell with an expression repressor comprises contacting the cell with a nucleic acid encoding the expression repressor under conditions that allow the cell to produce the expression repressor. In some embodiments, contacting a cell with an expression repressor comprises contacting an organism that comprises the cell with the expression repressor or a nucleic acid encoding the expression repressor under conditions that allow the cell to produce the expression repressor.

Without wishing to be bound by theory, a cell contacted with an expression repressor or an expression repression system described herein may exhibit: a decrease in expression of a target gene (e.g., MYC) and/or a modification of epigenetic markers associated with the target gene, e.g., MYC, a transcription control element operably linked to the target gene, e.g., MYC, or an anchor sequence proximal to the target gene or associated with an anchor sequence-mediated conjunction operably linked to the target gene, e.g., MYC compared to a similar cell that has not been contacted by the expression repressor or the expression repression system. In some embodiments, a cell exhibiting said decrease in expression of a target gene, e.g., MYC and/or modification of epigenetic markers does not comprise the expression repressor or the expression repression system. The decrease in expression and/or modification of epigenetic markers may persist, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after contact with the expression repressor or the expression repression system.

In some embodiments, a cell previously contacted by an the expression repressor or an expression repression system retains the decrease in expression and/or modification of epigenetic markers after the expression repressor or the expression repression system is no longer present in the cell, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after the expression repressor or the expression repression system is no longer present in the cell.

Methods of Making Expression Repression Systems and/or Expression Repressors

In some embodiments, an expression repressor comprises or is a protein and may thus be produced by methods of making proteins. In some embodiments, an expression repression system, e.g., the expression repressor(s) of an expression repression system, comprise one or more proteins and may thus be produced by methods of making proteins. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013), which is herein incorporated by reference in its entirety.

Proteins comprise one or more amino acids. Amino acids include any compound and/or substance that can be incorporated into a polypeptide chain, e.g., through formation of one or more peptide bonds. In some embodiments, an amino acid has the general structure H₂N—C(H)I—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

### Pharmaceutical Compositions, Formulation, Delivery, and Administration

The present disclosure is further directed, in part, to pharmaceutical compositions comprising an expression repressor or an expression repression system, e.g., expression repressor(s), described herein, to pharmaceutical compositions comprising nucleic acids encoding the expression repressor or the expression repression system, e.g., expression repressor(s), described herein, and/or to and/or compositions that deliver an expression repressor or an expression repression system, e.g., expression repressor(s), described herein to a cell, tissue, organ, and/or subject.

As used herein, the term “pharmaceutical composition” refers to an active agent (e.g., an expression repressor or nucleic acids of the expression receptor, e.g., an expression repression system, e.g., expression repressor(s) of an expression repressor system, or nucleic acid encoding the same), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, a pharmaceutical composition comprising an expression repressor of the present disclosure comprises an expression repressor or nucleic acid(s) encoding the same. In some embodiments, a pharmaceutical composition comprising an expression repression system of the present disclosure comprises or each of the expression repressors of the expression repression system or nucleic acid(s) encoding the same (e.g., if an expression repression system comprises a first expression repressor and a second expression repressor, the pharmaceutical composition comprises the first and second expression repressor). In some embodiments, a pharmaceutical composition comprises less than all of the expression repressors of an expression repression system comprising a plurality of expression repressors. For example, an expression repression system may comprise a first expression repressor and a second expression repressor, and a first pharmaceutical composition may comprise the first expression repressor or nucleic acid encoding the same and a second pharmaceutical composition may comprise the second expression repressor or nucleic acid encoding the same. In some embodiments, a pharmaceutical composition may comprise coformulation of one or more expression repressors, or nucleic acid(s) encoding the same.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate, and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, pharmaceutical compositions may be formulated for delivery to a cell and/or to a subject via any route of administration. Modes of administration to a subject may include injection, infusion, inhalation, intranasal, intraocular, topical delivery, inter-cannular delivery, or ingestion. Injection includes, without limitation, intravenous, intramuscular, intra-arterial, intrathecal, intraventricular, intracapsular, intra-orbital, intracardiac, intradermal, intraperitoneal, transtracheal, subcutaneous, subcuticular, intraarticular, sub capsular, subarachnoid, intraspinal, intra-cerebrospinal, and intra-sternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some embodiments, administration may be bronchial (e.g., by bronchial instillation), buccal, dermal (which may be or comprise, for example, one or more of topical to the dermis, intradermal, inter-dermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, six, eight, ten, 12, 15 or 20 or more administrations may be given to the subject during one treatment or over a period of time as a treatment regimen.

In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

In some embodiments, the nucleic acid encoding the expression repressor (e.g., bicistronic ZF9-MQ1_ZF3-KRAB) is present in the pharmaceutical composition at a concentration of about 0.5-2 mg/mL, e.g., about 0.5-1.5 mg/mL, e.g., about 1 mg/mL.

In some embodiments, the pharmaceutical composition (e.g., the pharmaceutical composition comprising the nucleic acid encoding the expression repressor, e.g., bicistronic ZF9-MQ1_ZF3-KRAB) is at a volume of about 2.5-7.5 ml, e.g., about 3-7 ml, about 4-6 ml, or about 5 ml. In some embodiments, the pharmaceutical composition comprises about 5 mg of nucleic acid encoding the expression repressor in a volume of about 5 ml.

In some embodiments, the pharmaceutical composition is diluted in normal saline to a total volume of 250 mL. In certain embodiments, the pharmaceutical composition comprising 1 mg/mL of nucleic acid encoding the expression repressor (e.g., bicistronic ZF9-MQ1_ZF3-KRAB) at a volume of 5 mL diluted in normal saline to a total volume of 250 mL.

Unless specified otherwise, when concentrations or doses of an expression repressor are provided using mg (e.g., in mg/ml or mg/kg), the mass in mg refers to the mass of the nucleic acid and the LNP together.

### Dosing Regimens

In methods of the inventions, the nucleic acid encoding an expression repressor, the compound of Formula (I) or Formula (II), or the nucleic acid and one of a compound of Formula (I) or Formula (II) may both be administered according to a defined dosing regimen. The dosing regimen may include a defined dose, a defined interval between doses, a defined period of dosing, or any combination thereof. The dosing regimen vary based on, e.g., the condition being treated, the severity of the disease, the subject's individual parameters, including age, physiological condition, size and weight, duration of treatment, the type of treatment to be performed (if any), the particular route of administration and similar factors. Thus, the dosing regimen of the agents described herein can depend on such various parameters. The dosing regimen of an administered composition may also vary depending upon other factors as the subject's sex, general medical condition, and severity of the disorder to be treated.

The dose may be within a defined range. For example, it may be desirable to provide the subject with a dose of a modulating agent disclosed herein that is in the range of from about 1 mg/kg to 6 mg/kg as a single intravenous infusion, although a lower or higher dose also may be administered as circumstances dictate. In some embodiments, doses may include, but are not limited to, 0.001 mg/kg-6 mg/kg, 0.001 mg/kg-5.5 mg/kg, 0.001 mg/kg-5 mg/kg, 0.001 mg/kg-4.5 mg/kg, 0.001 mg/kg-4 mg/kg, 0.001 mg/kg-3.5 mg/kg, 0.001 mg/kg-3 mg/kg, 0.001 mg/kg-2.5 mg/kg, 0.001 mg/kg-2 mg/kg, 0.001 mg/kg-1.5 mg/kg, 0.001 mg/kg-1 mg/kg, 0.001 mg/kg-0.5 mg/kg, 0.001 mg/kg-0.2 mg/kg, 0.001 mg/kg-0.1 mg/kg, 0.001 mg/kg-0.07 mg/kg, 0.001 mg/kg-0.03 mg/kg, 0.001 mg/kg-0.01 mg/kg, 0.001 mg/kg-0.009 mg/kg, 0.001 mg/kg-0.005 mg/kg, 0.002 mg/kg-6.0 mg/kg, 0.002 mg/kg-5.0 mg/kg, 0.002 mg/kg-4 mg/kg, 0.002 mg/kg-3.0 mg/kg, 0.002 mg/kg-2.0 mg/kg, 0.002 mg/kg-1.0 mg/kg, 0.002 mg/kg-0.05 mg/kg, 0.02 mg/kg-6 mg/kg, 0.02 mg/kg-5 mg/kg, 0.02 mg/kg-4 mg/kg, 0.02 mg/kg-3 mg/kg, 0.02 mg/kg-2 mg/kg, 0.02 mg/kg-1 mg/kg, 0.02 mg/kg-0.05 mg/kg, 0.05 mg/kg-6 mg/kg, 0.05 mg/kg-5 mg/kg, 0.05 mg/kg-4 mg/kg, 0.05 mg/kg-3 mg/kg, 0.05 mg/kg-2 mg/kg, 0.05 mg/kg-1 mg/kg, 0.05 mg/kg-0.5 mg/kg, 0.08 mg/kg-6 mg/kg, 0.08 mg/kg-5 mg/kg, 0.08 mg/kg-4 mg/kg, 0.08 mg/kg-3 mg/kg, 0.08 mg/kg-2 mg/kg, 0.08 mg/kg-1 mg/kg, 0.15 mg/kg-6 mg/kg, 0.15 mg/kg-5 mg/kg, 0.15 mg/kg-4 mg/kg, 0.15 mg/kg-3 mg/kg, 0.15 mg/kg-2 mg/kg, 0.15 mg/kg-1 mg/kg, 0.05 mg/kg-0.5 mg/kg, 1.0 mg/kg-6 mg/kg, 1.0 mg/kg-5 mg/kg, 1.0 mg/kg-4 mg/kg, 1.0-3.0 mg/kg, 1.5 mg/kg-3.0 mg/kg, 1.0 mg/kg-1.5 mg/kg, 1.5 mg/kg-3 mg/kg, 3 mg/kg-4 mg/kg, 4 mg/kg-5 mg/kg, or 5 mg/kg-6 mg/kg. In some embodiments, a modulating agent as disclosed herein may be administered at about 0.001 mg/kg to about 6 mg/kg, about 0.001 mg/kg to about 5.5 mg/kg, about 0.001 mg/kg to about 5 mg/kg, about 0.001 mg/kg to about 4.5 mg/kg, about 0.001 mg/kg to about 4 mg/kg, about 0.001 mg/kg to about 3.5 mg/kg, about 0.001 mg/kg to about 3 mg/kg, about 0.001 mg/kg to about 2.5 mg/kg, about 0.001 mg/kg to about 2 mg/kg, about 0.001 mg/kg to about 1.5 mg/kg, about 0.001 mg/kg to about 1 mg/kg, about 0.001 mg/kg to about 0.5 mg/kg, about 0.001 mg/kg to about 0.2 mg/kg, about 0.001 mg/kg to about 0.1 mg/kg, about 0.001 mg/kg to about 0.07 mg/kg, about 0.001 mg/kg to about 0.03 mg/kg, about 0.001 mg/kg to about 0.01 mg/kg, about 0.001 mg/kg to about 0.009 mg/kg, about 0.001 mg/kg to about 0.005 mg/kg, about 0.002 mg/kg to about 6 mg/kg, about 0.002 mg/kg to about 5.5 mg/kg, about 0.002 mg/kg to about 5 mg/kg, about 0.002 mg/kg to about 4.5 mg/kg, about 0.002 mg/kg to about 4 mg/kg, about 0.002 mg/kg to about 3.5 mg/kg, about 0.002 mg/kg to about 3 mg/kg, about 0.002 mg/kg to about 2.5 mg/kg, about 0.002 mg/kg to about 2 mg/kg, about 0.002 mg/kg to about 1.5 mg/kg, about 0.002 mg/kg to about 1 mg/kg, about 0.002 mg/kg to about 0.5 mg/kg, about 0.01 mg/kg to about 6 mg/kg, about 0.01 mg/kg to about 5.5 mg/kg, about 0.01 mg/kg to about 5 mg/kg, about 0.01 mg/kg to about 4.5 mg/kg, about 0.01 mg/kg to about 4 mg/kg, about 0.01 mg/kg to about 3.5 mg/kg, about 0.01 mg/kg to about 3 mg/kg, about 0.01 mg/kg to about 2.5 mg/kg, about 0.01 mg/kg to about 2 mg/kg, about 0.01 mg/kg to about 1.5 mg/kg, about 0.01 mg/kg to about 1 mg/kg, about 0.01 mg/kg to about 0.5 mg/kg, about 0.1 mg/kg to about 6 mg/kg, about 0.1 mg/kg to about 5.5 mg/kg, about 0.1 mg/kg to about 5 mg/kg, about 0.1 mg/kg to about 4.5 mg/kg, about 0.1 mg/kg to about 4 mg/kg, about 0.1 mg/kg to about 3.5 mg/kg, about 0.1 mg/kg to about 3 mg/kg, about 0.1 mg/kg to about 2.5 mg/kg, about 0.1 mg/kg to about 2 mg/kg, about 0.1 mg/kg to about 1.5 mg/kg, about 0.1 mg/kg to about 1 mg/kg, about 0.1 mg/kg to about 0.5 mg/kg, about 0.5 mg/kg to about 6 mg/kg, about 0.5 mg/kg to about 5.5 mg/kg, about 0.5 mg/kg to about 5 mg/kg, about 0.5 mg/kg to about 4.5 mg/kg, about 0.5 mg/kg to about 4 mg/kg, about 0.5 mg/kg to about 3.5 mg/kg, about 0.5 mg/kg to about 3 mg/kg, about 0.5 mg/kg to about 2.5 mg/kg, about 0.5 mg/kg to about 2 mg/kg, about 0.5 mg/kg to about 1.5 mg/kg, about 0.5 mg/kg to about 1 mg/kg, about 1 mg/kg to about 6 mg/kg, about 1 mg/kg to about 5.5 mg/kg, about 1 mg/kg to about 5 mg/kg, about 1 mg/kg to about 4.5 mg/kg, about 1 mg/kg to about 4 mg/kg, about 1 mg/kg to about 3.5 mg/kg, about 1 mg/kg to about 3 mg/kg, about 1 mg/kg to about 2.5 mg/kg, about 1 mg/kg to about 2 mg/kg, about 1 mg/kg to about 1.5 mg/kg, about 1.5 mg/kg to about 6 mg/kg, about 1.5 mg/kg to about 5.5 mg/kg, about 1.5 mg/kg to about 5 mg/kg, about 1.5 mg/kg to about 4.5 mg/kg, about 1.5 mg/kg to about 4 mg/kg, about 1.5 mg/kg to about 3.5 mg/kg, about 1.5 mg/kg to about 3 mg/kg, about 1.5 mg/kg to about 2.5 mg/kg, about 2 mg/kg to about 6 mg/kg, about 2 mg/kg to about 5.5 mg/kg, about 2 mg/kg to about 5 mg/kg, about 2 mg/kg to about 4.5 mg/kg, about 2 mg/kg to about 4 mg/kg, about 2 mg/kg to about 3.5 mg/kg, about 2 mg/kg to about 3 mg/kg, about 2 mg/kg to about 2.5 mg/kg, about 2.5 mg/kg to about 6 mg/kg, about 2.5 mg/kg to about 5.5 mg/kg, about 2.5 mg/kg to about 5 mg/kg, about 2.5 mg/kg to about 4.5 mg/kg, about 2.5 mg/kg to about 4 mg/kg, about 2.5 mg/kg to about 3.5 mg/kg, about 2.5 mg/kg to about 3 mg/kg, about 3 mg/kg to about 6 mg/kg, about 3 mg/kg to about 5.5 mg/kg, about 3 mg/kg to about 5 mg/kg, about 3 mg/kg to about 4.5 mg/kg, about 3 mg/kg to about 4 mg/kg, about 3 mg/kg to about 3.5 mg/kg, about 3.5 mg/kg to about 6 mg/kg, about 3.5 mg/kg to about 5.5 mg/kg, about 3.5 mg/kg to about 5 mg/kg, about 3.5 mg/kg to about 4.5 mg/kg, about 3.5 mg/kg to about 4 mg/kg, about 4 mg/kg to about 6 mg/kg, about 4 mg/kg to about 5.5 mg/kg, about 4 mg/kg to about 5 mg/kg, about 4 mg/kg to about 4.5 mg/kg, about 4.5 mg/kg to about 6 mg/kg, about 4.5 mg/kg to about 5.5 mg/kg, or about 4.5 mg/kg to about 5 mg/kg.

The dose may be a defined value. For example, the dose may be about 0.002 mg/kg, about 0.01 mg/kg, about 0.02 mg/kg, about 0.05 mg/kg, about 0.075 mg/kg, about 0.08 mg/kg, about 0.1 mg/kg, about 0.125 mg/kg, about 0.15 mg/kg, about 0.175 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.08 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, or about 6 mg/kg.

The dose may be administered for a defined dosing period (e.g., the length of time taken to administer a single infusion). For example, the dosing period may be about 5 minutes, about 10 minutes, about 15 minutes, about 20 minutes, about 30 minutes, about 45 minutes, about 60 minutes, about 75 minutes, about 90 minutes, about 2 hours, about 2.5 hours, about 3 hours, about 4 hours, or about 6 hours. In certain embodiments, the dosing period may be between about 80-120 minutes (±10 minutes).

Multiple doses may be administered at defined intervals. For example, the interval between doses may be about 1 days, about 2 days, about 3 days, about 5 days, about 7 days, about 10 days, about 12 days, about 13 days, about 14 days, about 2 weeks, about 3 weeks, about 4 weeks, about 5 weeks, about 6 weeks, about 8 weeks, about 10 weeks, or about 12 weeks. The dose may be repeated as needed, for example, once every day (e.g., for 1-30 days), once every 3 days (e.g., for 1-30 days) once every 5 days (e.g., for 1-30 days), once per week (e.g., for 1-6 weeks or for 2-5 weeks). The dose may be administered multiple times, e.g., once, or twice a week, or once every 1 or 2 weeks.

In some embodiments, the subject is provided with a dose of a modulating agent or combination of modulating agents disclosed herein that is in the range of from about 0.002 mg/kg to 6 mg/kg as multiple intravenous infusions although a lower or higher dose also may be administered as circumstances dictate. In some embodiments, the subject is provided with a dose of a modulating agent or combination of modulating agents disclosed herein that is in the range of from about 0.001 mg/kg to 6 mg/kg as multiple intravenous infusions although a lower or higher dose also may be administered as circumstances dictate. In some embodiments, the subject is provided with a dose of a modulating agent or combination of modulating agents disclosed herein that is in the range of from about 1 mg/kg to 6 mg/kg as multiple intravenous infusions although a lower or higher dose also may be administered as circumstances dictate.

A modulating agent or a combination of modulating agents as disclosed herein may be administered as one dose every 3-5 days, repeated for a total of at least 3 doses. Alternatively, a modulating agent or a combination of modulating agents as disclosed herein may be administered at 3 mg/kg every 5 days for 25 days. Alternatively, a modulating agent or a combination of modulating agents as disclosed herein may be administered at 1.0-5.0 mg/kg every 3-5 days for 1-10 doses. Alternatively, a modulating agent or a combination of modulating agents as disclosed herein may be administered at 1.0-3.0 mg/kg every 5 days for 3 doses then every 3 days for 3 doses. Alternatively, a modulating agent or a combination of modulating agents as disclosed herein may be administered at 1.0-3.0 mg/kg every 5 days for 4 doses then every 3 days for 3 doses. Alternatively, a modulating agent or a combination of modulating agents as disclosed herein may be administered at 0.001 mg/kg-1.5 mg/kg every 14 days. Alternatively, a modulating agent or a combination of modulating agents as disclosed herein may be administered at 0.002 mg/kg-1.5 mg/kg every 14 days. Alternatively, a modulating agent or a combination of modulating agents as disclosed herein may be administered at 6 mg/kg every 5 days for 1-10 doses. Alternatively, a modulating agent or a combination of modulating agents as disclosed herein may be administered at 3 mg/kg every 5 days for 1-10 doses. Alternatively, a modulating agent or a combination of modulating agents as disclosed herein may be administered at 1.5 mg/kg every 5 days for 2 doses, 3 mg/kg every 5 days for 3 doses, 3 mg/kg every 3 days for 1 dose. Alternatively, a modulating agent or a combination of modulating agents as disclosed herein may be administered at 6 mg/kg at every 5 days or at 1.5 mg/kg once a day for 5 days with 2 days off. The dosing schedule can optionally be repeated at other intervals and doses may be given through various parenteral routes, with appropriate adjustment of the dose and schedule.

In some embodiments, the dosing of modulating agents or a combination of modulating agents may include a dose of between 1.0 mg/kg to 6.0 mg/kg, optionally given either weekly, twice per week, or every other week. In some embodiments, the dosing of modulating agents or a combination of modulating agents may include a dose of between 0.001 mg/kg to 1.5 mg/kg, optionally given either weekly, twice per week, or every other week. In some embodiments, a modulating agent or a combination of modulating agents as disclosed herein may be administered at 0.001 mg/kg. In some embodiments, a modulating agent or a combination of modulating agents as disclosed herein may be administered at 0.002 mg/kg. In some embodiments, a modulating agent or a combination of modulating agents as disclosed herein may be administered at 0.02 mg/kg. In some embodiments, a modulating agent or a combination of modulating agents as disclosed herein may be administered at 0.05 mg/kg. In some embodiments, a modulating agent or a combination of modulating agents as disclosed herein may be administered at 0.08 mg/kg. In some embodiments, a modulating agent or a combination of modulating agents as disclosed herein may be administered at 0.125 mg/kg. In some embodiments, a modulating agent or a combination of modulating agents as disclosed herein may be administered at 0.15 mg/kg. In some embodiments, a modulating agent or a combination of modulating agents as disclosed herein may be administered every two weeks e.g., by way of intravenous infusion. In some embodiments, a modulating agent or a combination of modulating agents as disclosed herein may be administered as an intravenous infusion (e.g., over about 80-120 minutes (±10 minute)) every two weeks. In some embodiments, a modulating agent or a combination of modulating agents as disclosed herein may be administered as an intravenous infusion (e.g., over about 80-120 minutes (±10 minute)) every two weeks. In some embodiments, a modulating agent or a combination of modulating agents as disclosed herein may be administered as an intravenous infusion once every 2-weeks (e.g., administered on Day 1 and Day 15 of a 28-day/4-week cycle). In some embodiments, a modulating agent or a combination of modulating agents as disclosed herein may be administered at between about 0.02 mg/kg and 0.15 mg/kg as an intravenous infusion (e.g., over about 80-120 minutes (±10 minute)) once every 2-weeks over a 28-day cycle (e.g., administered on Day 1 and Day 15 of each 28-day cycle). In some embodiments, a modulating agent or a combination of modulating agents as disclosed herein may be administered at between about 0.002 mg/kg and 0.15 mg/kg as an intravenous infusion (e.g., over about 80-120 minutes (±10 minute)) once every 2-weeks over a 28-day cycle (e.g., administered on Day 1 and Day 15 of each 28-day cycle).

In some embodiments, when a subject experiences a grade 2 infusion-associate reaction, the intravenous infusion can be interrupted for treatment and/or infusion time may be extended. In certain embodiments, a subject experiencing a grade 2 infusion-associated reaction is administered a treatment of one or more of antihistamine(s), famotidine or the equivalent, corticosteroids, and bronchodilatory therapy.

In some embodiments, when a subject experiences a grade 3 or 4 infusion-associate reaction during intravenous infusion, the intravenous infusion is stopped. In certain embodiments, a subject experiencing a grade 3 or 4 infusion-associated reaction may be administered one or more of: intravenous saline, methylprednisolone (e.g., 100 mg, e.g., intravenously) or steroid equivalent, diphenhydramine (e.g., 50 mg, e.g., intravenously), bronchodilatory therapy, and epinephrine.

In some embodiments, when a subject experiences a grade 1 (e.g., mild transient reaction; infusion interruption not indicated; intervention not indicated) infusion reaction or hypersensitivity, the method of administration of the expression repressor disclosed herein comprises decreasing infusion rate by 50% or stop infusion until recovery of symptoms. In some embodiments, if the method of administration (e.g., IV infusion) is interrupted due to a subject experiencing a grade 1 (e.g., mild transient reaction; infusion interruption not indicated; intervention not indicated) infusion reaction or hypersensitivity, the method of administration comprises resuming at 50% of the rate prior to the reaction after the subjects symptoms have resolved and may be increased to full rate once 50% rate is tolerated.

In certain embodiments, when a subject experiences a grade 2 (e.g., therapy or infusion interruption indicated but responds promptly to symptomatic treatment) infusion reaction or hypersensitivity, the method of administration of the expression repressor disclosed herein comprises stopping administration. In certain embodiments, when the method of administration (e.g., IV infusion) is stopped due to a subject experiencing a grade 2 (e.g., therapy or infusion interruption indicated but responds promptly to symptomatic treatment) infusion reaction or hypersensitivity, the method of administration of the expression repressor disclosed herein may be restarted at 50% of the previous rate under continuous observation. In some embodiments, when the subject experiences a recurring grade 2 (e.g., therapy or infusion interruption indicated but responds promptly to symptomatic treatment) infusion reaction or hypersensitivity at a reinitiated infusion rate after premedication, the method of administration of the expression repressor disclosed herein is discontinued (e.g., permanently).

In certain embodiments, when a subject experiences a grade 3 (e.g., prolonged; recurrence of symptoms following initial improvement) or grade 4 infusion reaction or hypersensitivity, the method of administration of the expression repressor disclosed herein is discontinued (e.g., permanently). In certain embodiments, when a subject experiences a grade 3 (e.g., prolonged; recurrence of symptoms following initial improvement) or grade 4 infusion reaction or hypersensitivity, after the method of administration of the expression repressor disclosed herein is discontinued (e.g., permanently), the subject is administered supplemental oxygen, fluids, and resuscitative measures as needed.

In some embodiments, when a subject experiences a grade 2 (e.g., painless edema) infusion site extravasation, the method of administration of the expression repressor disclosed herein comprises stopping infusion (e.g., immediately). In some embodiments, if the method of administration (e.g., IV infusion) is interrupted due to a subject experiencing a grade 2 (e.g., painless edema) infusion site extravasation, the method of administration comprises resuming at full rate after a vascular access away from the initial affected site is established.

In certain embodiments, when a subject experiences a grade 3 (e.g., erythema with associated symptoms) infusion site extravasation, the method of administration of the expression repressor disclosed herein comprises stopping infusion (e.g., immediately). In certain embodiments, if the method of administration (e.g., IV infusion) is interrupted due to a subject experiencing a grade 3 (e.g., erythema with associated symptoms) infusion site extravasation, the subject receives one or both of: 1) an infectious disease consultation, and 2) a plastic surgery consultation. In certain embodiments, if the method of administration (e.g., IV infusion) is interrupted due to a subject experiencing a grade 3 (e.g., erythema with associated symptoms) infusion site extravasation, the method of administration may be resumed upon resolution to ≤grade 2 (e.g., painless edema). In certain embodiments, if the method of administration (e.g., IV infusion) is interrupted due to a subject experiencing a grade 3 (e.g., erythema with associated symptoms) infusion site extravasation, the method of administration comprises resuming at 50% of the previous rate after a vascular access away from the initial affected site is established concurrently with continuous observation. In certain embodiments, if the method of administration (e.g., IV infusion) is interrupted due to a subject experiencing a grade 3 (e.g., erythema with associated symptoms) infusion site extravasation, the method of administration is discontinued (e.g., permanently).

In certain embodiments, when a subject experiences a grade 4 infusion site extravasation, the method of administration of the expression repressor disclosed herein comprises stopping infusion (e.g., immediately), e.g., permanently. In certain embodiments, if the method of administration (e.g., IV infusion) is interrupted due to a subject experiencing a grade 4 infusion site extravasation, the subject receives one or both of: 1) an infectious disease consultation, and 2) a plastic surgery consultation.

In some embodiments, when a subject experiences a grade 1 ocular adverse event (e.g., uveitis, eye pain, blurred vision), the subject receives an ophthalmology consultation. In certain embodiments, when a subject experiences a grade 1 ocular adverse event (e.g., uveitis, eye pain, blurred vision), the method of administration of the expression repressor disclosed herein is continued.

In certain embodiments, when a subject experiences a grade 2 ocular adverse event (e.g., uveitis, eye pain, blurred vision), the method of administration of the expression repressor disclosed herein comprises stopping infusion. In some embodiments, when a subject experiences a grade 2 ocular adverse event (e.g., uveitis, eye pain, blurred vision), the subject receives an urgent ophthalmology consultation. In certain embodiments, when the method of administration (e.g., IV infusion) is interrupted due to the subject experiencing a grade 2 ocular adverse event (e.g., uveitis, eye pain, blurred vision), the method of administration of the expression repressor disclosed herein comprises resuming administration without dose reduction upon resolution of the ocular adverse event (e.g., uveitis, eye pain, blurred vision) to ≤grade 1 within 7 days and in consultation with ophthalmology. In certain embodiments, when the method of administration (e.g., IV infusion) is interrupted due to the subject experiencing a grade 2 ocular adverse event (e.g., uveitis, eye pain, blurred vision), the method of administration of the expression repressor disclosed herein comprises resuming administration at a dose reduction (e.g., the next lower dose) upon resolution of the ocular adverse event (e.g., uveitis, eye pain, blurred vision) to <grade 1 greater than 7 days but less than 14 days and in consultation with ophthalmology. In some embodiments, when the method of administration (e.g., IV infusion) is interrupted due to the subject experiencing a grade 2 ocular adverse event (e.g., uveitis, eye pain, blurred vision), the method of administration of the expression repressor disclosed herein comprises discontinuing administration (e.g., permanently) when a grade 2 treatment-related ocular adverse event (e.g., uveitis, eye pain, blurred vision) does not respond to topical therapy and does not improve to ≤grade 1 within 14 days.

In certain embodiments, when a subject experiences a grade 3 or 4 ocular adverse event (e.g., uveitis, eye pain, blurred vision), the method of administration of the expression repressor disclosed herein comprises stopping administration (e.g., permanently). In some embodiments, when a subject experiences a grade 3 or 4 ocular adverse event (e.g., uveitis, eye pain, blurred vision), the subject receives an urgent ophthalmology consultation.

In some embodiments, when a subject experiences a grade 1 (e.g., symptomatic; clinical or diagnostic observations only; intervention not indicated) pneumonitis, the method of administration of the expression repressor disclosed herein comprises either continuing administration or stopping administration. In one embodiment, when a subject experiences a grade 1 (e.g., symptomatic; clinical or diagnostic observations only; intervention not indicated) pneumonitis, the subject receives one or both of 1) a pulmonary consultation, and 2) a pulmonary and infection workup. In certain embodiments, if the method of administration (e.g., IV infusion) is interrupted due to a subject experiencing a grade 1 (e.g., symptomatic; clinical or diagnostic observations only; intervention not indicated) pneumonitis, the method of administration of the expression repressor disclosed herein may be resumed, e.g., upon radiographic evidence of improvement.

In certain embodiments, when a subject experiences a grade 2 (e.g., symptomatic; medical intervention indicated; limiting instrumental activities of daily living (ADL)) pneumonitis, the method of administration of the expression repressor disclosed herein comprises stopping administration. In one embodiment, when a subject experiences a grade 2 (e.g., symptomatic; medical intervention indicated; limiting instrumental ADL) pneumonitis, the subject receives one or both of 1) a pulmonary consultation, and 2) a pulmonary and infection workup. In certain embodiments, if the method of administration (e.g., IV infusion) is interrupted due to a subject experiencing a grade 1 (e.g., symptomatic; clinical or diagnostic observations only; intervention not indicated) pneumonitis, the method of administration of the expression repressor disclosed herein may be resumed upon resolution to ≤grade 1. In certain embodiments, if the method of administration (e.g., IV infusion) is interrupted due to a subject experiencing a grade 1 (e.g., symptomatic; clinical or diagnostic observations only; intervention not indicated) pneumonitis, the method of administration of the expression repressor disclosed herein may be resumed at the next lower dose.

In certain embodiments, when a subject experiences a grade 3 (e.g., severe symptoms; limiting self-care ADL; oxygen indicated) or grade 4 pneumonitis, the method of administration of the expression repressor disclosed herein comprises stopping administration permanently. In one embodiment, when a subject experiences a grade 3 (e.g., severe symptoms; limiting self-care ADL; oxygen indicated) or grade 4 pneumonitis, the subject receives one or both of 1) a pulmonary consultation, and 2) a pulmonary and infection workup.

In some embodiments, when a subject experiences a grade 2 (e.g., symptomatic, no intervention needed) claudication, the method of administration of the expression repressor disclosed herein comprises continuation without modification.

In certain embodiments, when a subject experiences a grade 3 or grade 4 (e.g., disabling) claudication, the method of administration of the expression repressor disclosed herein comprises permanent discontinuation.

In some embodiments, when a subject experiences a grade 2 hypertension (e.g., systolic BP 140-159 mmHg or diastolic BP 90-99 mmHg if previously within normal limits (WNL); change in baseline medical intervention indicated; recurrent or persistent (≥24 hrs); symptomatic increase by >20 mmHg (diastolic) or to >140/90 mmHg; monotherapy indicated initiated) hypertension, antihypertensive therapy may be administered. In some embodiments, when a subject experiences a grade 2 hypertension (e.g., systolic BP 140-159 mmHg or diastolic BP 90-99 mmHg if previously WNL; change in baseline medical intervention indicated; recurrent or persistent (≥24 hrs); symptomatic increase by >20 mmHg (diastolic) or to >140/90 mmHg; monotherapy indicated initiated) hypertension, the method of administration of the expression repressor disclosed herein comprises continuation without modification.

In some embodiments, when a subject experiences a grade 3 (e.g., systolic BP ≥160 mmHg or diastolic BP ≥100 mmHg; medical intervention indicated; more than one drug or more intensive therapy than previously used indicated) hypertension, the method of administration of the expression repressor disclosed herein comprises stopping administration if grade 3 hypertension persists >7 days despite optimal hypertensive therapy. In some embodiments, when a subject experiences a grade 3 (e.g., systolic BP ≥160 mmHg or diastolic BP ≥100 mmHg; medical intervention indicated; more than one drug or more intensive therapy than previously used indicated) hypertension, the method of administration of the expression repressor disclosed herein may be resumed upon resolution to ≤grade 2. In some embodiments, when a subject experiences a grade 3 (e.g., systolic BP ≥160 mmHg or diastolic BP ≥100 mmHg; medical intervention indicated; more than one drug or more intensive therapy than previously used indicated) hypertension, the method of administration of the expression repressor disclosed herein may be resumed at a lower dose.

In some embodiments, when a subject experiences a grade 4 hypertension, the method of administration of the expression repressor disclosed herein comprises stopping administration permanently. In some embodiments, when a subject experiences a grade 4 hypertension, the subject receives a cardiology consultation.

In some embodiments, when a subject experiences a grade 2 (Average QTc 450-480 ms) ECG QTc interval prolongation, the method of administration of the expression repressor disclosed herein comprises stopping administration until resolved or ≤grade 1 or baseline (e.g., ECG(s) collected prior to administration). In certain embodiments, when a subject experiences a grade 2 (Average QTc 450-480 ms) ECG QTc interval prolongation, the method of administration of the expression repressor disclosed herein may be resumed without modification.

In certain embodiments, when a subject experiences a grade 3 (average QTc 481-500 ms) ECG QTc interval prolongation, the method of administration of the expression repressor disclosed herein comprises stopping administration. In some embodiments, when a subject experiences a grade 3 (average QTc 481-500 ms) ECG QTc interval prolongation, the subject receives a cardiology consultation. In certain embodiments, when a subject experiences a grade 3 (average QTc 481-500 ms) ECG QTc interval prolongation, the method of administration of the expression repressor disclosed herein comprises resuming administration upon resolution to grade ≤1 or <30 ms difference from baseline QTc with ≤7 days. In certain embodiments, when a subject experiences a grade 3 (average QTc 481-500 ms) ECG QTc interval prolongation, the method of administration of the expression repressor disclosed herein comprises resuming administration at a lower dose.

In certain embodiments, when a subject experiences a grade 4 (Torsade de pointes; polymorphic ventricular tachycardia; signs/symptoms of serious arrhythmia) ECG QTc interval prolongation, the method of administration of the expression repressor disclosed herein comprises stopping administration permanently. In certain embodiments, when a subject experiences a grade 4 (Torsade de pointes; polymorphic ventricular tachycardia; signs/symptoms of serious arrhythmia) ECG QTc interval prolongation, the subject receives a cardiology consultation.

In some embodiments, when a subject experiences a grade 2 or above myocarditis or cardiac ischemia/infarction, the method of administration of the expression repressor disclosed herein comprises stopping administration permanently. In certain embodiments, when a subject experiences a grade 2 or above myocarditis or cardiac ischemia/infarction, the subject receives a cardiology consultation.

In some embodiments, when a subject experiences any grade of arterial thromboembolic event, the method of administration of the expression repressor disclosed herein comprises stopping administration permanently.

In some embodiments, when a subject experiences a grade 2 cardiovascular disorder, the method of administration of the expression repressor disclosed herein comprises stopping administration. In certain embodiments, when a subject experiences a grade 2 cardiovascular disorder, the method of administration of the expression repressor disclosed herein comprises resuming administration at a lower dose upon resolution to grade ≤1 or baseline. In certain embodiments, when a subject experiences a grade 2 cardiovascular disorder, the method of administration of the expression repressor disclosed herein comprises stopping administration permanently if the grade 2 cardiovascular disorder does not resolve to grade ≤1 or baseline.

In some embodiments, when a subject experiences a grade 3 or greater cardiovascular disorder, the method of administration of the expression repressor disclosed herein comprises stopping administration permanently. In certain embodiments, when a subject experiences a grade 3 cardiovascular disorder, the subject receives a cardiology consultation.

In some embodiments, when a subject experiences diarrhea or colitis, the subject is administered anti-diarrheal medication at the first sign of abdominal cramping, loose stools, or overt diarrhea. In one embodiments, when a subject experiences a grade 1 (e.g., increase of <4 stools per day over baseline; mild increase in ostomy output compared to baseline) diarrhea or colitis, the method of administration of the expression repressor disclosed herein comprises continuation without modification.

In certain embodiments, when a subject experiences a grade 2 (e.g., increase of 4-6 stools per day over baseline; moderate increase in ostomy output compared to baseline; limiting instrumental ADL) diarrhea or colitis, the method of administration of the expression repressor disclosed herein comprises stopping administration. In certain embodiments, when a subject experiences a grade 2 (e.g., increase of 4-6 stools per day over baseline; moderate increase in ostomy output compared to baseline; limiting instrumental ADL) diarrhea or colitis, the method of administration of the expression repressor disclosed herein comprises resuming administration without dose modification upon resolution to grade ≤1.

In some embodiments, when a subject experiences a grade 3 (e.g., increase of ≥7 stools per day over baseline; hospitalization indicated; severe increase in ostomy output compared to baseline; limiting self-care ADL) diarrhea or colitis, the method of administration of the expression repressor disclosed herein comprises stopping administration. In some embodiments, when a subject experiences a grade 3 (e.g., increase of ≥7 stools per day over baseline; hospitalization indicated; severe increase in ostomy output compared to baseline; limiting self-care ADL) diarrhea or colitis, the subject receives a GI consultation. In certain embodiments, when a subject experiences a grade 3 (e.g., increase of ≥7 stools per day over baseline; hospitalization indicated; severe increase in ostomy output compared to baseline; limiting self-care ADL) diarrhea or colitis, the method of administration of the expression repressor disclosed herein comprises resuming administration without dose modification upon resolution to grade ≤1. In certain embodiments, when a subject experiences a grade 3 (e.g., increase of ≥7 stools per day over baseline; hospitalization indicated; severe increase in ostomy output compared to baseline; limiting self-care ADL) diarrhea or colitis, the method of administration of the expression repressor disclosed herein comprises resuming administration at the next lower dose. In certain embodiments, when a subject experiences a grade 3 (e.g., increase of ≥7 stools per day over baseline; hospitalization indicated; severe increase in ostomy output compared to baseline; limiting self-care ADL) diarrhea or colitis, the method of administration of the expression repressor disclosed herein comprises stopping administration permanently if the grade 3 diarrhea or colitis does not resolve to grade ≤1.

In some embodiments, when a subject experiences a grade 4 diarrhea or colitis, the method of administration of the expression repressor disclosed herein comprises stopping administration permanently. In certain embodiments, when a subject experiences a grade 4 diarrhea or colitis, the subject receives a GI consultation.

In some embodiments, when a subject experiences a grade 1 gastrointestinal perforation, the method of administration of the expression repressor disclosed herein comprises stopping administration. In certain embodiments, when a subject experiences a grade 1 gastrointestinal perforation, the subject receives one of both of: 1) a GI consultation, and 2) a general surgery consultation. In certain embodiments, when a subject experiences a grade 1 gastrointestinal perforation, the method of administration of the expression repressor disclosed herein comprises resuming administration upon resolution of the grade 1 gastrointestinal perforation.

In some embodiments, when a subject experiences a grade 2, 3, or 4 gastrointestinal perforation, the method of administration of the expression repressor disclosed herein comprises stopping administration permanently.

In some embodiments, when a subject experiences a grade 2 (e.g., medical intervention indicated) hemorrhage or bleeding, the method of administration of the expression repressor disclosed herein comprises stopping administration. In certain embodiments, when a subject experiences a grade 2 (e.g., medical intervention indicated) hemorrhage or bleeding, the method of administration of the expression repressor disclosed herein comprises resuming administration without dose modification upon resolution to grade ≤1. In certain embodiments, when a subject experiences a grade 2 (e.g., medical intervention indicated) hemorrhage or bleeding, the method of administration of the expression repressor disclosed herein comprises resuming administration at the next lower dose. In certain embodiments, when a subject experiences a grade 2 (e.g., medical intervention indicated) hemorrhage or bleeding, the method of administration of the expression repressor disclosed herein comprises stopping administration permanently if the grade 2 hemorrhage or bleeding does not resolve to grade ≤1.

In certain embodiments, when a subject experiences a grade 3 (e.g., transfusion indicated) or grade 4 hemorrhage or bleeding, the method of administration of the expression repressor disclosed herein comprises stopping administration permanently.

In some embodiments, when a subject experiences a grade 2 AST and/or ALT elevation (e.g., >3.0-5.0×ULN if baseline was normal; >3.0-5.0× baseline if baseline was abnormal), the method of administration of the expression repressor disclosed herein comprises stopping administration. In certain embodiments, when a subject experiences a grade 2 AST and/or ALT elevation (e.g., >3.0-5.0×ULN if baseline was normal; >3.0-5.0× baseline if baseline was abnormal), the method of administration of the expression repressor disclosed herein comprises resuming administration without a modification (e.g., in the first or second occurrence) upon resolution to ≤grade 1. In certain embodiments, when a subject experiences a grade 2 AST and/or ALT elevation (e.g., >3.0-5.0×ULN if baseline was normal; >3.0-5.0× baseline if baseline was abnormal), the method of administration of the expression repressor disclosed herein comprises resuming administration at a lower dose. In certain embodiments, when a subject experiences a grade 2 AST and/or ALT elevation (e.g., >3.0-5.0×ULN if baseline was normal; >3.0-5.0× baseline if baseline was abnormal), the method of administration of the expression repressor disclosed herein comprises resuming administration without a modification (e.g., at the third occurrence).

In certain embodiments, when the subject experiences a grade 3 AST and/or ALT elevation (e.g., >5.0-20.0×ULN if baseline was normal; >5.0-20.0× baseline if baseline was abnormal), the method of administration of the expression repressor disclosed herein comprises stopping administration. In certain embodiments, when a subject experiences a grade 3 AST and/or ALT elevation (e.g., >5.0-20.0×ULN if baseline was normal; >5.0-20.0× baseline if baseline was abnormal), the method of administration of the expression repressor disclosed herein comprises resuming administration at the next lower dose (e.g., in the first occurrence) upon resolution to ≤grade 1 or baseline within 7 days. In certain embodiments, when a subject experiences a second occurrence of a grade 3 AST and/or ALT elevation (e.g., >5.0-20.0×ULN if baseline was normal; >5.0-20.0× baseline if baseline was abnormal), the method of administration of the expression repressor disclosed herein comprises stopping administration permanently.

In some embodiments, when the subject experiences a grade 4 AST and/or ALT elevation (>20.0×ULN if baseline was normal; >20.0× baseline if baseline was abnormal), the method of administration of the expression repressor disclosed herein comprises stopping administration permanently.

In some embodiments, when a subject experiences a grade 2 total bilirubin elevation (e.g., >1.5-3.0×ULN if baseline was normal; >1.5-3.0× baseline if baseline was abnormal), the method of administration of the expression repressor disclosed herein comprises stopping administration. In certain embodiments, when a subject experiences a grade 2 total bilirubin elevation (e.g., >1.5-3.0×ULN if baseline was normal; >1.5-3.0× baseline if baseline was abnormal), the method of administration of the expression repressor disclosed herein comprises resuming administration at the next lower dose (e.g., in the first occurrence) upon resolution to ≤grade 1. In certain embodiments, when a subject experiences a second occurrence of a grade 2 total bilirubin elevation (e.g., >1.5-3.0×ULN if baseline was normal; >1.5-3.0× baseline if baseline was abnormal), the method of administration of the expression repressor disclosed herein comprises stopping administration permanently.

In certain embodiments, when a subject experiences a grade 3 total bilirubin elevation (e.g., >3.0-10.0×ULN if baseline was normal; >3.0-10.0× baseline if baseline was abnormal) or grade 4 (>10.0×ULN if baseline was normal; >10.0× baseline if baseline was abnormal), the method of administration of the expression repressor disclosed herein comprises stopping administration permanently.

In certain embodiments, if grade 3 or grade 4 hyper-bilirubinemia is due to the indirect (non-conjugated) component only, and hemolysis as the etiology has been ruled out as per institutional guidelines (e.g., review of peripheral blood smear and haptoglobin determination), the method of administration of the expression repressor disclosed herein comprises stopping administration until the grade 3 or grade 4 hyper-bilirubinemia is resolved ≤grade 1, and optionally resume administration thereafter. In certain embodiments, the management of simultaneous AST/ALT and total bilirubin elevation should be based on the higher grade of the two.

In some embodiments, when a subject experiences a grade 3 asymptomatic amylase and/or lipase elevation (e.g., >5.0× upper limit of normal (ULN); not associated with symptoms or clinical manifestations of pancreatitis), the method of administration of the expression repressor disclosed herein comprises stopping administration. In certain embodiments, when a subject experiences a grade 3 asymptomatic amylase and/or lipase elevation, the subject receives a GI consultation. In certain embodiments, when a subject experiences a grade 3 asymptomatic amylase and/or lipase elevation (e.g., >5.0× upper limit of normal (ULN); not associated with symptoms or clinical manifestations of pancreatitis), the method of administration of the expression repressor disclosed herein comprises resuming administration at the next lower dose upon resolution to ≤grade 2 within 14 days. In certain embodiments, when a subject experiences a grade 3 asymptomatic amylase and/or lipase elevation (e.g., >5.0× upper limit of normal (ULN); not associated with symptoms or clinical manifestations of pancreatitis), the method of administration of the expression repressor disclosed herein comprises stopping administration permanently if the grade 3 asymptomatic amylase and/or lipase elevation does not resolve to ≤grade 2 within 14 days. In certain embodiments, a subject suspected of experiencing, or identified as experiencing, a ≥Grade 3 amylase and/or lipase receives a CT scan or other imaging study to assess the pancreas, liver, and gallbladder within 1 week of the first occurrence.

In some embodiments, when a subject experiences a grade 2 (e.g., confirmed by radiologic evidence) pancreatitis, the method of administration of the expression repressor disclosed herein comprises stopping administration. In certain embodiments, when a subject experiences a grade 2 (e.g., confirmed by radiologic evidence) pancreatitis, the subject receives a GI consultation. In some embodiments, when a subject experiences a grade 2 (e.g., confirmed by radiologic evidence) pancreatitis, the method of administration of the expression repressor disclosed herein comprises resuming administration at the next lower dose upon resolution to grade 1. In certain embodiments, when a subject experiences a grade 2 (e.g., confirmed by radiologic evidence) pancreatitis, the method of administration of the expression repressor disclosed herein comprises stopping administration permanently if the grade 2 pancreatitis does not resolve to grade 1.

In certain embodiments, when a subject experiences a grade 3 or 4 pancreatitis, the method of administration of the expression repressor disclosed herein comprises stopping administration permanently. In certain embodiments, when a subject experiences a grade 3 or 4 pancreatitis, the subject receives a GI consultation.

In some embodiments, when a subject experiences a grade 2 serum creatinine (e.g., >1.5-3.0× baseline; >1.5-3.0×ULN), the method of administration of the expression repressor disclosed herein comprises stopping administration. In certain embodiments, when a subject experiences a grade 2 serum creatinine (e.g., >1.5-3.0× baseline; >1.5-3.0×ULN), the method of administration of the expression repressor disclosed herein comprises resuming administration at the next lower dose upon resolution to ≤grade 1. In certain embodiments, when a subject experiences a grade 2 serum creatinine (e.g., >1.5-3.0× baseline; >1.5-3.0×ULN), the method of administration of the expression repressor disclosed herein comprises stopping administration permanently if the grade 2 serum creatinine does not resolve to grade 1.

In certain embodiments, when a subject experiences a grade 3 serum creatinine (e.g., >3.0× baseline; >3.0-6.0×ULN) or grade 4 serum creatinine (>6.0×ULN), the method of administration of the expression repressor disclosed herein comprises stopping administration permanently.

In some embodiments, when a subject experiences a grade 2 or grade 3 musculoskeletal, the method of administration of the expression repressor disclosed herein comprises stopping administration. In certain embodiments, when a subject experiences a grade 2 or grade 3 musculoskeletal, the subject receives a rheumatology consultation. In some embodiments, the method of administration of the expression repressor disclosed herein comprises resuming administration at the next lower dose upon resolution to ≤grade 1. In certain embodiments, when a subject experiences a grade 2 or grade 3 musculoskeletal, the method of administration of the expression repressor disclosed herein comprises stopping administration permanently if the grade 2 or grade 3 musculoskeletal does not resolve to ≤grade 1.

In certain embodiments, when a subject experiences a grade 4 musculoskeletal, the method of administration of the expression repressor disclosed herein comprises stopping administration. In certain embodiments, when a subject experiences a grade 4 musculoskeletal, the subject receives a rheumatology consultation. In some embodiments, when a subject experiences a grade 4 musculoskeletal, the method of administration of the expression repressor disclosed herein comprises stopping administration permanently. In certain embodiments, when a subject experiences a grade 4 musculoskeletal, the method of administration of the expression repressor disclosed herein comprises resuming administration at the next lower dose upon consultation with a rheumatologist.

In some embodiments, when a subject experiences a grade 2 hypothyroidism or hyperthyroidism, the method of administration of the expression repressor disclosed herein comprises continuing administration without dose modification.

In certain embodiments, when a subject experiences a grade 3 hypothyroidism or hyperthyroidism, the method of administration of the expression repressor disclosed herein comprises stopping administration. In certain embodiments, when a subject experiences a grade 3 hypothyroidism or hyperthyroidism, the subject receives an endocrinology consultation. In some embodiments, when a subject experiences a grade 3 hypothyroidism or hyperthyroidism, the method of administration of the expression repressor disclosed herein comprises resuming administration without dose modification upon resolution to ≤grade 1 with appropriate management.

In some embodiments, when a subject experiences a grade 4 hypothyroidism or hyperthyroidism, the method of administration of the expression repressor disclosed herein comprises stopping administration permanently.

In some embodiments, when a subject experiences a grade 2 or grade 3 endocrine disorder (e.g., thyroiditis, hypothyroidism, hypophysitis, insulin-deficient diabetes, primary adrenal insufficiency), the method of administration of the expression repressor disclosed herein comprises stopping administration. In certain embodiments, when a subject experiences a grade 2 or grade 3 endocrine disorder (e.g., thyroiditis, hypothyroidism, hypophysitis, insulin-deficient diabetes, primary adrenal insufficiency), the subject receives an endocrinology consultation. In some embodiments, when a subject experiences a grade 2 or grade 3 endocrine disorder (e.g., thyroiditis, hypothyroidism, hypophysitis, insulin-deficient diabetes, primary adrenal insufficiency), the method of administration of the expression repressor disclosed herein comprises resuming administration without dose modification upon resolution to ≤grade 1 with appropriate management.

In certain embodiments, when a subject experiences a grade 4 endocrine disorder, the method of administration of the expression repressor disclosed herein comprises stopping administration permanently. In certain embodiments, when a subject experiences a grade 4 endocrine disorder, the subject receives an endocrinology consultation. In some embodiments, when a subject experiences a grade 4 endocrine disorder (e.g., adrenal insufficiency, ACTH deficiency or glucose intolerance), which resolve or are adequately controlled with physiologic hormone replacement (e.g., corticosteroids or thyroid hormones) or glucose-controlling agents, respectively, may continue treatment with the expression repressor disclosed herein.

In some embodiments, when a subject experiences a grade 1 neurology adverse event, the method of administration of the expression repressor disclosed herein comprises stopping administration. In some embodiments, when a subject experiences a grade 1 neurology adverse event, the subject receives a neurology consultation. In certain embodiments, when a subject is suspected of having one or more of Guillain-Barre syndrome, encephalitis, aseptic meningitis, transverse myelitis, or peripheral neuropathy, the method of administration of the expression repressor disclosed herein comprises stopping administration. In some embodiments, when a subject is suspected of having one or more of Guillain-Barre syndrome, encephalitis, aseptic meningitis, transverse myelitis, or peripheral neuropathy, the subject receives a neurology consultation. In certain embodiments, when a subject experiences a grade 1 neurology adverse event, the method of administration of the expression repressor disclosed herein comprises continuing administration without dose modification. In some embodiments, when a subject experiences a grade 1 neurology adverse event, the method of administration of the expression repressor disclosed herein comprises and the administration is stopped, the method comprises resume administration without a dose modification upon resolution of the adverse event.

In certain embodiments, when a subject experiences a grade 2 neurology adverse event, the method of administration of the expression repressor disclosed herein comprises stopping administration. In some embodiments, when a subject experiences a grade 2 neurology adverse event, the subject receives a neurology consultation. In certain embodiments, the method of administration of the expression repressor disclosed herein comprises, after halting administration in response to a grade 2 neurology adverse event, resuming administration at the next lower dose. In certain embodiments, when a subject experiences a grade 2 neurology adverse event, the method of administration of the expression repressor disclosed herein comprises stopping administration permanently.

In certain embodiments, when a subject experiences a grade 3 or grade 4 neurology adverse event, the method of administration of the expression repressor disclosed herein comprises stopping administration permanently. In some embodiments, when a subject experiences a grade 3 or grade 4 neurology adverse event, the subject receives a neurology consultation.

In some embodiments, when a subject experiences a grade 1 dermatology adverse event, the method of administration of the expression repressor disclosed herein comprises continuing administration without dose modification. In certain embodiments, when a subject experiences a grade 1 dermatology adverse event, the subject receives a topical therapy.

In certain embodiments, when a subject experiences a grade 2 dermatology adverse event (e.g., in the first occurrence), the method of administration of the expression repressor disclosed herein comprises continuing administration without a dose modification. In certain embodiments, when a subject experiences a grade 2 dermatology adverse event (e.g., in the first occurrence), the subject receives a topical therapy. In certain embodiments, when a subject experiences a grade 2 dermatology adverse event (e.g., in the first occurrence) that does not resolve within 7 days after topical therapy, the method of administration of the expression repressor disclosed herein comprises stopping administration. In some embodiments, when a subject experiences a grade 2 dermatology adverse event (e.g., in the second or third occurrence), the method of administration of the expression repressor disclosed herein comprises stopping administration. In some embodiments, when a subject experiences a grade 2 dermatology adverse event (e.g., in the second or third occurrence), the method of administration of the expression repressor disclosed herein comprises resuming administration at the next lower dose upon resolution to ≤grade 1. In some embodiments, when a subject experiences a grade 2 dermatology adverse event (e.g., in the fourth occurrence), the method of administration of the expression repressor disclosed herein comprises stopping administration permanently.

In some embodiments, when a subject experiences a grade 3 dermatology adverse event, the method of administration of the expression repressor disclosed herein comprises stopping administration. In certain embodiments, when a subject experiences a grade 3 dermatology adverse event, the subject receives a topical therapy and/or systemic therapy. In certain embodiments, when a subject experiences a grade 2 dermatology adverse event (e.g., in the first occurrence), the subject receives a topical therapy. In certain embodiments, when a subject experiences a grade 3 dermatology adverse event, the subject receives a dermatology consultation. In some embodiments, when a subject experiences a grade 3 dermatology adverse event (e.g., in the first or second occurrence), the method of administration of the expression repressor disclosed herein comprises resuming administration at the next lower dose upon resolution to ≤grade 1. In some embodiments, when a subject experiences a grade 3 dermatology adverse event (e.g., in the third occurrence), the method of administration of the expression repressor disclosed herein comprises stopping administration permanently.

In some embodiments, when a subject experiences a grade 4 dermatology adverse event, the method of administration of the expression repressor disclosed herein comprises stopping administration permanently. In some embodiments, when a subject experiences a grade 4 dermatology adverse event, the subject receives a dermatology consultation.

In some embodiments, when a subject experiences a grade 1 or grade 2 bullous dermatitis, the method of administration of the expression repressor disclosed herein comprises stopping administration. In certain embodiments, when a subject experiences a grade 1 or grade 2 bullous dermatitis, the subject receives a dermatology consultation. In certain embodiments, the method of administration of the expression repressor disclosed herein comprises resuming administration. In certain embodiments, the method of administration of the expression repressor disclosed herein does not comprise resuming administration.

In certain embodiments, when a subject experiences a grade 3 or grade 4 bullous dermatitis, the method of administration of the expression repressor disclosed herein comprises stopping administration permanently. In certain embodiments, when a subject experiences a grade 3 or grade 4 bullous dermatitis, the subject receives a dermatology consultation.

In some embodiments, when a subject experiences any grade of Stevens-Johnson Syndrome (SJS), Lyell syndrome/toxic epidermal necrolysis (TEN), the method of administration of the expression repressor disclosed herein comprises stopping administration permanently. In certain embodiments, when a subject experiences any grade of Stevens-Johnson Syndrome (SJS), Lyell syndrome/toxic epidermal necrolysis (TEN), the subject receives a dermatology consultation.

In some embodiments, when a subject experiences a grade 3 or grade 4 neutropenia (e.g., absolute neutrophil count (ANC)), the method of administration of the expression repressor disclosed herein comprises stopping administration. In certain embodiments, when a subject experiences a grade 3 or grade 4 neutropenia, the method of administration of the expression repressor disclosed herein comprises resuming administration at the next lower dose upon resolution of the grade 3 or grade 4 neutropenia to ≤grade 1 or baseline within 7 days.

In some embodiments, when a subject experiences a grade 3 febrile neutropenia (e.g., NC<1000/mm³ with a single temperature of >38.3° C. (101° F.) or a sustained temperature of ≥38° C. (100.4° F.) for more than 1 hour), the method of administration of the expression repressor disclosed herein comprises stopping administration. In certain embodiments, when a subject experiences a grade 3 or grade 4 neutropenia, the method of administration of the expression repressor disclosed herein comprises resuming administration at the next lower dose upon resolution of the fever and improvement of the grade 3 febrile neutropenia (e.g., NC<1000/mm³ with a single temperature of >38.3° C. (101° F.) or a sustained temperature of ≥38° C. (100.4° F.) for more than 1 hour), to ≤grade 1 or baseline.

In certain embodiments, when a subject experiences a grade 4 febrile neutropenia (e.g., wherein the subject experiences ANC <500/mm3 with a single temperature of >38.5° C. (101° F.) or a sustained temperature of ≥38° C. (100.4° F.) for more than 1 hour), the method of administration of the expression repressor disclosed herein comprises stopping administration permanently.

In some embodiments, when a subject experiences a grade 3 thrombocytopenia (e.g., <50,000-25,000/mm³), the method of administration of the expression repressor disclosed herein comprises stopping administration. In some embodiments, when a subject experiences a grade 3 thrombocytopenia (e.g., <50,000-25,000/mm³), the method of administration of the expression repressor disclosed herein comprises resuming administration at the next lower dose upon resolution of the grade 3 thrombocytopenia (e.g., <50,000-25,000/mm³) to ≤grade 2 or baseline.

In certain embodiments, when a subject experiences a grade 4 thrombocytopenia (e.g., <25,000/mm³), the method of administration of the expression repressor disclosed herein comprises stopping administration permanently.

In some embodiments, when a subject experiences a grade 3 (<8.0 g/dL) or grade 4 anemia, the method of administration of the expression repressor disclosed herein comprises stopping administration. In certain embodiments, when a subject experiences a grade 3 (<8.0 g/dL) or grade 4 anemia, the method of administration of the expression repressor disclosed herein comprises resuming administration, without dose modification, upon resolution of the grade 3 (<8.0 g/dL) or grade 4 anemia to ≤grade 2 or baseline ≤7 days.

In some embodiments, when a subject experiences any grade lymphopenia, the method of administration of the expression repressor disclosed herein comprises continuing administration without dose modification.

In some embodiments, when a subject experiences a grade 3 adverse event not disclosed herein, the method of administration of the expression repressor disclosed herein comprises stopping administration. In certain embodiments, when a subject experiences a grade 3 adverse event not disclosed herein, the method of administration of the expression repressor disclosed herein comprises resuming administration, without dose modification, upon resolution of the adverse event to ≤grade 1. In certain embodiments, when a subject experiences a grade 3 adverse event not disclosed herein, the method of administration of the expression repressor disclosed herein comprises resuming administration at the next lower dose.

In certain embodiments, when a subject experiences a grade 4 adverse event not disclosed herein, the method of administration of the expression repressor disclosed herein comprises stopping administration permanently. In some embodiments, when a subject experiences an isolated grade 4 electrolyte abnormality not associated with clinical sequelae and corrected after appropriate management within 72 hours of their onset do not require stopping administration. In some embodiments, when a subject experiences an isolated grade 4 electrolyte abnormality that does not resolve to ≤grade 1 within 72 hours after appropriate management, the method of administration of the expression repressor disclosed herein comprises stopping administration permanently. In some embodiments, when a subject experiences an isolated grade 4 electrolyte abnormality associated with clinical sequelae, the method of administration of the expression repressor disclosed herein comprises stopping administration permanently.

The person of ordinary skill will realize that a variety of factors, such as age, sex, weight, severity of disorder to be treated may be considered in selecting a dosage of a modulating agent or a combination of modulating agents as disclosed herein, and that the dosage and/or frequency of administration may be increased or decreased during the course of therapy. The dosage may be repeated as needed, with evidence of reduction of tumor volume observed after as few as 2 to 8 doses. The doses and schedules of administration disclosed herein show minimal effect on overall weight of the subject compared to cisplatin, sorafenib, or a small molecule comparator. The subject methods may include use of CT and/or PET/CT, or MRI, to measure tumor response at regular intervals. Blood levels of tumor markers may also be monitored. Dosages and/or administration schedules may be adjusted as needed, according to the results of imaging and/or marker blood levels.

In some embodiments, the compositions disclosed herein may be administered in combination with one or more therapeutic agents or methods chosen from surgical resection, tyrosine kinase inhibitors (TKIs), e.g., sorafenib, bromodomain inhibitors, e.g., BET inhibitors, e.g., JQ1, e.g., BET672, e.g., birabresib, MEK inhibitors, (e.g., Trametinib), orthotopic liver transplantation, radiofrequency ablation, immunotherapy, immune checkpoint plus anti-vascular-endothelial-growth-factor combination therapy, photodynamic therapy (PDT), laser therapy, brachytherapy, radiation therapy, trans-catheter arterial chemo- or radio-embolization, stereotactic radiation therapy, chemotherapy, and/or systemic chemotherapy to treat a disease or disorder. Table 21 below discloses exemplary therapeutic agents.

TABLE 21
Small molecule compounds, e.g., for using in combination therapies with expression repressors described herein.

| | |
|---|---|
| sorafenib | 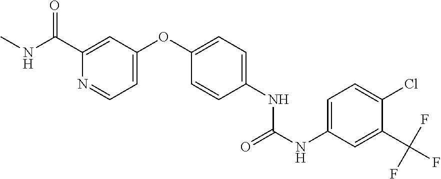 [SMILES: CNC(=O)c1cc(Oc2ccc(NC(=O)Nc3ccc(Cl)c(C(F)(F)F)c3)cc2)ccn1] |
| JQ1 | 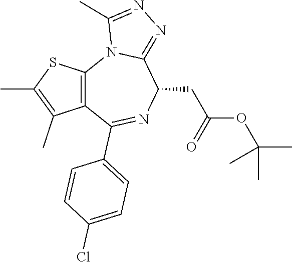 [SMILES: Cc1sc2c(c1C)C(c1ccc(Cl)cc1)=N[C@@H](CC(=O)OC(C)(C)C)c1nnc(C)n1-2] |
| BET762 | 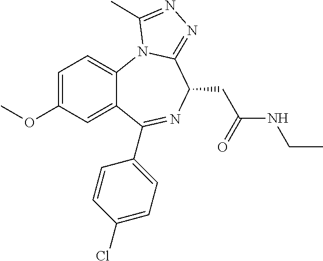 [SMILES: CCNC(=O)C[C@@H]1N=C(c2ccc(Cl)cc2)c2cc(OC)ccc2-n2c(C)nnc21] |
| birabresib | 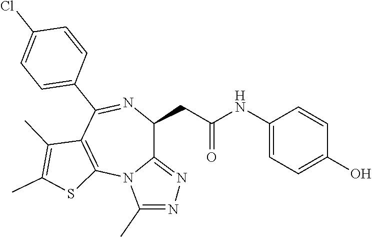 [SMILES: Cc1sc2c(c1C)C(c1ccc(Cl)cc1)=N[C@@H](CC(=O)Nc1ccc(O)cc1)c1nnc(C)n1-2] |
| trametinib | 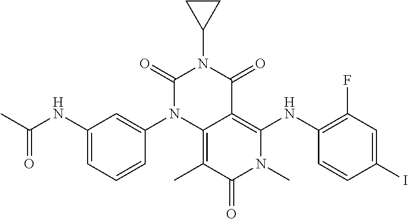 [SMILES: CC(=O)Nc1cccc(-n2c(=O)n(C3CC3)c(=O)c3c(Nc4ccc(I)cc4F)n(C)c(=O)c(C)c32)c1] |
| osimertinib | 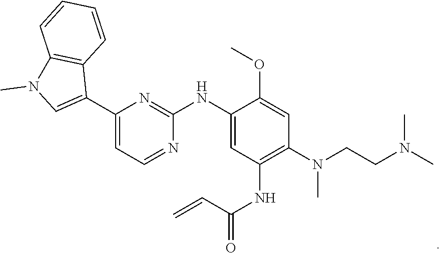 |

In some embodiments, the subject will be administered a premedication prior to each dose (e.g., prior to the start of infusion) of expression repressor. In certain embodiments, the premedication is provided at least 60 minutes prior to the start of the infusion. In some embodiments, the premedication is selected from one or more of oral acetaminophen (e.g., 500 mg), intravenous H1 blocker (e.g., diphenhydramine 50 mg or equivalent), and intravenous corticosteroids (e.g., dexamethasone 10 mg or equivalent). In some embodiments, the intravenous corticosteroid (e.g., dexamethasone 10 mg or equivalent) is provided at least 60 minutes prior to the start of the infusion.

In some embodiments, when bicistronic ZF9-MQ1_ZF3-KRAB and therapeutic agents are both administered, the therapeutic agent will be administered at least 1 hour, and up to 24 hours, after completion of the bicistronic ZF9-MQ1_ZF3-KRAB infusion.

In some embodiments, when bicistronic ZF9-MQ1_ZF3-KRAB and lenvatinib are both administered on the same day, the lenvatinib will be administered at least 1 hour, and up to 24 hours, after completion of the bicistronic ZF9-MQ1_ZF3-KRAB infusion.

In certain embodiments, the therapeutic agent comprises lenvatinib and is administered according to a dosing regimen comprising a defined dose administered over a defined period periodically at defined intervals.

The dose may be within a defined range. For example, the dose may be at about 8 mg to about 12 mg or about 8 mg to about 24 mg.

The dose may be a defined value. For example, the dose may be about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, or about 24 mg.

The dose may be a defined value based on a subject's actual body weight. For example, the dose may be about 8 mg wherein the subject's actual body weight is <60 kg. In some embodiments, the dose may be 12 mg wherein the subject's actual body weight is ≥60 kg.

The dose may be administered at defined intervals. For example, the interval between doses may be about 1 day, about 2 days, about 3 days, or about 4 days.

In certain embodiments, the therapeutic agent comprises lenvatinib and is administered at about 8 mg to about 12 mg (e.g., about 8 mg, about 9 mg, about 10 mg, about 11 mg, or about 12 mg) once daily. In certain embodiments, therapeutic agent comprises lenvatinib and is administered at about 8 mg wherein the subject is <60 kg. In some embodiments, the therapeutic agent comprises lenvatinib and is administered at 8 mg once daily wherein the subject is <60 kg. In some embodiments, the therapeutic agent comprises lenvatinib and is administered at about 12 mg, wherein the subject is ≥60 kg. In some embodiments, the therapeutic agent comprises lenvatinib and is administered at 12 mg once daily wherein the subject is ≥60 kg.

In some embodiments, the therapeutic agent comprises lenvatinib and is administered at about 18 mg once daily. In certain embodiments, the therapeutic agent comprises lenvatinib and is administered at about 20 mg once daily. In one embodiment, the therapeutic agent comprises lenvatinib and is administered at about is administered at about 24 mg once daily.

In some embodiments, when a subject receiving lenvatinib experiences a grade 1 or tolerable grade 2 non-hematologic toxicity, the method of administration of lenvatinib comprises continuing administration without dose modification. In some embodiments, grade 2 toxicities will be determined to be tolerable or intolerable by both the participant and Investigator. If grade 2 toxicity is determined to be intolerable, the dose of study drug will be reduced with or without dose interruption.

In certain embodiments, when a subject receiving lenvatinib experiences a grade 2 or grade 3 non-hematologic toxicity (e.g., a first occurrence), the method of administration of lenvatinib comprises stopping administration until the grade 2 or grade 3 non-hematologic toxicity is resolved to grade 0-1, or baseline. In some embodiments, when a subject receiving lenvatinib experiences a grade 2 or grade 3 non-hematologic toxicity, the method of administration of lenvatinib comprises resuming administration of lenvatinib at a reduced dose level (e.g., 1 reduced dose level) upon resolution of the grade 2 or grade 3 non-hematologic toxicity to grade 0-1 or baseline. In certain embodiments, when a subject receiving lenvatinib experiences a grade 2 or grade 3 non-hematologic toxicity (e.g., a second or third occurrence, e.g., the same toxicity or a new toxicity), the method of administration of lenvatinib comprises stopping administration until the grade 2 or grade 3 non-hematologic toxicity is resolved to grade 0-1, or baseline. In some embodiments, when a subject receiving lenvatinib experiences a grade 2 or grade 3 non-hematologic toxicity (e.g., a second or third occurrence, e.g., the same toxicity or a new toxicity), the method of administration of lenvatinib comprises resuming administration of lenvatinib at a reduced dose level (e.g., reduced by 1 or more reduced dose levels) upon resolution of the grade 2 or grade 3 non-hematologic toxicity (e.g., a second or third occurrence, e.g., the same toxicity or a new toxicity) to grade 0-1 or baseline. In certain embodiments, when a subject receiving lenvatinib experiences a grade 2 or grade 3 non-hematologic toxicity (e.g., a fourth occurrence, e.g., the same toxicity or a new toxicity), the method of administration of lenvatinib comprises stopping administration until the grade 2 or grade 3 non-hematologic toxicity is resolved to grade 0-1, or baseline. In some embodiments, when a subject receiving lenvatinib experiences a grade 2 or grade 3 non-hematologic toxicity (e.g., a fourth occurrence, e.g., the same toxicity or a new toxicity), the method of administration of lenvatinib comprises resuming administration of lenvatinib at a reduced dose level (e.g., after consultation) upon resolution of the grade 2 or grade 3 non-hematologic toxicity (e.g., a fourth occurrence, e.g., the same toxicity or a new toxicity) to grade 0-1 or baseline. In some embodiments, grade 2 toxicities will be determined to be tolerable or intolerable by both the participant and Investigator. If grade 2 non-hematologic toxicity is determined to be intolerable, the dose of study drug will be reduced with or without dose interruption. Interruption for grade 3 toxicities is mandatory. In certain embodiments, obese subjects do not need to return to baseline or grade 1 weight loss to resume administration of lenvatinib. In some embodiments, a subject receiving lenvatinib should undergo no weight loss for at least 1 week, and subjects should be started at the lower dose and normal BMI should be used for future dose reductions.

In some embodiments, when a subject receiving lenvatinib experiences a grade 4 non-hematologic toxicity, the method of administration of lenvatinib comprises stopping administration. In certain embodiments, when a subject receiving lenvatinib experiences a grade 4 non-hematologic toxicity, the method of administration of lenvatinib comprises stopping administration, excluding non-life threatening laboratory abnormalities which are treated as grade 3 non-hematologic toxicity.

In some embodiments, when a subject receiving lenvatinib experiences a grade 1 or tolerable grade 2 hematologic toxicity or proteinuria, the method of administration of lenvatinib comprises continuing administration without dose modification. In some embodiments, grade 2 toxicities will be determined to be tolerable or intolerable by both the participant and Investigator. If grade 2 toxicity is determined to be intolerable, the dose of study drug will be reduced with or without dose interruption.

In certain embodiments, when a subject receiving lenvatinib experiences a grade 3 hematologic toxicity or proteinuria (e.g., first occurrence), the method of administration of lenvatinib comprises stopping administration until the hematologic toxicity or proteinuria resolved to grade 0-2 or baseline. In certain embodiments, when a subject receiving lenvatinib experiences a grade 3 hematologic toxicity or proteinuria (e.g., a second occurrence, third occurrence, or fourth occurrence, e.g., same toxicity or new toxicity), the method of administration of lenvatinib comprises stopping administration until the hematologic toxicity or proteinuria resolved to grade 0-2 or baseline, and reducing lenvatinib administration by 1 dose level upon resuming administration. In some embodiments, when a subject receiving lenvatinib experiences a grade 3 hematologic toxicity or proteinuria (e.g., a fourth occurrence, e.g., the same toxicity or new toxicity), the method of administration of lenvatinib comprises stopping administration and the subject receives a consultation prior to dose modification.

In some embodiments, when a subject receiving lenvatinib experiences a grade 4 hematologic toxicity or proteinuria (e.g., first occurrence), the method of administration of lenvatinib comprises stopping administration until the hematologic toxicity or proteinuria resolved to grade 0-2 or baseline. In certain embodiments, when a subject receiving lenvatinib experiences a grade 4 hematologic toxicity or proteinuria (e.g., a second occurrence, third occurrence, or fourth occurrence, e.g., same toxicity or new toxicity), the method of administration of lenvatinib comprises stopping administration until the hematologic toxicity or proteinuria resolved to grade 0-2 or baseline, and reducing lenvatinib administration by 1 dose level upon resuming administration. In some embodiments, when a subject receiving lenvatinib experiences a grade 4 hematologic toxicity or proteinuria (e.g., a fourth occurrence, e.g., the same toxicity or new toxicity), the method of administration of lenvatinib comprises stopping administration and the subject receives a consultation prior to dose modification.

In some embodiments, when a subject experiences a grade 2 serum creatinine (e.g., >1.5-3.0× baseline; >1.5-3.0×ULN), the method of administration of lenvatinib comprises stopping administration. In certain embodiments, when a subject experiences a grade 2 serum creatinine (e.g., >1.5-3.0× baseline; >1.5-3.0×ULN), the method of administration of lenvatinib comprises resuming administration at the next lower dose upon resolution to ≤grade 1. In certain embodiments, when a subject experiences a grade 2 serum creatinine (e.g., >1.5-3.0× baseline; >1.5-3.0×ULN), the method of administration of the lenvatinib comprises stopping administration permanently if the grade 2 serum creatinine does not resolve to grade 1.

In some embodiments, the adverse event grade is not applicable to abnormal clinical laboratory values that are not clinically relevant, e.g., based on the judgment of one of skill in the art (e.g., alanine aminotransferase (ALT), aspartate aminotransferase (AST), γ-glutamyl transferase (γ-GTP) values <10× upper limit of normal (ULN), and sodium (Na)). In certain embodiments, when a subject receiving lenvatinib experiences a ≥grade 3 elevations of amylase and lipase, the subject receives consultation prior to continuing lenvatinib administration.

In some embodiments, a method of treatment described herein comprises imaging a tumor of the subject. In some embodiments, a computed tomography (CT) scan, e.g., with contrast or magnetic resonance imaging (MRI), is performed to image a tumor of the subject. A CT scan may be performed, e.g., every 6 weeks (±1 week) or every 3 months (±3 weeks). In some embodiments, a subject's response is assessed according to modified Response Evaluation Criteria in Solid Tumors (mRECIST) for HCC or by RECIST 1.1 for a non-HCC solid tumor. In some embodiments, a subject is considered to have achieved a complete response (CR) based on 2 tumor imaging assessments conducted at least 4 weeks apart.

In some embodiments, a method of treatment described herein comprises determining the incidence or severity of adverse events (including SAEs), laboratory abnormalities, electrocardiogram (ECG) changes, cytokines, C-reactive protein (CRP), and complement. In some embodiments, a method of treatment described herein comprises collecting plasma samples, blood samples, or tumor biopsy samples.

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein a modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to hepatic, epithelial, connective, muscular, reproductive, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof.

In some embodiments, a pharmaceutical composition as provided herein is administered systemically.

In some embodiments, administration is non-parenteral and a therapeutic is a parenteral therapeutic.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

Pharmaceutical uses of the present disclosure may include compositions (e.g., modulating agents, e.g., disrupting agents) as described herein.

In some embodiments, a pharmaceutical composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to an active agent alone. In some embodiments, a pharmaceutical composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to an active agent alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to an active agent alone.

Pharmaceutical compositions described herein may be formulated for example including a carrier, such as a pharmaceutical carrier and/or a polymeric carrier, e.g., a nanoparticle, a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. Doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

### Lipid Nanoparticles

Expression repressors or expression repression systems as described herein can be delivered using any biological delivery system/formulation including a particle, for example, a nanoparticle delivery system. Nanoparticles include particles with a dimension (e.g. diameter) between about 1 and about 1000 nanometers, between about 1 and about 500 nanometers in size, between about 1 and about 100 nm, between about 30 nm and about 200 nm, between about 50 nm and about 300 nm, between about 75 nm and about 200 nm, between about 100 nm and about 200 nm, and any range therebetween. A nanoparticle has a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In some embodiments, nanoparticles have a greatest dimension ranging between 25 nm and 200 nm. Nanoparticles as described herein comprise delivery systems that may be provided in any form, including but not limited to solid, semi-solid, emulsion, or colloidal nanoparticles. A nanoparticle delivery system may include but not limited to lipid-based systems, liposomes, micelles, micro-vesicles, exosomes, or gene gun. In one embodiment, the nanoparticle is a lipid nanoparticle (LNP). In some embodiments, the LNP is a particle that comprises a plurality of lipid molecules physically associated with each other by intermolecular forces.

In some embodiments, an LNP may comprise multiple components, e.g., 3-4 components. In one embodiment, the expression repressor or a pharmaceutical composition comprising said expression repressor (or a nucleic acid encoding the same, or pharmaceutical composition comprising said expression repressor nucleic acid) is encapsulated in an LNP. In one embodiment, the expression repression system or a pharmaceutical composition comprising said expression repression system (or a nucleic acid encoding the same, or pharmaceutical composition comprising said expression repression system nucleic acid) is encapsulated in an LNP. In some embodiments, the nucleic acid encoding the first expression repressor and the nucleic acid encoding the second expression repressor are present in same LNP. In some embodiments, the nucleic acid encoding the first expression repressor and the nucleic acid encoding the second expression repressor are present in different LNPs. Preparation of LNPs and the modulating agent encapsulation may be used/and or adapted from Rosin et al, Molecular Therapy, vol. 19, no. 12, pages 1286-2200, December 2011). In some embodiments, lipid nanoparticle compositions disclosed herein are useful for expression of protein encoded by mRNA. In some embodiments, nucleic acids, when present in the lipid nanoparticles, are resistant in aqueous solution to degradation with a nuclease.

In some embodiments, the biomolecule (e.g., nucleic acid) in the LNP is in an aqueous environment. In some embodiments, the biomolecule (e.g., nucleic acid) in the LNP is in a hydrophobic environment.

In some embodiments, the LNP formulations may include a CCD lipid, a neutral lipid, and/or a helper lipid. In some embodiments, the LNP formulation comprises an ionizable lipid. In some embodiments, an ionizable lipid may be a cationic lipid, an ionizable cationic lipid, or an amine-containing lipid that can be readily protonated. In some embodiments, the lipid is a cationic lipid that can exist in a positively charged or neutral form depending on pH. In some embodiments, the cationic lipid is a lipid capable of being positively charged, e.g., under physiological conditions. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids.

In some embodiments, LNP formulation (e.g., MC3 and/or SSOP) includes cholesterol, PEG, and/or a helper lipid. The LNPs may be, e.g., microspheres (including unilamellar and multilamellar vesicles, lamellar phase lipid bilayers that, in some embodiments, are substantially spherical.

In some embodiments, the LNP can comprise an aqueous core, e.g., comprising a nucleic acid encoding an expression repressor or a system as disclosed herein. In some embodiments of the present disclosure, the cargo for the LNP formulation includes at least one guide RNA. In some embodiments, the cargo, e.g., a nucleic acid encoding an expression repressor, or a system as disclosed herein, may be adsorbed to the surface of an LNP, e.g., an LNP comprising a cationic lipid. In some embodiments, the cargo, e.g., a nucleic acid encoding an expression repressor, or a system as disclosed herein may be associated with the LNP. In some embodiments, the cargo, e.g., a nucleic acid encoding an expression repressor, or a system as disclosed herein, may be encapsulated, e.g., fully encapsulated and/or partially encapsulated in an LNP.

In some embodiments, an LNP comprising a cargo may be administered for systemic delivery, e.g., delivery of a therapeutically effective dose of cargo that can result in a broad exposure of an active agent within an organism. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery. In some embodiments, an LNP comprising a cargo may be administered for local delivery, e.g., delivery of an active agent directly to a target site within an organism. In some embodiments, an LNP may be locally delivered into a disease site, e.g., a tumor, other target site, e.g., a site of inflammation, or to a target organ, e.g., the liver, lung, stomach, colon, pancreas, uterus, breast, lymph nodes, and the like. In some embodiments, an LNP as disclosed herein may be locally delivered to a specific cell, e.g., hepatocytes, stellate cells, Kupffer cells, endothelial, alveolar, and/or epithelial cells. In some embodiments, an LNP as disclosed herein may be locally delivered to a specific tumor site, e.g., subcutaneous, orthotopic.

The LNPs may be formulated as a dispersed phase in an emulsion, micelles, or an internal phase in a suspension. In some embodiments, the LNPs are biodegradable. In some embodiments, the LNPs do not accumulate to cytotoxic levels or cause toxicity in vivo at a therapeutically effective dose. In some embodiments, the LNPs do not accumulate to cytotoxic levels or cause toxicity in vivo after repeat administrations at a therapeutically effective dose. In some embodiments, the LNPs do not cause an innate immune response that leads to a substantially adverse effect at a therapeutically effective dose.

In some embodiments, the LNP used, comprises the formula (6Z,9Z,28Z,31Z)-heptatriacont-6,9,28,31-tetraene-19-yl 4-(dimethylamino)butanoate or ssPalmO-phenyl-P4C2 (ssPalmO-Phe, SS-OP). In some embodiments, the LNP formulation comprises the formula, (6Z,9Z,28Z,31Z)-heptatriacont-6,9,28,31-tetraene-19-yl 4-(dimethylamino)butanoate (MC3), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., MC3 LNP or ssPalmO-phenyl-P4C2 (ssPalmO-Phe, SS-OP), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., SSOP-LNP.

Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. Doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. Doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

### Uses

The present disclosure is further directed to uses of the expression repressors or expression repressor systems disclosed herein. Among other things, in some embodiments such provided technologies may be used to achieve modulation, e.g., repression, of target gene, e.g., MYC expression and, for example, enable control of target gene, e.g., MYC activity, delivery, and penetrance, e.g., in a cell. In some embodiments, a cell is a mammalian, e.g., human, cell. In some embodiments, a cell is a somatic cell. In some embodiments, a cell is a primary cell. For example, in some embodiments, a cell is a mammalian somatic cell. In some embodiments, a mammalian somatic cell is a primary cell. In some embodiments, a mammalian somatic cell is a non-embryonic cell.

In some embodiments, the expression repressors or expression repressor systems disclosed herein can be used to treat cancer in a subject in need thereof. In some embodiments, the cancer is hepatocellular carcinoma (HCC), Fibrolamellar Hepatocellular Carcinoma (FHCC), cholangiocarcinoma, Angiosarcoma, secondary liver cancer, non-small cell lung cancer (NSCLC), adenocarcinoma, small cell lung cancer (SCLC), large cell (undifferentiated) carcinoma, triple negative breast cancer, gastric adenocarcinoma, endometrial carcinoma, or pancreatic carcinoma. In some embodiments, the cancer is metastatic, advanced (non-resectable, or recurrent solid tumor types known for association with the MYC oncogene (e.g., including, but not limited to, hepatocellular carcinoma (HCC), bladder, lung, pancreatic, ovarian, uterine, endometrial, gastric, esophageal, hepatobiliary, colorectal cancer, and neuroblastoma), which has progressed on, relapsed after, are refractory to, or intolerant of standard of care or no alternative treatment exists.

In some embodiments, the subject having a cancer (e.g., a cancer described herein), has progressed on, relapsed after, is refractory to, or intolerant of, at least 1 prior systemic therapy. In certain embodiments, there is no available standard of care for the subject.

In certain embodiments, the subject has at least 1 measurable lesion according to RECIST 1.1.

In some embodiments, the subject comprises an Eastern Cooperative Oncology Group (ECOG) performance status of 0 or 1, wherein Grade 0=Fully active, able to carry on all pre-disease performance without restriction; Grade 1=Restricted in physically strenuous activity but ambulatory and able to carry out work of a light or sedentary nature, e.g., light housework or office work; Grade 2=Ambulatory and capable of all self-care but unable to carry out any work activities, and/or up and about more than 50% of waking hours; Grade 3=Capable of only limited self-care, and/or confined to bed or chair more than 50% of waking hours; Grade 4=Completely disabled, and/or cannot carry on any self-care, and/or totally confined to bed or chair; Grade 5=deceased.

In some embodiments, the subject has a predicted life expectancy of less than 3 months (e.g., less than 2 months, less than 1 month, less than 25 days, less than 20 days, less than 10 days).

In some embodiments, the subject has not received chemotherapy or immunotherapy, had surgery, or received locoregional therapy (TACE, TAE, HAI, radiation, radioembolization or ablation), in at least 28 days. In certain embodiments, the subject has recovered from any toxicity associated with prior treatments. In some embodiments, the subject has recovered from any toxicities related to prior therapies to ≤Grade 1 or baseline.

In certain embodiments, the subject has, or has been identified as having, chronic hepatitis B and must have received antiviral therapy for hepatitis B virus (HBV) for at least 12 weeks and HBV viral load must be <500 IU/mL prior to first dose of study drug. In some embodiment, subjects on active HBV therapy with viral loads <500 IU/mL are to stay on the same therapy throughout treatment.

In some embodiments, subjects having, or identified as having, hepatitis C virus (HCV) have not received anti-hepatitis C therapy. IN certain embodiments, subject having, or identified as having, HCV have received anti-hepatitis C therapy but have not reached seroconversion.

In certain embodiments, subject having, or identified as having, an HBV/HCV coinfection must fulfill treatment and viral load requirements for hepatitis B (e.g., must have received antiviral therapy for hepatitis B virus (HBV) for at least 12 weeks and HBV viral load must be <500 IU/mL prior to first dose of study drug).

In some embodiments, the subject has adequate organ function. In some embodiments, adequate organ function includes, but is not limited to:
a. Absolute neutrophil count (ANC) ≥1500/mm3;
b. Hemoglobin ≥8.5 g/dL without transfusion or erythropoietin within 7 days of first dose of study drug;
c. Platelet count ≥75,000/mm3 without transfusion;
d. Total bilirubin ≤1.5 mg/dL and/or ≤2.0 mg/dL;
e. AST and ALT ≤3×ULN and/or ≤5×ULN;
f. International normalized ratio (INR)≤1.5;
g. Estimated glomerular filtration rate (eGFR) ≥30 mL/min/1.73 m2 by Modification of Diet in Renal Disease (MDRD) equation (e.g., GFR (mL/min/1.73 m2)=175×(Scr)− 1.154×(Age)−0.203×(0.742 if female)×(1.212 if African American) (Levey et al. A new equation to estimate glomerular filtration rate, Ann Intern Med, 150: 604-12 (2009))).

In some embodiments, the subject has HCC. In certain embodiments, the HCC has been confirmed radiographically, histologically, or cytologically. In some embodiments, the HCC is BCLC Stage B (intermediate state) or C (advanced stage) that is not amenable to locoregional therapy, refractory to locoregional therapy, and/or not amenable to curative treatment. In certain embodiments, the subject has neurotrophic tyrosine receptor kinase (NTRK) fusion-positive HCC. In some embodiments, the subject having NTRK-HCC has progressed on, relapsed after, is refractory to, or intolerant of NTRK-directed therapy. In certain embodiments, the subject has hepatic cirrhosis (e.g., underlying hepatic cirrhosis). In some embodiments, the hepatic cirrhosis (e.g., underlying hepatic cirrhosis) is well compensated (e.g., C-P Class A: Score 5-6). In certain embodiments, the subject has no evidence of hepatic encephalopathy (e.g., confirmed within 7 days of administration of the expression repressor or expression repressor system.

In some embodiments, the subject has received at least one, but not more than 3, prior systemic therapies for HCC, without available subsequent standard of care. In certain embodiments, subjects with known NTRK fusion-positive.

In some embodiments, the subject has total triiodothyronine (T3) or free T3, and free thyroxine (T4) within normal limits. In some embodiments, the subject has preexisting hypothyroidism that is well controlled with thyroid replacement therapy.

In some embodiments, a subject does not have one or more conditions excluding the subject from treatment using the expression repressors or expression repressor systems described herein. In certain embodiment, the subject does not have a history of central nervous system (CNS) metastases or carcinomatous meningitis. In certain embodiments, the subject does not have a history of CNS metastases or carcinomatous meningitis. In certain embodiments, the subject has a history of CNS metastases or carcinomatous meningitis and have completed local therapy and discontinued the use of corticosteroids at least 4 weeks (e.g., at least 4 weeks, at least 5 weeks, at least 6 weeks, or more) prior to administration of the expression repressor or expression repressor systems described herein. In some embodiment, the subject does not have a history of other malignancies within the last 3 years (e.g., within the last 2.5 years, within the last 2 years, within the last 1.5 years, within the last 1 year, or within the last 0.5 years).

In some embodiments, the subject does not have a history of non-melanoma skin cancer (squamous or basal cell), carcinoma in situ of the breast or cervix. In some embodiments, the subject has a history of non-melanoma skin cancer (squamous or basal cell), carcinoma in situ of the breast or cervix, which has been adequately treated.

In some embodiments, the subject is receiving anticancer hormonal therapy for a previously treated non-HCC cancer.

In certain embodiments, the subject does not have an active, uncontrolled bacterial, viral, or fungal infection requiring systemic therapy, or an active COVID-19 infection within 14 days prior to administration of a first dose of an expression repressor or expression repressor system described herein. In certain embodiments, the subject has not tested positive for SARS-CoV2 within 14 days prior to administration of a first dose of an expression repressor or expression repressor system described herein.

In some embodiments, the subject does not have, or has not been identified as having, human immunodeficiency virus (HIV). In certain embodiments, the subject does not have a history of prior solid organ transplantation. In certain embodiments, the subject does not have a history of hematopoietic stem cell transplantation (HSCT).

In certain embodiments, the subject does not have a history of congestive heart failure >Class II New York Heart Association (NYHA), active coronary artery disease (myocardial infarction, unstable angina, coronary artery bypass graft [CABG] or percutaneous transluminal coronary angioplasty [PTCA]) within the past 6 months, ventricular arrhythmias requiring treatment, uncontrolled hypertension (systolic blood pressure >160 mmHg and/or diastolic blood pressure >100 mmHg), or history of hypertensive crisis. In certain embodiments, the subject does not have a QTc interval of >450 ms (male) or >470 ms (female). In certain embodiments, the subject has a QTc interval of ≤450 ms (male) or ≤470 ms (female).

In certain embodiments, the subject has not received prior MYC-targeting therapy (e.g., MYC promotor bromodomain inhibitors, MYC mRNA-degrading therapies, MC proteasomal degradation-targeting therapies, and therapies specifically inhibiting the transcription, translation, or biosynthesis of MYC).

In certain embodiments, the subject has, or is identified as having, HCC, and the subject does not have, or has been identified as not having, a) mixed histology cholangiocarcinoma and HCC or fibrolamellar variant HCC; b) imaging finding with one or more of (e.g., one, two, or all of): i) hepatocellular carcinoma with ≥50% liver occupation, ii) clear invasion into the bile duct, iii) portal vein invasion with Vp4 (e.g., thrombus involving the main trunk of the portal vein); c) clinically apparent ascites on physical exam; d) history of ascites requiring paracentesis within the past 3 months; e) esophageal or gastric variceal bleeding in the past 3 months; f) history of hepatic encephalopathy in the past 3 months. In certain embodiments, the subject does not receive or take rifaximin or lactulose to control hepatic encephalopathy.

In some embodiments, the subject has not received live vaccine within 30 days of administration of the first dose of the expression repressor or expression repressor system described herein. In some embodiments, the subject has not received an mRNA vaccine within 14 days of administration of the first dose of the expression repressor or expression repressor system described herein.

In certain embodiments, the subject does not have an active autoimmune disease requiring systemic treatment. In certain embodiments, subject have, or are identified as having, autoimmune hypothyroidism, diabetes mellitus, pituitary or adrenal insufficiency that requires replacement therapy may be enrolled.

In some embodiments, the subject does not have a history of allergic reactions/hypersensitivity to monoclonal antibodies that resulted in discontinuation of the therapy.

In some embodiments, the subject has not previously received lenvatinib. In certain embodiments, the subject does not have, or has not been identified as having, a hypersensitivity to any component of lenvatinib. In some embodiments, the subject does not have gastrointestinal malabsorption or surgical resection that may affect absorption, e.g., absorption of lenvatinib. In certain embodiments, the subject has not received treatment with a strong CYP3A4 inducer(s) within 14 days prior to the first dose of the expression repressors or expression repressor systems described herein. In certain embodiments, the subject has not received (e.g., within 10 days prior to the first dose of the expression repressors or expression repressor systems described herein) or is not currently receiving use of full dose anticoagulants for therapeutic purpose. In certain embodiments, the subject may be receiving prophylactic anticoagulation (e.g., for potency of venous access) provided subjects international normalized ratio (INR)≤2.0. In certain embodiments, the subject has not recently received (e.g., between 1 day to 28 days) or concurrently receiving an anti-platelet drug. In some embodiments, the subject does not have a non-healing wound or ulcer.

In some embodiments, the subject has not previously received sorafenib. In certain embodiments, the subject does not have, or has not been identified as having, a hypersensitivity to any component of sorafenib. In some embodiments, the subject does not have gastrointestinal malabsorption or surgical resection that may affect absorption, e.g., absorption of sorafenib. In certain embodiments, the subject has not received treatment with a strong CYP3A4 inducer(s) within 14 days prior to the first dose of the expression repressors or expression repressor systems described herein. In certain embodiments, the subject has not received (e.g., within 10 days prior to the first dose of the expression repressors or expression repressor systems described herein) or is not currently receiving use of full dose anticoagulants for therapeutic purpose. In certain embodiments, the subject may be receiving prophylactic anticoagulation (e.g., for potency of venous access) provided subjects international normalized ratio (INR)≤2.0. In certain embodiments, the subject has not recently received (e.g., between 1 day to 28 days) or concurrently receiving an anti-platelet drug. In some embodiments, the subject does not have a non-healing wound or ulcer.

In certain embodiments, the subject receiving the therapy described herein does not experience one or more of:
i) Any Grade 5 hematologic adverse event (AE);
ii) Grade 4 neutropenia and thrombocytopenia ≥7 days;
iii) Grade 3+ febrile neutropenia lasting more than 48 hours; or
iv) Grade 3+ thrombocytopenia associated with bleeding/hemorrhage that require transfusion.

In some embodiments, the subject receiving the therapy described herein does not experience one or more of:
i) Grade 2 eye pain or reduction of visual acuity that does not improve to Grade 1 within 7 days after giving topical therapy or requires systemic treatment;
ii) Grade 2 pneumonitis that does not improve to Grade 1 within 7 days after giving corticosteroids;
iii) Grade 2 myocarditis;
iv) Any Grade 4 or Grade 5 non-hematologic AE;
v) Grade 3 non-laboratory value toxicity lasting >72 hours (excluding nausea, vomiting, diarrhea, and/or dermatological toxicities controlled by medical intervention, fatigue, fever, or chills);
vi) Grade 3 hypertension not controlled by medication;
vii) Grade 2 gastrointestinal (GI) tract perforation;
viii) Grade 3 wound dehiscence that requires surgical intervention;
ix) Grade 3 thromboembolic event;
x) Any Grade 3 non-hematologic laboratory value that requires medical intervention or hospitalization;
xi) Aspartate aminotransferase (AST)/alanine aminotransferase (ALT) >10× upper limit of normal (ULN); or
xii) Grade 3 hepatic failure.

In some embodiments, a subject receiving a therapy described herein receives one or more of: 1) a tumor assessments before the beginning of the therapy, 2) every 6 weeks (±1 week), e.g., for the first year, and 3) every 3 months (±3 weeks), e.g., beginning 1 year after the start of the treatment until disease progression.

In certain embodiments, a subject receiving, or expected to receive, a therapy described herein is analyzed for PK/PD, e.g., pharmacokinetics or pharmacodynamics, of the therapy. In some embodiments, the subject has a baseline AFP level of <400 ng/mL. In certain embodiments, the subject has a baseline AFP level of ≥400 ng/mL.

In some embodiments, the subject receiving, or expected to receive, a therapy described herein has, or is identified as having, HCC caused by HBV. In some embodiments, the subject receiving, or expected to receive, a therapy described herein has, or is identified as having, HCC caused by HCV.

In some embodiments, after treatment with a therapy described herein, the subject experiences an improvement in score on the European Organisation for Research and Treatment of Cancer (EORTC) QLC-C30 or EORTC QLQ-HCC18.

In some embodiments, the subject does not produce a detectable level of anti-drug antibodies.

### Combination Therapies

The present disclosure provides, for instance, combination therapies comprising an expression repressor described herein and a kinase inhibitor.

In some aspects, the present disclosure provides a method of treating a cancer in a subject in need thereof, the method comprising administering to the subject:
(1) a nucleic acid (e.g., RNA, e.g., mRNA) encoding an expression repressor described herein, and
(2) a compound having the general structure of Formula (I):

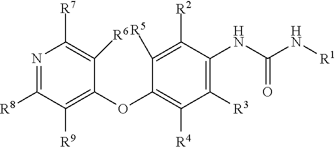

wherein:
R¹ is alkyl, cycloalkyl, or aryl;
R² is halo, alkyl, or H;
R³, R⁴, R⁵, R⁶, R⁷, are each independently H or alkyl;
R⁸ and R⁹ together form an optionally substituted aromatic ring;
   or a pharmaceutically acceptable salt, tautomer or stereoisomer thereof.

In some aspects, the present disclosure provides a method of treating a cancer in a subject in need thereof, the method comprising administering to the subject:
(1) a nucleic acid (e.g., RNA, e.g., mRNA) encoding an expression repressor described herein, and
(2) a compound having the general structure of Formula (I):

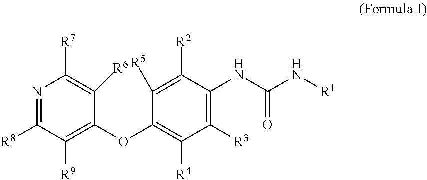

wherein:
R¹ is alkyl or cycloalkyl;
R² is halo, alkyl, or H;
R³, R⁴, R⁵, R⁶, R⁷, are each independently Hs or alkyl;
R⁸ and R⁹ together form an optionally substituted aromatic ring, or R⁸=CH₃NHCO— and R⁹=H; or a pharmaceutically acceptable salt, tautomer or stereoisomer thereof.

In some aspects, the present disclosure provides a method of treating a cancer in a subject in need thereof, the method comprising administering to the subject:
(1) a nucleic acid (e.g., RNA, e.g., mRNA) encoding an expression repressor described herein, and
(2) a compound having the general structure of Formula (I):

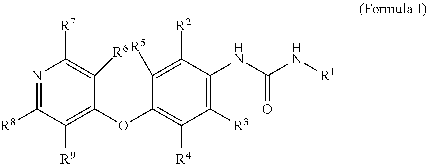

wherein:
R¹ is alkyl, cycloalkyl, or aryl;
R² is halo;
R³, R⁴, R⁵, R⁶, R⁷, are each independently Hs or alkyl;
R⁸ and R⁹ together form an optionally substituted aromatic ring, or R⁸=CH₃NHCO— and R⁹=H;
   or a pharmaceutically acceptable salt, tautomer or stereoisomer thereof.

In some embodiments, R¹ is alkyl or cycloalkyl, In some embodiments, R¹=cyclopropane.

In some embodiments, R² is halo. In some embodiments, R²=Cl.

In some embodiments, R³, R⁴, R⁵, R⁶, R⁷, are each H.

In some embodiments, the substituted aromatic ring is a substituted phenyl ring.

In some embodiments, the compound of Formula (I) comprises:

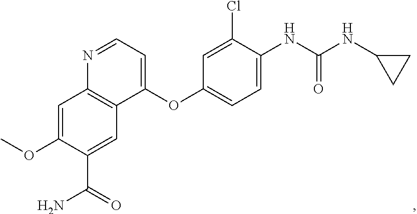 [SMILES: COc1cc2nccc(Oc3ccc(NC(=O)NC4CC4)c(Cl)c3)c2cc1C(N)=O]

or a pharmaceutically acceptable salt, tautomer or stereoisomer thereof.

In some embodiments, the compound of Formula (I) comprises lenvatinib.

In some embodiments, the compound of Formula (I) is administered orally. In some embodiments, the compound of Formula (I) is administered at a dose of 6-10 (e.g., about 8) or 10-14 (e.g., about 12) mg per day.

In some embodiments, the compound of Formula (I) specifically inhibits kinase activity of VEGFR1-3.

In some embodiments, administration of the combination therapy results in decreased vascular endothelial proliferation in a tumor of the subject.

In some embodiments, cancer cells of the subject are capable of undergoing apoptosis. In some embodiments, cancer cells of the subject have a functional apoptosis pathway.

In some embodiments, the compound of Formula (I) is administered prior to the nucleic acid encoding the expression repressor. In some embodiments, the compound of Formula (I) is administered after the nucleic acid encoding the expression repressor. In some embodiments, the compound of Formula (I) is administered concurrently with the nucleic acid encoding the expression repressor.

In some aspects, the present disclosure provides a method of treating a cancer in a subject in need thereof, the method comprising administering to the subject:
(1) a nucleic acid (e.g., RNA, e.g., mRNA) encoding an expression repressor described herein, and
(2) a compound having the general structure of Formula (II):

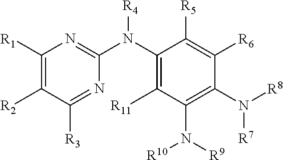

wherein R₁ is:

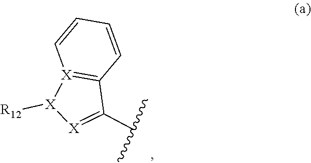

wherein X=C or N, given that one or two Xs are Ns; and R₁₂ is an electron pair, H or methyl;
   (b) halogen; or
   (c) methyl halogen;
R₂ is H or methyl;
R₃ is H or methyl;
R₄ is H or methyl;
R₅ is —OH or —O-methyl;
R₆ is H or methyl;
R₇ is H or methyl;
R₈ is —(CH₂)ₙ—N(R₁₃)₂, wherein n=2-6, and R₁₃=H or methyl;
R₉ is H or methyl;
R₁₀ is —(CO)—R₁₄, wherein R₁₄ is alkyl or alkene; and
R₁₁ is H or methyl,
or a pharmaceutically acceptable salt, tautomer, or stereoisomer thereof.

In certain embodiments, R₁ of Formula (II) is:

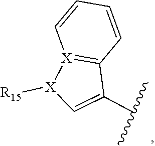

wherein X=C or N, given that one or two Xs are N; R₁₅ is an electron pair, H or methyl.

In some embodiments, R₁ of Formula (II) is:

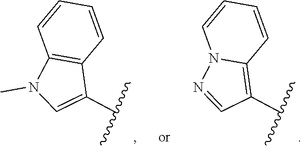 [SMILES: *c1cn(C)c2ccccc12 ; *c1cnn2ccccc12]

In certain embodiments, R₁ of Formula (II) is:

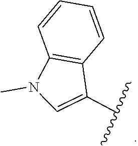 [SMILES: *c1cn(C)c2ccccc12]

In some embodiments, the compound is a compound having the general structure of Formula (II):

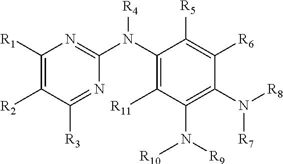

wherein:
R₂ is H;
R₃ is H;
R₄ is H;
R₅ is —O-methyl;
R₆ is H;
R₇ is methyl;
R₈ is —(CH₂)₂—N(methyl)₂, wherein n=2-6, and R₁₃=H or methyl;
R₉ is H;
R₁₀ is —(CO)-ethylene; and
R₁₁ is H.

In some embodiments, R₂ of Formula (II) is H. In some embodiments, R₃ of Formula (II) is H. In certain embodiments, R₄ of Formula (II) is H. In certain embodiments, R₅ of Formula (II) is —O-methyl. In some embodiments, R₆ of Formula (II) is H. In certain embodiments, R₇ of Formula (II) is methyl. In certain embodiments, R₈ of Formula (II) is —(CH₂)₂—N(methyl)₂, wherein n=2-6, and R₁₃=H or methyl. In some embodiments, R₉ of Formula (II) is H. In certain embodiments, R₁₀ of Formula (II) is —(CO)-ethylene. In certain embodiments, R₁₁ of Formula (II) is H.

In some aspects, the present disclosure provides a method of treating a cancer in a subject in need thereof, the method comprising administering to the subject:
(1) a nucleic acid (e.g., RNA, e.g., mRNA) encoding an expression repressor described herein, and
(2) a compound having the general structure of Formula (II):

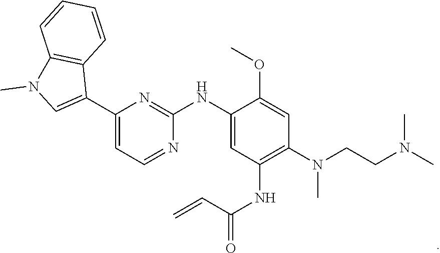

In some embodiments, the compound of Formula (II) comprises osimertinib.

In some embodiments, the compound of Formula (II) is administered orally. In some embodiments, the compound of Formula (II) is administered at a dose of 40-100 (e.g., about 80) mg per day. In some embodiments, the compound of Formula (II) is dispersed in 40-80 mL (e.g., about 60 mL) of non-carbonated water prior to oral administration.

In some embodiments, the kinase inhibitor comprises VEGFR1-3 inhibitor. In certain embodiments, VEGFR1-3 inhibitor comprises lenvatinib (CAS Registry Number: 417716-92-8). Alternative names for lenvatinib are LENVIMA®, E-7080, and ER-203492-00.

In some embodiments, the kinase inhibitor comprises a tyrosine kinase inhibitor (TKI). In certain embodiments, the TKI is sorafenib.

In some embodiments, the kinase inhibitor comprises an ERK inhibitor. In certain embodiments, the ERK inhibitor comprises ulixertinib (CAS Registry Number: 1956366-10-1). Alternative names for Ulixertinib are BVD-523 and VRT-72271.

In some embodiments, the kinase inhibitor comprises an AKT inhibitor. In certain embodiments, the AKT inhibitor is an allosteric AKT inhibitor. In certain embodiments, the AKT inhibitor is selected from one or more of MK-2206, ARQ092, or TAS-117. In some embodiments, the AKT inhibitor comprises MK-2206 (CAS Registry Number: 1032350-13-2). In certain embodiments, the AKT inhibitor is ARQ092, alternatively known as Miransertib (CAS Registry Number: 1313881-70-7). In some embodiments, the AKT inhibitor is TAS-117 (CAS Registry Number: 1402602-94-1).

In some embodiments, the kinase inhibitor comprises a MEK inhibitor. In certain embodiments, the MEK inhibitor comprises Trametinib (CAS Registry Number: 871700-17-3). Alternative names for Trametinib are GSK1120212 and JTP-74057. In certain embodiments, the MEK inhibitor comprises Cobimetinib (CAS Registry Number: 934660-93-2). Alternative names for Cobimetinib are GDC-0973, RG-7420, and XL518.

In certain embodiments, the kinase inhibitor is an epidermal growth factor receptor (EGFR) tyrosine kinase inhibitor. In some embodiments, the EGFR inhibitor is selected from one or more of erlotinib, afatinib, gefitinib, and osimertinib. In certain embodiments, the EGFR TKI is osimertinib (CAS Registry Number: 1421373-65-0). Alternative names for osimertinib are AZD9291 and mereletinib. In some embodiments, the EGFR inhibitor is erlotinib (CAS Registry Number: 183321-74-6). An alternative name for erlotinib is NSC 718781. In some embodiments, the EGFR inhibitor is afatinib (CAS Registry Number: 850140-72-6). An alternative name for afatinib is BIBW 2992. In some embodiments, the EGFR inhibitor is gefitinib (CAS Registry Number: 184475-35-2).

### Modulating Gene Expression

The present disclosure is further directed, in part, to a method of modulating, e.g., decreasing, expression of a target gene, e.g., MYC, comprising providing an expression repressor (or a nucleic acid encoding the same, or pharmaceutical composition comprising said expression repressor nucleic acid) or an expression repression system described herein (or a nucleic acid encoding the same, or pharmaceutical composition comprising said expression repression system or nucleic acid), and contacting the target gene e.g., MYC, and/or operably linked transcription control element(s) with the expression repressor or the expression repression system. In some embodiments, modulating, e.g., decreasing expression of a target gene, e.g., MYC comprises modulation of transcription of a target gene, e.g., MYC as compared with a reference value, e.g., transcription of a target gene, e.g., MYC in absence of the expression repressor or the expression repression system. In some embodiments, the method of modulating, e.g., decreasing, expression of a target gene, e.g., MYC are used ex vivo, e.g., on a cell from a subject, e.g., a mammalian subject, e.g., a human subject. In some embodiments, the method of modulating, e.g., decreasing, expression of a target gene, e.g., MYC are used in vivo, e.g., on a mammalian subject, e.g., a human subject. In some embodiments, the method of modulating, e.g., decreasing, expression of a target gene, e.g., MYC are used in vitro, e.g., on a cell or cell line described herein.

The present disclosure is further directed, in part to a method of treating a condition associated with mis-regulation, e.g., over-expression of a target gene, e.g., MYC in a subject, comprising administering to the subject an expression repressor (or a nucleic acid encoding the same, or pharmaceutical composition comprising said expression repressor nucleic acid) or an expression repression system described herein (or a nucleic acid encoding the same, or pharmaceutical composition comprising said expression repression system or nucleic acid). Conditions associated with over-expression of particular genes are known to those of skill in the art. Such conditions include, but are not limited to, metabolic disorders, cancer (e.g., solid tumors), and hepatitis.

Methods and compositions as provided herein may treat a condition associated with over-expression or mis-regulation of a target gene, e.g., MYC by stably or transiently altering (e.g., decreasing) transcription of a target gene, e.g., MYC. In some embodiments, such a modulation persists for at least about 1 hour to about 30 days, or at least about 2 hours, 6 hours, 12 hours, 18 hours, 24 hours, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., permanently or indefinitely). Optionally, such a modulation persists for no more than 10, 9, 8, 7, 6, 5, 4, 3, 2, or 1 years.

In some embodiments, a method or composition provided herein may decrease expression of a target gene, e.g., MYC in a cell by at least 10, 20, 30, 40, 50, 60, 70, 80, 90, or 100% (and optionally up to 100%) relative to expression of the target gene in a cell not contacted by the composition or treated with the method.

In some embodiments, a method provided herein may modulate, e.g., decrease, expression of a target gene, e.g., MYC by disrupting a genomic complex, e.g., an anchor sequence-mediated conjunction, associated with said target gene.

In some embodiments, modulating expression of a gene, e.g., MYC comprises altering accessibility of a transcriptional control sequence to a gene, e.g., MYC. A transcriptional control sequence, whether internal or external to an anchor sequence-mediated conjunction, can be an enhancing sequence or a silencing (or repressive) sequence.

In some embodiments, such provided technologies may be used to treat a gene mis-regulation disorder e.g., MYC gene mis-regulation disorder e.g., a symptom associated with a MYC gene mis-regulation in a subject, e.g., a patient, in need thereof. In some embodiments, such provided technologies may be used to treat a MYC gene mis-regulation disorder or a symptom associated with a MYC gene mis-regulation disorder in a subject, e.g., a patient, in need thereof. In some embodiments, the disorder is associated with MYC mis-regulation, e.g., MYC overexpression. In some embodiments, the disorder is associated with AFP mis-regulation, e.g., AFP overexpression. In some embodiments, such provided technologies may be used to methylate the promoter of a target gene, e.g., MYC, to treat a gene mis-regulation disorder e.g., MYC gene mis-regulation disorder, e.g., a symptom associated with a MYC gene mis-regulation in a subject, e.g., a patient, in need thereof. In some embodiments, such provided technologies may selectively affect the viability of a cell which aberrantly expresses a polypeptide encoded by a target gene, e.g., MYC.

In some embodiments, such provided technologies may be used to treat a hepatic disorder or a disorder e.g. a symptom associated with a hepatic disorder in a subject, e.g., a patient, in need thereof. In some embodiments, such provided technologies may be used to treat a pulmonary disorder or a disorder e.g. a symptom associated with a hepatic disorder in a subject, e.g., a patient, in need thereof. In some embodiments, such provided technologies may be used to treat a neoplasia disorder e.g. a disorder or, a symptom associated with a neoplasia disorder in a subject, e.g., a patient, in need thereof. In some embodiments, such provided technologies may be used to treat a viral infection related disorder e.g. a disorder or a symptom associated with viral infection related disorder in a subject, e.g., a patient, in need thereof. In some embodiments, such provided technologies may be used to treat an alcohol misuse related disorder e.g. a disorder or a symptom associated with an alcohol misuse related disorder in a subject, e.g., a patient, in need thereof. In some embodiments, such provided technologies may be used to treat a neoplasia disorder associated with a viral infection or alcohol misuse, e.g., a disorder or a symptom associated with a neoplasia disorder that is associated with a viral infection or alcohol misuse in a subject, e.g., a patient, in need thereof.

In some embodiments, the condition treated is neoplasia. In some embodiments, the condition treated is tumorigenesis. In some embodiments, the condition treated is cancer. In some embodiments, the cancer is associated with poor prognosis. In some embodiments, the cancer is associated with MYC mis-regulation, e.g., MYC overexpression. In some embodiments, the cancer is associated with AFP mis-regulation, e.g., AFP overexpression. In some embodiments, the cancer is a breast, a hepatic, a colorectal, a lung, a pancreatic, a gastric, and/or a uterine cancer. In some embodiments, the cancer is associated with an infection, e.g., viral, e.g., bacterial. In some embodiments, the cancer is associated with alcohol abuse. In some embodiments, the cancer is hepatocarcinoma.

In some embodiments, the cancer cells are lung cancer cells, gastric, gastrointestinal, colorectal, pancreatic or hepatic cancer cells. In some embodiments, the cancer is hepatocellular carcinoma (HCC), Fibrolamellar Hepatocellular Carcinoma (FHCC), cholangiocarcinoma, Angiosarcoma, secondary liver cancer, non-small cell lung cancer (NSCLC), adenocarcinoma, small cell lung cancer (SCLC), large cell (undifferentiated) carcinoma, triple negative breast cancer, gastric adenocarcinoma, endometrial carcinoma, or pancreatic carcinoma. In some embodiments, the cancer is metastatic, advanced (non-resectable, or recurrent solid tumor types known for association with the MYC oncogene (e.g., including, but not limited to, hepatocellular carcinoma (HCC), bladder, lung, pancreatic, ovarian, uterine, endometrial, gastric, esophageal, hepatobiliary, colorectal cancer, and neuroblastoma), which has progressed on, relapsed after, are refractory to, or intolerant of standard of care or no alternative treatment exists.

In some embodiments, the cancer is HCC classified by the commonly used Barcelona Clinic Liver Cancer (BCLC) staging system. The BCLC staging system classifies HCC patients into the following categories: very early stage (0), Early Stage (A), Intermediate Stage (B), Advanced Stage (C), and Terminal Stage (D), which guides management decisions. The BCLC classification is based on the extent of the tumor burden, the severity of liver disease (Child-Turcotte-Pugh classification (“C-P Score”)) and patient's performance status.

Patients with early-stage (BCLC Stage 0 or A) are managed by surgical resection of the tumor. Transarterial therapies are considered for patients who have intermediate stage (BCLC Stage B) HCC. Systemic therapies are recommended for patients who have advanced disease (BCLC Stage C) or have intermediate stage disease (BCLC Stage B) and have progressed following transarterial therapies. First-line systemic therapies include atezolizumab plus bevacizumab, sorafenib, or lenvatinib. Potential second-line therapies for patients who progress or are intolerant of first-line therapies include sorafenib, regorafenib, lenvatinib, ramucirumab, cabozantinib, and checkpoint inhibitors (pembrolizumab or nivolumab plus ipilimumab). Accordingly, in some embodiments, the subject to be treated with a therapy described herein is a subject that has progressed after receiving, has relapsed after, is refractory to, or is intolerant of, a therapy selected from atezolizumab plus bevacizumab, sorafenib, lenvatinib, sorafenib, regorafenib, lenvatinib, ramucirumab, cabozantinib, and checkpoint inhibitors (pembrolizumab or nivolumab plus ipilimumab). The optimal regimen after progression following first-line therapy is unknown. There are very limited options for patients with Terminal (Stage D) HCC, who receive supportive care only. The BCLC staging system takes into account the following variables:
Tumor stage;
Functional status of the liver;
Physical status; and
Cancer-related symptoms.
   Five stages (0 and A through D) are identified based on the variables mentioned above.

The C-P Score is used to estimate the risk of mortality in patients with cirrhosis and consists of the following factors: Total bilirubin level, albumin, Prothrombin Time and INR, degree of ascites and the grade of hepatic encephalopathy. In some embodiments, the C-P Score is performed at patient screening, on Cycle Day 1 predose, every 6 weeks for the first year, every 3 months until disease progression, at the end of treatment/early termination visit, and as clinically indicated. In some embodiments, the C-P Score is performed frequently in accordance with clinical practice if the participant develops hepatic adverse events of special interest until the AE resolves. Hepatic encephalopathy graded according to West Haven criteria for semi-quantitative grading of Mental Status: Grade 1: Trivial lack of awareness; euphoria or anxiety; shortened attention span; impaired performance of addition or subtraction; Grade 2: Lethargy or apathy; minimal disorientation for time or place; subtle personality change; inappropriate behavior; Grade 3: Somnolence to semi-stupor, but responsive to verbal stimuli. Confusion; Gross disorientation; Grade 4: Coma (unresponsive to verbal or noxious stimuli).

In some embodiments the condition treated is a hepatic disease. In some embodiments the condition treated is associated with MYC mis-regulation, e.g., MYC overexpression. In some embodiments the condition treated is a chronic disease. In some embodiments the condition treated is a chronic liver disease. In some embodiments the condition treated is a viral infection. In some embodiments, the condition treated is an alcohol misuse associated disorder.

In some embodiments the condition treated is a pulmonary disease. In some embodiments the condition treated is associated with MYC mis-regulation, e.g., MYC overexpression. In some embodiments the condition treated is a chronic disease. In some embodiments the condition treated is a chronic pulmonary disease. In some embodiments, such provided technologies may be used to treat or reduce lung cancer growth, metastasis, drug resistance, and/or cancer stem cell (CSC) maintenance. In some embodiments, the condition treated is a carcinoma, e.g., non-small cell lung cancer (NSCLC). In some embodiments, the chronic pulmonary disease is associated with tobacco misuse.

In some embodiments, the cancer hepatocarcinoma subtype S1 (HCC S1), hepatocarcinoma subtype S2 (HCC S2), or hepatocarcinoma subtype S3 (HCC S2). In some embodiments, the HCC subtype is associated with MYC overexpression. In some embodiments, the cancer is HCC S1 or HCC S2. In some embodiments, the cancer subtype is associated with aggressive tumor and poor clinical outcome.

In some embodiments, the disclosure provides a treatment regimen that may be devised for the subject on the basis of the HCC subtype in the subject, e.g., a personalized approach to tailor the aggressiveness of treatment based on HCC subtype on a subject. In some embodiments, the disclosure provides a method of treatment using the expression repressors or expression repressor systems disclosed herein, the method comprising, identifying the HCC subtype in a patient and determine a dose or dosage and administration schedule of said expression repressors and/or expression repressor systems based on the HCC subtype identification.

Methods are described herein to deliver agents, or a composition as disclosed herein to a subject for treatment of a disorder such that the subject suffers minimal side effects or systemic toxicity in comparison to chemotherapy treatment. In some embodiments, the subject does not experience any significant side effects typically associated with chemotherapy, when treated with the agents and/or compositions described herein. In some embodiments, the subject does not experience a significant side effect including but not limited to alopecia, nausea, vomiting, poor appetite, soreness, neutropenia, anemia, thrombocytopenia, dizziness, fatigue, constipation, oral ulcers, itchy skin, peeling, nerve and muscle damage, auditory changes, weight loss, diarrhea, immunosuppression, bruising, heart damage, bleeding, liver damage, kidney damage, edema, mouth and throat sores, infertility, fibrosis, epilation, moist desquamation, mucosal dryness, vertigo and encephalopathy when treated with the agents and/or compositions described herein. In some embodiments, the subject does not show a significant loss of body weight when treated with the agents and/or compositions described herein.

The agents and compositions described herein can be administered to a subject, e.g., a mammal, e.g., in vivo, to treat or prevent a variety of disorders as described herein. This includes disorders involving cells characterized by altered expression patterns of MYC.

### Epigenetic Modification

The present disclosure is further directed, in part, to a method of epigenetically modifying a target gene, a transcription control element operably linked to a target gene, or an anchor sequence (e.g., an anchor sequence proximal to a target gene or associated with an anchor sequence-mediated conjunction operably linked to a target gene), the method comprising providing an expression repressor (or nucleic acid encoding the same) or an expression repression system (e.g., expression repressor(s)), or nucleic acid encoding the same or pharmaceutical composition comprising said an expression repressor (or nucleic acid encoding the same) or expression repression system or nucleic acid; and contacting the target gene or a transcription control element operably linked to the target gene with the expression repressor or the expression repression system, thereby epigenetically modifying the target gene, e.g., MYC or a transcription control element operably linked to the target gene, e.g., MYC.

In some embodiments, a method of epigenetically modifying a target gene, e.g., MYC or a transcription control element operably linked to a target gene, e.g., MYC comprises increasing or decreasing DNA methylation of the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises increasing or decreasing histone methylation of a histone associated with the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises decreasing histone acetylation of a histone associated with the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises increasing or decreasing histone sumoylation of a histone associated with the target gene, e.g., MYC or a transcription control element operably linked to a target gene, e.g., MYC. In some embodiments, a method of epigenetically modifying a target gene, e.g., MYC or a transcription control element operably linked to a target gene, e.g., MYC comprises increasing or decreasing histone phosphorylation of a histone associated with the target gene, e.g., MYC or a transcription control element operably linked to a target gene, e.g., MYC.

In some embodiments, a method of epigenetically modifying a target gene, e.g., MYC or a transcription control element operably linked to a target gene, e.g., MYC may decrease the level of the epigenetic modification by at least 10, 20, 30, 40, 50, 60, 70, 80, 90, or 100% (and optionally up to 100%) relative to the level of the epigenetic modification at that site in a cell not contacted by the composition or treated with the method. In some embodiments, a method of epigenetically modifying a target gene, e.g., MYC or a transcription control element operably linked to a target gene, e.g., MYC may increase the level of the epigenetic modification by at least 10, 20, 30, 40, 50, 60, 70, 80, 90, 100, 120, 140, 160, 180, 200, 300, 400, 500, 600, 700, 800, 900, or 1000% (and optionally up to 200, 300, 400, 500, 600, 700, 800, 900, 1000, or 2000%) relative to the level of the epigenetic modification at that site in a cell not contacted by the composition or treated with the method. In some embodiments, epigenetic modification of a target gene, e.g., MYC or a transcription control element operably linked to a target gene, e.g., MYC may modify the level of expression of the target gene, e.g., MYC, e.g., as described herein.

In some embodiments, an epigenetic modification produced by a method described herein persists for at least about 1 hour to about 30 days, or at least about 2 hours, 6 hours, 12 hours, 18 hours, 24 hours, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, an expression repressor, or an expression repression system for use in a method of epigenetically modifying a target gene, e.g., MYC or a transcription control element operably linked to a target gene, e.g., MYC comprises an expression repressor comprising an effector moiety that is or comprises an epigenetic modifying moiety.

For example, a effector moiety may be or comprise an epigenetic modifying moiety with DNA methyltransferase activity, and an endogenous or naturally occurring target sequence (e.g. a target gene, e.g., MYC or transcription control element) may be altered to increase its methylation (e.g., decreasing interaction of a transcription factor with a portion of target gene, e.g., MYC or transcription control element, decreasing binding of a nucleating protein to an anchor sequence, and/or disrupting or preventing an anchor sequence-mediated conjunction), or may be altered to decrease its methylation (e.g., increasing interaction of a transcription factor with a portion of a target gene, e.g., MYC or transcription control element, increasing binding of a nucleating protein to an anchor sequence, and/or promoting or increasing strength of an anchor sequence-mediated conjunction).

### Kits

The present disclosure further directed, in part, to a kit comprising an expression repressor or an expression repression system, e.g., expression repressor(s), described herein. In some embodiments, a kit comprises an expression repressor or an expression repression system (e.g., the expression repressor(s) of the expression repression system) and instructions for the use of said an expression repressor or expression repression system. In some embodiments, a kit comprises a nucleic acid encoding the expression repressor or a nucleic acid encoding the expression repression system or a component thereof (e.g., the expression repressor(s) of the expression repression system) and instructions for the use of said expression repressor (and/or said nucleic acid) and/or said expression repression system (and/or said nucleic acid). In some embodiments, a kit comprises a cell comprising a nucleic acid encoding the expression repressor or a nucleic acid encoding the expression repression system or a component thereof (e.g., the expression repressor(s) of the expression repression system) and instructions for the use of said cell, nucleic acid, and/or said expression repressor or expression repression system.

The present disclosure further provides, in part, a kit comprising: a) a container comprising a composition described herein, and b) a set of instructions comprising at least one method for modulating the expression of a target gene, e.g., MYC within a cell with said composition.

In some aspects, the kit comprises a) a container comprising a composition comprising a system comprising two expression repressors, comprising a first expression repressor comprising a first targeting moiety and optionally a first effector moiety, wherein the first expression repressor binds to a transcription regulatory element (e.g., a promoter or transcription start site (TSS)) operably linked to target gene, e.g., MYC or to a sequence proximal to the transcription regulatory element and an expression repressor comprising a second targeting moiety and optionally a second effector moiety, wherein the second expression repressor binds to an anchor sequence of an anchor sequence mediated conjunction (ASMC) comprising target gene, e.g., MYC or to a sequence proximal to the anchor sequence.

In some aspects, the kit comprises a) a container comprising a composition comprising a system comprising two expression repressors, comprising a first expression repressor comprising a first targeting moiety and optionally a first effector moiety, wherein the first expression repressor binds to a transcription regulatory element (e.g., a promoter or transcription start site (TSS)) operably linked to target gene, e.g., MYC or to a sequence proximal to the transcription regulatory element and an expression repressor comprising a second targeting moiety and optionally a second effector moiety, wherein the second expression repressor binds to a genomic locus located in a super enhancer region of a target gene, e.g., MYC.

In some embodiments the kit further comprises b) a set of instructions comprising at least one method for treating a disease or modulating, e.g., decreasing the expression of target gene, e.g., MYC within a cell with said composition. In some embodiments, the kits can optionally include a delivery vehicle for said composition (e.g., a lipid nanoparticle). The reagents may be provided suspended in the excipient and/or delivery vehicle or may be provided as a separate component which can be later combined with the excipient and/or delivery vehicle. In some embodiments, the kits may optionally contain additional therapeutics to be co-administered with the compositions to affect the desired target gene expression, e.g., MYC gene expression modulation. While the instructional materials typically comprise written or printed materials, they are not limited to such. Any medium capable of storing such instructions and communicating them to an end user is contemplated. Such media include but are not limited to electronic storage media (e.g., magnetic discs, tapes, cartridges, chips), optical media (e.g., CD ROM), and the like. Such media may include addresses to internet sites that provide such instructional materials.

In some embodiments, a kit comprises a unit dosage of an expression repressor an expression repression system, e.g., expression repressor(s), described herein, or a unit dosage of a nucleic acid, e.g., a vector, encoding an expression repression system, e.g., expression repressor(s), described herein.

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### EXAMPLES

### Example 1: Combination of Bicistronic ZF9-MQ1_ZF3-KRAB Drug Substance with Lenvatinib in HCC Cells

This study demonstrates the use of lenvatinib in combination with bicistronic ZF9-MQ1_ZF3-KRAB in HCC cells. MYC repression is known to sensitize HCC cells to established chemotherapies. Bicistronic ZF9-MQ1_ZF3-KRAB Drug substance (DS) treatment was combined with HCC standard of care treatment, lenvatinib. Lenvatinib is a multiple kinase inhibitor specifically targeting VEGFR1-3, decreasing vascular endothelial proliferation in tumors. It is currently approved as a treatment for HCC patients in the US and European Union after studies showed it is comparable to sorafenib in terms of overall survival with significant improvement on drug tolerability. HCC cells have been shown to upregulate C-MYC after Lenvatinib treatment as a potential resistance mechanism, suggesting combination treatment with C-MYC targeted DS could improve lenvatinib sensitivity. This study evaluates how bicistronic ZF9-MQ1_ZF3-KRAB alters the IC₅₀ of lenvatinib in HCC cells.

Hep 3B and SK-HEP-1 cells were treated with lenvatinib (0.004 to 25 μM using 1:3 dilutions) in combination with bicistronic ZF9-MQ1_ZF3-KRAB drug substance (DS, HA-tagged; MR-30723, SEQ ID NO: 112) formulated in SSOP (0.1 μg/mL or 0.6 μg/ml). Cell viability (Celltiter-Glo) levels were assessed 72 hours after treatment. Bliss synergy scores calculated using SyngeryFinder 2.0 were used to determine combinatorial effect.

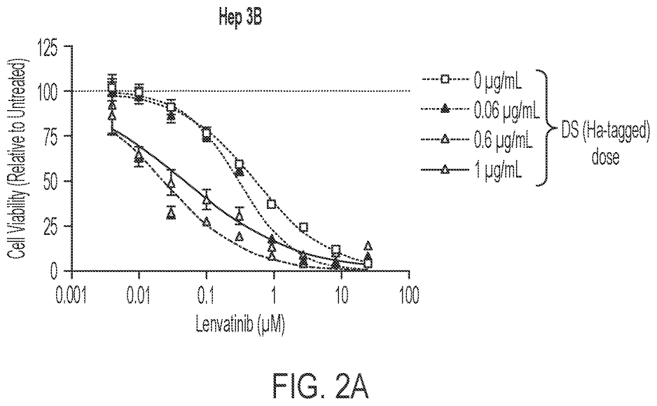

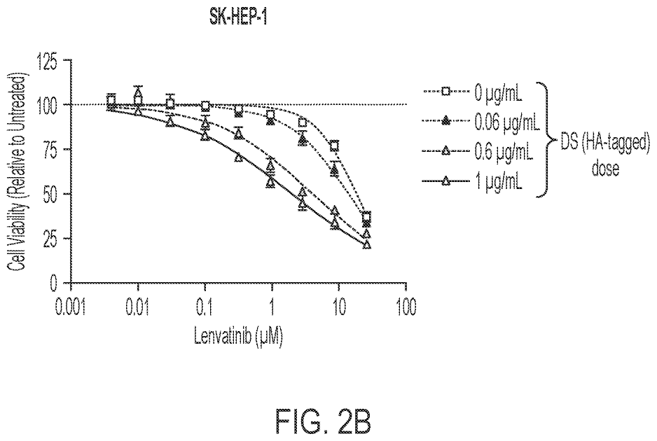

The combination of 0.6 μg/mL and 1 μg/mL DS plus lenvatinib reduced the half maximal inhibitory concentration (IC₅₀) of lenvatinib in Hep 3B from 0.533 μM to 0.023 μM and 0.048 μM, respectively. The combination of 0.6 μg/mL and 1 μg/mL DS plus lenvatinib reduced the IC₅₀ of lenvatinib in SK-HEP-1 from 17.69 μM to 3.857 μM and 1.910 μM, respectively (FIGS. 2A and 2B and Table 22). Bliss synergy scores calculated using SynergyFinder 2.0 were used to determine combinatorial effect. The degree of combination synergy, or antagonism, is quantified by comparing the observed drug combination response against the expected response, calculated using a reference model that assumes no interaction between drugs. The Bliss independence model assumes a stochastic process in which two drugs elicit their effects independently, and the expected combination effect can be calculated based on the probability of independent events. In general, if the synergy score is <−10 the interaction between two drugs is likely to be antagonistic; scores between −10 to 10 suggest the interaction between two drugs is likely to be additive: at scores >10 the interaction between two drugs is likely to be synergistic. Bliss synergy scores plus lenvatinib for Hep 3B and SK-HEP-1 were 5.37 and 12.77.

TABLE 22
bicistronic ZF9-MQ1_ZF3-KRAB in combination with lenvatinib

| Hep 3B | | | SK-HEP-1 | | |
|---|---|---|---|---|---|
| ZF9-MQ1_{—} | | | ZF9-MQ1_{—} | | |
| ZF3-KRAB | | Bliss | ZF3-KRAB | | Bliss |
| Concen- | Lenvatinib | Synergy | Concen- | Lenvatinib | Synergy |
| tration | IC₅₀ | Score | tration | IC₅₀ | Score |
| 0 μg/mL | 0.533 μM | 5.37 | 0 μg/mL | 17.69 μM | 12.77 |
| 0.06 μg/mL | 0.309 μM | Addi- | 0.1 μg/mL | 13.28 μM | Synergy |
| 0.6 μg/mL | 0.023 μM | tive | 0.6 μg/mL | 3.857 μM | |
| 1.0 μg/mL | 0.048 μM | | 1.0 μg/mL | 1.910 μM | |

Legend: Bliss Synergy Score
Antagonism: <−10
Additive: −10 to +10
Synergy: >10

### Example 2: Bicistronic ZF9-MQ1_ZF3-KRAB Increases Efficacy of AKT and ERK Inhibitors In Vitro

SK-HEP-1 cells are less sensitive to MYC repression due to their derivation from the S1 subtype of HCC. Therefore, we utilized SK-HEP-1 cells to understand possible resistance mechanisms to MYC targeted expression repressors. SK-HEP-1 cells treated with MYC targeted expression repressors revealed upregulation of AKT and ERK signaling, known cell survival pathways. We hypothesized that inhibition of these survival pathways may work effectively in combination with bicistronic ZF9-MQ1_ZF3-KRAB.

SK-HEP-1 cells were treated with bicistronic ZF9-MQ1_ZF3-KRAB and AKT inhibitor MK-2206 or ERK inhibitor ulixertinib. Cells were plated in 96 well plates at 10,000 cells per well in EMEM media with fetal bovine serum (FBS). Cells were then treated independently with AKT inhibitor MK-2206 or ERK inhibitor ulixertinib at multiple doses. The dose range for MK-2206 and ulixertinib is between 0.01 and 25 μM, with each dose being serially diluted 1:2. Lipid mix carrying bicistronic ZF9-MQ1_ZF3-KRAB (MR-30723 in SSOP, SEQ ID NO: 112) was then added to a subset of the wells at a dose of 0.6 μg/mL. A negative control non-coding mRNA (SNC=short non-coding) was also added to a subset of wells at 0.6 μg/mL. A subset of cells was also left untreated a control for the effect of the inhibitors alone. Cells were then incubated for 72 hrs at 37 C, 5% CO2. Following treatment, cells were lysed with the Cell Titer Glo reagent and luminescence was quantified using the Glo Max where high luminescence values indicates high cell viability and low values indicate low cell viability. Relative cell viability was calculated by averaging the untreated values and dividing each experimental luciferase value by that average.

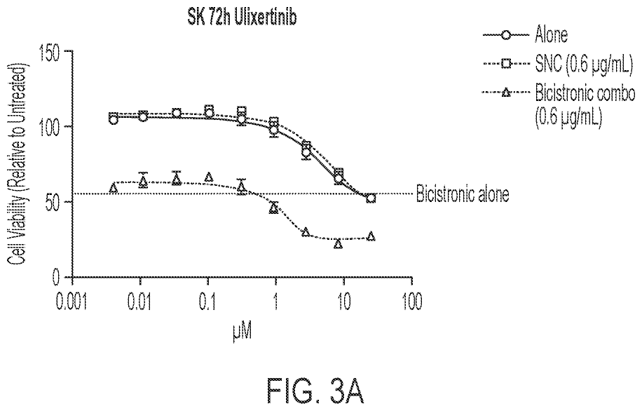

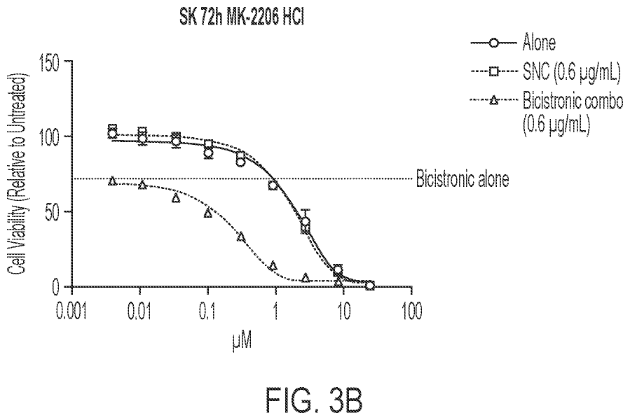

The combination of bicistronic ZF9-MQ1_ZF3-KRAB with ERK inhibitor ulixertinib revealed a combinatorial efficacy between these 2 agents with bicistronic ZF9-MQ1_ZF3-KRAB decreasing the IC₅₀ of ulixertinib (FIG. 3A). Similarly, combination of bicistronic ZF9-MQ1_ZF3-KRAB with AKT inhibitor MK-2206 revealed a combinatorial efficacy with the IC₅₀ of MK-2206 being decreased when combined with bicistronic ZF9-MQ1_ZF3-KRAB (FIG. 3B).

### Example 3: Bicistronic ZF9-MQ1_ZF3-KRAB in Combination with Osimertinib or Trametinib, In Vitro

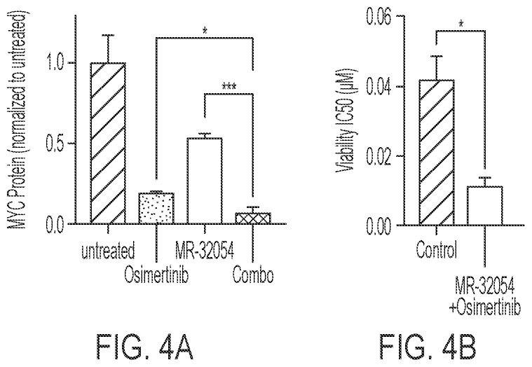

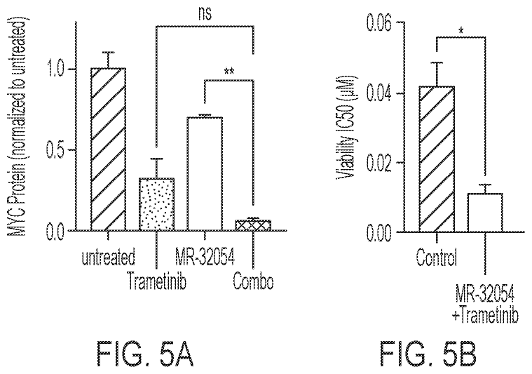

Myc protein was assessed in PC9 cells 48 h after treatment with PBS (untreated), 1 μM osimertinib, 0.5 μg/ml MR-32054 (bicistronic ZF9-MQ1_ZF3-KRAB mRNA) formulated in SSOP LNPs, or combination of 1 μM osimertinib plus 0.5 μg/ml MR-32054 formulated in LNPs (FIG. 4A). In addition, MYC protein was assessed in H1975 cells 48 h after treatment with PBS (untreated), 1 μM trametinib, 0.5 μg/ml MR-32054 (bicistronic ZF9-MQ1_ZF3-KRAB mRNA), or a combination of 1 μM trametinib plus 0.5 μg/ml MR-32054 (bicistronic ZF9-MQ1_ZF3-KRAB mRNA) formulated in SSOP LNPs (FIG. 5A). Cell viability was assessed 72 h after treatment with varying concentration of osimertinib or trametinib in combination with 1 μg/ml of control (short non-coding) or MR-32054 (bicistronic ZF9-MQ1_ZF3-KRAB mRNA) in PC9 and H1975 cells, respectively (FIG. 4B and FIG. 5B, respectively).

The CellTiter Glo (Promega) assay was used according to manufacturer's instructions to assess cell viability. IC₅₀ was analyzed using GraphPad Prism Western blot to assess MYC protein levels: β-actin antibody was stained with a secondary antibody tagged with fluorophore emitting at 594 nm wavelength and MYC antibody (Abcam) was detected with secondary antibody tagged with fluorophore emitting at a wavelength of 488 nm. Odyssey CLx Imaging System (LICOR) using near-infrared (NIR) fluorescence was used to capture and quantify the protein images.

These data show that in EGFR mutant PC9 cells, combination treatment of osimertinib and bicistronic ZF9-MQ1_ZF3-KRAB (MR-32054) 1) significantly reduces Myc protein levels compared to osimertinib or bicistronic ZF9-MQ1_ZF3-KRAB (MR-32054) alone (93% in combo vs. 80% or 47% in osimertinib or bicistronic ZF9-MQ1_ZF3-KRAB (MR-32054) alone treated, respectively) (FIG. 4A), and 2) significantly enhances reduction of viability compared to osimertinib monotherapy (IC₅₀ of combo: 0.011 μM vs. IC₅₀ of osimertinib alone: 0.42 μM) (FIG. 4B). These data also show that in EGFR mutant H1975 cells, combination treatment of trametinib and MR-32054, 1) significantly reduces Myc protein levels compared to bicistronic ZF9-MQ1_ZF3-KRAB (MR-32054) alone (93% in combo treated vs. 30% in bicistronic ZF9-MQ1_ZF3-KRAB alone treated, respectively) (FIG. 5A), and 2) significantly enhances growth inhibition compared to trametinib monotherapy (IC₅₀ of combo: 1.5 μM vs. IC₅₀ of trametinib alone: 0.14 μM) (FIG. 5B).

### Example 4: MYC Overexpression is Associated with Decreased Survival Probability with Osimertinib Treatment in NSCLC

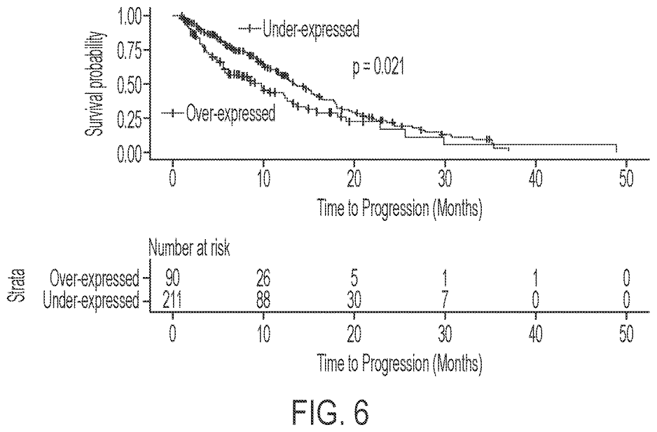

Analysis of human patient NSCLC tumor data from Tempus labs. Biopsies from tumor samples were analyzed for MYC mRNA levels and compared to data reporting on time to progression. FIG. 6 shows a decrease in survival probability of patients with over-expressed MYC compared to under-expressed. These results suggest that patients with higher MYC mRNA levels have poorer survival on Osimertinib (statistically significant difference).

### Example 5: Bicistronic ZF9-MQ1_ZF3-KRAB Dose Monotherapy

Participants will be considered evaluable for a dose limiting toxicity (DLT) if they received at least 1 dose of bicistronic ZF9-MQ1_ZF3-KRAB and completed the 28-day DLT observation period (unless the participant does not complete the DLT period because he or she experiences a Grade 5 toxicity prior to the end of the DLT period, in which case they will remain evaluable for DLT).

After providing informed consent, participants will undergo screening assessments (up to 28 days prior to enrollment).

Once enrolled in the study, Bicistronic ZF9-MQ1_ZF3-KRAB will be administered via intravenous infusion once every 2 weeks (Day 1 and Day 15 of each 28-day cycle). Bicistronic ZF9-MQ1_ZF3-KRAB will be an LNP aqueous emulsion comprising a unit dose of 5 mg/5 ml (1 mg/mL concentration) for intravenous administration. For the first dose level in Part 1, premedication will be administered prior to each dose of bicistronic ZF9-MQ1_ZF3-KRAB at least 60 minutes prior to the start of infusion including, but not limited to, oral acetaminophen (e.g., 500 mg), intravenous H1 blocker (e.g., diphenhydramine, e.g., 50 mg, or equivalent), and intravenous corticosteroids (e.g., dexamethasone, e.g., 10 mg, or equivalent). For dose level 2 and beyond, the same premedication is required, with the exception of intravenous corticosteroids (e.g., dexamethasone, e.g., 10 mg, or equivalent), which may be added at the Investigator's discretion after discussion with the Sponsor Medical Monitor.

The first dose of bicistronic ZF9-MQ1_ZF3-KRAB will be administered in the hospital setting during dose escalation. A sentinel dosing strategy will be used during dose escalation. Dose escalation will begin at 0.02 mg/kg and is planned to proceed to the provisional dose levels of 0.05 mg/kg, 0.08 mg/kg, 0.125 mg/kg, and 0.15 mg/kg (approximately 5 dose levels total).

After the second cycle is completed, individual participants may be considered for treatment at a dose of bicistronic ZF9-MQ1_ZF3-KRAB higher than the dose to which they were assigned. The participant must not have experienced any Common Terminology Criteria for Adverse Events (CTCAE) Grade ≥2 adverse event at the initial dose level assigned for at least 2 cycles of therapy in order to be treated at a higher dose. Intermediate dose levels within the proposed range may be used based upon pharmacokinetics, pharmacodynamics, clinical observations, and/or safety observations. Any intermediate dose levels will be lower than the next planned dose escalation level. The MTD is the dose level at which ≤1/6 participants experience a DLT, or the highest dose level tested if DLTs are not observed at any of the dose levels.

Tumor imaging will be performed by computed tomography (CT) scan with contrast or magnetic resonance imaging (MRI) at baseline and every 6 weeks (±1 week) for the first year, then every 3 months (±3 weeks) until disease progression. In addition, if disease progression is suspected due to clinical or symptomatic deterioration, tumor imaging will be performed to confirm disease progression. Responses will be assessed according to modified Response Evaluation Criteria in Solid Tumors (mRECIST) for HCC and RECIST 1.1 for non-HCC solid tumor participants based on Investigator assessment.

Bicistronic ZF9-MQ1_ZF3-KRAB will be administered until disease progression, unacceptable toxicity (including DLT), withdrawal of consent, Investigator decision to withdraw the participant, death, start of subsequent treatment, or up to 2 years, whichever comes first. A participant may continue study treatment after confirmation of disease progression as long as the participant is deriving clinical benefit, as judged by the Investigator.

Participants who achieve a complete response (CR) by 2 tumor imaging assessments conducted at least 4 weeks apart may discontinue study treatment at the Investigator's discretion after confirmation of the CR.

Disease status for participants who discontinue study drugs due to reasons other than disease progression will continue to be monitored every 12 weeks until start of new anticancer therapy, disease progression, withdrawal of consent, death or end of study, whichever comes first. Participants will be followed for OS until death, withdrawal of consent, up to 2 years, or end of the study, whichever comes first.

Safety assessments will include the incidence, severity of adverse events (including SAEs), laboratory abnormalities, electrocardiogram (ECG) changes, cytokines, C-reactive protein (CRP), and complement. Plasma samples will be collected to assess PK. Blood samples and tumor biopsy samples (if available) may be collected.

Adverse events will be graded according to the National Cancer Institute (NCI) CTCAE version 5.0. Progression of the underlying disease will not be considered an AE unless considered related to the study drug by the Investigator. Adverse events will be collected from the time of signing informed consent until 30 days after last administration of study drug or initiation of new anticancer therapy, whichever occurs first. After stopping study drug, adverse events that are >Grade 1 will be followed until resolution of the AE to ≤Grade 1 or baseline or until the start of new anticancer therapy, whichever occurs first.

### Example 6. Combination of Bicistronic ZF9-MQ1_ZF3-KRAB and a Kinase Inhibitor for Treatment of a Subject

Participants will receive bicistronic ZF9-MQ1_ZF3-KRAB via intravenous infusion every 2 weeks (Day 1 and Day 15 of each 28 days/4-week cycle). Lenvatinib will be taken starting on Cycle 1 Day 1. On the same days bicistronic ZF9-MQ1_ZF3-KRAB is administered, lenvatinib is administered at least 1 hour and preferably less than 3 hours after completion of the bicistronic ZF9-MQ1_ZF3-KRAB infusion.

Lenvatinib should be taken orally once daily, with or without food, at the same time each day. Lenvatinib will be administered at 12 mg (subject ≥60 kg) or 8 mg (subject <60 kg), or the local approved dose. On days when bicistronic ZF9-MQ1_ZF3-KRAB and lenvatinib are both administered, lenvatinib will be taken at least 1 hour (up to 24 hours) after completion of the bicistronic ZF9-MQ1_ZF3-KRAB infusion.

Similar to Example 5, for the first dose level in Part 2, premedication will be administered prior to each dose of bicistronic ZF9-MQ1_ZF3-KRAB at least 60 minutes prior to the start of infusion including, but not limited to, oral acetaminophen (e.g., 500 mg), intravenous H1 blocker (e.g., diphenhydramine, e.g., 50 mg, or equivalent), and intravenous corticosteroids (e.g., dexamethasone, e.g., 10 mg, or equivalent). For dose level 2 and beyond, the same premedication is required, with the exception of intravenous corticosteroids (e.g., dexamethasone, e.g., 10 mg, or equivalent), which may be added at the Investigator's discretion after discussion with the Sponsor Medical Monitor.

During the safety run-in, the first dose of bicistronic ZF9-MQ1_ZF3-KRAB will be administered in the hospital and participants will be observed for at least 24 hours after their initial infusion and then discharged home if clinically stable at the Investigator's discretion. Subsequent doses (post-initial dose) for a given participant during the safety run-in and all doses during dose expansion may be administered as an outpatient in an infusion center, and the participant may be discharged home if clinically stable during and after the infusion. Enrollment and treatment of all 3 participants in the safety run-in may occur simultaneously.

Tumor imaging will be performed by CT scan with contrast or MRI every 6 weeks (±1 week) for the first year, then every 3 months (±3 weeks) until disease progression. In addition, if disease progression is suspected due to clinical or symptomatic deterioration, tumor imaging will be performed to confirm disease progression. Administration of study drugs will continue until disease progression, unacceptable toxicity (including DLT), withdrawal of consent, Investigator decision to withdraw participant, death, start of subsequent treatment, or up to 2 years, whichever comes first. Participants who achieve a CR by 2 tumor imaging assessments conducted at least 4 weeks apart may discontinue study treatment at the Investigator's discretion after confirmation of the CR. A participant may continue study treatment after confirmation of disease progression as long as the participant is deriving clinical benefit, as judged by the Investigator.

Disease status for participants who discontinue study drugs due to reasons other than disease progression will continue to be monitored every 12 weeks (±3 weeks) until start of new anticancer therapy, disease progression, withdrawal of consent, death or end of study, whichever comes first. Participants will be followed for OS until death, withdrawal of consent, up to 2 years, or end of the study, whichever comes first.

Adverse events will be collected from the time of signing informed consent until 30 days after the last administration of study drugs or initiation of new anticancer therapy, whichever comes first.

### Example 7. In Vitro Generated Osimertinib Resistant H1975 Cell Lines are Resistant to Osimertinib and Retain NSCLC Expression Repressor Sensitivity

This example demonstrates the use of expression repressors in an osimertinib resistant model for cancer.

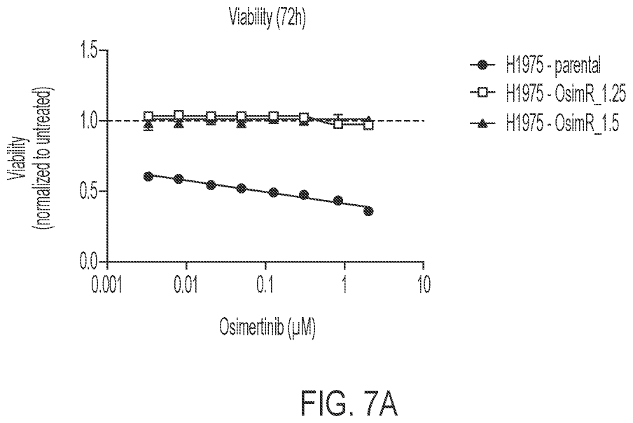

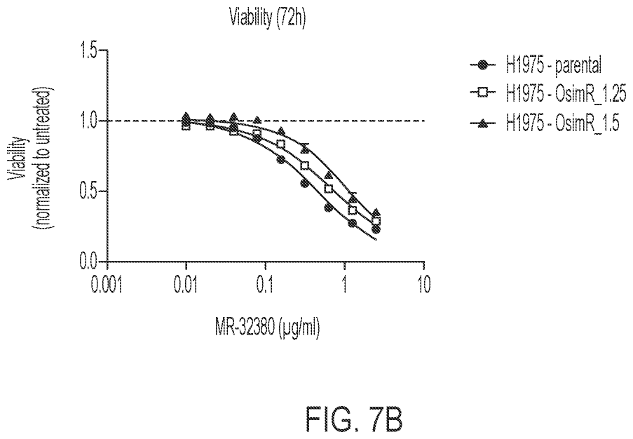

In this example, cell viability was assessed in H1975 parental cells and two osimertinib-resistant cell lines derived from H1975 (OsimR_1.25 and OsimR_1.5), 72 hours after treatment with increasing doses of Osimertinib (FIG. 7A) or NSCLC expression repressor (MR-32380; SEQ ID NO: 208) formulated in SSOP (FIG. 7B), as described herein.

Cell viability was assessed using the CellTiter Glo (Promega) assay used according to manufacturer's instructions.

These data show that osimertinib resistant H1975 cell lines are resistant to osimertinib and retain NSCLC expression repressor (e.g., MR-32380, SEQ ID NO: 208) sensitivity.

### Example 8. MYC Protein Levels are not Affected by Osimertinib, but Reduced by NSCLC Expression Repressor in Osimertinib Resistant H1975 Cell Lines

This example demonstrates the use of expression repressors in reducing MYC protein levels in an osimertinib resistant model for cancer.

MYC protein was assessed in H1975 parental cells and the two osimertinib-resistant cell lines derived from H1975 (OsimR_1.25 and OsimR_1.5), 48 hours after treatment with vehicle, 1 μM osimertinib or 1 μg/ml of NSCLC expression repressor (MR-32380) formulated in SSOP.

MYC protein levels were assess using a JESS system (Protein Simple). A 12-230 kDa pre-filled plate (Protein Simple) was loaded with protein lysate, antibody mix (MYC: Cell signaling technologies, actin: Cell signaling technologies, Goat anti-rabbit of mouse secondary antibodies conjugated with HRP, or fluorescent marker), and luminol master mix. The loaded plate and capillary cartridge (Protein Simple) were then transferred to the JESS system (Protein Simple) for imaging and quantification following manufacturer's protocol.

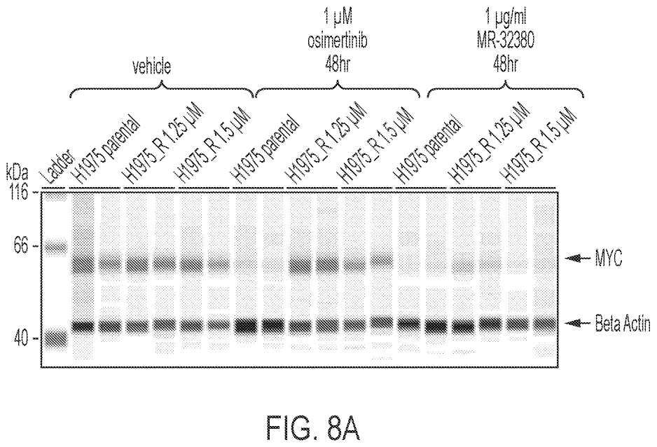

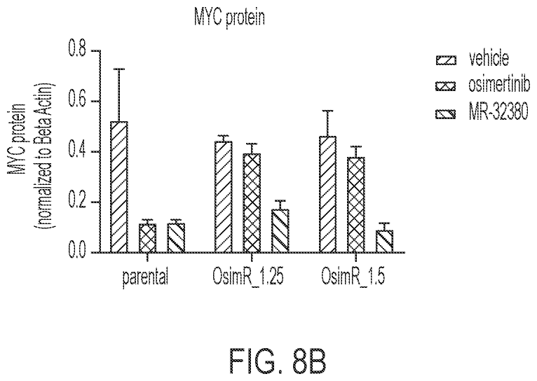

These data show that after 48 hours of 1 μM osimertinib treatment decreased MYC protein level in H1975 parental cells, while MYC protein levels were not affected by the same treatment in osimertinib resistant H1975 cells (FIGS. 8A and 8B). These data also shows that 48 hours of 1 μg/ml NSCLC OEC treatment decreased MYC protein expression in both H1975 parental cells and osimertinib-resistant H1975 cells.

### Example 9. NSCLC Expression Repressor Combination Treatment with Osimertinib Enhances MYC Protein Downregulation Compared to Monotherapy in PC9 Cells with Activating EGFR Mutation

MYC protein expression was assessed in PC9 cells 48 h after treatment with PBS (untreated), 0.1 μM or 1 μM osimertinib, 0.5 μg/ml MR-32054 (bicistronic ZF9-MQ1_ZF3-KRAB, mRNA SEQ ID NO: 196), or combination of osimertinib and MR-32054.

MYC protein levels were assess using a Western blot. β-actin antibody was stained with a secondary antibody tagged with fluorophore emitting at 594 nm wavelength and MYC antibody (Abcam) was detected with secondary antibody tagged with fluorophore emitting at a wavelength of 488 nm. Odyssey CLx Imaging System (LICOR) using near-infrared (NIR) fluorescence was used to capture and quantify the protein images.

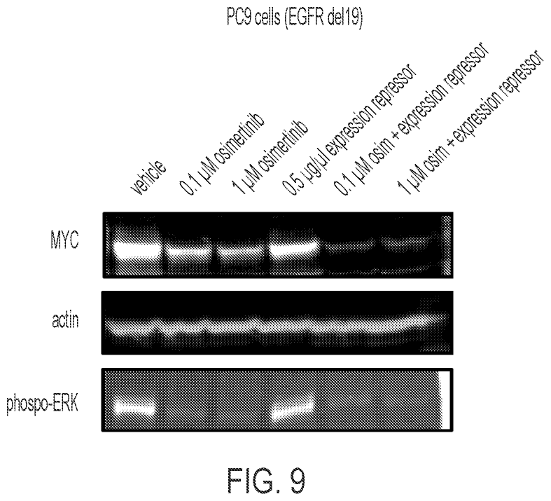

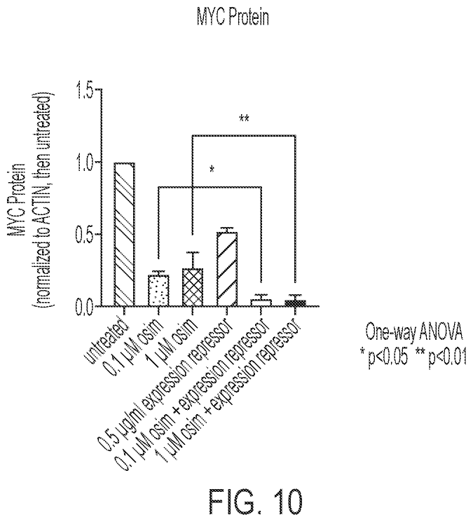

These data show that in PC9 cells with sensitizing EGFR mutation (del 19), combination treatment of osimertinib and MR-32054 showed significantly greater reduction of MYC protein level compared to osimertinib or MR-32054 alone (FIG. 9 and FIG. 10).

### Example 10: NSCLC Expression Repressor Synergizes with Osimertinib to Reduce H1975 Cell Viability

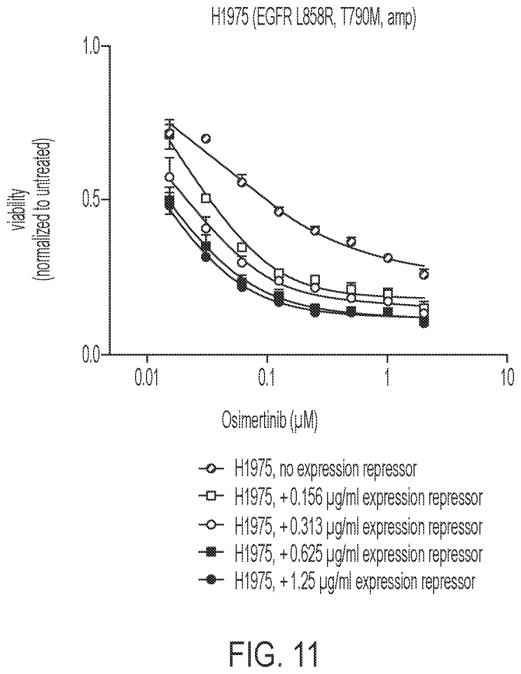

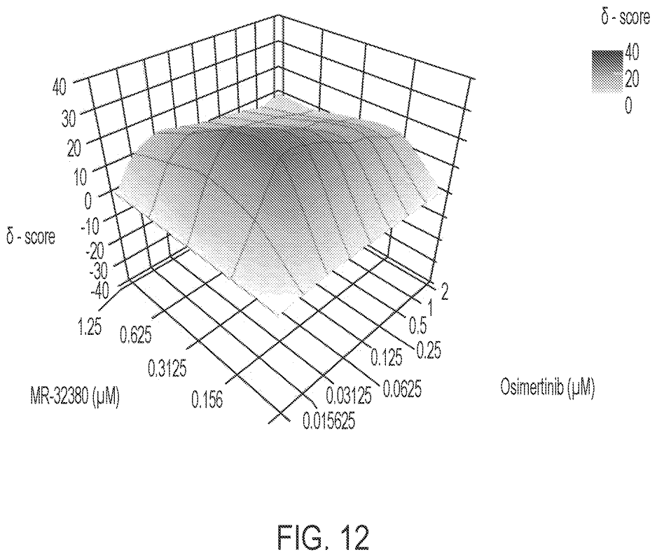

Cell viability was assessed in H1975 cells 72 hours after treatment with PBS (no MR-32380) or increasing doses of MR-32380 (0.156, 0.313, 0.625, 1.25 μg/mL) formulated in SSOP provided alone or in combination with increasing concentrations of osimertinib (FIG. 11). Bliss synergy score was also calculated (FIG. 12).

Cell viability was assessed using a CellTiter Glo assay. The CellTiter Glo (Promega) assay was used according to manufacturer's instructions to assess cell viability. Bliss synergy score was calculated using the Synergy finder (available at the world wide web address: synergyfinder.com.fimm.fi).

These data show that in H1975 cells with sensitizing EGFR mutations (L858R, T790M), combination treatment of osimertinib and MR-32380 reduces cell viability to an extent greater than either monotherapy. The Bliss synergy score of the most synergistic area of MR-32380/osimertinib combination was 27 for combinations of between 0.156-0.625 μg/mL of MR-32380 and between 0.031-0.125 μM osimertinib, indicating that the interaction between the two drugs is likely to be synergistic.

### Example 11: NSCLC Expression Repressor Synergizes with Osimertinib to Reduce PC-9 Cell Viability

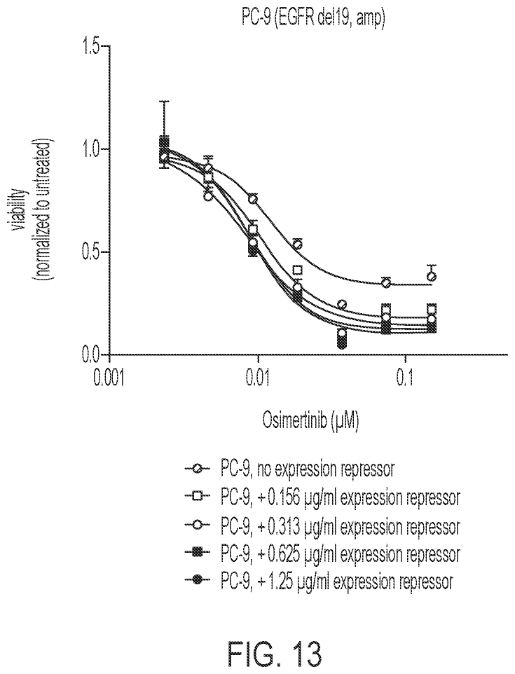

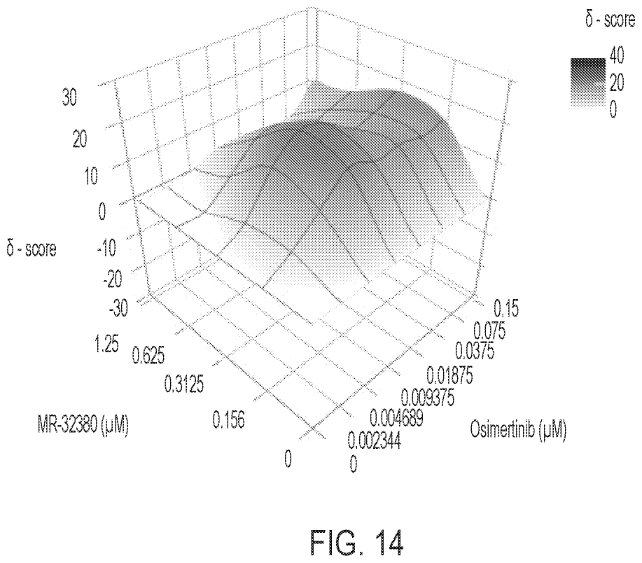

Cell viability was assessed in PC9 cells 72 hours after treatment with PBS (no OEC) or increasing doses of MR-32380 (0.156, 0.313, 0.625, 1.25 μg/mL) formulated in SSOP provided alone or in combination with increasing concentrations of osimertinib (FIG. 13). Bliss synergy score was also calculated (FIG. 14).

Cell viability was assessed using a CellTiter Glo assay, The CellTiter Glo (Promega) assay was used according to manufacturer's instructions to assess cell viability. Bliss synergy score was calculated using the Synergy finder (available at the world wide web address: synergyfinder.com.fimm.fi).

These data show that in PC9 cells with sensitizing EGFR mutation (del 19), combination treatment of osimertinib and MR-32380 reduces cell viability to an extent greater than either monotherapy. The Bliss synergy score of the most synergistic area of MR-32380/osimertinib combination was 16 for certain concentrations as indicated in FIG. 14, indicating that the interaction between the two drugs is likely to be synergistic.

### Example 12: The Combination of Expression Repressors Plus Osimertinib Shows Benefit and Tolerability in Reducing Tumor Burden of H1975 Subcutaneous Tumor Model with Sensitizing EGFR Mutations (L858R, T790M)

In this example, the ability of MR-32380 to reduce in vivo H1975 EGFR sensitized (L858, T790M) tumor burden in combination with osimertinib was tested.

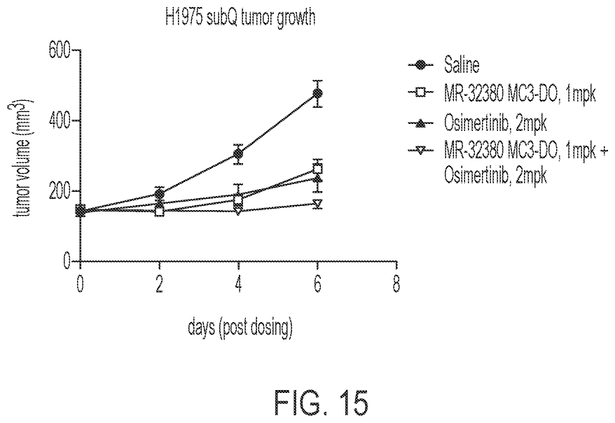

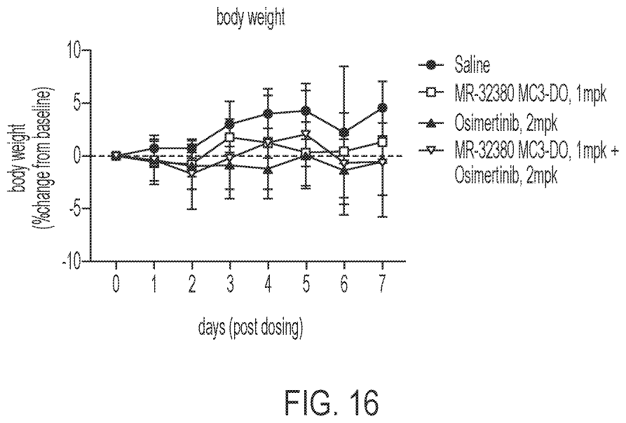

NUDE mice bearing subcutaneous H1975 tumors were treated with Saline (vehicle), 1 mg/kg of MR-32380 (formulated in MC3 LNP), 2 mg/kg of osimertinib, or a combination of 1 mg/kg MR-32380 and 2 mg/kg osimertinib. Tumor growth was measured throughout the experiment (FIG. 15). Saline and MR-32380 were administered intravenously, and osimertinib was administered orally. Mouse body weights were also measured on the day of dosing of MR-32380 and daily post dosing (FIG. 16).

Saline and MR-32380 were dosed Q5D and osimertinib was administered every day.

The length and width of tumors were measured three times weekly. Tumor volumes were calculated as width²×length/2. Body weights were measured daily.

These results show that either 1 mg/kg of MR-32380 or 2 mg/kg of osimertinib treatment alone inhibit tumor growth in the H1975 subcutaneous tumor model with sensitizing EGFR mutations (L858R, T790M), and combination treatment of the two drugs reduces tumor growth to an extent greater than either monotherapy.

### EQUIVALENTS

It is to be understood that while the invention has been described in conjunction with the detailed description thereof, the foregoing description is intended to illustrate and not limit the scope of the invention, which is defined by the scope of the appended claims. Some aspects, advantages, and modifications are within the scope of the following claims.

We claim:

1. A method of treating a cancer in a subject in need thereof, the method comprising administering to the subject:
(1) a nucleic acid (e.g., RNA, e.g., mRNA) encoding an expression repressor, wherein the expression repressor comprises:
(a) a targeting moiety that binds to a MYC promoter, and
(b) optionally, an effector moiety,
wherein the expression repressor is capable of decreasing expression of MYC; and
(2) a compound having the general structure of Formula (I): 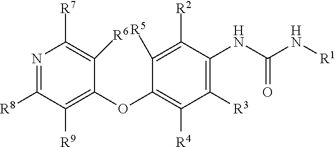
wherein:
R¹ is alkyl, cycloalkyl, or aryl;
R² is halo, alkyl, or H;
R³, R⁴, R⁵, R⁶, R⁷, are each independently H or alkyl;
R⁸ and R⁹ together form an optionally substituted aromatic ring;
or a pharmaceutically acceptable salt, tautomer or stereoisomer thereof.

2. A method of treating a cancer in a subject in need thereof, the method comprising administering to the subject:
(1) a nucleic acid (e.g., RNA, e.g., mRNA) encoding an expression repressor, wherein the expression repressor comprises:
(a) a first targeting moiety that binds a genomic locus comprising at least 16, 17, 18, 19, or 20 nucleotides of the sequence of any of SEQ ID NOs: 83, 2, 3, 75-86, 97-107, 109, 110, 190-192, or 199-202, and
(b) optionally, a first effector moiety,
wherein the expression repressor is capable of decreasing expression of MYC; and
(2) a compound having the general structure of Formula (I): 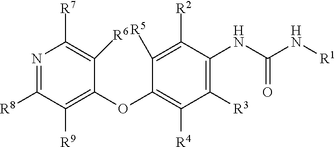
wherein:
R¹ is alkyl, cycloalkyl, or aryl;
R² is halo, alkyl, or H;
R³, R⁴, R⁵, R⁶, R⁷, are each independently H or alkyl;
R⁸ and R⁹ together form an optionally substituted aromatic ring;
or a pharmaceutically acceptable salt, tautomer or stereoisomer thereof.

3. The method of claim 1 or 2, wherein R¹ is alkyl or cycloalkyl.

4. The method of any of the preceding claims, wherein R¹=cyclopropane.

5. The method of any of the preceding claims, wherein R² is halo.

6. The method of any of the preceding claims, wherein R²=Cl.

7. The method of any of the preceding claims, wherein R³, R⁴, R⁵, R⁶, R⁷, are each H.

8. The method of any of the preceding claims, wherein the substituted aromatic ring is a substituted phenyl ring.

9. The method of any of the preceding claims, wherein the compound of Formula (I) comprises: 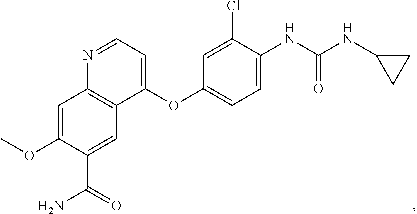 [SMILES: COc1cc2nccc(Oc3ccc(NC(=O)NC4CC4)c(Cl)c3)c2cc1C(N)=O]
or a pharmaceutically acceptable salt, tautomer or stereoisomer thereof.

10. The method of any of the preceding claims, wherein the compound of Formula (I) is administered orally.

11. The method of any of the preceding claims, wherein the compound of Formula (I) is administered at a dose of 6-10 (e.g., about 8) or 10-14 (e.g., about 12) mg per day.

12. A method of treating a cancer in a subject in need thereof, the method comprising administering to the subject:
(1) a nucleic acid (e.g., RNA, e.g., mRNA) encoding an expression repressor, wherein the expression repressor comprises:
(a) a targeting moiety that binds to a MYC promoter, and
(b) optionally, an effector moiety,
wherein the expression repressor is capable of decreasing expression of MYC; and
(2) a compound having the general structure of Formula (II): 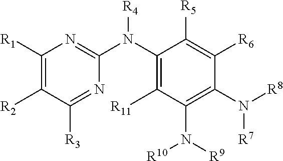
wherein R₁ is: 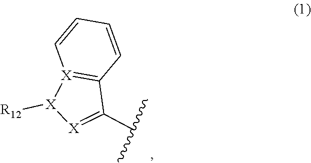
wherein X=C or N, given that one or two Xs are Ns; and R₁₂ is an electron pair, H or methyl;
(2) halogen; or
(3) methyl halogen;
R₂ is H or methyl;
R₃ is H or methyl;
R₄ is H or methyl;
R₅ is —OH or —O-methyl;
R₆ is H or methyl;
R₇ is H or methyl;
R₈ is —(CH₂)ₙ—N(R₁₃)₂, wherein n=2-6, and R₁₃=H or methyl;
R₉ is H or methyl;
R₁₀ is —(CO)—R₁₄, wherein R₁₄ is alkyl or alkene; and
R₁₁ is H or methyl;
or a pharmaceutically acceptable salt, tautomer or stereoisomer thereof.

13. A method of treating a cancer in a subject in need thereof, the method comprising administering to the subject:
(1) a nucleic acid (e.g., RNA, e.g., mRNA) encoding an expression repressor, wherein the expression repressor comprises:
(a) a first targeting moiety that binds a genomic locus comprising at least 16, 17, 18, 19, or 20 nucleotides of the sequence of any of SEQ ID NOs: 83, 2, 3, 75-86, 97-107, 109, 110, 190-192, or 199-202, and
(b) optionally, a first effector moiety,
wherein the expression repressor is capable of decreasing expression of MYC; and
(2) a compound having the general structure of Formula (II): 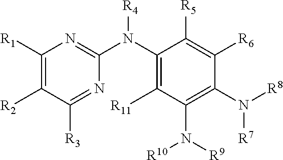
wherein R₁ is: 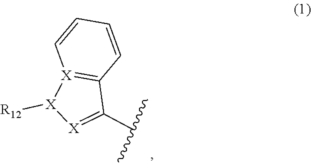
wherein X=C or N, given that one or two Xs are Ns; and R₁₂ is an electron pair, H or methyl;
(2) halogen; or
(3) methyl halogen;
R₂ is H or methyl;
R₃ is H or methyl;
R₄ is H or methyl;
R₅ is —OH or —O-methyl;
R₆ is H or methyl;
R₇ is H or methyl;
R₈ is —(CH₂)ₙ—N(R₁₃)₂, wherein n=2-6, and R₁₃=H or methyl;
R₉ is H or methyl;
R₁₀ is —(CO)—R₁₄, wherein R₁₄ is alkyl or alkene; and
R₁₁ is H or methyl;
or a pharmaceutically acceptable salt, tautomer or stereoisomer thereof.

14. The method of claim 12 or 13, wherein R₁ is: 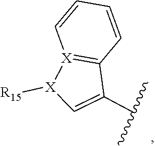
wherein X=C or N, given that one or two Xs are N; and R₁₅ is an electron pair, H or methyl.

15. The method of any of claims 12-14, wherein R₁ is:

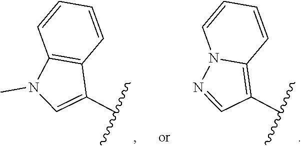 [SMILES: *c1cn(C)c2ccccc12 ; *c1cnn2ccccc12]

16. The method of any of claims 12-15, wherein R₁ is:

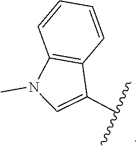 [SMILES: *c1cn(C)c2ccccc12]

17. The method of any of claims 12-16, wherein:
R₂ is H;
R₃ is H;
R₄ is H;
R₅ is —O-methyl;
R₆ is H;
R₇ is methyl;
R₈ is —(CH₂)₂—N(methyl)₂, wherein n=2-6, and R₁₃=H or methyl;
R₉ is H;
R₁ is —(CO)-ethylene; and
R₁₁ is H.

18. The method of any of claims 12-17, wherein Formula (II) is:

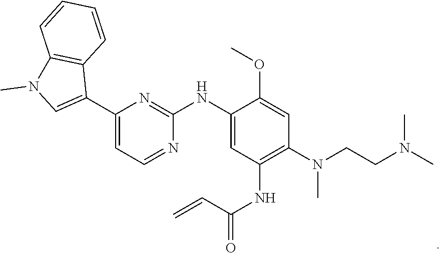

19. The method of any of claims 12-18, wherein the compound of Formula (II) is administered orally.

20. The method of any of claims 12-19, wherein the compound of Formula (II) is administered at a dose of 40-100 (e.g., about 80) mg per day.

21. A method of treating a cancer in a subject in need thereof, the method comprising administering to the subject:
(I) a nucleic acid (e.g., RNA, e.g., mRNA) encoding an expression repressor, wherein the expression repressor comprises:
(a) a targeting moiety that binds to a MYC promoter, and
(b) optionally, an effector moiety,
wherein the expression repressor is capable of decreasing expression of MYC; and
(II) an ERK inhibitor (e.g., ulixertinib), an AKT inhibitor (e.g., MK-2206), or a MEK inhibitor (e.g., trametinib).

22. A method of treating a cancer in a subject in need thereof, the method comprising administering to the subject:
(I) a nucleic acid (e.g., RNA, e.g., mRNA) encoding an expression repressor, wherein the expression repressor comprises:
(a) a first targeting moiety that binds a genomic locus comprising at least 16, 17, 18, 19, or 20 nucleotides of the sequence of any of SEQ ID NOs: 83, 2, 3, 75-86, 97-107, 109, 110, 190-192, or 199-202, and
(b) optionally, a first effector moiety,
wherein the expression repressor is capable of decreasing expression of MYC; and
(II) an ERK inhibitor (e.g., ulixertinib), an AKT inhibitor (e.g., MK-2206), or a MEK inhibitor (e.g., trametinib).

23. A method of treating a cancer in a subject in need thereof, the method comprising administering to the subject:
a nucleic acid (e.g., RNA, e.g., mRNA) encoding an expression repressor, wherein the expression repressor comprises:
(a) a targeting moiety that binds to a MYC promoter, and
(b) optionally, an effector moiety,
wherein the expression repressor is capable of decreasing expression of MYC;
wherein the cancer is resistant to osimertinib.

24. A method of treating a cancer in a subject in need thereof, the method comprising administering to the subject:
a nucleic acid (e.g., RNA, e.g., mRNA) encoding an expression repressor, wherein the expression repressor comprises:
(a) a first targeting moiety that binds a genomic locus comprising at least 16, 17, 18, 19, or 20 nucleotides of the sequence of any of SEQ ID NOs: 83, 2, 3, 75-86, 97-107, 109, 110, 190-192, or 199-202, and
(b) optionally, a first effector moiety,
wherein the cancer is resistant to osimertinib.

25. The method of any of the preceding claims wherein the cancer is a lung cancer, e.g., NSCLC.

26. The method of any of the preceding claims, wherein the cancer is a hepatocellular carcinoma (HCC), Fibrolamellar Hepatocellular Carcinoma (FHCC), Cholangiocarcinoma, Angiosarcoma, or secondary liver cancer.

27. The method of any of claims 1-26, wherein the nucleic acid comprises an RNA, e.g., an mRNA.

28. The method of any of claims 2-27, wherein:
the first targeting moiety binds a genomic locus comprising at least 16, 17, 18, 19, or 20 nucleotides of the sequence of SEQ ID NO: 83, and
the expression repressor comprises the first effector moiety, wherein the first effector moiety comprises a DNA methyltransferase.

29. The method of claim 28, wherein the first targeting moiety comprises a zinc finger domain.

30. The method of claim 28 or 29, wherein the first targeting moiety comprises an amino acid sequence according to SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

31. The method of any of claims 28-30, wherein the first effector moiety comprises MQ1 or a functional variant or fragment thereof.

32. The method of any of claims 28-31, wherein the first effector moiety comprises a sequence of SEQ ID NO: 19 or 87, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

33. The method of any of claims 28-32, wherein the first effector moiety comprises a sequence of SEQ ID NO: 129, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

34. The method of any of claim 28, wherein the RNA comprises a nucleotide sequence encoding the first targeting moiety, wherein the nucleotide sequence encoding the first targeting moiety comprises a sequence according to SEQ ID NO: 131 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

35. The method of any of claims 28-34, wherein the RNA comprises a nucleotide sequence encoding the first effector moiety, wherein the nucleotide sequence encoding the first effector moiety comprises a sequence according to SEQ ID NO: 132, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

36. The method of any of claims 28-35, wherein the RNA comprises a nucleotide sequence according to SEQ ID NO: 130, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

37. The method of any of claims 28-36, wherein the RNA further encodes a second expression repressor, wherein the second expression repressor comprises:
a second targeting moiety that binds a second genomic locus, and
a second effector moiety.

38. The method of claim 37, wherein the second targeting moiety binds a second genomic locus comprising at least 14, 15, 16, 17, 18, 19, or 20 nucleotides of the sequence of SEQ ID NO: 77.

39. The method of claim 37 or 38, wherein the second targeting moiety comprises a zinc finger domain.

40. The method of any of claims 37-39, wherein the second targeting moiety comprises an amino acid sequence according to SEQ ID NO: 7, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

41. The method of any of claims 37-40, wherein the second effector moiety comprises KRAB or a functional variant or fragment thereof.

42. The method of any of claims 37-41, wherein the second effector moiety comprises an amino acid sequence according to SEQ ID NO: 18, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

43. The method of any of claims 37-42, wherein the second expression repressor comprises an amino acid sequence according to SEQ ID NO: 24 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

44. The method of any of claims 37-43, wherein the RNA comprises a nucleotide sequence according to SEQ ID NOs: 113, 196, or 208.

45. The method of any of the preceding claims, wherein the nucleic acid is formulated in lipid nanoparticles (LNPs), wherein optionally the nucleic acid is encapsulated inside of the LNPs.

46. The method of any of the preceding claims, wherein the nucleic acid (e.g., RNA, e.g., mRNA) encoding the expression repressor is administered as an IV infusion, e.g., over 80-120 minutes, e.g., every 2 weeks.

47. The method of any of the preceding claims, wherein the nucleic acid is administered at a dose of about 0.001 mg/kg to 1.5 mg/kg or about 0.002 mg/kg to 1.5 mg/kg, e.g., administered every 1, 2, 3, 4, 5, 6 or 7 weeks.

48. The method of any of the preceding claims, wherein the nucleic acid (e.g., RNA, e.g., mRNA) encoding the expression repressor and the compound having the general structure of Formula (I) or Formula (II) are administered on different days.

49. The method of any of the preceding claims, wherein the nucleic acid (e.g., RNA, e.g., mRNA) encoding the expression repressor and the compound having the general structure of Formula (I) or Formula (II) are administered on the same day.

50. The method of claim 49, wherein the of the nucleic acid (e.g., RNA, e.g., mRNA) encoding the expression repressor is administered prior to administration of the compound having the general structure of Formula (I) or Formula (II).

51. The method of claim 49 or 50, wherein the compound having the general structure of Formula (I) or Formula (II) is administered between 1-24 hours after completion of administration of the nucleic acid (e.g., RNA, e.g., mRNA) encoding an expression repressor.
